(19) United States
(12) Patent Application Publication
(10) Pub. No.: US 2022/0265691 A1
(43) Pub. Date: Aug. 25, 2022

(54) METHODS FOR USE OF GENE EXPRESSION AS AN INDICATOR OF E-SELECTIN INHIBITOR EFFICACY AND CLINICAL OUTCOME FOR MULTIPLE TUMOR TYPES

(71) Applicant: GLYCOMIMETICS, INC., Rockville, MD (US)

(72) Inventors: John L. MAGNANI, Gaithersburg, MD (US); William E. FOGLER, Lutherville-Timonium, MD (US); Helen M. THACKRAY, Bethesda, MD (US); Eric J. FELDMAN, New York, NY (US); David STEWART, Eldersburg, MD (US)

(73) Assignee: GLYCOMIMETICS, INC., Rockville, MD (US)

(21) Appl. No.: 17/626,399

(22) PCT Filed: Jul. 12, 2020
(86) PCT No.: PCT/US2020/041740

§ 371 Date: Jan. 11, 2022

Related U.S. Application Data
(60) Provisional application No. 62/873,634, filed on Jul. 12, 2019.
(60) Provisional application No. 62/881,312, filed on Jul. 31, 2019.
(60) Provisional application No. 62/898,530, filed on Sep. 10, 2019.
(60) Provisional application No. 62/914,812, filed on Oct. 14, 2019.
(60) Provisional application No. 62/944,343, filed on Dec. 5, 2019.
(60) Provisional application No. 63/032,683, filed on May 31, 2020.

(51) Int. Cl. A61K 31/7034; C12Q 1/6886; G01N 33/574; A61P 35/00

(52) U.S. Cl. CPC A61K 31/7034; C12Q 1/6886; G01N 33/574; A61P 35/00; G01N 2800/52; C12Q 2600/158; C12Q 2600/156; G01N 2333/70564; C12Q 2600/106

(57) ABSTRACT

Cancer patients that express high levels of the E-selectin ligand (sialyl Le^{a/x}) on their tumors have a poorer outcome. Interestingly, relapsed/refractory acute myeloid leukemia (AML) patients expressing high levels of sialyl Le^{x} on their blasts show the greatest therapeutic response when treated with the E-selection inhibitor compound of Formula I. Transcriptome profiling of E-selectin ligand-forming glycosylation genes showed that ST3GAL4 and FUT7 were consistently expressed in the majority of cancers evaluated. Poor survival outcomes of FLT3-mutated AML patients that express high levels of ST3GAL4 and FUT7 implicated E-selectin in this disease state. These genes may be predictive biomarkers in AML patients. Methods of treatment of cancer comprising screening AML patients for expression of genes that contribute to the synthesis of the E-selectin ligand sialyl Le^{x}, then treating those patients with an E-selection inhibitor, are disclosed.

This application claims priority to U.S. Provisional Patent Application No. 62/873,634, filed Jul. 12, 2019; U.S. Provisional Patent Application No. 62/881,312, filed Jul. 31, 2019; U.S. Provisional Patent Application No. 62/898,530, filed Sep. 10, 2019; U.S. Provisional Patent Application No. 62/914,812, filed Oct. 14, 2019; U.S. Provisional Patent Application No. 62/944,343, filed Dec. 5, 2019; and U.S. Provisional Patent Application No. 63/032,683, filed May 31, 2020; the disclosures of all of which are incorporated herein by reference in their entireties.

Selectins are a class of cell adhesion molecules that have well-characterized roles in leukocyte homing. One of these, E-selectin (endothelial selectin), is expressed by endothelial cells at sites of inflammation or injury. Recent investigations have suggested that cancer cells are immunostimulatory and interact with selectins to extravasate and metastasize.

The most common types of cancer include prostate, breast, lung, colorectal, melanoma, bladder, non-Hodgkins lymphoma, kidney, thyroid, leukemias, endometrial and pancreatic cancers based on estimated incidence data.

The cancer with the highest expected incidence is prostate cancer. The highest mortality rate is for patients who have lung cancer. Despite enormous investment of financial and human resources, cancers such as colorectal cancer remain one of the major causes of death. Colorectal cancer is the second leading cause of cancer-related deaths in the United States of cancers that affect both men and women. Over the last several years, more then 50,000 patients with colorectal cancer have died every year.

The four hematological cancers that are most common are acute lymphocytic leukemia (ALL), chronic lymphocytic leukemia (CLL), chronic myelogenous leukemia (CML) and acute myelogenous leukemia (AML). Leukemias and other cancers of the blood, bone marrow, and lymphatic system affect 10 times more adults than children. However, leukemia is one of the most common childhood cancers and 75% of childhood leukemias are ALL.

AML is a cancer of myeloid stem cells, characterized by the rapid growth of abnormal cells that build up in the bone marrow and blood and interfere with normal blood cells. Symptoms may include fatigue, shortness of breath, easy bruising and bleeding, and increased risk of infection. It is an acute form of leukemia, which can progress rapidly and is typically fatal within weeks or months if left untreated. AML is the most common leukemia in adults. Approximately 47,000 new cases are diagnosed every year and approximately 23,500 people die every year from leukemia. The 5-year survival rate for AML is 27.4%. It accounts for roughly 1.8% of cancer deaths in the United States.

The underlying mechanism of AML is believed to involve uncontrolled expansion of immature myeloid cells in the bone marrow, which results in a drop in counts of red blood cells, platelets, and normal white blood cells. Diagnosis is generally based on bone marrow aspiration and specific blood tests. AML has several subtypes for which treatments and outcomes may vary.

First-line treatment of AML consists primarily of chemotherapy with an anthracycline/cytarabine combination and is divided into two phases: induction and post-remission (or consolidation) therapy. The goal of induction therapy is to achieve a complete remission by reducing the number of leukemic cells to an undetectable level; the goal of consolidation therapy is to eliminate any residual undetectable disease and achieve a cure. The specific genetic mutations present within the cancer cells may guide therapy, as well as determine how long that person is likely to survive.

Despite advances in our understanding of the pathogenesis of AML, the short- and long-term outcomes for AML patients have remained unchanged over three decades (Roboz et al., (2012) Curr. Opin. Oncol., 24: 711-719). The median age at diagnosis is 66 years with cure rates of less than 10% and median survival of less than 1 year (Burnett et al., (2010), J. Clin. Oncol., 28: 586-595). Although 70-80% of patients younger than 60 years achieve complete remission, most eventually relapse, and overall survival is only 40-50% at 5 years (Fernandez et al., (2009) N. Engl. J. Med., 361: 1249-1259; Mandelli et al., (2009) J. Clin. Oncol., 27: 5397-5403; Ravandi et al., (2006) Clin. Can. Res., 12(2): 340-344). Relapse is thought to occur due to leukemic stem cells that escape initial induction therapy and drive reoccurrence of AML (Dean et al., (2005) Nat. Rev. Cancer, 5(4): 275-294; Guan et al., (2003) Blood, 101(8): 3142; and Konopleva et al., (2002) Br. J. Haematol. 118(2): 521-534). Chemoresistance, the ability of cancer cells to evade or to cope in the presence of therapeutics, is also a key challenge for therapeutic success.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is a transmembrane adhesion protein expressed on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

With few exceptions, E-selectin is not normally expressed in the vasculature but must be stimulated to be synthesized and expressed by inflammatory mediators. One of those exceptions is the microvasculature of the bone marrow (BM) where E-selectin is constitutively expressed.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the prophetic synthesis of compound 11.

FIG. 2 is a diagram illustrating the prophetic synthesis of compound 14.

FIG. 3 is a diagram illustrating the prophetic synthesis of multimeric compounds 21 and 22.

FIG. 4 is a diagram illustrating the prophetic synthesis of multimeric compounds 36 and 37.

FIG. 5 is a diagram illustrating the prophetic synthesis of multimeric compounds 44, 45, and 46.

FIG. 6 is a diagram illustrating the prophetic synthesis of multimeric compounds 55 and 56.

FIG. 7 is a diagram illustrating the prophetic synthesis of compound 60.

FIG. 8 is a diagram illustrating the prophetic synthesis of compound 65.

FIG. 9 is a diagram illustrating the prophetic synthesis of multimeric compounds 66, 67, and 68.

FIG. 10 is a diagram illustrating the prophetic synthesis of multimeric compounds 72 and 73.

FIG. 11 is a diagram illustrating the prophetic synthesis of multimeric compounds 76, 77, and 78.

FIG. 12 is a diagram illustrating the prophetic synthesis of multimeric compounds 86 and 87.

FIG. 13 is a diagram illustrating the prophetic synthesis of multimeric compound 95.

FIG. 14 is a diagram illustrating the prophetic synthesis of multimeric compound 146.

FIG. 15 is a diagram illustrating a prophetic synthesis of multimeric compound 197.

FIG. 16 is a diagram illustrating a synthesis of compound 205.

FIG. 17 is a diagram illustrating the synthesis of multimeric compound 206.

FIG. 18 is a diagram illustrating the synthesis of compound 214.

FIG. 19 is a diagram illustrating the synthesis of multimeric compounds 218, 219, and 220.

FIG. 20 is a diagram illustrating the synthesis of multimeric compound 224.

FIG. 21 is a diagram illustrating the prophetic synthesis of compound 237.

FIG. 22 is a diagram illustrating the prophetic synthesis of compound 241.

FIG. 23 is a diagram illustrating the prophetic synthesis of compound 245.

FIG. 24 is a diagram illustrating the prophetic synthesis of multimeric compound 257.

FIG. 25 is a diagram illustrating the prophetic synthesis of multimeric compounds 261, 262, and 263.

FIG. 26 is a diagram illustrating the prophetic synthesis of multimeric compounds 274, 275, and 276.

FIG. 27 is a diagram illustrating the prophetic synthesis of compound 291.

FIG. 28 is a diagram illustrating the prophetic synthesis of multimeric compounds 294 and 295.

FIG. 29 is a diagram illustrating the prophetic synthesis of multimeric compounds 305, 306, and 307.

FIG. 30 is a diagram illustrating the synthesis of compound 316.

FIG. 31 is a diagram illustrating the synthesis of compound 318.

FIG. 32 is a diagram illustrating the synthesis of compound 145.

FIG. 33 is a diagram illustrating the synthesis of compound 332.

FIG. 34 is a diagram illustrating experimental results of human CD34+ AML cell line KG1a cells cultured for 24 hrs in contact with vascular adhesion molecules (PECAM-1/CD31, VCAM-1, E-selectin) in the presence of cytarabine chemotherapy+NF-κB inhibitor BMS-345541.

FIG. 35 is a diagram illustrating how the NF-κB pathway induces chemoresistance in cancer patients.

FIG. 36 is a diagram illustrating experimental results showing that mice engrafted with MLL-AF9 AIL cells showed higher expression of E-selectin on the surface of bone marrow endothelial cells than control animals.

FIG. 37 is a diagram illustrating experimental results of expression of E-selectin ligand on AML blasts of patients that are newly diagnosed versus patients that have relapsed.

FIG. 38 is a list of 24 identified genes for AML patient biopsy screening that code for either glycosyltransferase or glycosidase enzymes.

FIG. 39 is a diagram showing the expression levels of the 24 identified genes for AML patient biopsy screening that code for either glycosyltransferase or glycosidase enzymes.

FIG. 40 is a table showing a univariate Cox model for overall survival (OS) using gene expression as a continuous coefficient (N=1,061). Of the genes assessed, 7 were significantly associated with increased risk (p<0.05).

FIG. 41 is a diagram illustrating the process by which the sialyltransferase product of ST3GAL4 and the fucosyltransferase product of FUT7 synthesize the E-selectin ligand sialyl Le^{x}.

FIG. 42 is a diagram showing the overall survival of patients expressing high and low levels of FUT7 and high and low levels of ST3GAL4.

FIG. 43 is a diagram showing the results of patients highly expressing both genes ST3GAL4 and FUT7 (SF high), those that did not highly express either gene (SF low), and patients with high expression of only one of the two genes (SF inter).

FIG. 44 is a diagram showing the expression levels from leukemic specimens from SF high and SF low patients using two MDF assays.

FIG. 45 is a diagram illustrating correlation of E-selectin ligand expression (as detected by antibody HECA-452) on blasts in the bone marrow of AML relapsed/refractory patients with the degree of those patients' response to the compound of Formula I and chemotherapy.

FIG. 46 is a diagram illustrating correlation of E-selectin ligand expression (as detected by antibody HECA-452) on blasts in the peripheral blood of AML relapsed/refractory patients with the degree of those patients' response to the compound of Formula I and chemotherapy at 12 hrs and 48 hrs post treatment with the compound of Formula I.

FIG. 47 is a diagram illustrating overall survival (OS) of patients with less than 10% of AML blasts expressing E-selectin ligand (as detected by antibody HECA-452) compared with patients with greater than 10% of blasts expressing E-selectin ligand.

FIG. 48A is a diagram illustrating experimental results of circulating TNFα levels in the peripheral blood (PB) of AML patients expressing various subtypes of AML blasts.

FIG. 48B is a diagram illustrating experimental results of TNFα mRNA expression levels in AML leukemic cells (LC) of AML patients expressing various subtypes of AML blasts.

FIG. 49 is a diagram illustrating overall survival (OS) and event-free survival of FLT3-ITD AML patients expressing high (i.e., greater than or equal to 10 pg/mL) or low (i.e., less than 10 pg/mL) serum levels of TNFα.

FIG. 50 is a diagram illustrating experimental results of expression of E-selectin ligand on AML blasts of patients with the FLT3-ITD mutation versus those without the mutation.

FIG. 51A is a diagram illustrating overall survival (OS) of FLT3-ITD AML patients expressing high (i.e., greater than median) or low (i.e., less than median) levels of FUT7.

FIG. 51B is a diagram illustrating overall survival (OS) of FLT3-ITD AML patients expressing high (i.e., greater than median) or low (i.e., less than median) levels of ST3GAL4.

FIG. 52 is a diagram illustrating the correlation of expression of ST3GAL4 and FUT7 with overall survival.

FIG. 53 is a diagram illustrating the correlation of expression of both ST3GAL4 and FUT7, one of ST3GAL4 or FUT7, or neither gene, with overall survival.

FIG. 54 is a diagram showing the number of patients shared between the highest-expressing quartile of ST3GAL4 and FUT7.

FIG. 55 is a chart of the cancer types in the PanCanAtlas of The Cancer Genome.

FIG. 56A is a diagram illustrating log 2 transformed expression levels of FUT7 in cancer types in the PanCanAtlas.

FIG. 56B is a diagram illustrating log 2 transformed expression levels of ST3GAL4 cancer types in the PanCanAtlas.

FIG. 57A is a diagram illustrating expression levels of FUT7 in cancer types in the Cancer Cell Line Encyclopedia.

FIG. 57B is a diagram illustrating expression levels of ST3GAL4 in cancer types in the Cancer Cell Line Encyclopedia.

FIG. 58A is a diagram illustrating expression levels of FUT7 in the TCGA-LAML FLT3 data set.

FIG. 58B is a diagram illustrating expression levels of ST3GAL4 in the TCGA-LAML FLT3 data set.

In order to better understand the disclosure, certain exemplary embodiments are discussed herein. In addition, certain terms are discussed to aid in the understanding.

Disclosed herein are methods of screening cancer patients for treatment, and upon screening the patients, treating a subset of them meeting certain criteria with an E-selectin inhibitor for purposes of treating the cancer and lengthening overall survival.

According to one embodiment, a method of screening a cancer patient may include obtaining or having obtained a biological sample from the cancer patient.

The biological sample may be any sample that is taken from a cancer patient. Examples include, but are not limited to, blood, plasma, saliva, pleural fluid, sweat, ascitic fluid, bile, urine, serum, pancreatic juice, stool, cervical smear samples, tumor biopsies, or any other sample that contains nucleic acids such as DNA and RNA.

In these embodiments, the method of screening the cancer patient may include performing or having performed an assay on the biological sample obtained from the cancer patient to determine the gene expression level of one or more E-selectin ligand-forming genes in the sample.

In these embodiments, performing the assay may further comprise measuring the number of mRNA transcripts or the amount of protein expressed.

The assay may be any assay that allows determination of a gene expression level, including but not limited to Sanger sequencing, high throughput sequencing, quantitative polymerase chain reaction, reverse transcriptase qPCR, RNA sequencing, microarray analysis, Northern blots, RNA-seq, high coverage mRNA sequencing, flow analysis, flow cytometry, immunohistology, immunostaining, immunohistochemistry, affinity purification, mass spectrometry, Western blotting, enzyme-linked immunoadsorbent assay, and multidimensional flow cytometry.

In some embodiments, the assay may use reagents chosen from a HECA-452-FITC monoclonal antibody, an E-selectin/hIg chimera, and chimera/PE.

In some embodiments, if the biological sample has an increased gene expression level for one or more particular genes relative to the expression level for that particular gene in a cancer-free subject, a newly diagnosed cancer subject, or a subject diagnosed with the same cancer as the patient, the method of screening the cancer patient may include selecting the patient for treatment comprising one or more E-selectin inhibitors. In some embodiments, the gene is an E-selectin ligand-forming gene.

In some embodiments, if at least 10%, at least 15%, at least 20%, or at least 25% of the blast cells in the biological sample express one or more particular genes, the method of screening the cancer patient may include selecting the patient for treatment comprising one or more E-selectin inhibitors. In some embodiments, the gene is an E-selectin ligand-forming gene.

In another embodiment, a method of treating a cancer patient may include obtaining or having obtained a biological sample from the cancer patient.

The biological sample may be any sample that is taken from a cancer patient. Examples include, but are not limited to, blood, plasma, saliva, pleural fluid, sweat, ascitic fluid, bile, urine, serum, pancreatic juice, stool, cervical smear samples, tumor biopsies, or any other sample that contains nucleic acids such as DNA and RNA.

In these embodiments, the method of treating the cancer patient may include performing or having performed an assay on the biological sample obtained from the cancer patient to determine the gene expression level of one or more E-selectin ligand-forming genes in the sample.

In these embodiments, performing the assay may further comprise measuring the number of mRNA transcripts or the amount of protein expressed.

The assay may be any assay that allows determination of a gene expression level, including but not limited to Sanger sequencing, high throughput sequencing, quantitative polymerase chain reaction, reverse transcriptase qPCR, RNA sequencing, microarray analysis, Northern blots, RNA-seq, high coverage mRNA sequencing, flow analysis, flow cytometry, immunohistology, immunostaining, immunohistochemistry, affinity purification, mass spectrometry, Western blotting, enzyme-linked immunoadsorbent assay, and multidimensional flow cytometry.

In some embodiments, the assay may use reagents chosen from a HECA-452-FITC monoclonal antibody, an E-selectin/hIg chimera, and chimera/PE.

In some embodiments, if the biological sample has an increased gene expression level for one or more particular genes relative to the expression level for that particular gene in a non-cancer subject, a newly diagnosed cancer subject, or a subject diagnosed with the same cancer as the patient, the method of screening the cancer patient may include selecting the patient for treatment comprising one or more E-selectin inhibitors. In some embodiments, the gene is an E-selectin ligand-forming gene.

In some embodiments, if at least 10%, at least 15%, at least 20%, or at least 25% of the blast cells in the biological sample express one or more particular genes, the method of screening the cancer patient may include selecting the patient for treatment comprising one or more E-selectin inhibitors. In some embodiments, the gene is ane E-selectin ligand-forming gene.

In these embodiments, the method of treating a cancer patient may include administering a therapeutically effective amount of a composition comprising one or more E-selectin inhibitors.

### Definitions

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

As used herein, the singular forms of a word also include the plural form of the word, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

“About” can be understood as within +/−10%, e.g., +/−10%, 9%, 8%, 7%, 6%, 5%, 4%, 3%, 2%, 1%, 0.5%, 0.1%, 0.05%, or 0.01% of the stated value. When used in reference to a percentage value, “about” can be understood as within ±1% (e.g., “about 5%” can be understood as within 4%-6%). All ranges used herein encompass the endpoints.

As used herein, the terms “treatment,” “treating,” and the like, refer to obtaining a desired pharmacologic and/or physiologic effect. The effect can be prophylactic in terms of completely or partially preventing a disease or symptom thereof from occurring in the first place and/or can be therapeutic in terms of a partial or complete cure for a disease and/or adverse effects attributable to the disease. As an example, the term “treatment” and the like, as used herein, encompasses any treatment of cancers such as AML or any of its subtypes and related hematologic cancers in a mammal, particularly in a human, and includes: (a) preventing the disease from occurring in a subject, e.g., a subject identified as predisposed to the disease or at risk of acquiring the disease but has not yet been diagnosed as having it; (b) delaying onset or progression of the disease, e.g., as compared to the anticipated onset or progression of the disease in the absence of treatment: (c) inhibiting the disease, i.e., arresting its development; and/or (d) relieving the disease, i.e., causing regression of the disease. In some embodiments, “treating” refers to administering e.g., subcutaneously, an effective dose, or effective multiple doses of a composition e.g., a composition comprising an inhibitor, e.g., an E-selectin inhibitor, as disclosed herein to an animal (including a human being) suspected of suffering or already suffering from AML or another related cancer. It can also refer to reducing, eliminating, or at least partially arresting, as well as to exerting any beneficial effect, on one or more symptoms of the disease and/or associated with the disease and/or its complications.

As used herein, the terms “blasts” and “blast cells” are used interchangeably to refer to undifferentiated, precursor blood stem cells. As used herein, the term “blast count” refers to the number of blast cells in a sample.

The terms “acute myeloid leukemia,” “acute myelogenous leukemia,” “acute myeloblastic leukemia,” “acute granulocytic leukemia,” and “acute nonlymphocytic leukemia,” and “AML” are used interchangeably and as used herein, refer to a cancer of the bone marrow characterized by abnormal proliferation of myeloid stem cells. AML as used herein, refers to any or all known subtypes of the disease, including but not limited to subtypes classified by the World Health Organization (WHO) 2016 classification of AML, e.g., AML with myelodysplasia-related changes or myeloid sarcoma, and the French-American-British (FAB) classification system, e.g., M0 (acute myeloblastic leukemia, minimally differentiated) or M1 (acute myeloblastic leukemia, without maturation). Falini et al., (2010) Discov. Med., 10(53): 281-92; Lee et al., (1987) Blood, 70(5): 1400-1406.

The term “E-selectin ligand” as used herein, refers to a carbohydrate structure that contains the epitope shared by sialyl Le^{a} and sialyl Le^{x}. Carbohydrates are secondary gene products synthesized by enzymes known as glycosyltransferases which are the primary gene products coded for by DNA. Each glycosyltransferase adds a specific monosaccharide in a specific stereochemical linkage to a specific donor carbohydrate chain.

The terms “E-selectin antagonist” and “E-selectin inhibitor” are used interchangeably herein. E-selectin inhibitors are known in the art. Some E-selectin inhibitors are specific for E-selectin only. Other E-selectin inhibitors have the ability to inhibit not only E-selectin but additionally P-selectin or L-selectin or both P-selectin and L-selectin. In some embodiments, an E-selectin inhibitor inhibits E-selectin, P-selectin, and L-selectin.

In some embodiments, an E-selectin inhibitor is a specific glycomimetic antagonist of E-selectin. Examples of E-selectin inhibitors (specific for E-selectin or otherwise) are disclosed in U.S. Pat. No. 9,109,002, the disclosure of which is expressly incorporated by reference in its entirety.

In some embodiments, the E-selectin antagonists suitable for the disclosed compounds and methods include pan-selectin antagonists.

Non-limiting examples of suitable E-selectin antagonists include small molecules, such as nucleic acids, peptides, polypeptides, peptidomimetics, carbohydrates, glycomimetics, lipids and other organic (carbon containing) or inorganic molecules. Suitably, the selectin antagonist is selected from antigen-binding molecules that are immuno-interactive with a selectin, peptides that bind to the selectin and that block cell-cell adhesion, and carbohydrate or peptide mimetics of selectin ligands. In some embodiments, the E-selectin antagonist reduces the expression of a selectin gene or the level or functional activity of an expression product of that gene. For example, the E-selectin antagonist may antagonize the function of the selectin, including reducing or abrogating the activity of at least one of its ligand-binding sites.

In some embodiments, the E-selectin antagonist inhibits an activity of E-selectin or inhibits the binding of E-selectin to one or more E-selectin ligands (which in turn may inhibit a biological activity of E-selectin).

E-selectin antagonists include the glycomimetic compounds described herein. E-selectin antagonists also include antibodies, polypeptides, peptides, peptidomimetics, and aptamers which bind at or near the binding site on E-selectin to inhibit E-selectin interaction with sialyl Le^{a} (sLe^{a}) or sialyl Le^{x} (sLe^{x}).

Further disclosure regarding E-selectin antagonists suitable for the disclosed methods and compounds may be found in U.S. Pat. No. 9,254,322, issued Feb. 9, 2016, and U.S. Pat. No. 9,486,497, issued Nov. 8, 2016, which are both hereby incorporated by reference in their entireties. In some embodiments, the selectin antagonist is chosen from E-selectin antagonists disclosed in U.S. Pat. No. 9,109,002, issued Aug. 18, 2015, which is hereby incorporated by reference in its entirety. In some embodiments, the E-selectin antagonist is chosen from heterobifunctional antagonists disclosed in U.S. Pat. No. 8,410,066, issued Apr. 2, 2013, and US Publication No. US2017/0305951, published Oct. 26, 2017, which are both hereby incorporated by reference in their entireties. Further disclosure regarding E-selectin antagonists suitable for the disclosed methods and compounds may be found in PCT Publication Nos. WO2018/068010, published Apr. 12, 2018, WO2019/133878, published Jul. 4, 2019, and WO2020/139962, published Jul. 2, 2020, which are hereby incorporated by reference in their entireties.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

E-selectin antagonists, such as the compound of Formula I, which interrupt leukemic cell homing to the vascular niche and increase susceptibility to cytotoxic therapies, can be potent adjuncts to therapeutics.

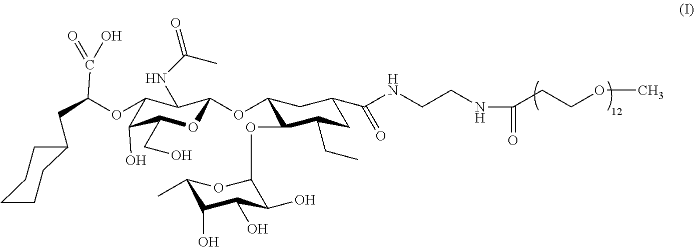

The pre-screening of patients amenable to treatment with an E-selectin inhibitor such as the compound of Formula I is also contemplated, e.g., according to the methods of identifying cancers disclosed herein, as well as the administration of treatment to patients identified according to criteria disclosed herein. In some embodiments, one or more diagnostic assays may be used to pre-screen cancer patients amenable to treatment with an E-selectin inhibitor. In some embodiments, the cancer patients amenable to treatment with an E-selectin inhibitor have leukemia. In some embodiments the cancer patients amenable to treatment with an E-selectin inhibitor have AML. In some embodiments, the AML patients may have one or more genetic mutations to the FLT3 gene. In some embodiments, the one or more diagnostic assays may be used to identify FLT3 patients expressing E-selectin ligand on their AML cells.

Pre-screening of patients who are likely to benefit from the treatments disclosed herein are also contemplated. Without being bound by theory, patients who express high amounts of E-selectin ligands on blast cells are chemo-resistant (relapsed/refractory) by a mechanism involving E-selectin, and therefore treatment with E-selectin antagonists shows greater efficacy. Accordingly, expression levels of genes involved in the synthesis or degradation of E-selectin ligands may be useful in pre-screening patients who may be more likely to benefit from treatment with E-selectin antagonists, e.g., the compound of Formula I. The disclosure herein is based on the surprising discovery that while AML patients with the highest expression of genes involved in synthesis or degradation of E-selectin ligands, e.g., ST3GAL4 and FUT7 genes, have poorer outcomes and shorter overall survival, relapsed/refractory patients expressing higher levels of these genes have better outcomes when treated with a combination of chemotherapy and the compositions disclosed herein.

Methods to measure gene expression levels are known to persons of skill in the art. Gene expression may be measured by the number of mRNA transcripts or the amount of protein expressed. Exemplary methods to measure the amount of mRNA include but are not limited to Sanger sequencing, high throughput sequencing, quantitative polymerase chain reaction (qPCR), reverse transcriptase qPCR (RT-qPCR), RNA sequencing, microarray analysis, and Northern blots. In some embodiments, gene expression level is measured by RNA-seq. In some embodiments, gene expression level is measured by high coverage mRNA sequencing.

In some embodiments, gene expression level is measured by the amount of mRNA. In some embodiments, the method comprises measuring the amount of mRNA encoding one or more of the following genes in a patient sample: FUT3, FUT4, FUT5, FUT6, FUT7, FUT8, FUT9, ST3GAL1, ST3GAL2, ST3GAL3, ST3GAL4, ST3GAL5, ST3GAL6, NEU1, NEU2, NEU3, NEU4, FUCA1, and/or FUCA2.

Gene expression may also be measured by the amount of protein in a patient sample. Exemplary methods to measure the amount of protein include but are not limited to immunostaining, immunohistochemistry, affinity purification, mass spectrometry, Western blotting, and enzyme-linked immunoadsorbent assay (ELISA).

In some embodiments, gene expression level is measured by the amount of protein in a patient sample. In some embodiments, the method comprises measuring the amount of one or more of the following proteins in a patient sample: FUT3 protein, FUT4 protein, FUT5 protein, FUT7 protein, FUT8 protein, FUT9 protein, ST3GAL1 protein, ST3GAL2 protein, ST3GAL3 protein, ST3GAL4 protein, ST3GAL5 protein, ST3GAL6 protein, NEU1 protein, NEU2 protein, NEU3 protein, NEU4 protein, FUCA1 protein, and/or FUCA2 protein.

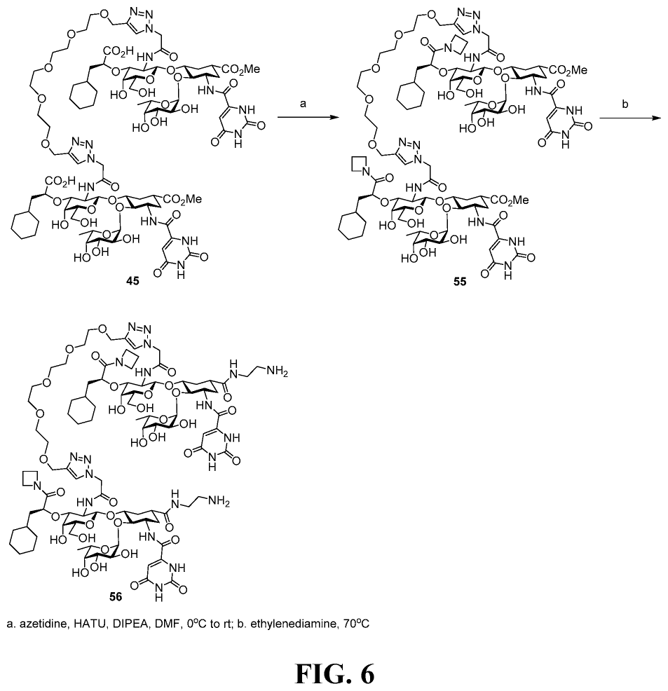

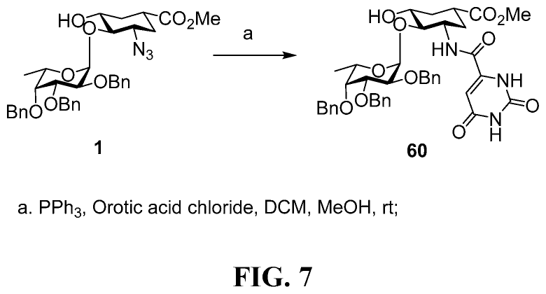

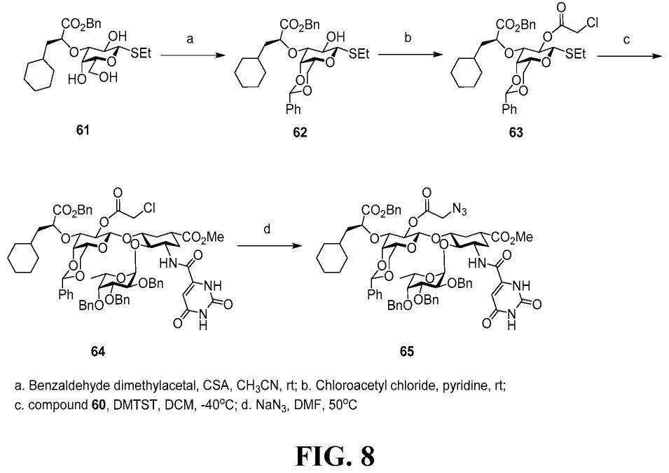

In some embodiments, high coverage single strand mRNA sequencing may be performed on clinical samples from pediatric AML patients (0 to 30 years old). In some embodiments, the data from this analysis may then be screened for expression of the 24 different genes listed in FIGS. 6-7. In some embodiments, the observed expression may then be correlated with the clinical outcome of overall survival (OS).

In some embodiments, the one or more diagnostic assays may comprise assays to detect expression of E-selectin ligand on the surface of FLT3 AML cells, and may include flow analysis, flow cytometry, or immunohistology using the appropriate reagents. In some embodiments, the reagents for immunohistology may include a HECA-452-FITC monoclonal antibody, or similar reagents. In other embodiments, the reagents for immunohistology may include an E-selectin/hIg chimera/PE, or similar reagents.

In some embodiments, the expression level of a gene involved in the synthesis of sialic acids is measured. In some embodiments, the sialic acid is an α2-3 sialic acid. In some embodiments, the expression level of a gene involved in the degradation of sialic acids is measured. In some embodiments, the expression level of a gene involved in the synthesis of fucose linkages in E-selectin ligands is measured. In some embodiments, the expression level of a gene involved in the degradation of fucose linkages in E-selectin ligands is measured. In some embodiments, the expression level of a gene that encodes a glycotransferase in a patient is measured. In some embodiments, the expression level of a gene that encodes a glycosidase in a patient is measured. In some embodiments, 24 different genes (i.e., those shown in FIGS. 6-7) that code for enzymes that either build carbohydrate chains (glycosyltransferases) or enzymes that destroy carbohydrate chains (glycosidases) may be analyzed for expression of the E-selectin ligand.

In some embodiments, the method comprises measuring the expression level(s) of one or more of the following genes in a patient sample: FUT3, FUT4, FUT5, FUT6, FUT7, FUT8, FUT9, ST3GAL1, ST3GAL2, ST3GAL3, ST3GAL4, ST3GAL5, ST3GAL6, NEU1, NEU2, NEU3, NEU4, FUCA1, and/or FUCA2.

In some embodiments, one or more diagnostic assays may be used to identify cancer patients likely to benefit from treatment with an E-selectin inhibitor. In some embodiments, the cancer patients likely to benefit from treatment with an E-selectin inhibitor have leukemia. In some embodiments the cancer patients likely to benefit from treatment with an E-selectin inhibitor have AML. In some embodiments the cancer patients likely to benefit from treatment with an E-selectin inhibitor have ALL. In some embodiments the cancer patients likely to benefit from treatment with an E-selectin inhibitor have CLL. In some embodiments the cancer patients likely to benefit from treatment with an E-selectin inhibitor have CML. In some embodiments the cancer patients likely to benefit from treatment with an E-selectin inhibitor have non-Hodgkins lymphoma. In some embodiments the cancer patients likely to benefit from treatment with an E-selectin inhibitor have Hodgkins lymphoma. In some embodiments the cancer patients likely to benefit from treatment with an E-selectin inhibitor have multiple myeloma. In some embodiments the cancer patients likely to benefit from treatment with an E-selectin inhibitor have colorectal cancer. In some embodiments the cancer patients likely to benefit from treatment with an E-selectin inhibitor have liver cancer. In some embodiments the cancer patients likely to benefit from treatment with an E-selectin inhibitor have gastric cancer. In some embodiments the cancer patients likely to benefit from treatment with an E-selectin inhibitor have lung cancer. In some embodiments the cancer patients likely to benefit from treatment with an E-selectin inhibitor have brain cancer. In some embodiments the cancer patients likely to benefit from treatment with an E-selectin inhibitor have kidney cancer. In some embodiments the cancer patients likely to benefit from treatment with an E-selectin inhibitor have bladder cancer. In some embodiments the cancer patients likely to benefit from treatment with an E-selectin inhibitor have thyroid cancer. In some embodiments the cancer patients likely to benefit from treatment with an E-selectin inhibitor have prostrate cancer. In some embodiments the cancer patients likely to benefit from treatment with an E-selectin inhibitor have ovarian cancer. In some embodiments the cancer patients likely to benefit from treatment with an E-selectin inhibitor have cervical cancer. In some embodiments the cancer patients likely to benefit from treatment with an E-selectin inhibitor have uterine cancer. In some embodiments the cancer patients likely to benefit from treatment with an E-selectin inhibitor have endometrial cancer. In some embodiments the cancer patients likely to benefit from treatment with an E-selectin inhibitor have melanoma. In some embodiments the cancer patients likely to benefit from treatment with an E-selectin inhibitor have breast cancer. In some embodiments the cancer patients likely to benefit from treatment with an E-selectin inhibitor have pancreatic cancer. In some embodiments, the one or more diagnostic assays comprises quantitative PCR (polymerase chain reaction).

In some aspects, a method of treating a patient suffering from cancer comprises: (a) determining the gene expression level of one or more genes in the patient or a sample from the patient; (b) comparing the gene expression level from (a) to a control sample from a cancer-free subject, a newly diagnosed cancer subject, or a subject diagnosed with the same cancer as the patient, and when the gene expression level exceeds that in the control sample; then (c) administering one or more doses of a pharmaceutical composition comprising an E-selectin inhibitor to the patient. In some embodiments, the one or more genes is chosen from ST3GAL4, FUT5, and FUT7. In some embodiments, the E-selectin inhibitor is administered in combination with an anti-cancer agent. In some embodiments, gene expression level is determined by high coverage single-strand mRNA sequencing. In some embodiments, the sample from the patient is peripheral blood.

In some aspects, a method of treating a cancer patient comprises: (a) obtaining or having obtained a biological sample comprising blast cells from the cancer patient; (b) performing or having performed an assay on the biological sample to determine the gene expression level of one or more E-selectin ligand-forming genes in the sample; and (c) if the blast cells in the sample have an increased gene expression level of the one or more E-selectin ligand-forming genes relative to a control sample from a non-cancer subject, a newly-diagnosed cancer subject, or a subject having the same cancer as the patient, then administering a therapeutically effective amount of a composition comprising one or more E-selectin inhibitors.

In some embodiments, the control sample is from a person diagnosed with the same cancer as that of the patient. In some embodiments, the control sample is the distribution of gene expression levels of ST3GAL4 in a population of people diagnosed with the same cancer as that of the patient. In some embodiments, the threshold is the 90^{th} percentile, 85^{th} percentile, 80^{th} percentile, 75^{th} percentile, 70^{th} percentile, 65^{th} percentile, 60^{th} percentile, 55^{th} percentile, or 50^{th} percentile level of expression of ST3GAL4 in a population of people diagnosed with the same cancer as that of the patient.

In some embodiments, the control sample is from a person diagnosed with the same cancer as that of the patient. In some embodiments, the control sample is the distribution of gene expression levels of FUT5 in a population of people diagnosed with the same cancer as that of the patient. In some embodiments, the threshold is the 90^{th} percentile, 85^{th} percentile, 80^{th} percentile, 75^{th} percentile, 70^{th} percentile, 65^{th} percentile, 60^{th} percentile, 55^{th} percentile, or 50^{th} percentile level of expression of FUT5 in a population of people diagnosed with the same cancer as that of the patient.

In some embodiments, the control sample is from a person diagnosed with the same cancer as that of the patient. In some embodiments, the control sample is the distribution of gene expression levels of FUT7 in a population of people diagnosed with the same cancer as that of the patient. In some embodiments, the threshold is the 90^{th} percentile, 85^{th} percentile, 80^{th} percentile, 75^{th} percentile, 70^{th} percentile, 65^{th} percentile, 60^{th} percentile, 55^{th} percentile, or 50^{th} percentile level of expression of FUT7 in a population of people diagnosed with the same cancer as that of the patient.

In some aspects, a method of treating a patient suffering from cancer comprises: (a) determining the gene expression level of one or more genes in the patient or a sample from the patient; and (b) administering one or more doses of a pharmaceutical composition comprising an E-selectin inhibitor to the patient if at least 10% of the blast cells in the patient or a sample from the patient express the one or more genes. In some embodiments, the one or more genes are chosen from ST3GAL4, FUT5, and FUT7. In some embodiments, the E-selectin inhibitor is administered in combination with an anti-cancer agent. In some embodiments, gene expression level is determined by high coverage single-strand mRNA sequencing. In some embodiments, the sample from the patient is peripheral blood.

In some aspects, a method of treating a cancer patient comprises: (a) obtaining or having obtained a biological sample comprising blast cells from the cancer patient; (b) performing or having performed an assay on the biological sample to determine the gene expression level of one or more E-selectin ligand-forming genes in the sample; and (c) if at least 10% of the blast cells in the sample express the one or more E-selectin ligand-forming genes, then administering a therapeutically effective amount of a composition comprising one or more E-selectin inhibitors.

In some embodiments, one or more doses of a pharmaceutical composition comprising an E-selectin inhibitor, e.g., the compound of Formula I, is administered in combination with an anti-cancer agent to a patient who has been pre-screened by the criteria as disclosed herein as having, e.g., increased expression of ST3GAL4, FUT5, or FUT7.

In some aspects, a method of selecting a patient to treat for cancer comprises: (a) determining the gene expression level of one or more genes in the patient or a sample from the patient; (b) selecting the patient for treatment when the patient or sample from the patient has an increased gene expression level relative to a control sample; and (c) treating the patient by administering one or more doses of a pharmaceutical composition comprising an E-selectin inhibitor. In some embodiments, the one or more genes are chosen from ST3GAL4, FUT5, and FUT7. In some embodiments, the E-selectin inhibitor is administered in combination with an anti-cancer agent. In some embodiments, gene expression level is determined by high coverage single-strand mRNA sequencing. In some embodiments, the sample from the patient is peripheral blood.

In some aspects, a method of screening a cancer patient for treatment comprises: (a) obtaining or having obtained a biological sample comprising blast cells from the cancer patient; (b) performing or having performed an assay on the biological sample to determine the gene expression level of one or more E-selectin ligand-forming genes in the sample; and (cxi) if the blast cells in the sample have an increased expression level of the one or more E-selectin ligand-forming genes relative to a control sample from a non-cancer subject, a newly-diagnosed cancer subject, or a subject having the same cancer as the patient, or (c)(ii) if at least 10% of the blast cells in the sample express the one or more E-selectin ligand-forming genes, then (d) selecting the patient for treatment comprising one or more E-selectin inhibitors.

In some embodiments, the control sample is from a patient suffering from AML. In some embodiments, the control sample is the distribution of gene expression levels of ST3GAL4 in a population of patients suffering from AML. In some embodiments, the threshold is the 90^{th} percentile, 85^{th} percentile, 80^{th} percentile, 75^{th} percentile, 70^{th} percentile, 65^{th} percentile, 60^{th} percentile, 55^{th} percentile, or 50^{th} percentile level of expression of ST3GAL4 in a population of AML patients. In some embodiments, the control sample is the distribution of gene expression levels of FUT5 in a population of patients suffering from AML. In some embodiments, the threshold is the 90^{th} percentile, 85^{th} percentile, 80th percentile, 75^{th} percentile, 70^{th} percentile, 65^{th} percentile, 60^{th} percentile, 55^{th} percentile, or 50^{th} percentile level of expression of FUT5 in a population of AML patients. In some embodiments, the control sample is the distribution of gene expression levels of FUT7 in a population of patients suffering from AML. In some embodiments, the threshold is the 90th percentile, 85^{th} percentile, 80^{th} percentile, 75^{th} percentile, 70^{th} percentile, 65^{th} percentile, 60th percentile, 55^{th} percentile, or 50^{th} percentile level of expression of FUT7 in a population of AML patients.

In some embodiments, the treated patient has expression of ST3GAL4 greater than that of 55%, 60%, 65%, 70%, 75%, 80%, 85%, 90%, or 95% of patients with relapsed/refractory AML. In some embodiments, the treated patient has expression of FUT5 greater than that of 55%, 60%, 65%, 70%, 75%, 80%, 85%, 90%, or 95% of patients with relapsed/refractory AML. In some embodiments, the treated patient has expression of FUT7 greater than that of 55%, 60%, 65%, 70%, 75%, 80%, 85%, 90%, or 95% of patients with relapsed/refractory AML. In some embodiments, the treated patient has expression of ST3GAL4 and FUT5 greater than that of 55%, 60%, 65%, 70%, 75%, 80%, 85%, 90%, or 95% of patients with relapsed/refractory AML. In some embodiments, the treated patient has expression of ST3GAL4 and FUT7 greater than that of 55%, 60%, 65%, 70%, 75%, 80%, 85%, 90%, or 95% of patients with relapsed/refractory AML. In some embodiments, the treated patient has expression of FUT5 and FUT7 greater than that of 55%, 60%, 65%, 70%, 75%, 80%, 85%, 90%, or 95% of patients with relapsed/refractory AML. In some embodiments, the treated patient has expression of ST3GAL4, FUT5, and FUT7 greater than that of 55%, 60⁰%, 65%, 70%, 75%, 80%, 85%, 90%, or 95% of patients with relapsed/refractory AML.

In some aspects, a method of selecting a patient to treat for cancer comprises: (a) determining the gene expression level of one or more genes in the patient or a sample from the patient; (b) selecting the patient for treatment when at least 10% of the blast cells from the patient or sample from the patient expresses the one or more genes; and (c) treating the patient by administering one or more doses of a pharmaceutical composition comprising an E-selectin inhibitor. In some embodiments, the one or more genes are chosen from ST3GAL4, FUT5, and FUT7. In some embodiments, the E-selectin inhibitor is administered in combination with an anti-cancer agent. In some embodiments, gene expression level is determined by high coverage single-strand mRNA sequencing. In some embodiments, the sample from the patient is peripheral blood.

In some embodiments, a method of treating FLT3 AML patients with antagonists of E-selectin is disclosed, the method comprising administering to a FLT3 AML patient an effective amount of at least one E-selectin antagonist and/or a pharmaceutical composition comprising at least one E-selectin antagonist. In some embodiments, the at least one E-selectin antagonist is the compound of Formula I.

In some embodiments, the method further comprises administering at least one additional therapeutic agent. In some embodiments, the at least one additional therapeutic agent is chosen from chemotherapy agents and kinases inhibitors targeting FLT3.

Methods of treating AML comprising administering to a subject in need thereof an effective amount of the compound of Formula I and compositions comprising the same have been reported. See, e.g., PCT/US2019/020574. The compound of Formula I was rationally designed based on the bioactive conformation of sialyl Le^{a/x} in the binding site of E-selectin and is a potent and specific glycomimetic antagonist of E-selectin.

Contemplated herein are compositions for treating cancer patients in need thereof, comprising E-selectin inhibitors. E-selectin is a transmembrane adhesion protein expressed on the surface of endothelial cells lining the blood vessel. E-selectin recognizes and binds to sialylated carbohydrates, e.g., members of the Lewis X and Lewis A families found on monocytes, granulocytes, and T-lymphocytes. When expressed, it causes cells which express E-selectin ligands on their surface to adhere.

As discussed in detail herein, the disease or disorder to be treated is a cancer and related metastasis and includes cancers that comprise solid tumors and cancers that comprise liquid tumors. E-selectin plays a central role in the progression of cancer. The invasive properties of cancer cells depend, at least in part, on the capability of cancer cells to breach the endothelial barrier. Cancer cells, for example, colon cancer cells, may express E-selectin ligands that are capable of binding to endothelial cells that express E-selectin on their cell surface. Without wishing to be limited to any theory, binding of cancer cells to the endothelial cells can contribute to extravasation of the cancer cells.

Cancers that may be prevented from metastasizing include cancers that comprise solid tumors and those that comprise liquid tumors (e.g., hematological malignancies). Examples of solid tumors that may be treated with the agents described herein include colorectal cancer, liver cancer, gastric cancer, lung cancer, brain cancer, kidney cancer, bladder cancer, thyroid cancer, prostrate cancer, ovarian cancer, cervical cancer, uterine cancer, endometrial cancer, melanoma, breast cancer and pancreatic cancer. Liquid tumors occur in the blood, bone marrow, and lymph nodes and include leukemia (e.g., AML, ALL, CLL, and CML), lymphoma (e.g., non-Hodgkins lymphoma and Hodgkins lymphoma) and myeloma (e.g., multiple myeloma). Reports have described that liquid tumors such as multiple myeloma follow a similar invasion-metastasis cascade as observed with solid tumors and that E-selectin ligands are present on liquid tumor cells, such as myeloma cells. Others have observed that ligands of E-selectin may be important for extravascular infiltration of leukemia cells. Liquid tumor cells may also adhere to bone marrow, which may further lead to sequestration and quiescence of the tumor cells to chemotherapy, which phenomenon is referred to as adhesion mediated drug resistance. Studies have also indicated that bone marrow contains anatomic regions that comprise specialized endothelium, which expresses the E-selectin. Accordingly, an E-selectin antagonist, such as those described herein, may be useful for inhibiting metastasis of cancers that comprise either a solid or liquid tumor by inhibiting binding of an E-selectin ligand to E-selectin.

Methods of treating cancer are known to a skilled artisan, and may include, but are not limited to chemotherapy, radiation therapy, chemotherapy with stem cell transplant, other drugs such as arsenic trioxide and all-trans retinoic acid, and targeted therapy (e.g. a monoclonal antibody).

Contemplated herein are methods of treating cancer patients in need thereof, comprising administering a therapeutically effective amount of a composition comprising an E-selectin inhibitor, e.g., the compound of Formula I. The composition disclosed herein may be administered by parenteral, topical, intradermal, intravenous, oral, subcutaneous, intraperitoneal, intranasal or intramuscular means for prophylactic and/or therapeutic treatment.

Methods of treating cancer comprising administering to a subject in need thereof an effective amount of a compound of Formula I and compositions comprising the same have been reported. See, e.g., PCT/US2019/020574, the disclosure of which is expressly incorporated by reference in its entirety. The compound of Formula I was rationally designed based on the bioactive conformation of sialyl Le^{a/x} in the binding site of E-selectin and is a potent and specific glycomimetic antagonist of E-selectin.

In some embodiments, the composition is delivered by subcutaneous delivery. In some embodiments, the composition is delivered by subcutaneous delivery to the upper arm. In some embodiments, the composition is delivered by subcutaneous delivery to the abdomen. In some embodiments, the composition is delivered by subcutaneous delivery to the thigh. In some embodiments, the composition is delivered by subcutaneous delivery to the upper back. In some embodiments the composition is delivered by subcutaneous delivery to the buttock.

In some embodiments, the composition is delivered by intravenous infusion.

In some embodiments, the composition is delivered in combination with one or more anti-cancer agents. In some embodiments, the composition is delivered in combination with chemotherapy. Chemotherapy may comprise one or more chemotherapeutic agent(s). For example, chemotherapy agents, radiotherapy agents, inhibitors of phosphoinoditide-3 kinase (PI3K), and inhibitors of VEGF may be used in combination with an agent described herein. Examples of inhibitors of PI3K include the compound named Exelixis as “XL499”. Examples of VEGF inhibitors include the compound “cabo” (previously known as XL184). Many other chemotherapeutics are small organic molecules. As understood by a person skilled in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents, antimetabolites, anthracyclines, plant alkaloids and topoisomerase inhibitors. Examples of therapeutic agents administered for chemotherapy are well known to the skilled artisan. In some embodiments, the composition is delivered in combination with induction chemotherapy. In some embodiments, the composition is delivered in combination with mitoxantrone. In some embodiments, the composition is delivered in combination with etoposide. In some embodiments, the composition is delivered in combination with cytarabine. In some embodiments, the composition is delivered together with at least one of mitoxantrone, etoposide, and cytarabine. In some embodiments, the composition is delivered in combination with consolidation chemotherapy. In some embodiments, the composition is delivered in combination with daunomycin. In some embodiments, the composition is delivered in combination with idarubicin. In some embodiments, the composition is delivered in combination with MEC (mitoxantrone, etoposide, cytarabine) chemotherapy. In some embodiments, the composition is delivered in combination with 7+3 (cytarabine for 7 days then daunorubicin, idarubicin, or mitoxantrone for 3 days) chemotherapy.

In some embodiments, the anti-cancer agents are anti-leukemic agents. Examples of anti-leukemic agents are well-known to the skilled artisan, and include but are not limited to cyclophosphamide, methotrexate, and etoposide. In some embodiments, the composition is delivered in combination with 6-mercaptopurine. In some embodiments, the composition is delivered in combination with 6-thioguanine. In some embodiments, the composition is delivered in combination with aminopterin. In some embodiments, the composition is delivered in combination with arsenic trioxide. In some embodiments, the composition is delivered in combination with asparaginase. In some embodiments, the composition is delivered in combination with cladribine. In some embodiments, the composition is delivered in combination with clofarabine. In some embodiments, the composition is delivered in combination with cyclophosphamide. In some embodiments, the composition is delivered in combination with cytosine arabinoside. In some embodiments, the composition is delivered in combination with dasatinib. In some embodiments, the composition is delivered in combination with decitabine. In some embodiments, the composition is delivered in combination with dexamethasone. In some embodiments, the composition is delivered in combination with fludarabine. In some embodiments, the composition is delivered in combination with gemtuzumab ozogamicin. In some embodiments, the composition is delivered in combination with imatinib mesylate. In some embodiments, the composition is delivered in combination with interferon-α. In some embodiments, the composition is delivered in combination with interleukin-2. In some embodiments, the composition is delivered in combination with melphalan. In some embodiments, the composition is delivered in combination with methotrexate. In some embodiments, the composition is delivered in combination with nelarabine. In some embodiments, the composition is delivered in combination with nilotinib. In some embodiments, the composition is delivered in combination with oblimersen. In some embodiments, the composition is delivered in combination with pegaspargase. In some embodiments, the composition is delivered in combination with pentostatin. In some embodiments, the composition is delivered in combination with ponatinib. In some embodiments, the composition is delivered in combination with prednisone. In some embodiments, the composition is delivered in combination with rituximab. In some embodiments, the composition is delivered in combination with tretinoin. In some embodiments, the composition is delivered in combination with vincristine.

In some embodiments, the anti-cancer agent may be radiation. In some embodiments, the composition may be delivered in combination with external beam radiation.

In various embodiments, the composition is administered over one or more doses, with one or more intervals between doses. In some embodiments, the composition is administered over 1, 2, 3, 4, 5, 6, 7, 8, 9, 10, 11, 12, 13, 14, 15, 16, 17, 18, 19, or 20 doses. In some embodiments, the composition is administered at 6-hour, 12-hour, 18-hour, 24-hour, 48-hour, 72-hour, or 96-hour intervals. In some embodiments, the composition is administered at one interval, and then administered at a different interval, e.g., 1 dose 24 hours before chemotherapy, then twice-daily doses throughout chemotherapy. In some embodiments, the composition is administered at 1 dose 24 hours before chemotherapy, then twice-daily doses throughout chemotherapy up till 48 hours post-chemotherapy.

In some embodiments, the methods and materials disclosed herein are indicated for and can be used in the treatment of AML, e.g., by subcutaneous or intravenous administration to a patient showing the symptoms of the disease. In some embodiments, the methods and materials disclosed herein are indicated for and can be used in the treatment of ALL. In some embodiments, the methods and materials disclosed herein are indicated for and can be used in the treatment of CLL. In some embodiments, the methods and materials disclosed herein are indicated for and can be used in the treatment of CML. In some embodiments, the methods and materials disclosed herein are indicated for and can be used in the treatment of non-Hodgkins lymphoma. In some embodiments, the methods and materials disclosed herein are indicated for and can be used in the treatment of Hodgkins lymphoma. In some embodiments, the methods and materials disclosed herein are indicated for and can be used in the treatment of multiple myeloma. In some embodiments, the methods and materials disclosed herein are indicated for and can be used in the treatment of colorectal cancer. In some embodiments, the methods and materials disclosed herein are indicated for and can be used in the treatment of liver cancer. In some embodiments, the methods and materials disclosed herein are indicated for and can be used in the treatment of gastric cancer. In some embodiments, the methods and materials disclosed herein are indicated for and can be used in the treatment of lung cancer. In some embodiments, the methods and materials disclosed herein are indicated for and can be used in the treatment of brain cancer. In some embodiments, the methods and materials disclosed herein are indicated for and can be used in the treatment of kidney cancer. In some embodiments, the methods and materials disclosed herein are indicated for and can be used in the treatment of bladder cancer. In some embodiments, the methods and materials disclosed herein are indicated for and can be used in the treatment of thyroid cancer. In some embodiments, the methods and materials disclosed herein are indicated for and can be used in the treatment of prostrate cancer. In some embodiments, the methods and materials disclosed herein are indicated for and can be used in the treatment of ovarian cancer. In some embodiments, the methods and materials disclosed herein are indicated for and can be used in the treatment of In some embodiments, the methods and materials disclosed herein are indicated for and can be used in the treatment of cervical cancer. In some embodiments, the methods and materials disclosed herein are indicated for and can be used in the treatment of uterine cancer. In some embodiments, the methods and materials disclosed herein are indicated for and can be used in the treatment of endometrial cancer. In some embodiments, the methods and materials disclosed herein are indicated for and can be used in the treatment of melanoma. In some embodiments, the methods and materials disclosed herein are indicated for and can be used in the treatment of breast cancer. In some embodiments, the methods and materials disclosed herein are indicated for and can be used in the treatment of pancreatic cancer.

In some embodiments, an effective dose is a dose that partially or fully alleviates (i.e., eliminates or reduces) at least one symptom associated with the disorder/disease state being treated, that slows, delays, or prevents onset or progression to a disorder/disease state, that slows, delays, or prevents progression of a disorder/disease state, that diminishes the extent of disease, that reverse one or more symptom, that results in remission (partial or total) of disease, and/or that prolongs survival. Examples of disease states contemplated for treatment are set out herein. In some embodiments, the patient currently has cancer, was once treated for cancer and is in remission, or is at risk of relapsing after treatment for the cancer.

In some embodiments, a pharmaceutical composition as disclosed herein is administered, e.g., subcutaneously or intravenously, to a patient in need of treatment for AML. In some embodiments, the patient has been diagnosed with AML as per the World Health Organization (WHO) criteria. Arber D A et al., “The 2016 revision to the World Health Organization classification of myeloid neoplasms and acute leukemia.” Blood (2016) 127(20):2391-2405. In some embodiments, the patients are ≥18 years of age with relapsed or refractory AML after ≤2 prior induction regiments, at least one containing anthracyclines. In some embodiments, the patient is ≥60 years of age with newly diagnosed AML. In some embodiments, the patient has an absolute blast count 9ABC) of ≤40,000/mm. In some embodiments, the patient is medically eligible to receive MEC chemotherapy. In some embodiments, the patient is medically eligible to receive 7+3 cytarabine/idarubicin chemotherapy. In some embodiments, the patient has an Eastern Cooperative Oncology Group (ECOG) performance status of 0-2. In some embodiments, the patient has hemodynamically stable and adequate organ function. In some embodiments, the patient does not have acute promyelocytic leukemia. In some embodiments, the patient does not have acute leukemia of ambiguous lineage. In some embodiments, the patient does not have active signs or symptoms of CNS involvement by malignancy. In some embodiments, the patient has no prior G-CSF, GM-CSF or plerixafor within 14 days of treatment with the pharmaceutical composition disclosed herein. In some embodiments, the patient has no known history or evidence of active hepatitis A, B, or C or HIV. In some embodiments, the patient does not have uncontrolled acute life-threatening bacterial, viral, or fungal infection. In some embodiments, the patient does not have active graft versus host disease (GVHD)≥Grade 2 or extensive chronic GVHD requiring immunosuppressive therapy. In some embodiments, the patient does not have hematopoietic stem cell transplantation ≤4 months prior to the treatments disclosed herein. In some embodiments, the patient does not have clinically significant cardiovascular disease.

In some embodiments, the E-selectin inhibitor is chosen from the compound of Formula I, prodrugs of the compound of Formula I, and pharmaceutically acceptable salts of any of the foregoing. In some embodiments, the E-selectin inhibitor is the compound of Formula I. In some embodiments, the E-selectin inhibitor is chosen from pharmaceutically acceptable salts of the compound of Formula I. In some embodiments, the pharmaceutically acceptable salt is a sodium salt.

In some embodiments, the E-selectin antagonist is chosen from compounds of Formula Ix:

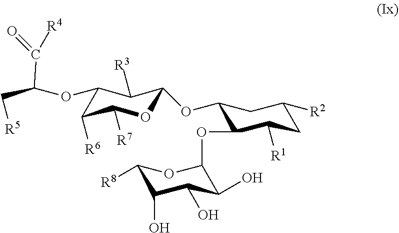

prodrgus of Formula Ix, and pharmaceutically acceptable salts of any of the foregoing, wherein:
R¹ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R² is chosen from H, -M, and -L-M;
R³ is chosen from —OH, —NH₂, —OC(═O)Y′, —NHC(═O)Y′, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups, wherein Z¹ and Z² may join together to form a ring;
R⁵ is chosen from C₃-C₈ cycloalkyl groups;
R⁶ is chosen from —OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁷ is chosen from —CH₂OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁸ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

In some embodiments, the E-selectin antagonist is chosen from compounds of Formula Ix, wherein the non-glycomimetic moiety comprises polyethylene glycol.

In some embodiments, the E-selectin antagonist is chosen from compounds of Formula Ix, wherein the linker is —C(═O)NH(CH₂)₁₋₄NHC(═O)— and the non-glycomimetic moiety comprises polyethylene glycol.

In some embodiments, the E-selectin inhibitor is chosen from the compound of Formula Ix, prodrugs of compounds of Formula Ix and pharmaceutically acceptable salts of any of the foregoing. In some embodiments, the E-selectin inhibitor is the compound of Formula Ix. In some embodiments, the E-selectin inhibitor is chosen from pharmaceutically acceptable salts of the compound of Formula Ix.

In some embodiments, the E-selectin antagonist is chosen from compounds of Formula Ia:

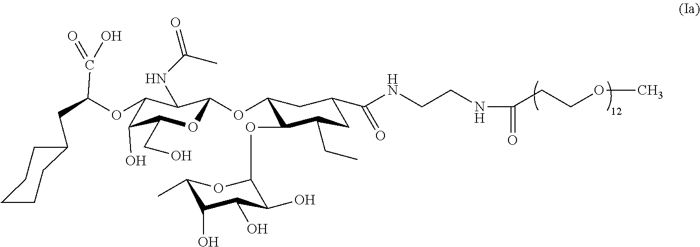

and pharmaceutically acceptable salts thereof, wherein n is chosen from integers ranging from 1 to 100. In some embodiments, n is chosen from 4, 8, 12, 16, 20, 24, and 28. In some embodiments n is 12.

In some embodiments, the E-selectin antagonist is a heterobifunctional antagonist chosen from compounds of Formula II:

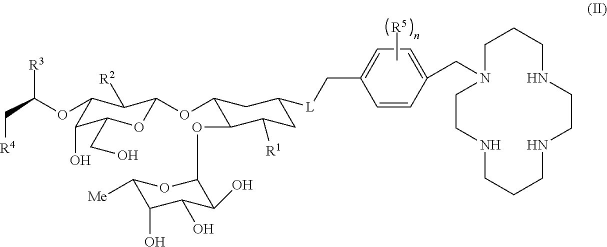

prodrugs of compounds of Formula II, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y₁ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁-8 haloalkyl, C₂₋₈ haloalkenyl, and C₂₋₈ haloalkynyl groups, wherein Z¹ and Z² may join together to form a ring;
R⁴ is chosen from C₃₋₈ cycloalkyl groups;
R¹ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

In some embodiments, the E-selectin antagonist is a heterobifunctional antagonist chosen from compounds of Formula IIa:

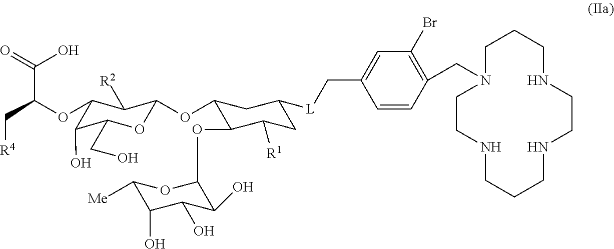

and pharmaceutically acceptable salts thereof.

In some embodiments, the linker groups of Formula Ix and/or Formula II are independently chosen from groups comprising spacer groups, such spacer groups as, for example, —(CH₂)ₚ— and —O(CH₂)ₚ—, wherein p is chosen from integers ranging from 1 to 30. In some embodiments, p is chosen from integers ranging from 1 to 20.

Other non-limiting examples of spacer groups include carbonyl groups and carbonyl-containing groups such as, for example, amide groups. A non-limiting example of a spacer group is

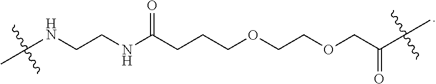 [SMILES: *NCCNC(=O)CCCOCCOCC(*)=O]

In some embodiments, the linker groups of Formula Ix and/or Formula II are independently chosen from

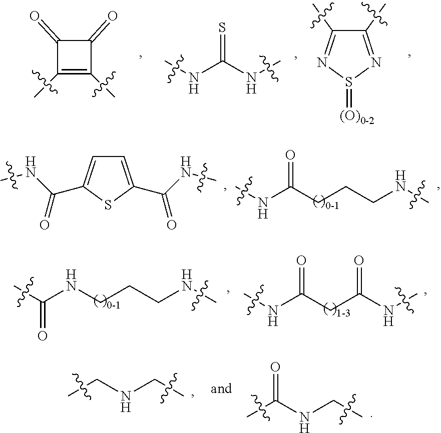

Other linker groups, such as, for example, polyethylene glycols (PEGs) and —C(═O)—NH—(CH₂)ₚ—C(═O)—NH—, wherein p is chosen from integers ranging from 1 to 30, or wherein p is chosen from integers ranging from 1 to 20, will be familiar to those of ordinary skill in the art and/or those in possession of the present disclosure.

In some embodiments, at least one linker group of Formula Ix and/or Formula II is

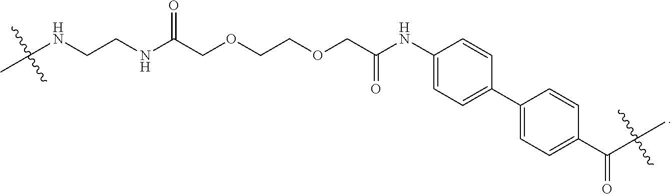 [SMILES: *NCCNC(=O)COCCOCC(=O)Nc1ccc(-c2ccc(C(*)=O)cc2)cc1]

In some embodiments, at least one linker group of Formula Ix and/or Formula II is

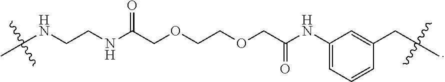 [SMILES: *Cc1cccc(NC(=O)COCCOCC(=O)NCCN*)c1]

In some embodiments, at least one linker group of Formula Ix and/or Formula II is chosen from —C(═O)NH(CH₂)₂NH—, —CH₂NHCH₂—, and —C(═O)NHCH₂—. In some embodiments, at least one linker group is —C(═O)NH(CH₂)₂NH—.

In some embodiments, the E-selectin antagonist is chosen from Compound B:

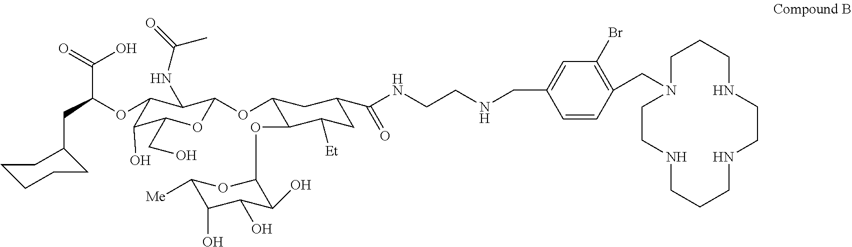 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.

In some embodiments, the E-selectin antagonist is chosen from compounds of Formula III:

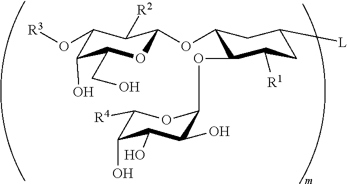

prodrugs of compounds of Formula III, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

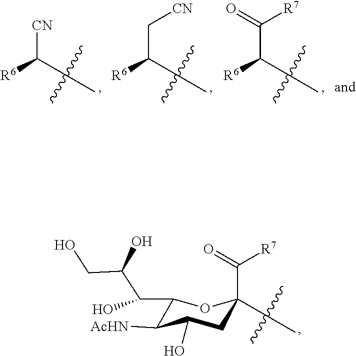

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

In some embodiments, the E-selectin antagonist is chosen from compounds of Formula IV:

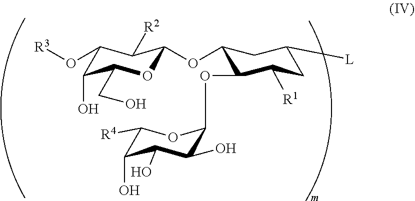

prodrugs of compounds of Formula IV, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y₁ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y₁ and Y² may join together along with the nitrogen atom to which they are attached to form a ring;
each R³, which may be identical or different, is independently chosen from

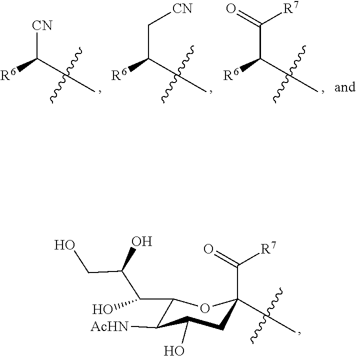

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

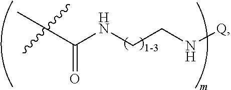

wherein Q is a chosen from

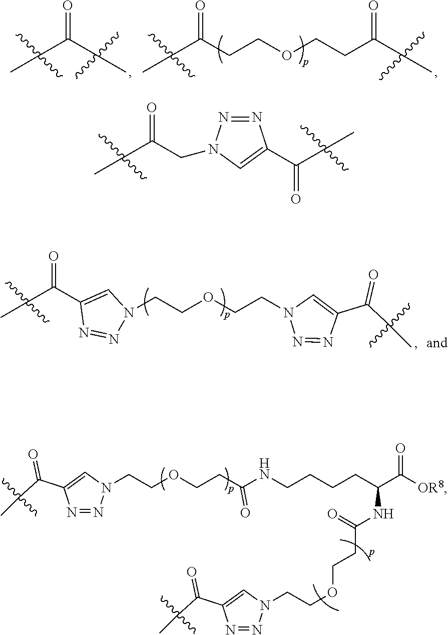

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

In some embodiments, the E-selectin antagonist of Formula III or Formula IV is chosen from compounds of the following Formula IIIa/IVa (see definitions of L and m for Formula III or IV above):

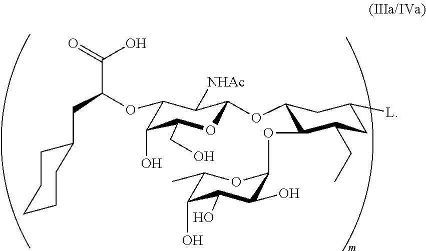

In some embodiments, the E-selectin antagonist of Formula III or Formula IV is chosen from compounds of the following Formula IIIb/IVb (see definitions of L and m for Formula III or IV above):

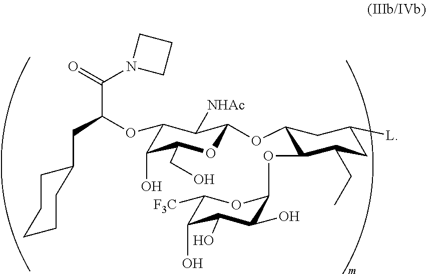

In some embodiments, the E-selectin antagonist is Compound C:

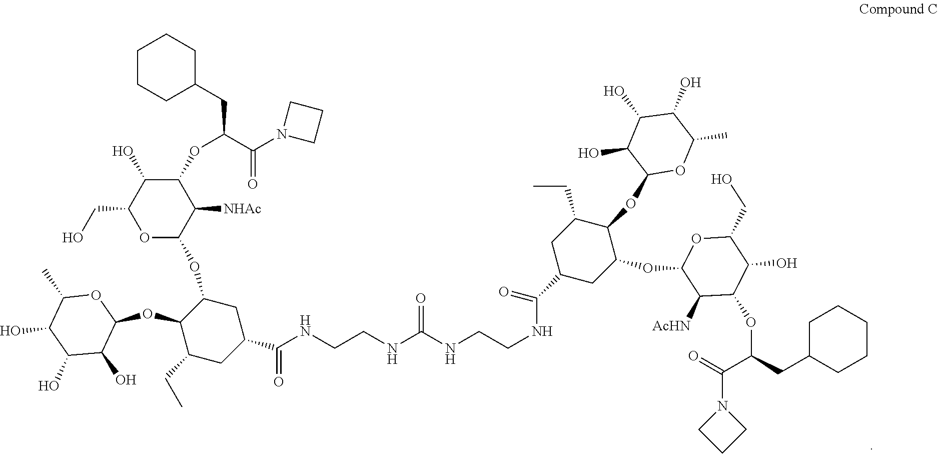

In some embodiments, the E-selectin antagonist is a heterobifunctional inhibitor of E-selectin and Galectin-3, chosen from compounds of Formula V:

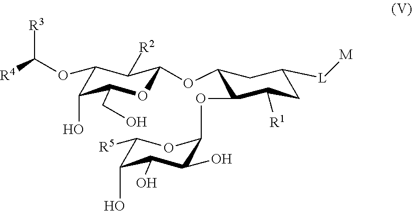

prodrugs of compounds of Formula V, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

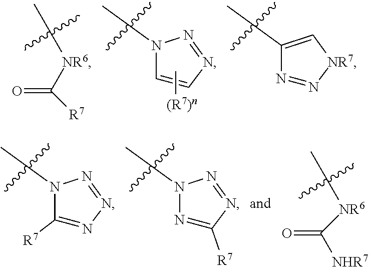

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, wherein Y¹ and Y² may join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇-12 arylalkyl groups, wherein Z¹ and Z² may join together along with the nitrogen atom to which they are attached to form a ring; R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

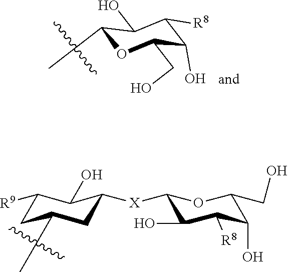

groups, wherein X is chosen from O and S, and R⁸ and R⁹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y⁴ groups, wherein Y⁴ is chosen from C₁₋₈ alkyl, C₂₋₁₂ heterocyclyl, C₆₋₁₈ aryl, and C₁₋₁₃ heteroaryl groups; and
L is chosen from linker groups.

In some embodiments, the E-selectin antagonist is chosen from compounds having the following Formulae:

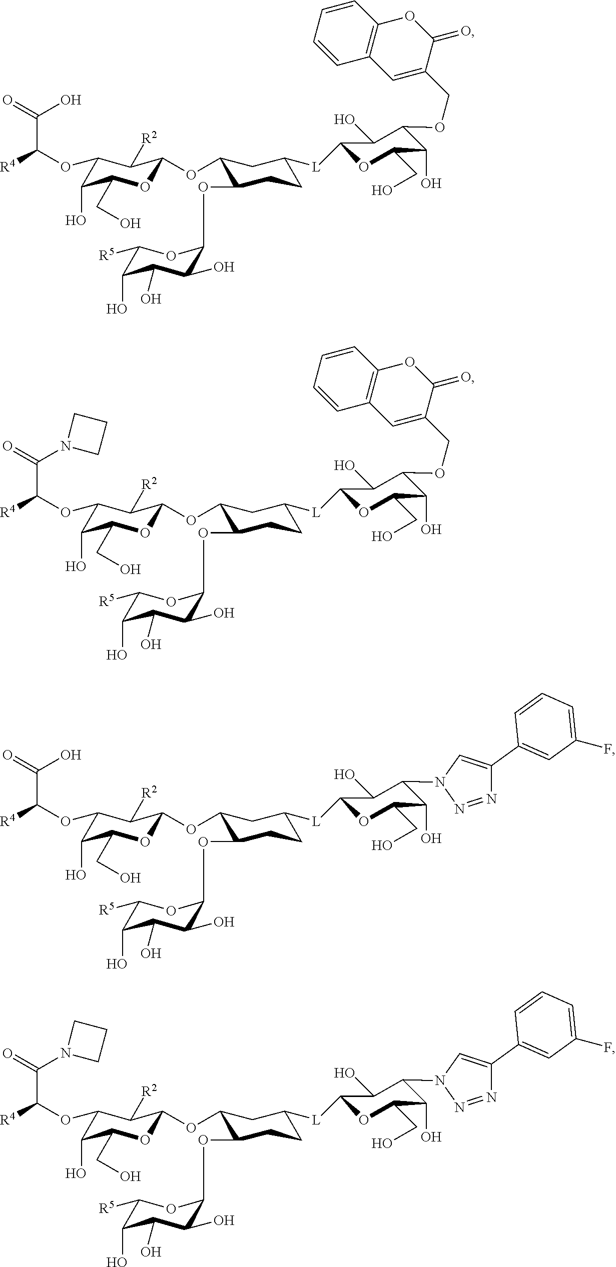

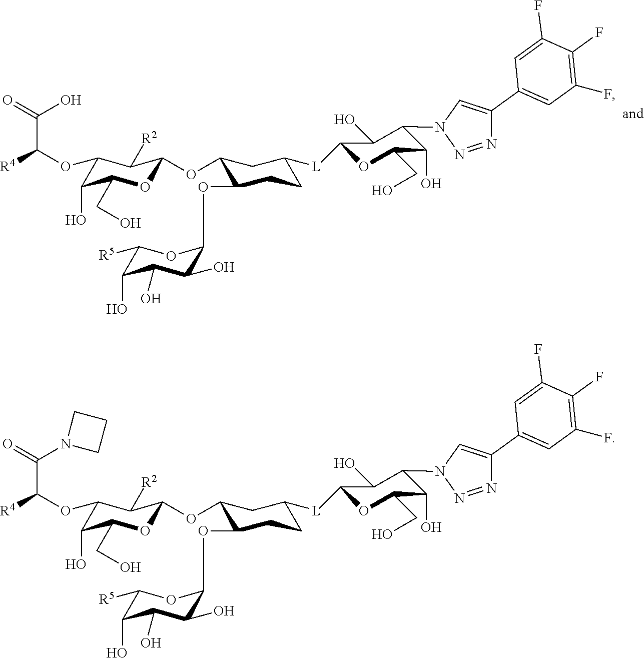

In some embodiments, the E-selectin antagonist is chosen from compounds having the following Formulae:

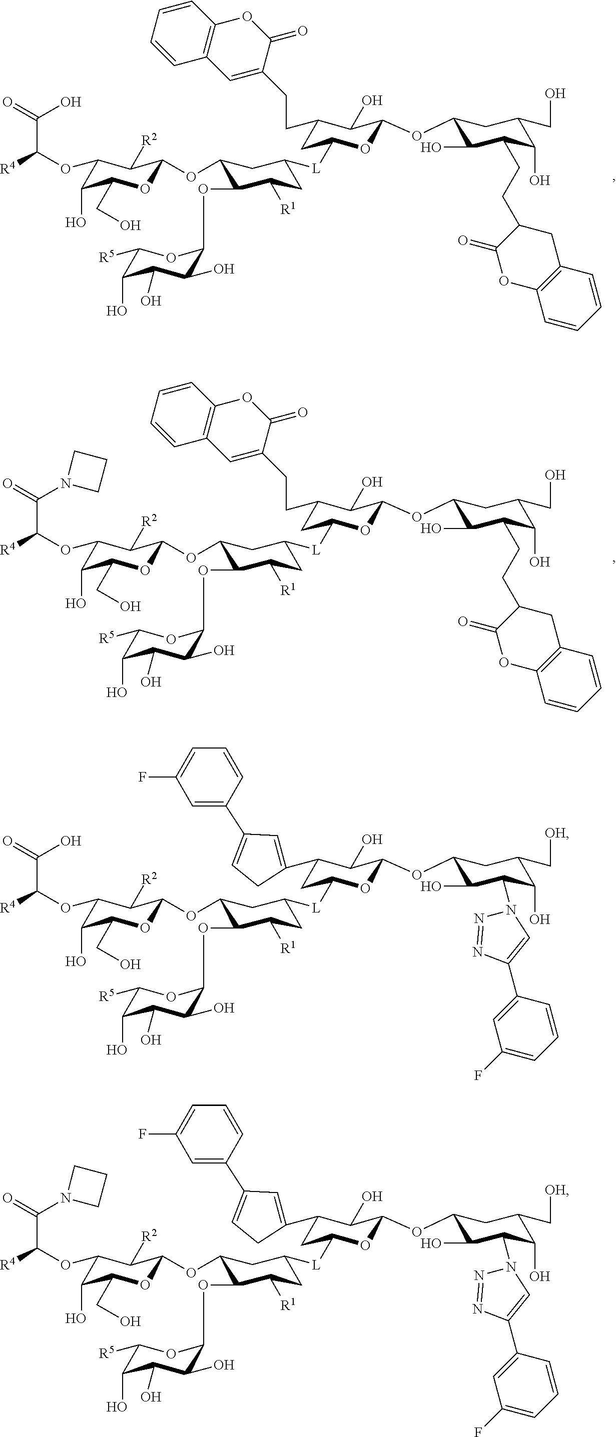

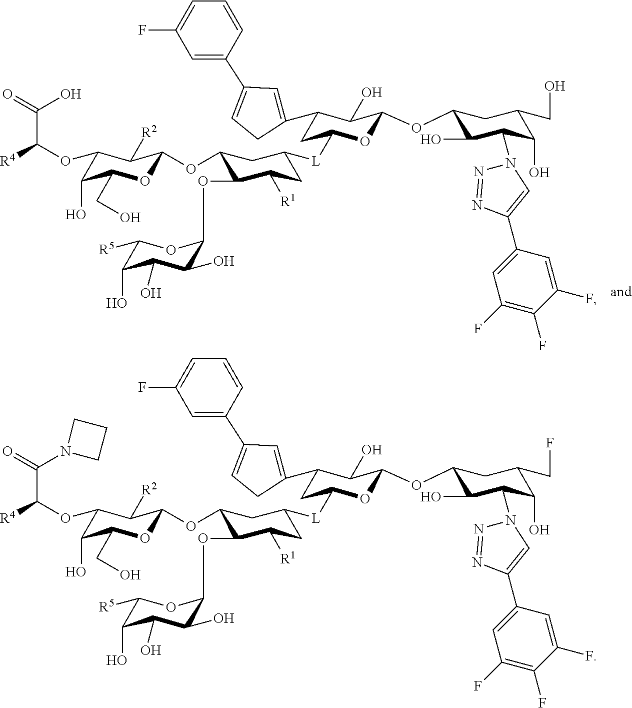

In some embodiments, the E-selectin antagonist is Compound D:

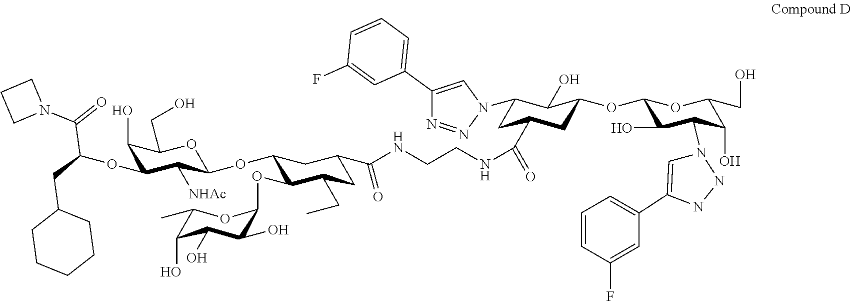

In some embodiments, the E-selectin antagonist is chosen from compounds of Formula VI:

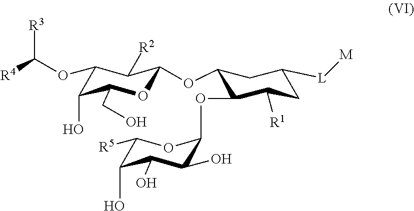

prodrugs of compounds of Formula VI, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

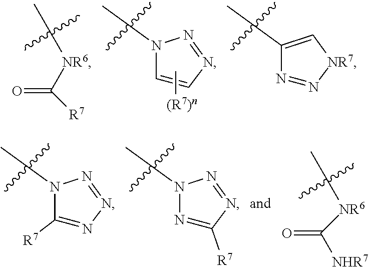

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY′, halo, —NH₂, —NY¹Y², —OC(═O)Y¹, —NHC(═O)Y¹, and —NHC(═O)NHY¹ groups, wherein YI and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or YI and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇-12 arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups; R⁵ is chosen from —CN, C₁₋₈ alkyl, and C₁₋₄ haloalkyl groups;
M is chosen from

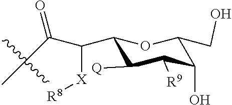

groups,
wherein
X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
Q is chosen from H, halo, and —OZ³ groups, wherein Z3 is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z⁷ groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring, and
wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments of Formula VI, M is chosen from

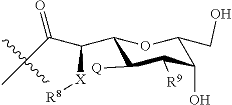

groups.

In some embodiments of Formula VI, M is chosen from

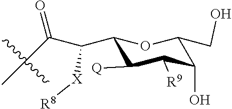

groups.

In some embodiments of Formula VI, linker groups may be chosen from groups comprising spacer groups, such spacer groups as, for example, —(CH₂)— and —O(CH₂)—, wherein t is chosen from integers ranging from 1 to 20. Other non-limiting examples of spacer groups include carbonyl groups and carbonyl-containing groups such as, for example, amide groups. A non-limiting example of a spacer group is

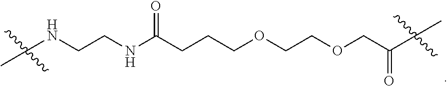 [SMILES: *NCCNC(=O)CCCOCCOCC(*)=O]

In some embodiments of Formula VI, the linker group is chosen from

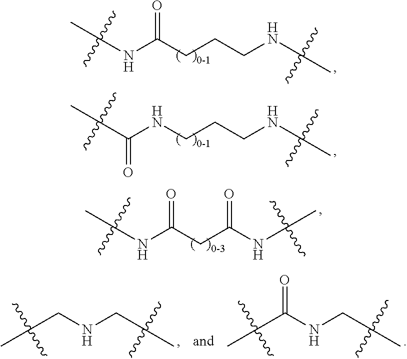

In some embodiments of Formula VI, the linker group is chosen from polyethylene glycols (PEGs), —C(═O)NH(CH₂)ᵥO—, —C(═O)NH(CH₂)ᵥNHC(═O)—, —C(═O)NHC(═O)(CH₂)NH—, and —C(═O)NH(CH₂)ᵥC(═O)NH— groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments of Formula VI, the linker group is

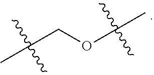 [SMILES: *CO*]

In some embodiments of Formula VI, the linker group is

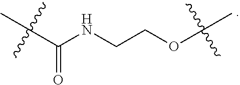 [SMILES: *OCCNC(*)=O]

In some embodiments of Formula VI, the linker group is

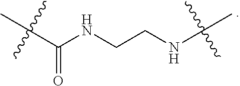 [SMILES: *NCCNC(*)=O]

In some embodiments of Formula VI, the linker group is

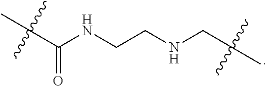 [SMILES: *CNCCNC(*)=O]

In some embodiments of Formula VI, the linker group is

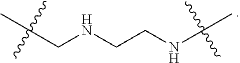 [SMILES: *CNCCN*]

In some embodiments of Formula VI, the linker group is

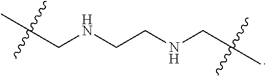 [SMILES: *CNCCNC*]

In some embodiments of Formula VI, the linker group is

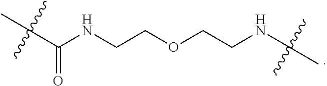 [SMILES: *NCCOCCNC(*)=O]

In some embodiments of Formula VI, the linker group is

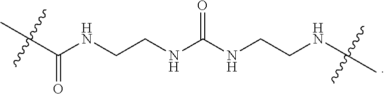 [SMILES: *NCCNC(=O)NCCNC(*)=O]

In some embodiments of Formula VI, the linker group is

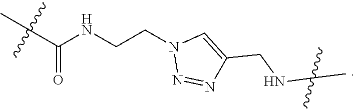 [SMILES: *NCc1cn(CCNC(*)=O)nn1]

In some embodiments, the E-selectin antagonist is a multimeric inhibitor of E-selectin, Galectin-3, and/or CXCR4, chosen from compounds of Formula VII:

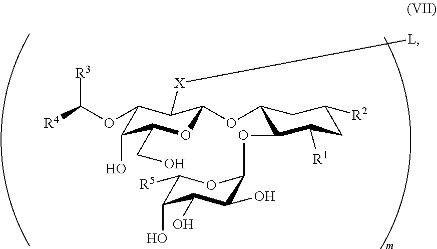

prodrugs of compounds of Formula VII, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl,

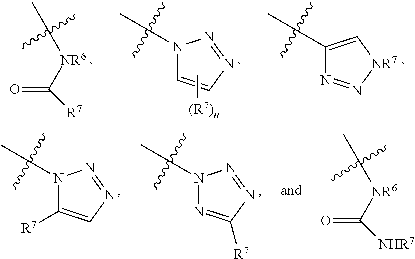

groups, wherein each n, which may be identical or different, is chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

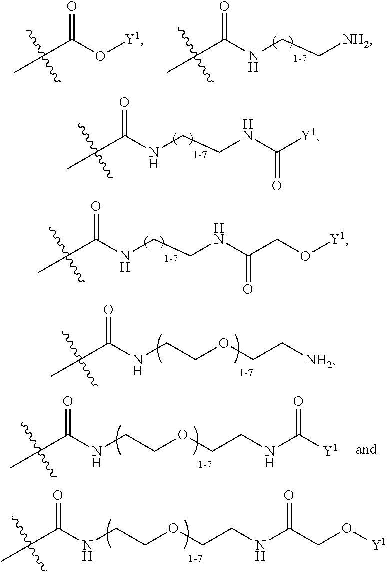

groups,
wherein each Y, which may be identical or different, is independently chosen from C₁₋₄ alkyl, C₂-4 alkenyl, and C₂-4 alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

In some embodiments of Formula VII, at least one linker group is chosen from groups comprising spacer groups, such spacer groups as, for example, —(CH₂)_{z}— and —O(CH₂)_{z}—, wherein z is chosen from integers ranging from 1 to 250. Other non-limiting examples of spacer groups include carbonyl groups and carbonyl-containing groups such as, for example, amide groups. A non-limiting example of a spacer group is

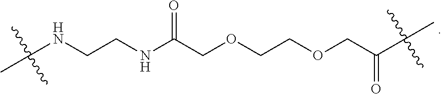 [SMILES: *NCCNC(=O)COCCOCC(*)=O]

In some embodiments of Formula VII, at least one linker group is chosen from

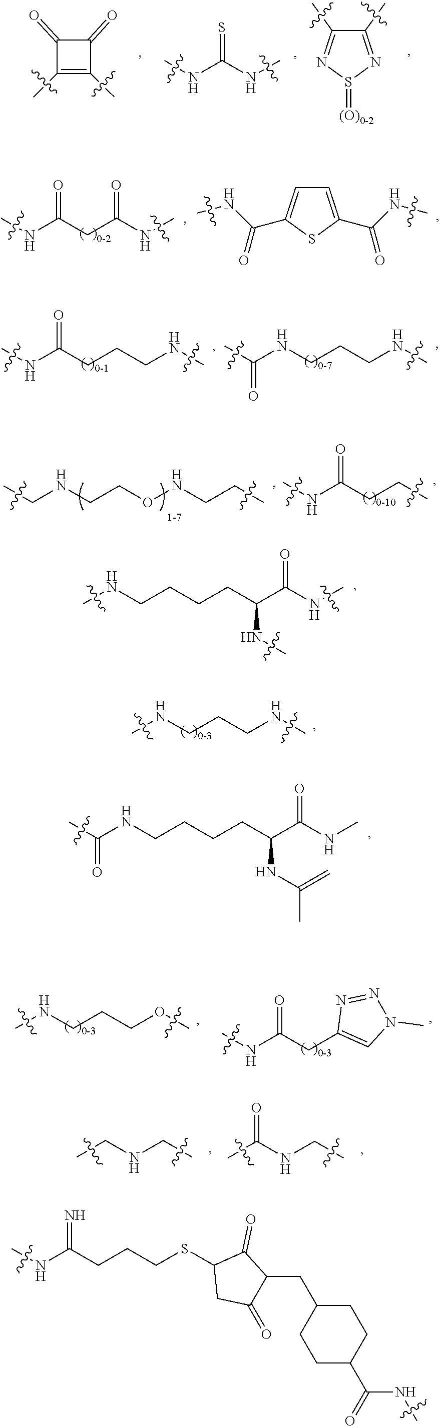

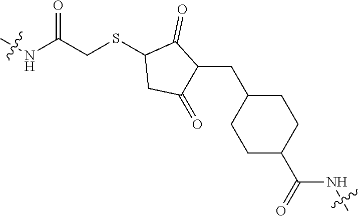

groups.

Other linker groups for certain embodiments of Formula VII, such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments of Formula VII, at least one linker group is

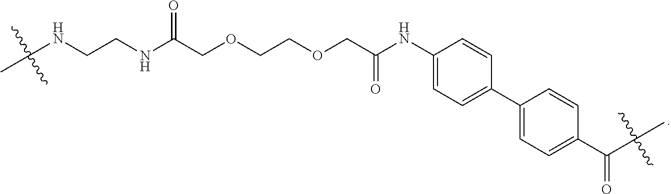 [SMILES: *NCCNC(=O)COCCOCC(=O)Nc1ccc(-c2ccc(C(*)=O)cc2)cc1]

In some embodiments of Formula VII, at least one linker group is

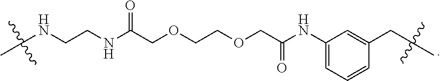 [SMILES: *Cc1cccc(NC(=O)COCCOCC(=O)NCCN*)c1]

In some embodiments of Formula VII, at least one linker group is chosen from —C(═O)NH(CH₂)₂NH—, —CH₂NHCH₂—, and —C(═O)NHCH₂—. In some embodiments of Formula VII, at least one linker group is —C(═O)NH(CH₂)₂NH—.

In some embodiments of Formula VII, L is chosen from dendrimers. In some embodiments of Formula VII, L is chosen from polyamidoamine (“PAMAM”) dendrimers. In some embodiments of Formula VII, L is chosen from PAMAM dendrimers comprising succinamic. In some embodiments of Formula VII, L is PAMAM GO generating a tetramer. In some embodiments of Formula VII, L is PAMAM G1 generating an octamer. In some embodiments of Formula VII, L is PAMAM G2 generating a 16-mer. In some embodiments of Formula VII, L is PAMAM G3 generating a 32-mer. In some embodiments of Formula VII, L is PAMAM G4 generating a 64-mer. In some embodiments, L is PAMAM G5 generating a 128-mer.

In some embodiments of Formula VII, m is 2 and L is chosen from

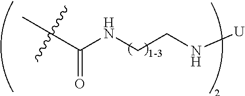

groups,
wherein U is chosen from

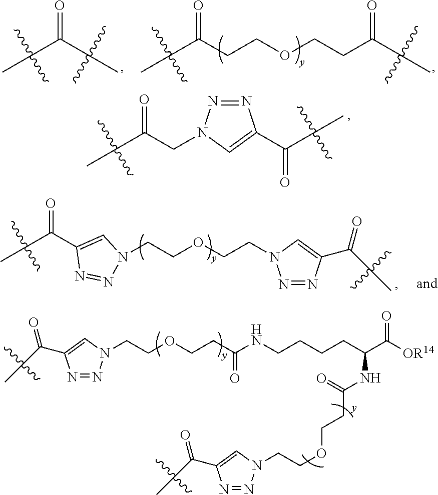

groups,
wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments of Formula VII, R¹⁴ is chosen from C₁₋₈ alkyl. In some embodiments of Formula VII, R¹⁴ is chosen from C₇-9 arylalkyl. In some embodiments of Formula VII, R¹⁴ is H. In some embodiments of Formula VII, R¹⁴ is benzyl.

In some embodiments of Formula VII, L is chosen from

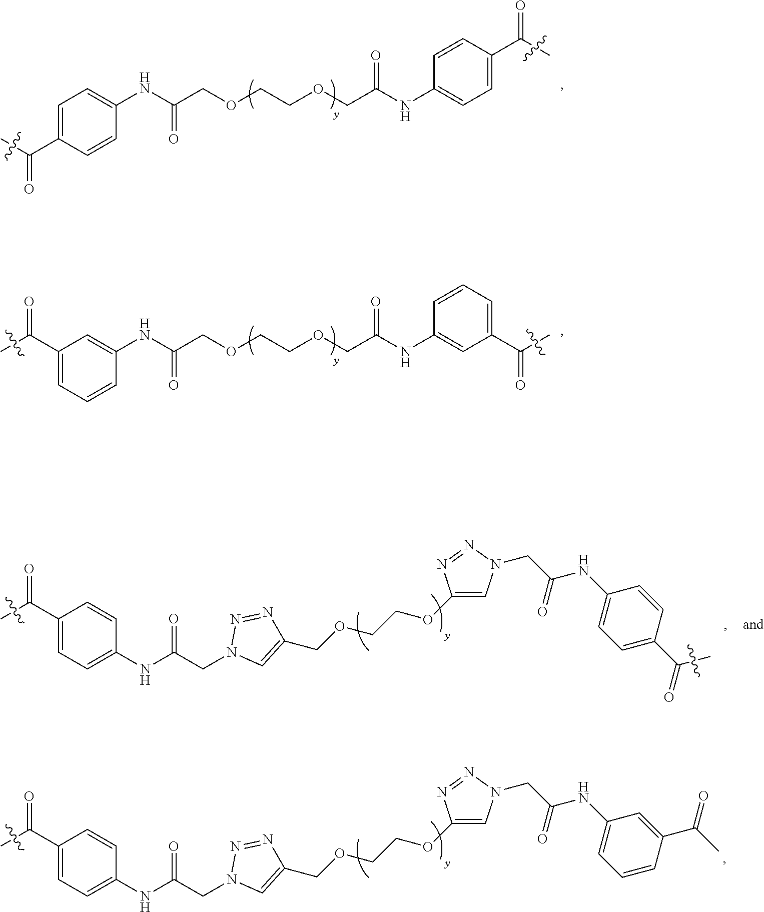

wherein y is chosen from integers ranging from 0 to 250.

In some embodiments of Formula VII, L is chosen from

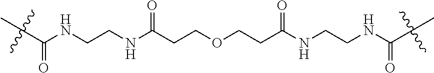 [SMILES: *C(=O)NCCNC(=O)CCOCCC(=O)NCCNC(*)=O]

groups,
wherein y is chosen from integers ranging from 0 to 250.

In some embodiments of Formula VII, L is

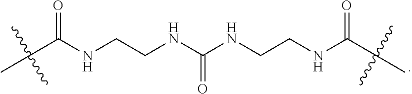 [SMILES: *C(=O)NCCNC(=O)NCCNC(*)=O]

In some embodiments of Formula VII, L is chosen from

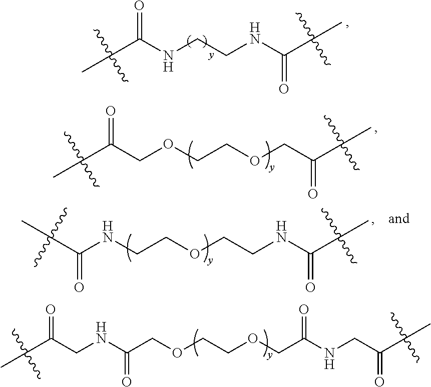

groups,
wherein y is chosen from integers ranging from 0 to 250.

In some embodiments of Formula VII, L is chosen from

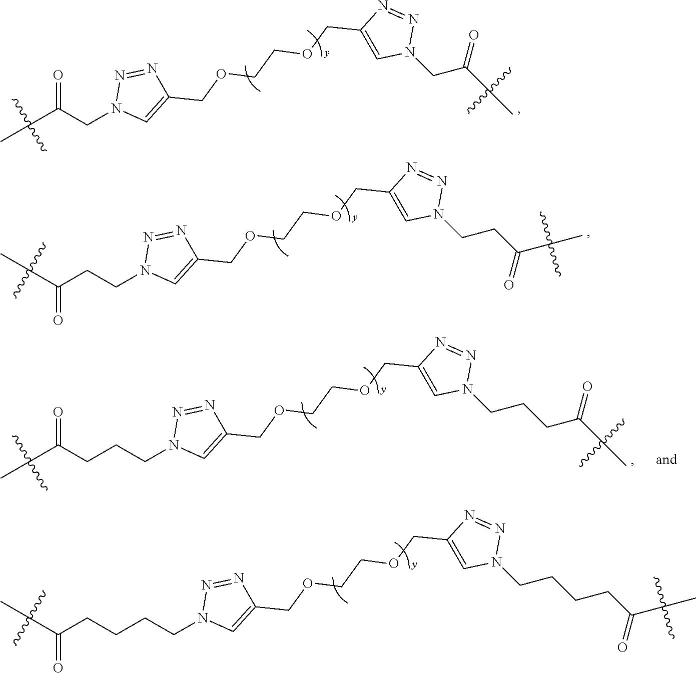

groups,
wherein y is chosen from integers ranging from 0 to 250.

In some embodiments of Formula VII, L is chosen from

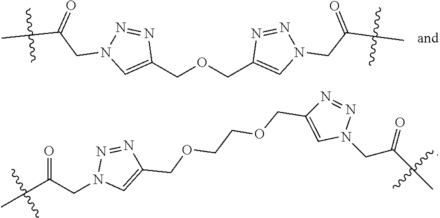

In some embodiments of Formula VII, L is

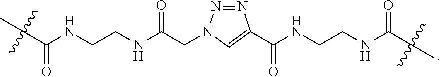 [SMILES: *C(=O)NCCNC(=O)Cn1cc(C(=O)NCCNC(*)=O)nn1]

In some embodiments of Formula VII, L is chosen from

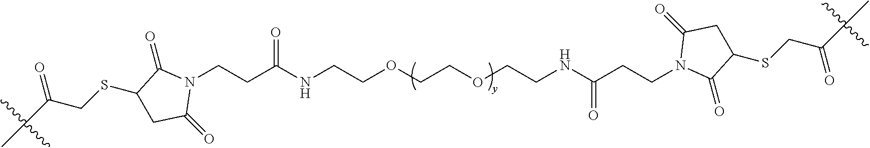

groups,
wherein y is chosen from integers ranging from 0 to 250.

In some embodiments of Formula VII, L is

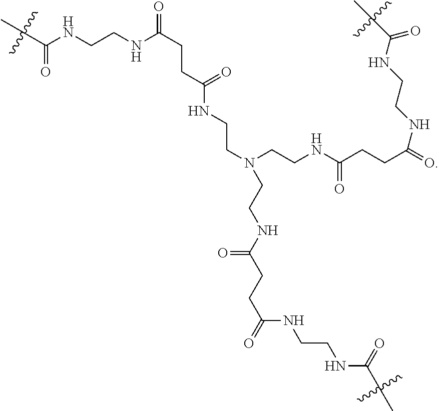 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments of Formula VII, L is

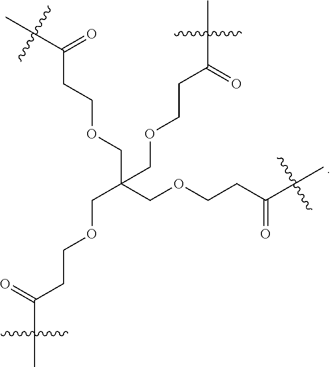 [SMILES: *C(=O)CCOCC(COCCC(*)=O)(COCCC(*)=O)COCCC(*)=O]

In some embodiments of Formula VII, L is

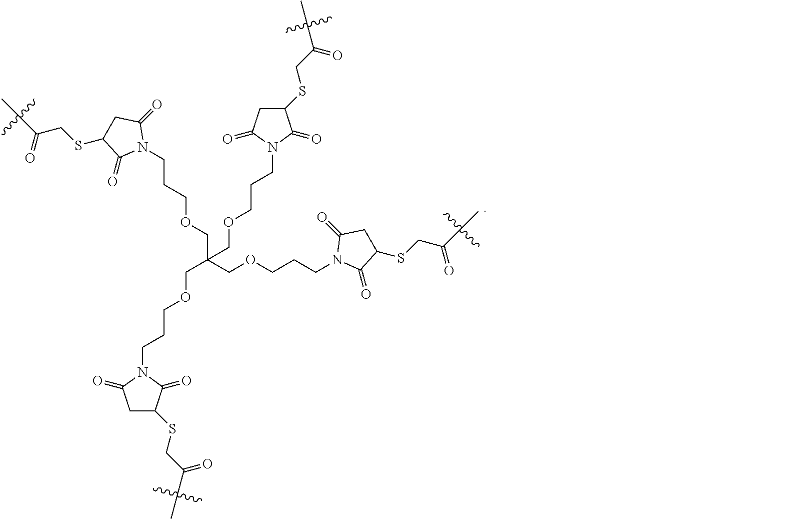 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments of Formula VII, L is chosen from

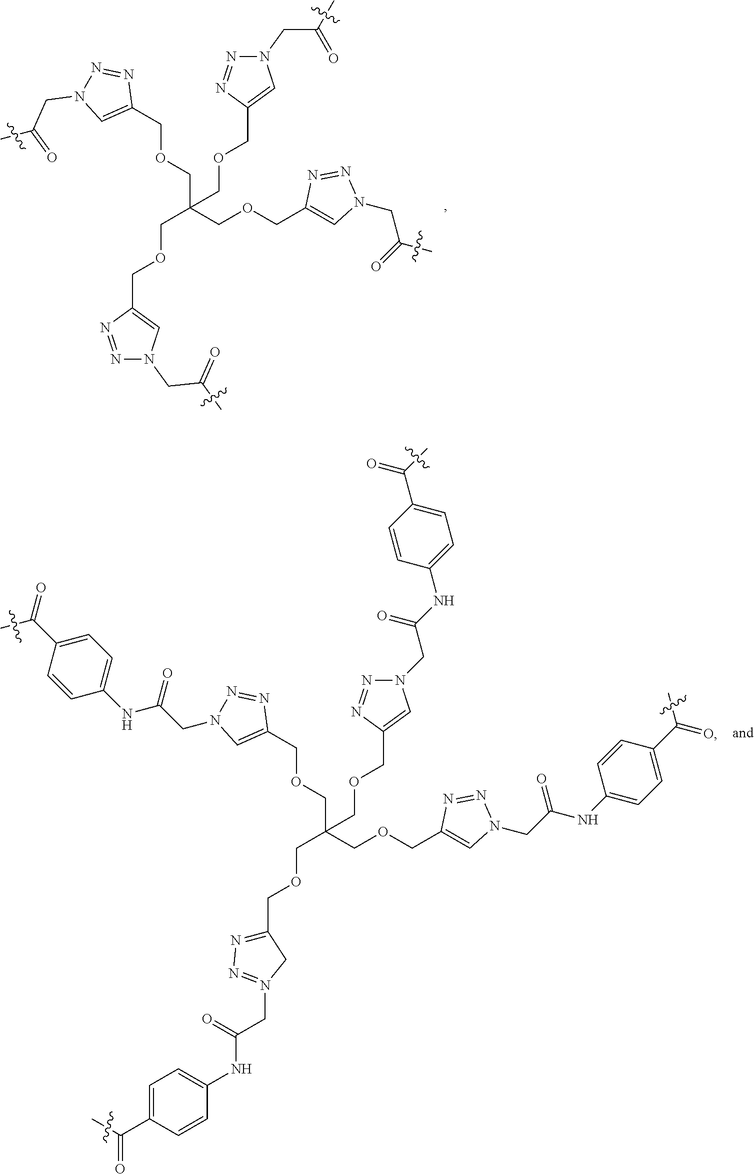

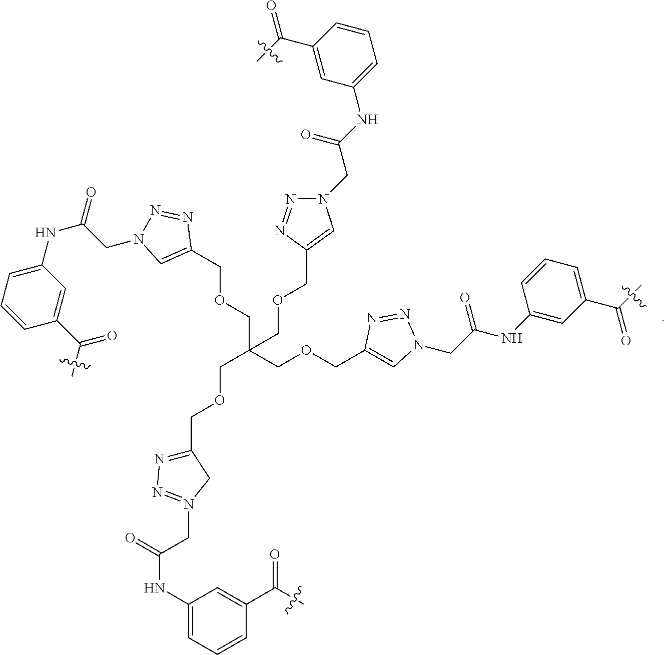

In some embodiments of Formula VII, L is

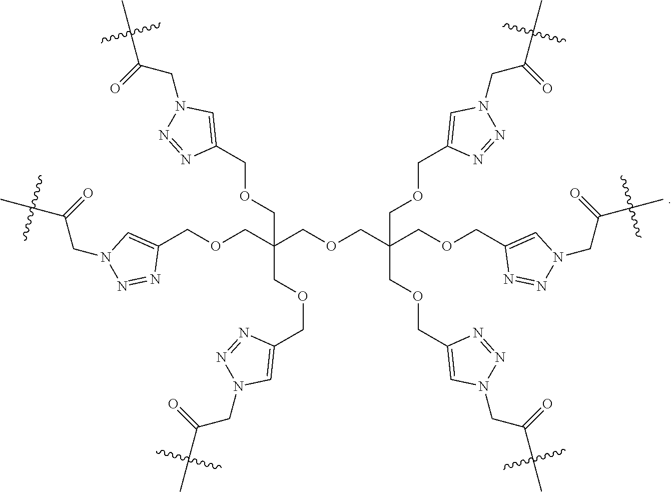 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments of Formula VII, L is chosen from

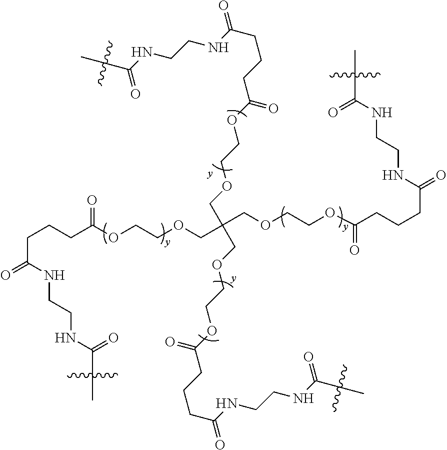

wherein each y, which may be identical or different, is independently chosen from integers ranging from 0 to 250.

In some embodiments of Formula VII, L is chosen from

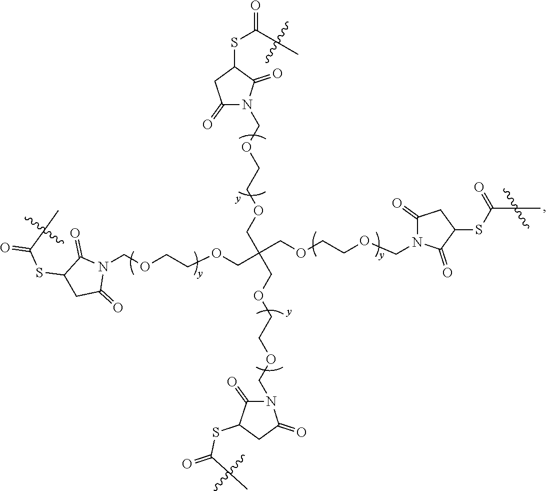

wherein each y, which may be identical or different, is independently chosen from integers ranging from 0 to 250.

In some embodiments of Formula VII, L is chosen from

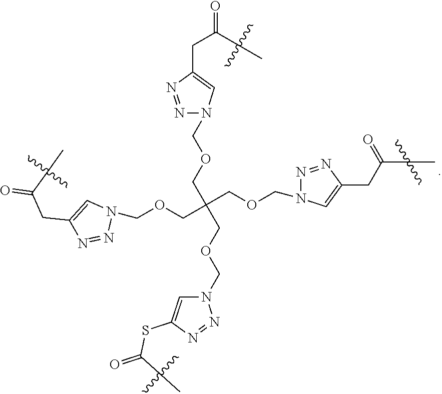 [SMILES: *C(=O)Cc1cn(COCC(COCn2cc(CC(*)=O)nn2)(COCn2cc(CC(*)=O)nn2)COCn2cc(SC(*)=O)nn2)nn1]

In some embodiments, at least one compound is chosen from compounds of Formula VII, wherein each R¹ is identical, each R² is identical, each R³ is identical, each R⁴ is identical, each R⁵ is identical, and each X is identical. In some embodiments, at least one compound is chosen from compounds of Formula VII, wherein said compound is symmetrical.

Provided are pharmaceutical compositions comprising at least one compound chosen from compounds of Formula Ix, Ia, II, IIa, III, IV, IIIa/IVa, IIIb/IVb, V, VI, and VII, and pharmaceutically acceptable salts of any of the foregoing. Also provided are pharmaceutical compositions comprising at least one compound chosen from the compound of Formula I, compound B, compound C, and compound D, and pharmaceutically acceptable salts of any of the foregoing. These compounds and compositions may be used in the methods described herein.

### EXAMPLES

### Example 1

### Prophetic Synthesis of Multimeric Compound 21

Compound 3: A mixture of compound 1 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 3.

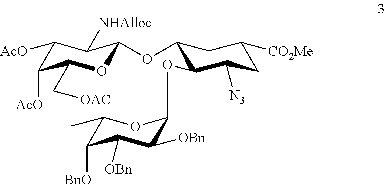

Compound 4: Compound 3 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 4.

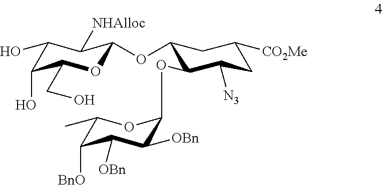

Compound 5: To a solution of compound 4 in dichloromethane cooled on an ice bath is added DABCO (1.5 eq) followed by monomethoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 5.

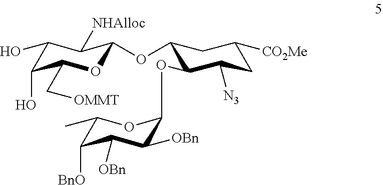

Compound 7: To a solution of compound 5 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 7.

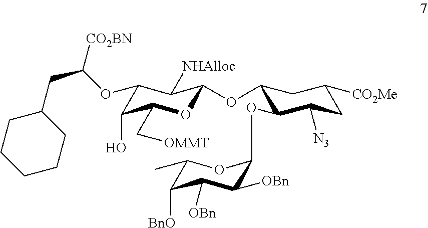

Compound 8: To a degassed solution of compound 7 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 8.

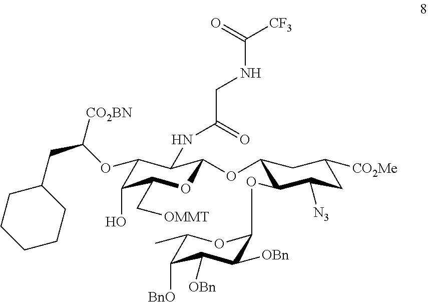

Compound 9: To a stirred solution of compound 8 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 9.

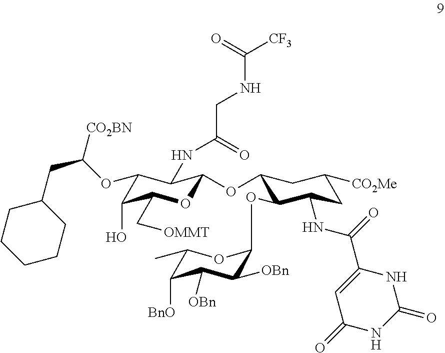

Compound 10: Compound 9 is dissolved in methanol and degassed. To this solution is added Pd(OH)₂/*C. The reaction mixture is vigorously stirred under a hydrogen atmosphere for 12 hours. The reaction mixture is filtered through a Celite pad. The filtrate is concentrated under reduced pressure to give compound 10.

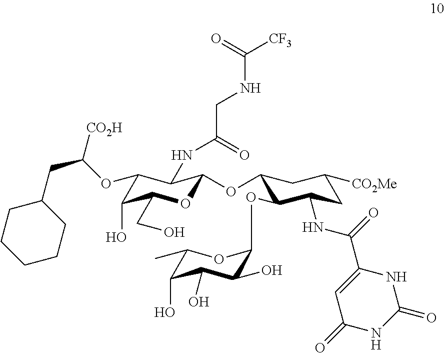

Compound 11: Compound 10 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 11.

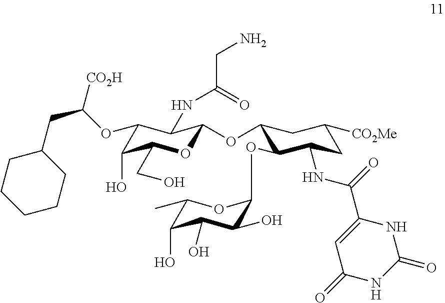

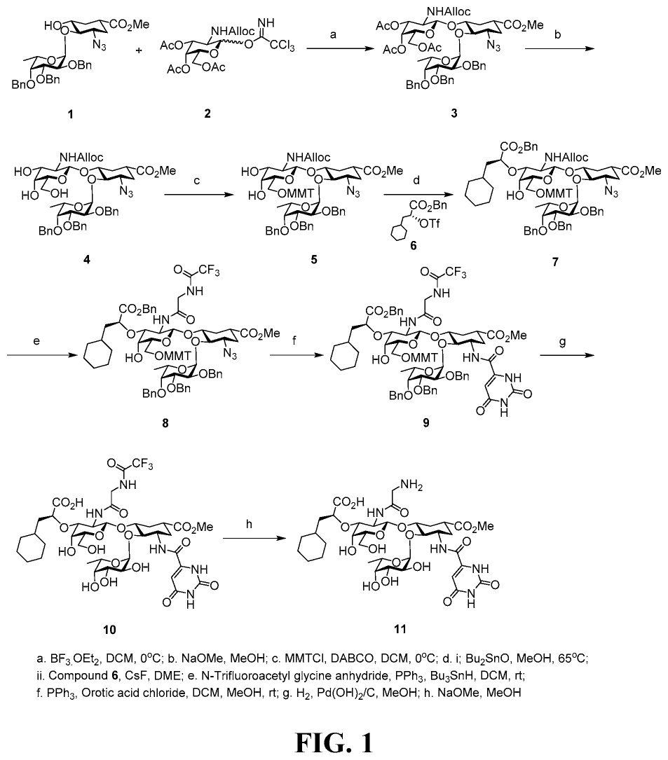

Compound 12: Compound 12 can be prepared in an analogous fashion to FIG. 1 by substituting (acetylthio)acetyl chloride for N-trifluoroacetyl glycine anhydride in step e.

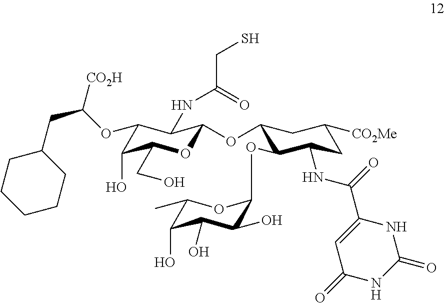

Compound 13: Compound 10 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 13.

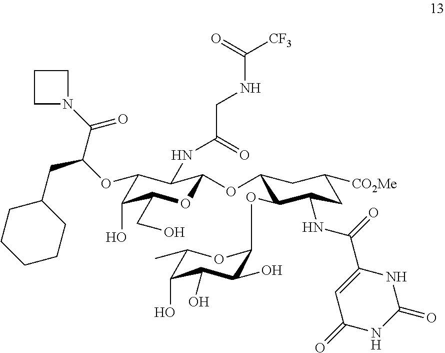

Compound 14: Compound 13 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 14.

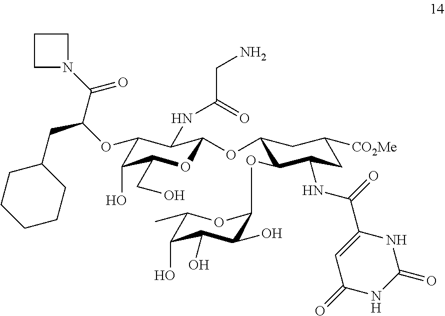

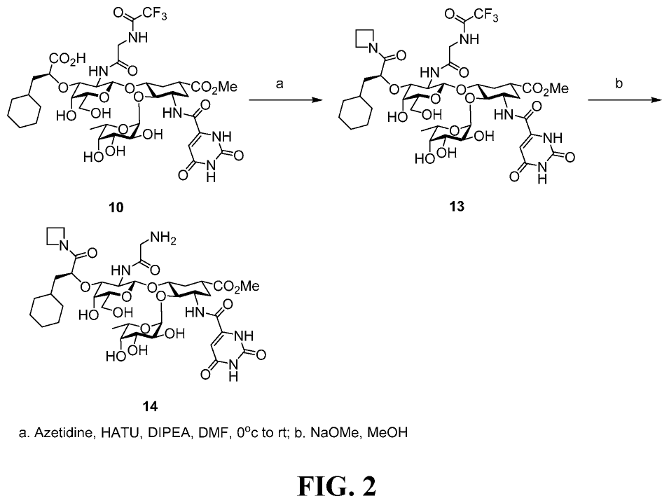

Compound 15: Compound 15 can be prepared in an analogous fashion to FIG. 2 by using methylamine in place of azetidine in step a.

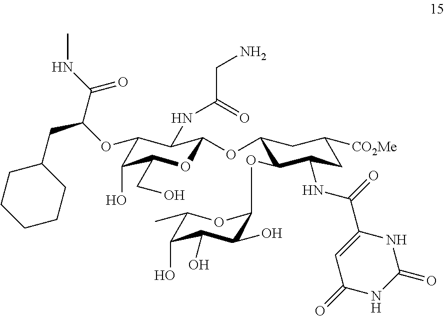

Compound 16: Compound 16 can be prepared in an analogous fashion to FIG. 2 by using dimethylamine in place of azetidine in step a.

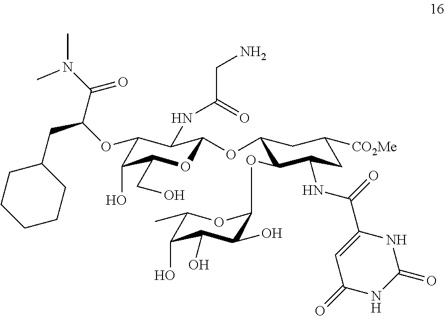

Compound 17: Compound 17 can be prepared in an analogous fashion to FIG. 2 by using 2-methoxyethylamine in place of azetidine in step a.

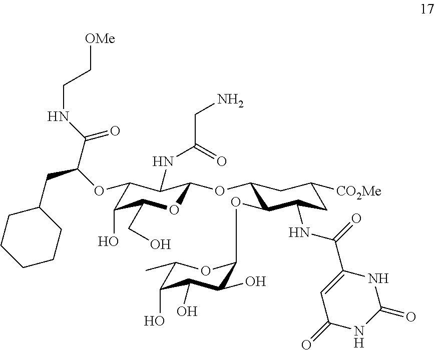

Compound 18: Compound 18 can be prepared in an analogous fashion to FIG. 2 by using piperidine in place of azetidine in step a.

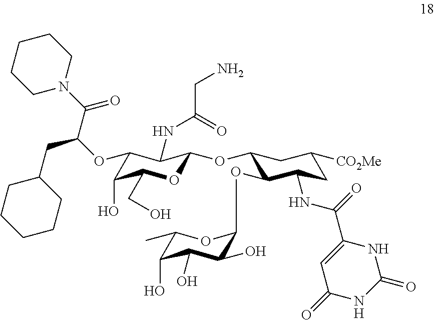

Compound 21: Compound 19 can be prepared in an analogous fashion to FIG. 2 by using morpholine in place of azetidine in step a.

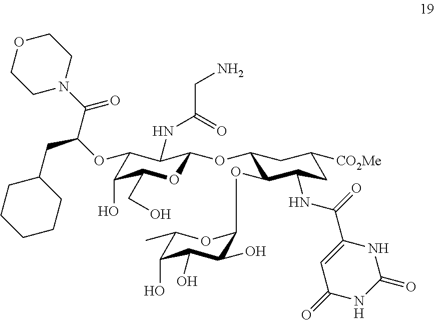

Compound 21: A solution of compound 20 (0.4 eq) in DMSO is added to a solution of compound 11 (1 eq) and DIPEA (10 eq) in anhydrous DMSO at room temperature. The resulting solution is stirred overnight. The solution is dialyzed against distilled water for 3 days with dialysis tube MWCO 1000 while distilled water is changed every 12 hours. The solution in the tube is lyophilized to give compound 21.

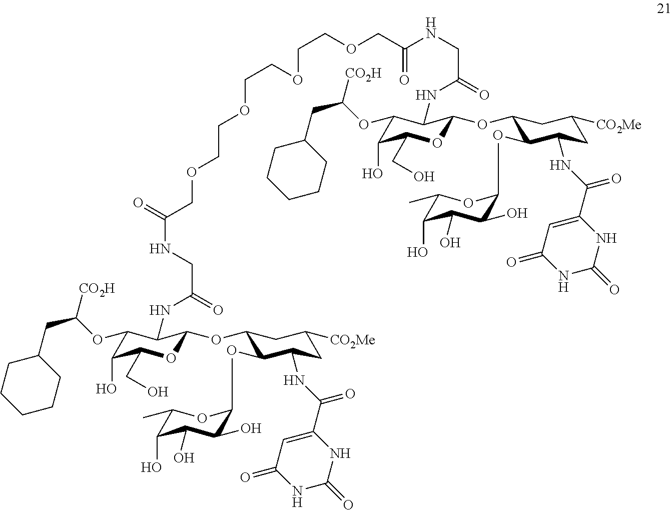

### Example 2

### Prophetic Synthesis of Multimeric Compound 22

Compound 22: A solution of compound 21 in ethylenediamine is stirred overnight at 70° C. The reaction mixture is concentrated under reduced pressure and the residue is purified by reverse phase chromatography to give compound 22.

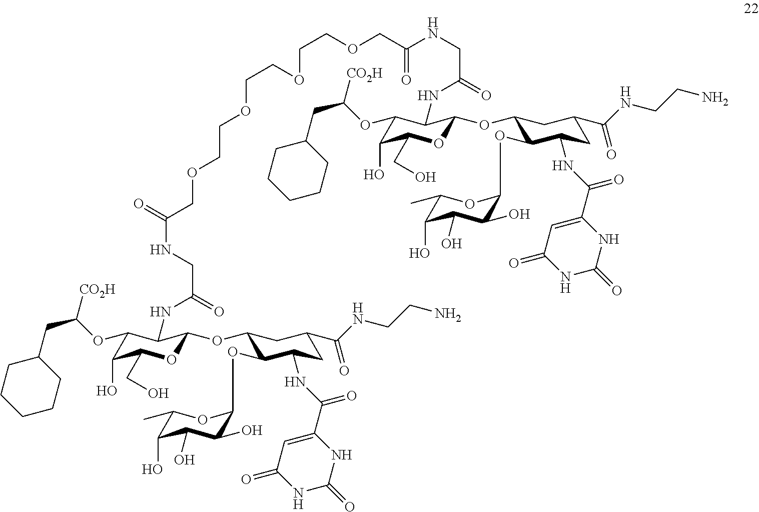

### Example 3

### Prophetic Synthesis of Multimeric Compound 23

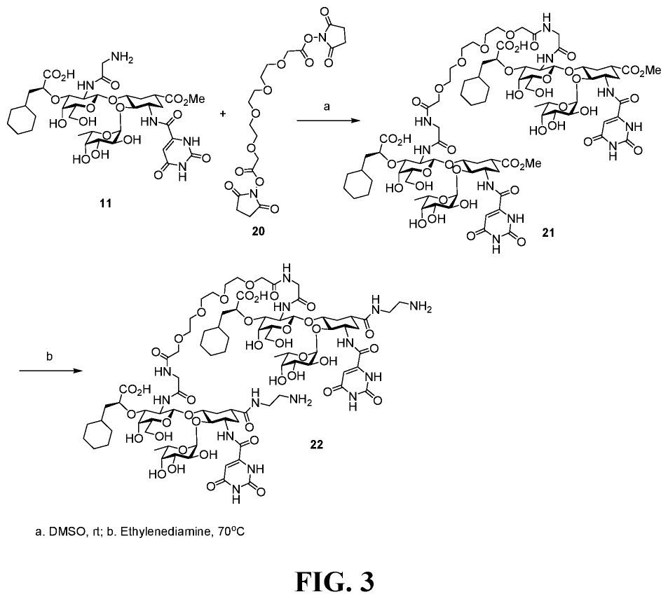

Compound 23: Compound 23 can be prepared in an analogous fashion to FIG. 3 by replacing compound 20 with PEG-11 diacetic acid di-NHS ester in step a.

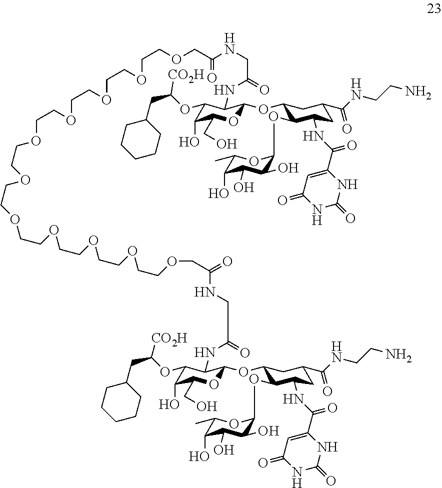

### Example 4

### Prophetic Synthesis of Multimeric Compound 24

Compound 24: Compound 24 can be prepared in an analogous fashion to FIG. 3 by replacing compound 20 with PEG-15 diacetic acid di-NHS ester in step a.

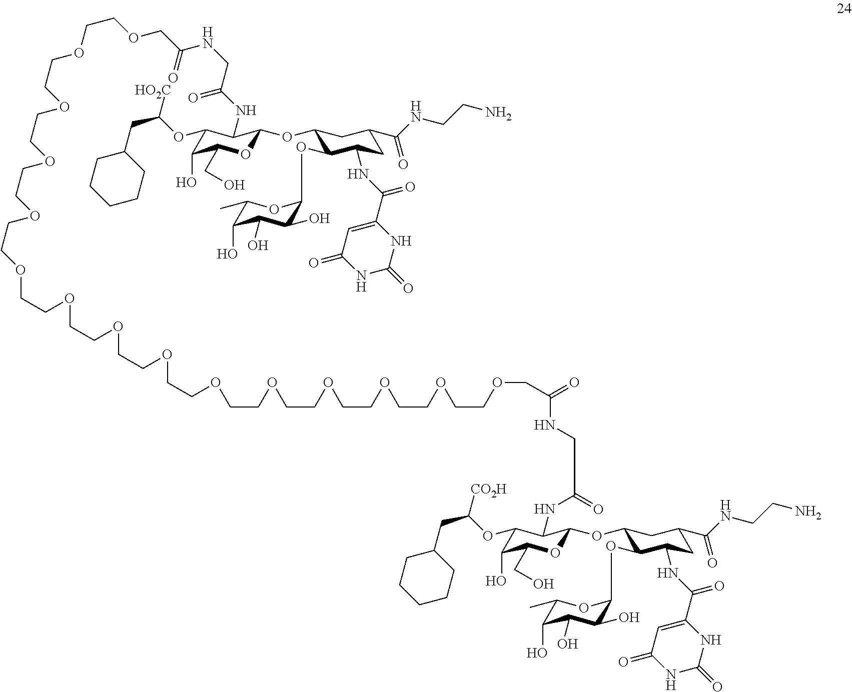

### Example 5

### Prophetic Synthesis of Multimeric Compound 25

Compound 25: Compound 25 can be prepared in an analogous fashion to FIG. 3 by replacing compound 20 with ethylene glycol diacetic acid di-NHS ester in step a.

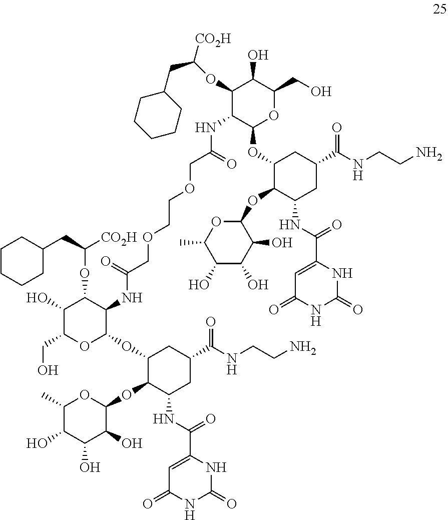 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 6

### Prophetic Synthesis of Multimeric Compound 26

Compound 26: Compound 26 can be prepared in an analogous fashion to FIG. 3 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester in step a.

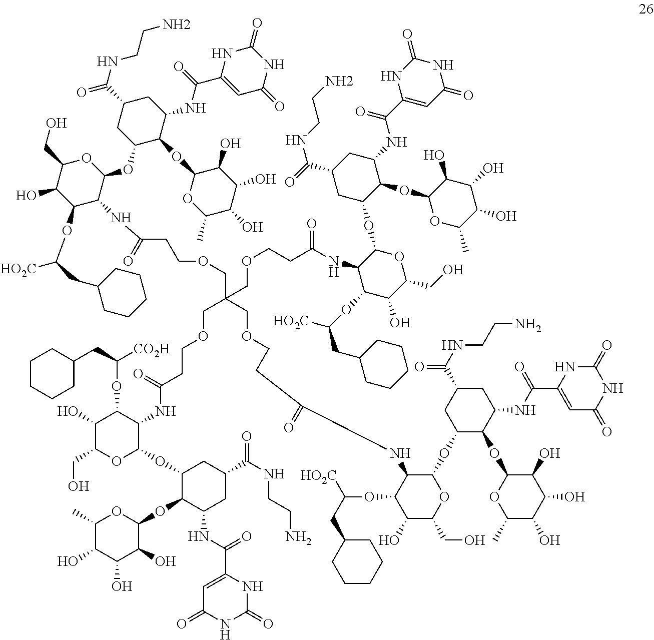

### Example 7

### Prophetic Synthesis of Multimeric Compound 27

Compound 27: Compound 27 can be prepared in an analogous fashion to FIG. 3 by replacing ethylenediamine with 2-aminoethyl ether in step b.

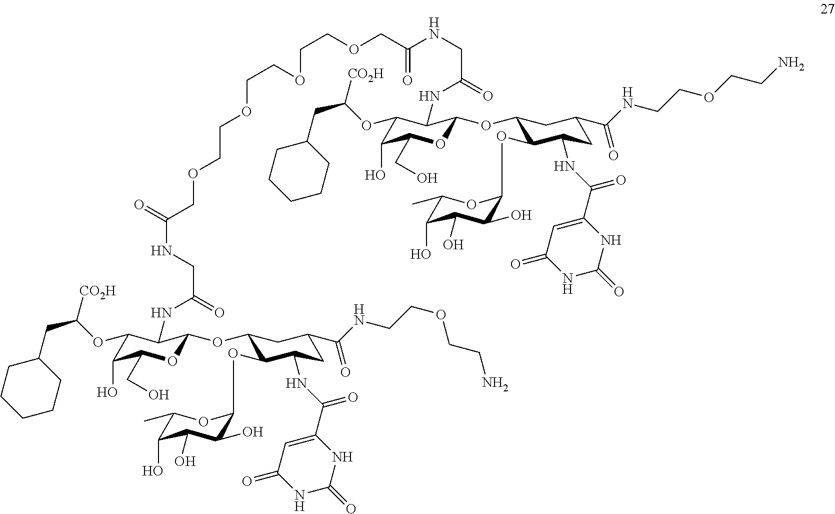

### Example 8

### Prophetic Synthesis of Multimeric Compound 28

Compound 28: Compound 28 can be prepared in an analogous fashion to FIG. 3 by replacing ethylenediamine with 1,5-diaminopentane in step b.

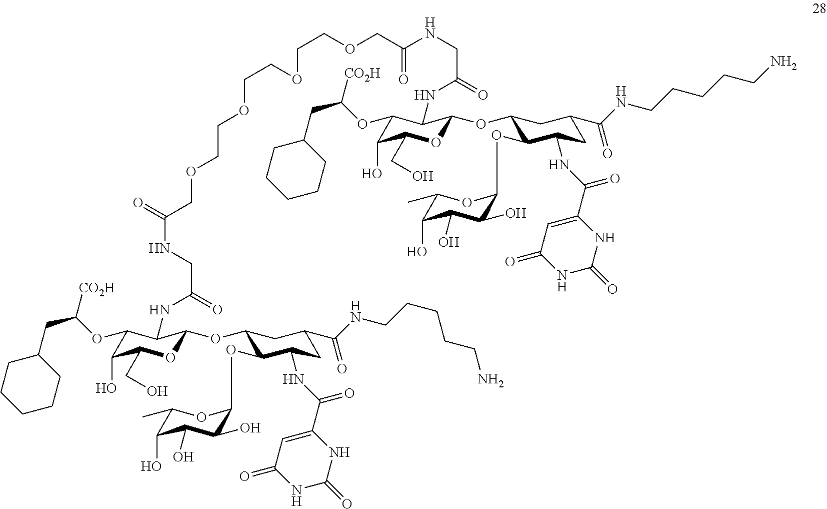

### Example 9

### Prophetic Synthesis of Multimeric Compound 29

Compound 29: Compound 29 can be prepared in an analogous fashion to FIG. 3 by replacing ethylenediamine with 1,2-bis(2-aminoethoxy)ethane in step b.

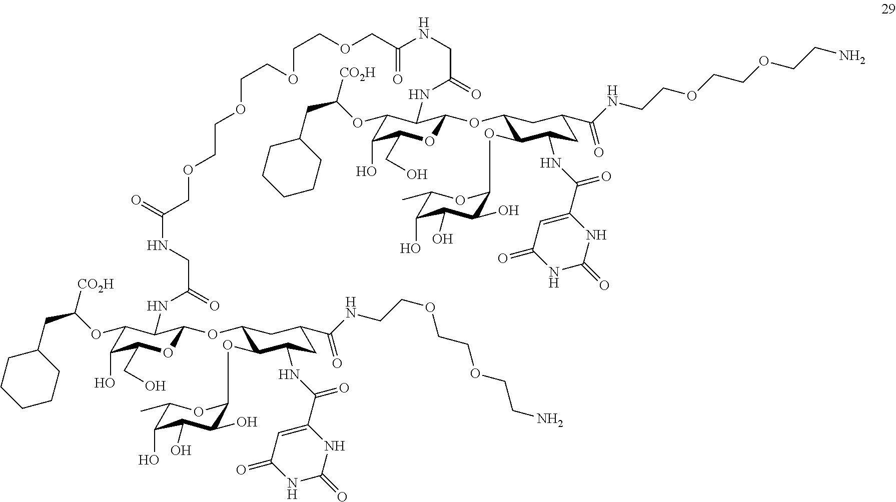

### Example 10

### Prophetic Synthesis of Multimeric Compound 30

Compound 30: Compound 30 can be prepared in an analogous fashion to FIG. 3 by replacing compound 11 with compound 14 and compound 20 with PEG-1 diacetic acid di-NHS ester in step a.

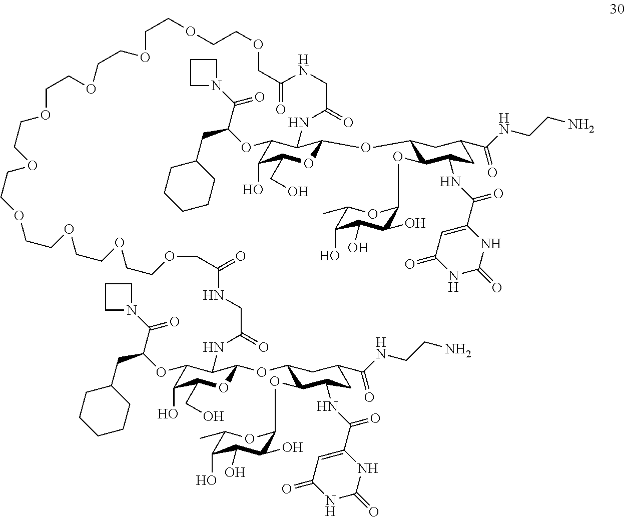

### Example 11

### Prophetic Synthesis of Multimeric Compound 31

Compound 31: Compound 31 can be prepared in an analogous fashion to FIG. 3 by replacing compound 11 with compound 15 in step a.

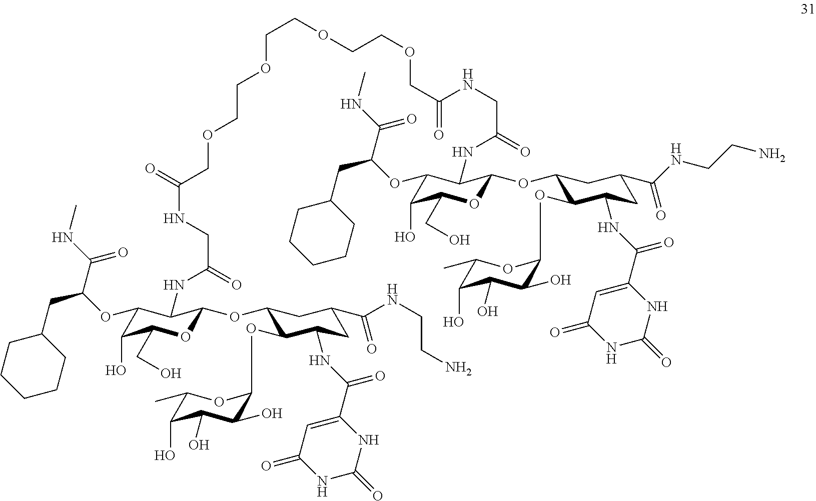

### Example 12

### Prophetic Synthesis of Multimeric Compound 32

Compound 32: Compound 32 can be prepared in an analogous fashion to FIG. 3 by replacing compound 11 with compound 17 and compound 20 with PEG-15 diacetic acid di-NHS ester in step a.

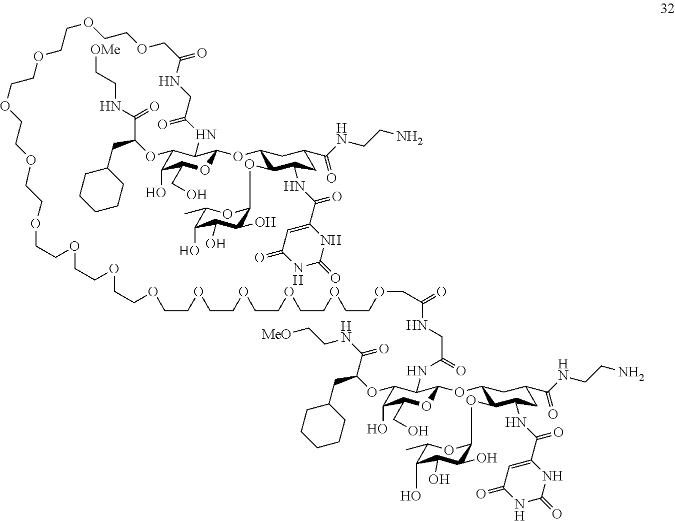

### Example 13

### Prophetic Synthesis of Multimeric Compound 33

Compound 33: Compound 33 can be prepared in an analogous fashion to FIG. 3 by replacing compound 11 with compound 16 and compound 20 with ethylene glycol diacetic acid di-NHS ester in step a.

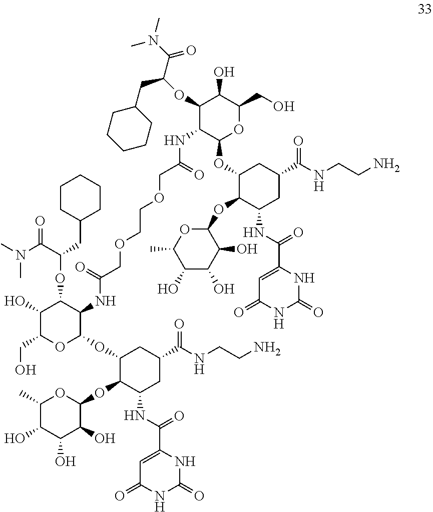 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 14

### Prophetic Synthesis of Multimeric Compound 24

Compound 34: Compound 34 can be prepared in an analogous fashion to FIG. 3 by replacing compound 11 with compound 18 in step a and replacing ethylenediamine with 2-aminoethyl ether in step b.

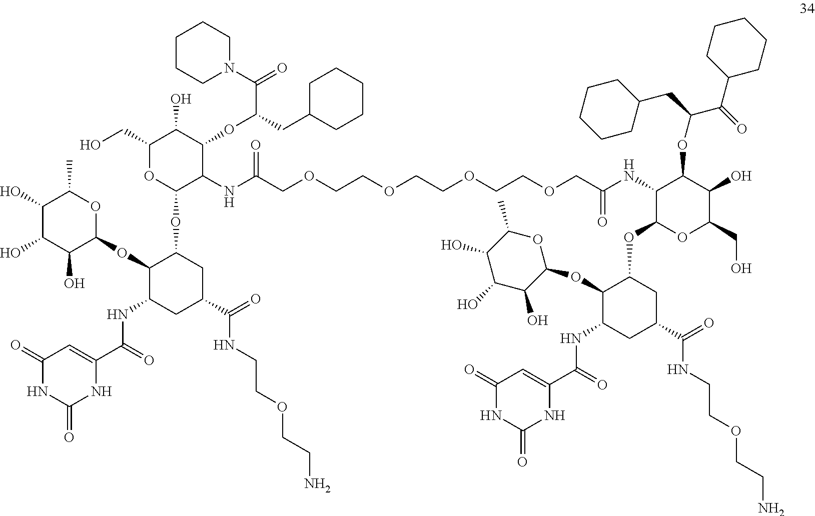 [SMILES: C[C@@H]1O[C@@H](O[C@@H]2[C@@H](NC(=O)c3cc(=O)[nH]c(=O)[nH]3)C[C@@H](C(=O)NCCOCCN)C[C@H]2O[C@@H]2O[C@H](CO)[C@H](O)[C@H](O[C@@H](CC3CCCCC3)C(=O)N3CCCCC3)C2NC(=O)COCCOCCOCCOCC(=O)N[C@H]2[C@H](O[C@@H]3C[C@H](C(=O)NCCOCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)C2CCCCC2)[C@@H](O)[C@H](O)[C@@H]1O]

### Example 15

### Prophetic Synthesis of Multimeric Compound 36

Compound 36: To a solution of compound 12 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 36.

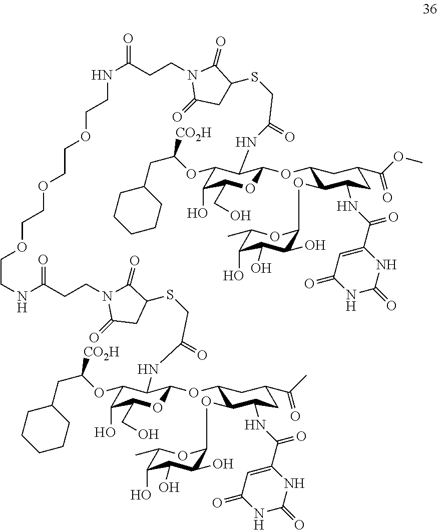

### Example 16

### Prophetic Synthesis of Multimeric Compound 37

Compound 37: Compound 36 is dissolved in ethylenediamine and the reaction mixture is stirred overnight at 70° C. The reaction mixture is concentrated under reduced pressure and the residue is purified by reverse phase chromatography to give compound 37.

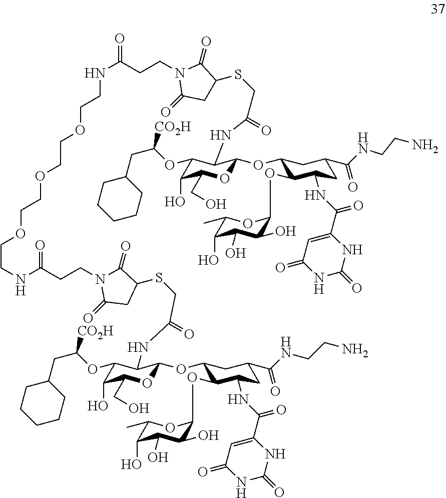

### Example 17

### Prophetic Synthesis of Multimeric Compound 38

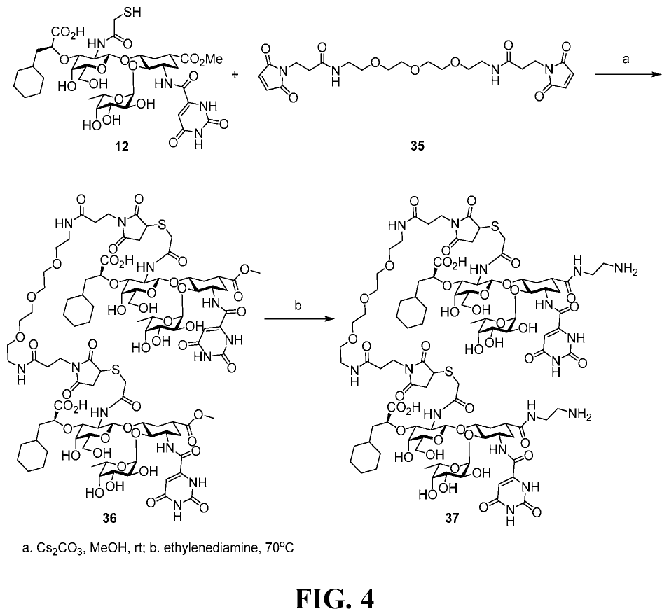

Compound 38: Compound 38 can be prepared in an analogous fashion to FIG. 4 by substituting PEG-6-bis maleimidylpropionamide for compound 35 in step a.

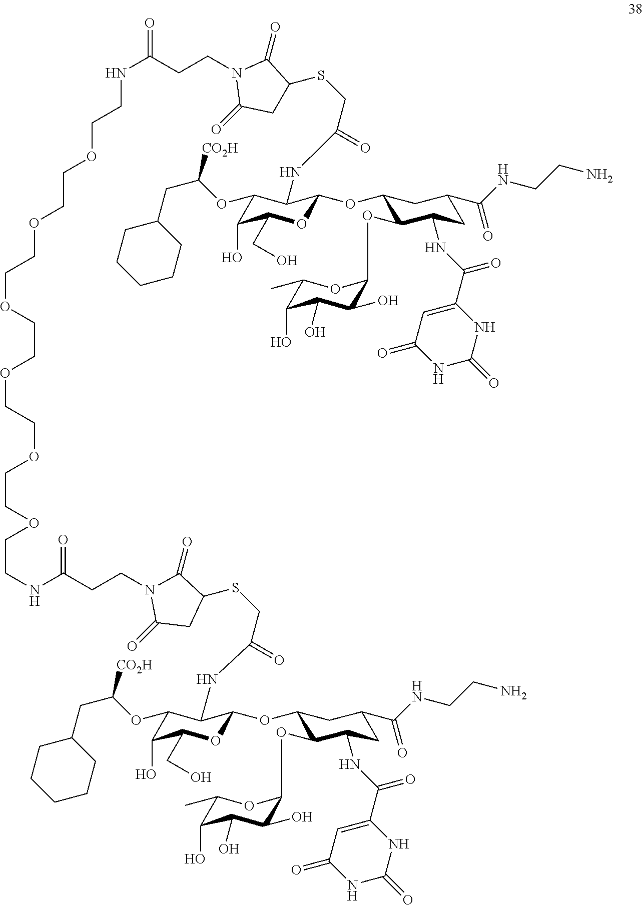

### Example 18

### Prophetic Synthesis of Multimeric Compound 39

Compound 39: Compound 39 can be prepared in an analogous fashion to FIG. 4 by substituting compound 35 for, 1,1′-[[2,2-bis[[3-(2,5-dihydro-2,5-dioxo-1H-pyrrol-1-yl) propoxy]methyl]-1,3-propanediyl]bis(oxy-3,1-propanediyl)]bis-1H-pyrrole-2,5-dione in step a.

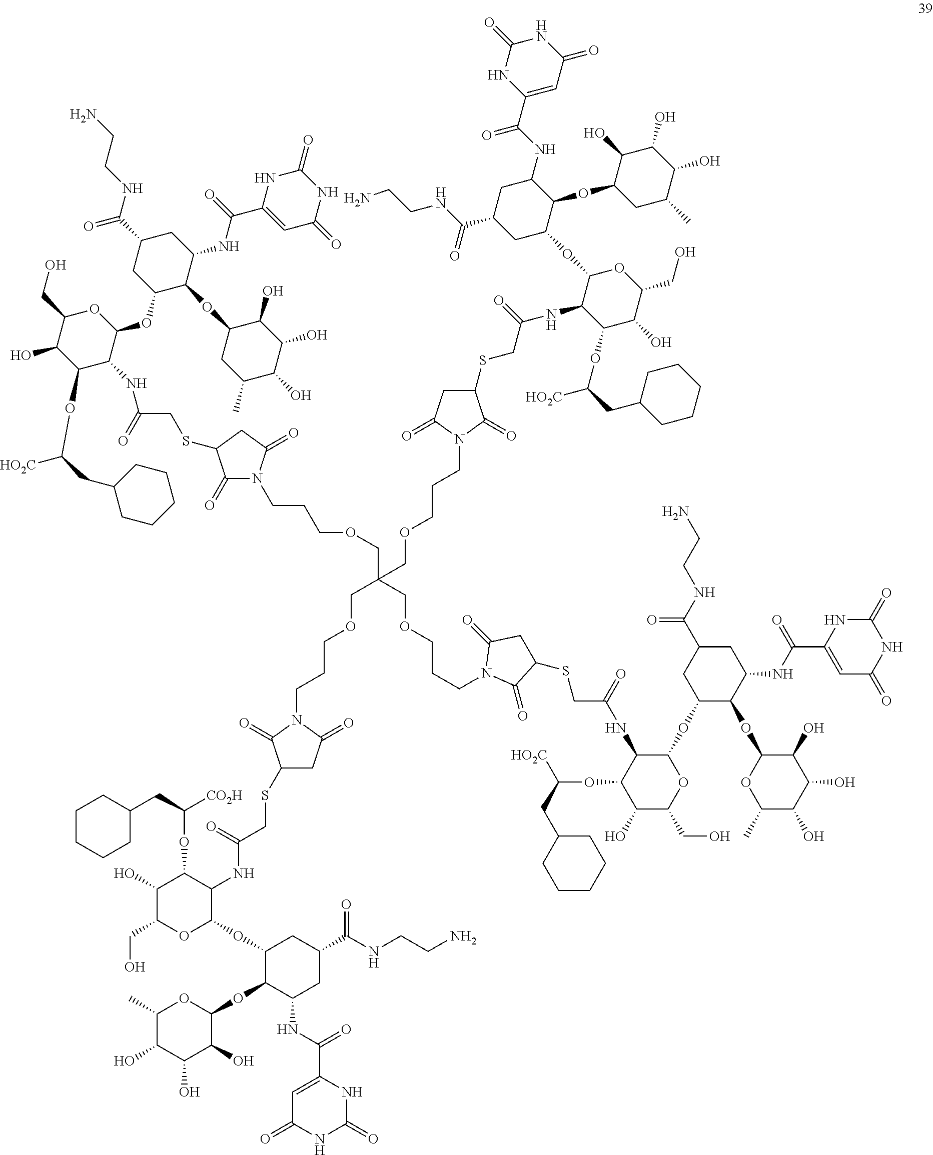

### Example 19

### Prophetic Synthesis of Multimeric Compound 40

Compound 40: Compound 40 can be prepared in an analogous fashion to FIG. 4 by substituting propylenediamine for ethylenediamine in step b.

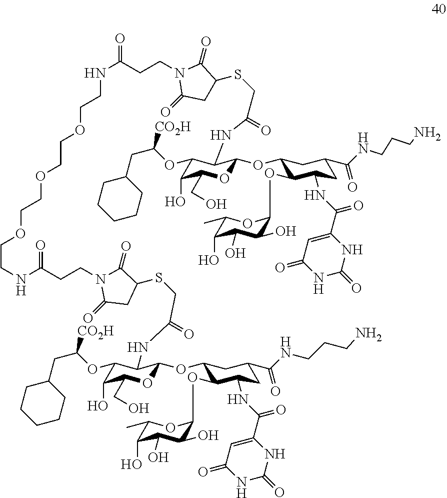

### Example 20

### Prophetic Synthesis of Multimeric Compound 44

Compound 41: To a stirred solution of compound 7 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 41.

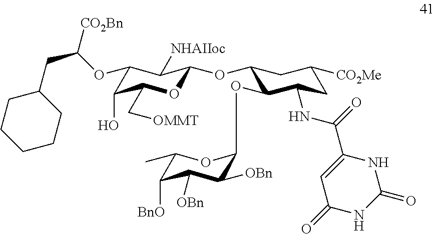

Compound 42: To a degassed solution of compound 41 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 42.

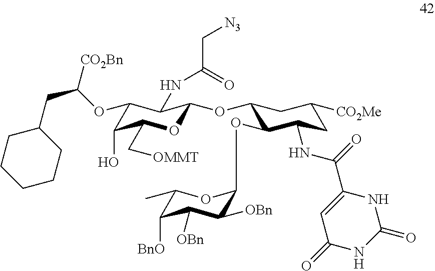

Compound 44: A solution of bispropagyl PEG-5 (compound 43) and compound 42 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 44.

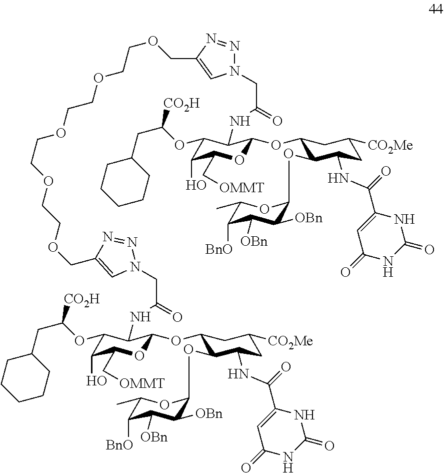

### Example 21

### Prophetic Synthesis of Multimeric Compound 45

Compound 45: Compound 44 is dissolved in MeOH/i-PrOH (2/1) and hydrogenated in the presence of Pd(OH)₂ (20 wt %) at 1 atm of H2 gas pressure for 24 hrs at room temperature. The solution is filtered through a Celite pad. The filtrate is concentrated to give compound 45.

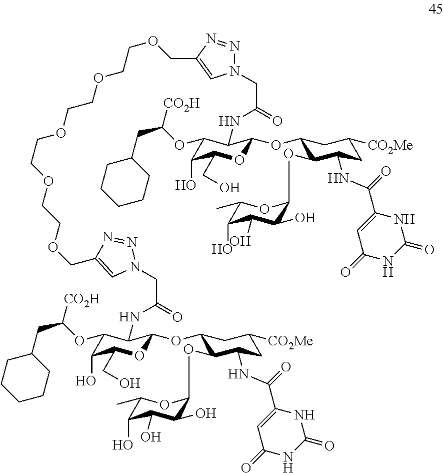

### Example 22

### Prophetic Synthesis of Multimeric Compound 46

Compound 46: Compound 45 is dissolved in ethylenediamine and stirred for 12 hrs at 70° C. The reaction mixture is concentrated under reduced pressure. The crude product is purified by C-18 column chromatography followed by lyophilization to give a compound 46.

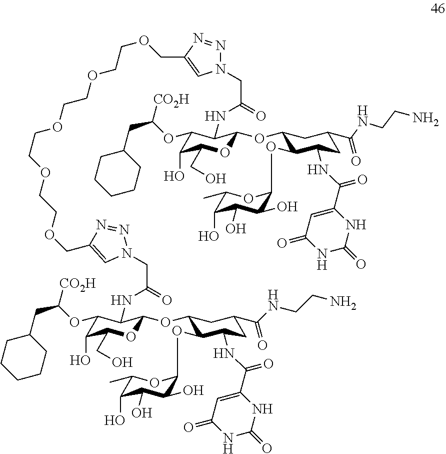

### Example 23

### Prophetic Synthesis of Multimeric Compound 47

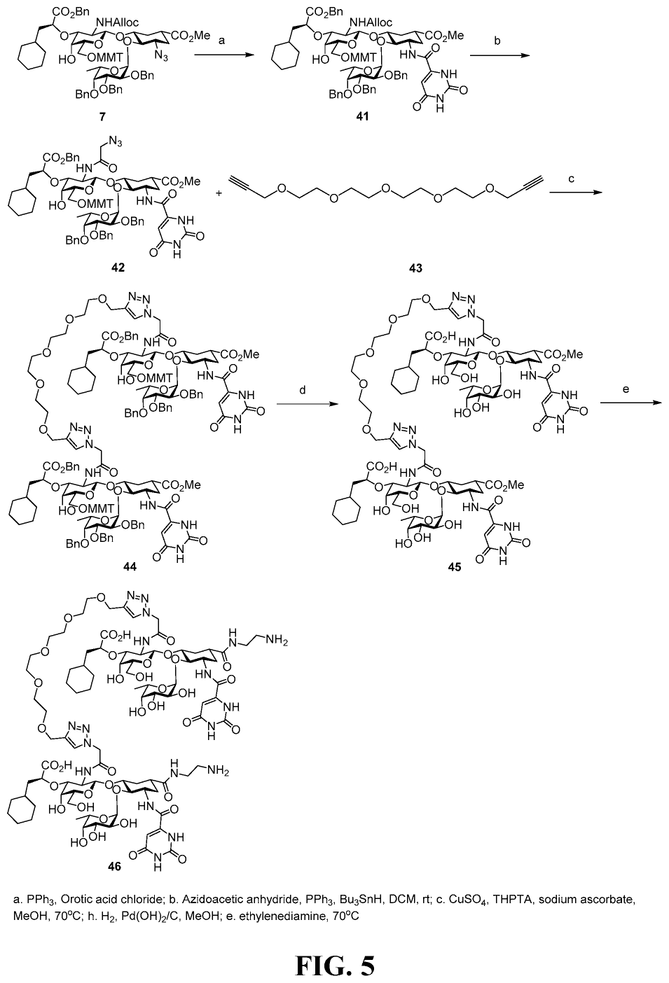

Compound 47: Compound 47 can be prepared in an analogous fashion to FIG. 5 using 3-azidopropionic anhydride (Yang, C. et. al. JACS, (2013) 135(21), 7791-7794) in place of azidoacetic anhydride in step b.

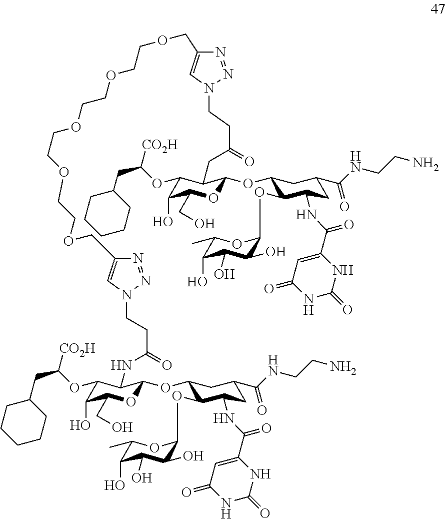

### Example 24

### Prophetic Synthesis of Multimeric Compound 48

Compound 48: Compound 48 can be prepared in an analogous fashion to FIG. 5 using 4-azidobutanoic anhydride (Yang, C. el. al. JACS, (2013) 135(21), 7791-7794) in place of azidoacetic anhydride in step b.

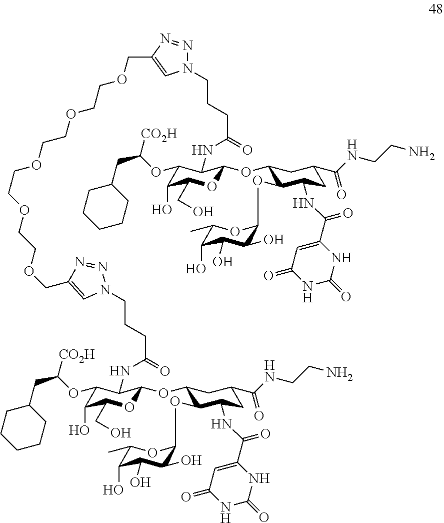

### Example 25

### Prophetic Synthesis of Multimeric Compound 49

Compound 49: Compound 49 can be prepared in an analogous fashion to FIG. 5 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 43 in step c.

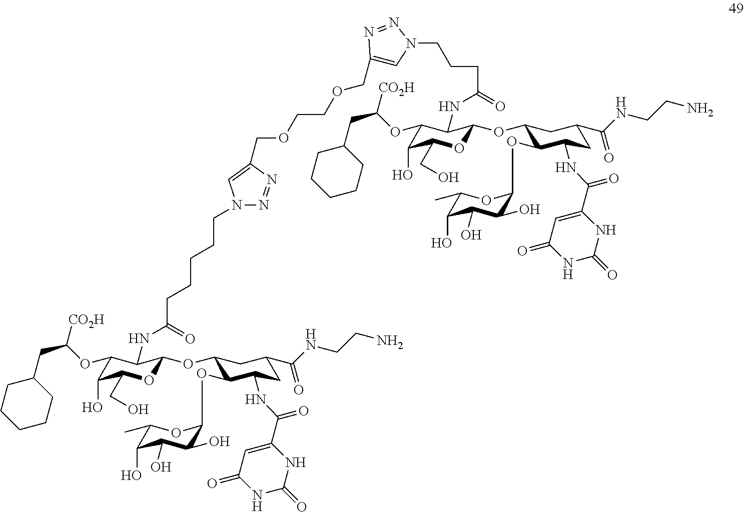

### Example 26

### Prophetic Synthesis of Multimeric Compound 50

Compound 50: Compound 50 can be prepared in an analogous fashion to FIG. 5 using 4,7,10,13,16,19,22,25,28,31-decaoxatetratriaconta-1,33-diyne in place of compound 43 in step c.

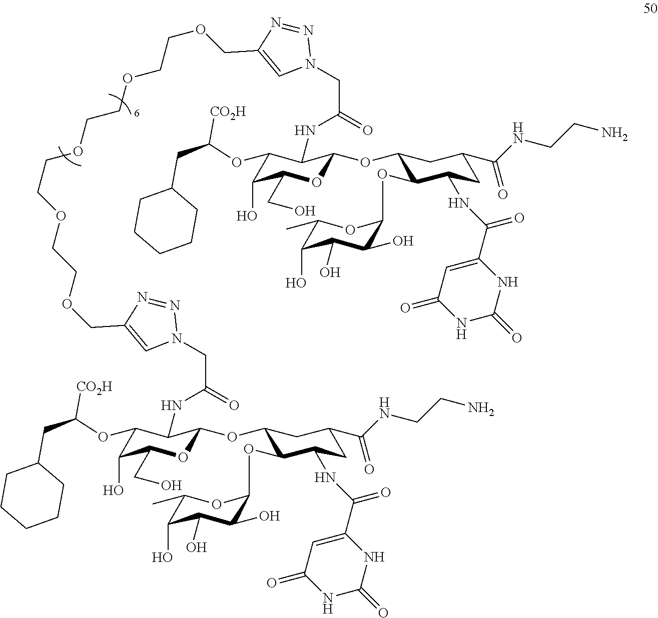

### Example 27

### Prophetic Synthesis of Multimeric Compound 51

Compound 51: Compound 51 can be prepared in an analogous fashion to FIG. 5 using 3,3′-[[2,2-bis[(2-propyn-1-yloxy)methyl]-1,3-propanediyl]bis(oxy)]bis-1-propyne in place of compound 43 in step c.

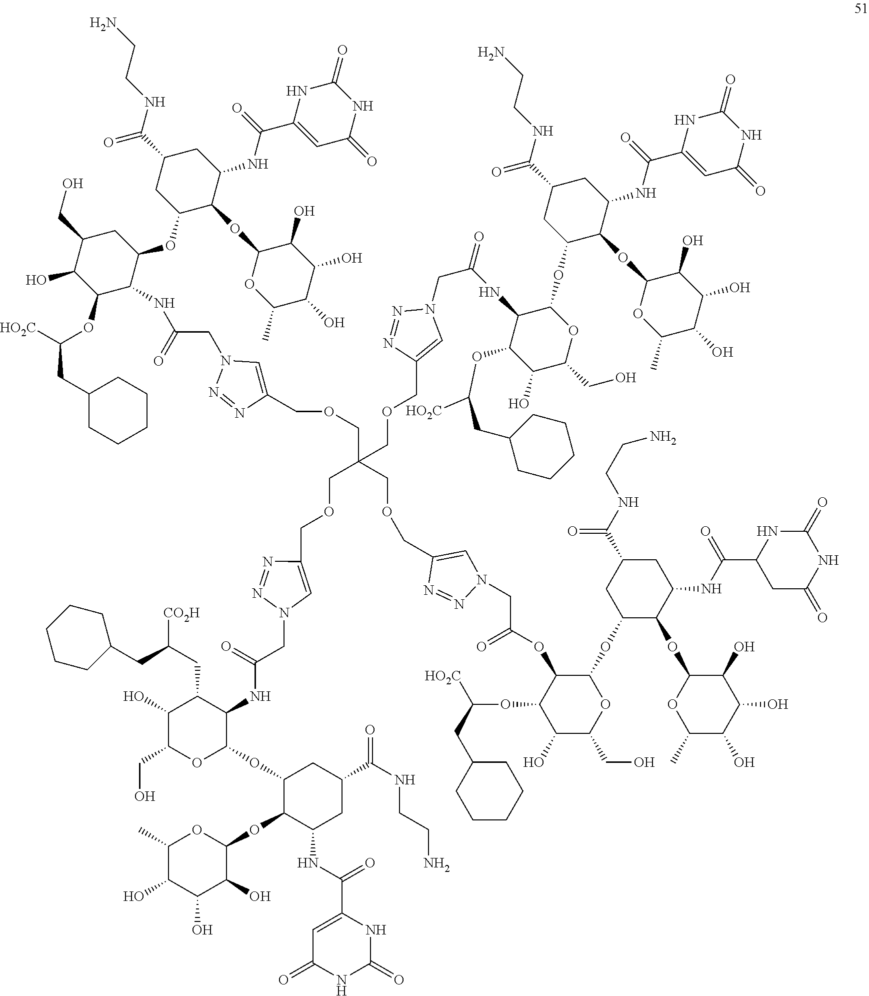

### Example 28

### Prophetic Synthesis of Multimeric Compound 52

Compound 52: Compound 52 can be prepared in an analogous fashion to FIG. 5 using 3,3′-[oxybis[[2,2-bis[(2-propyn-1-yloxy)methyl]-3,1-propanediyl]oxy]]bis-1-propyne in place of compound 43 in step c.

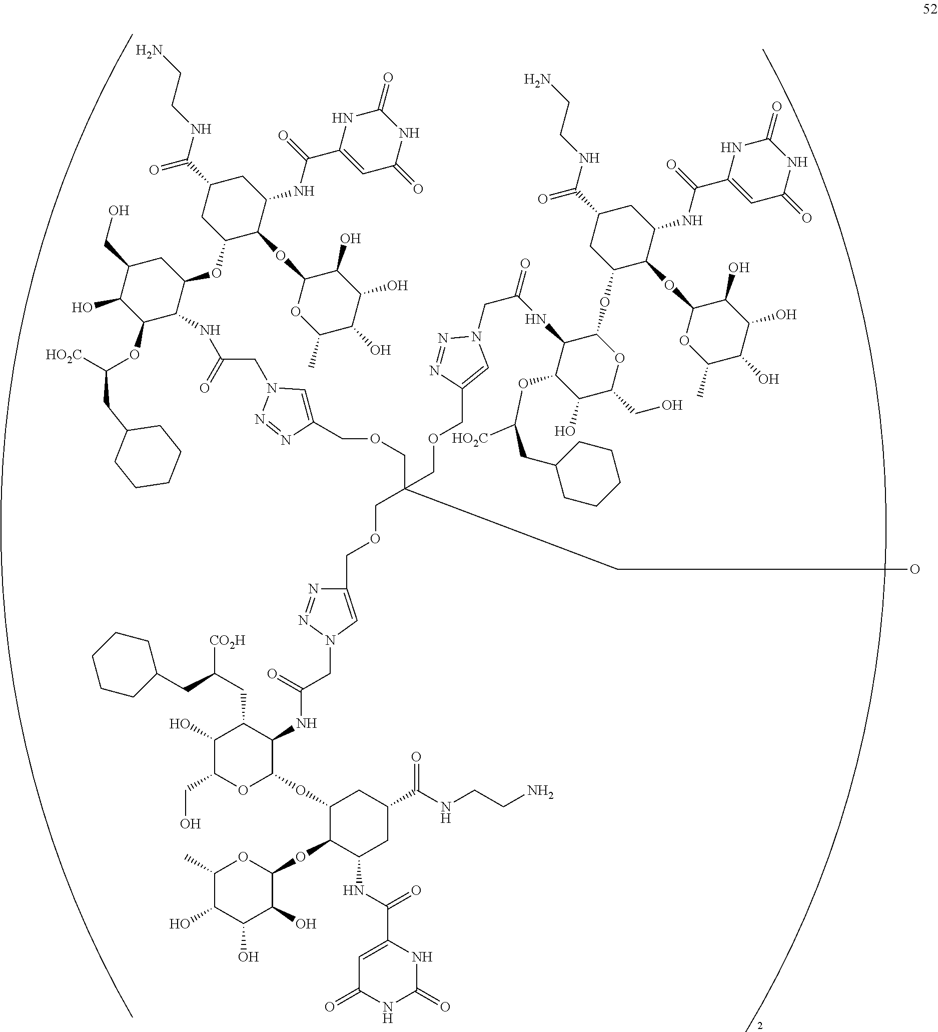

### Example 29

### Prophetic Synthesis of Multimeric Compound 53

Compound 53: Compound 53 can be prepared in an analogous fashion to FIG. 5 using butylenediamine in place of ethylenediamine in step e.

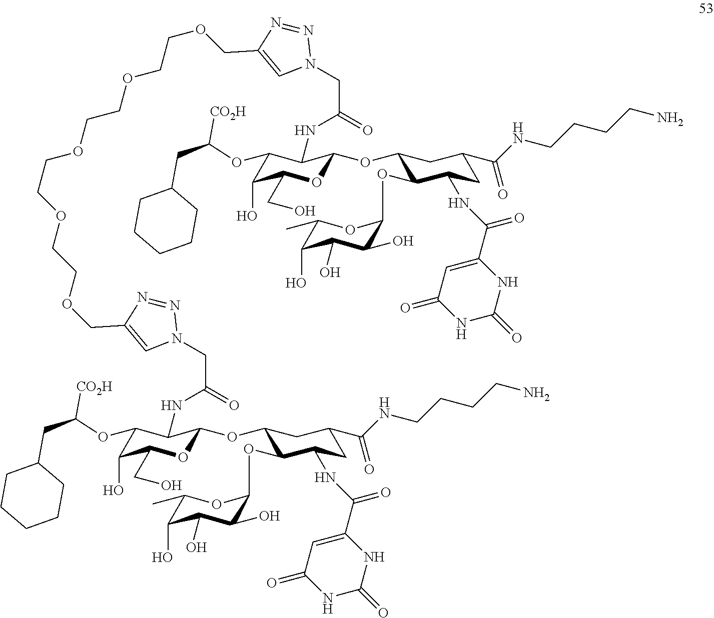

### Example 30

### Prophetic Synthesis of Multimeric Compound 54

Compound 54: Compound 54 can be prepared in an analogous fashion to FIG. 5 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 43 in step c and using 2-aminoethyl ether in step e.

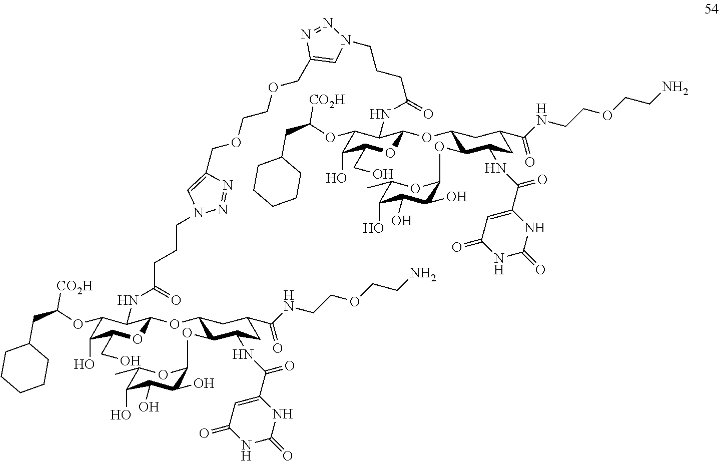

### Example 31

### Prophetic Synthesis of Multimeric Compound 55

Compound 55: Compound 54 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 55.

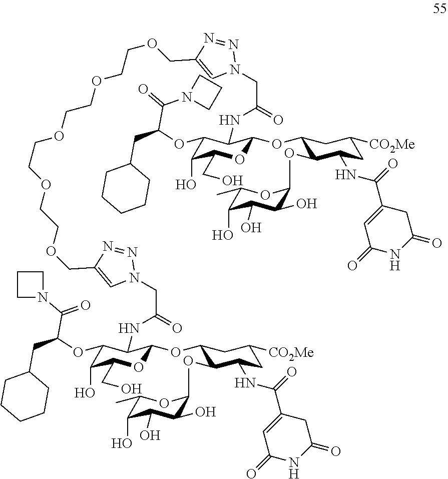

### Example 32

### Prophetic Synthesis of Multimeric Compound 56

Compound 56: Compound 55 is dissolved in ethylenediamine and stirred for 12 hrs at 70° C. The reaction mixture is concentrated under reduced pressure. The crude product is purified by C-18 column chromatography followed by lyophilization to give a compound 56.

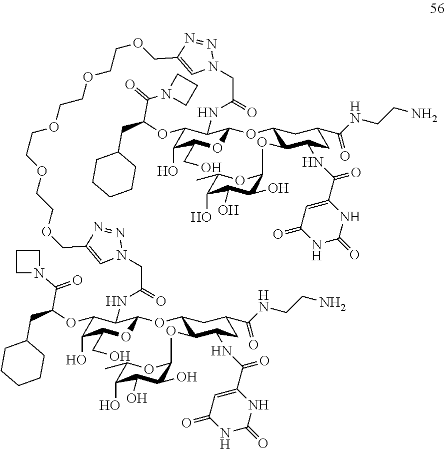

### Example 33

### Prophetic Synthesis of Multimeric Compound 57

Compound 57: Compound 57 can be prepared in an analogous fashion to FIG. 6 using ethylamine in place of azetidine in step a.

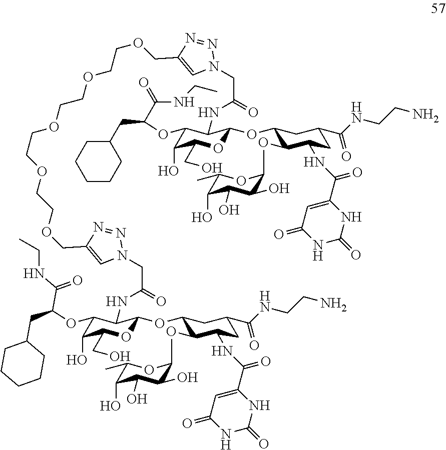

### Example 34

### Prophetic Synthesis of Multimeric Compound 58

Compound 58: Compound 58 can be prepared in an analogous fashion to FIG. 6 using dimethylamine in place of azetidine in step a.

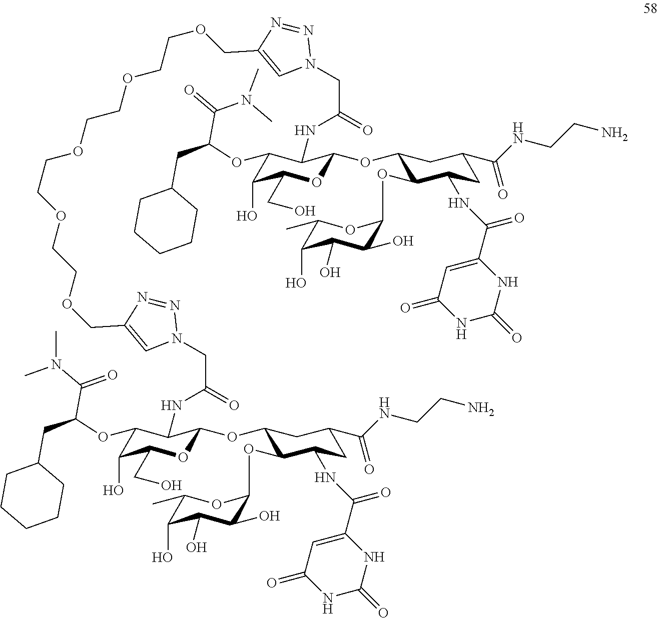

### Example 35

### Prophetic Synthesis of Multimeric Compound 59

Compound 59: Compound 59 can be prepared in an analogous fashion to FIG. 6 using 1,2-bis(2-aminoethoxy)ethane in place of ethylenediamine in step b.

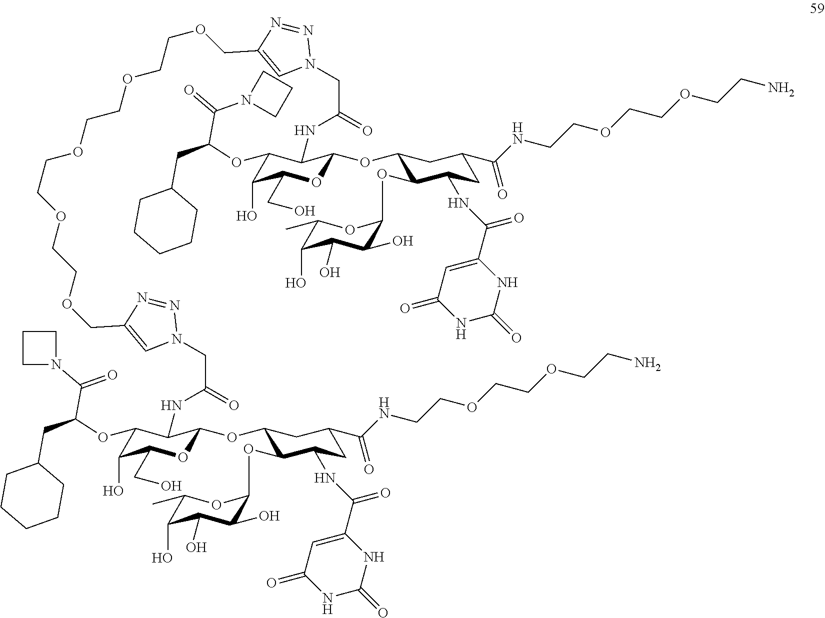

### Example 36

### Prophetic Synthesis of Multimeric Compound 66

Compound 60: To a stirred solution of compound 1 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 60.

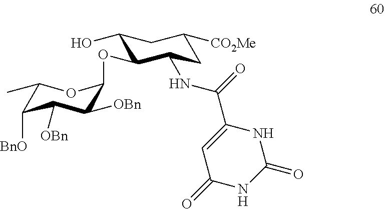

Compound 62: Compound 61 is dissolved in acetonitrile at room temperature. Benzaldehyde dimethylacetal (1.1 eq) is added followed by camphorsulfonic acid (0.2 eq). The reaction mixture is stirred until completion. Triethylamine is added. The solvent is removed and the residue separated by flash chromatography to afford compound 62.

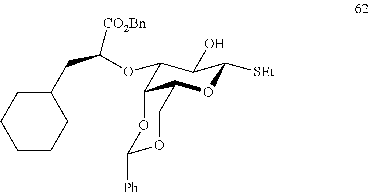

Compound 63: Compound 62 is dissolved in pyridine at room temperature. Dimethylaminopyridine (0.01 eq) is added followed by chloroacetyl chloride (2 eq). The reaction mixture is stirred until completion. The solvent is removed under educed pressure. The residue is dissolved in ethyl acetate, transferred to a separatory funnel and washed two times with 0.1N HCl and two times with water. The organic phase is dried over sodium sulfate, filtered, and concentrated. The residue is separated by column chromatograph to afford compound 63.

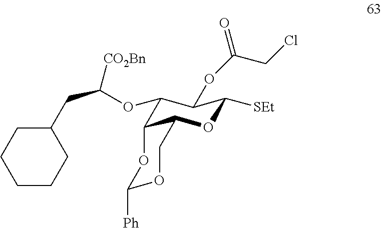

Compound 64: Activated powdered 4 Å molecular sieves are added to a solution of compound 60 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 64.

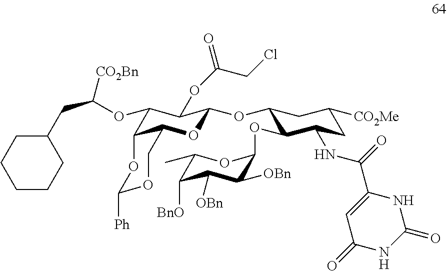

Compound 65: Compound 64 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 65.

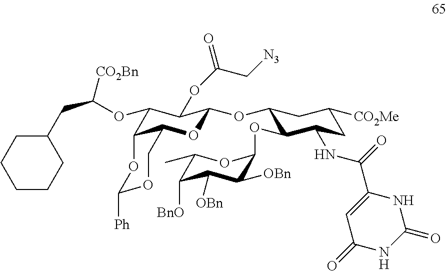

Compound 66: A solution of bispropagyl PEG-5 (compound 43) and compound 65 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 66.

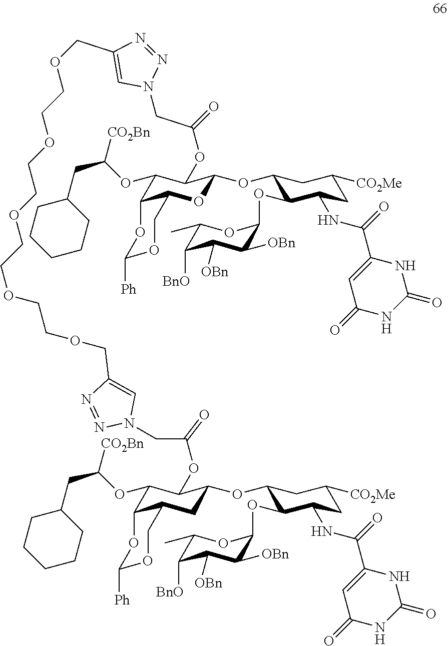

### Example 37

### Prophetic Synthesis of Multimeric Compound 67

Compound 67: To a solution of compound 66 in dioxane/water (4/1) is added Pd(OH)₂/C. The reaction mixture is stirred vigorously overnight under a hydrogen atmosphere. The reaction mixture is filtered through Celite and concentrated. The residue is purified by C-19 reverse phase column chromatography to afford compound 67.

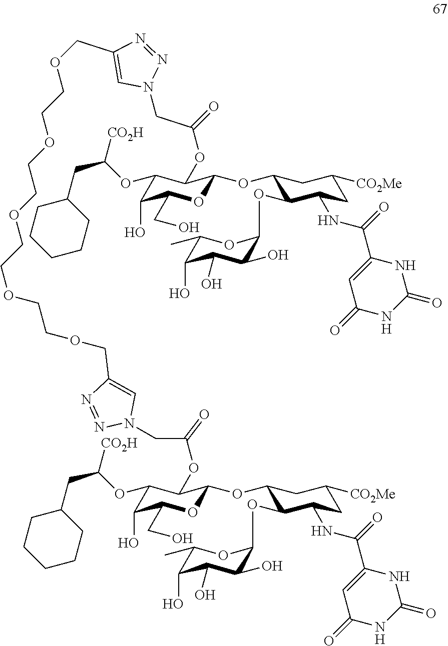

### Example 38

### Prophetic Synthesis of Multimeric Compound 68

Compound 68: Compound 67 is dissolved in ethylenediamine and stirred for 12 hrs at 70° C. The reaction mixture is concentrated under reduced pressure. The crude product is purified by C-18 column chromatography followed by lyophilization to afford compound 68.

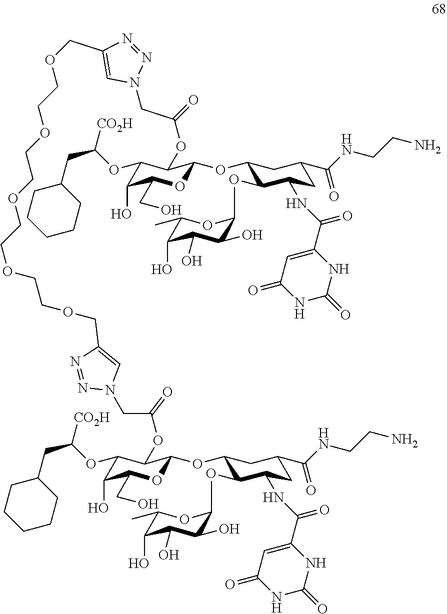

### Example 39

### Prophetic Synthesis of Multimeric Compound 69

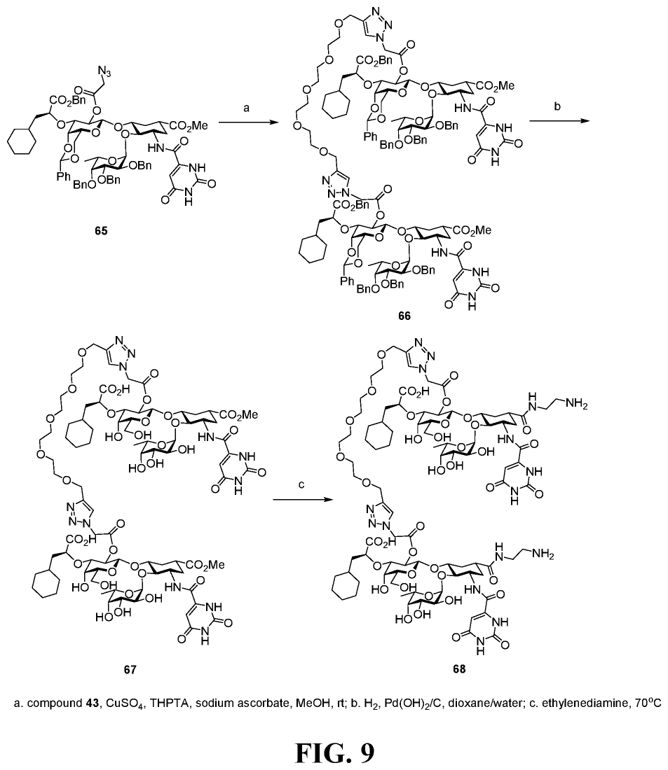

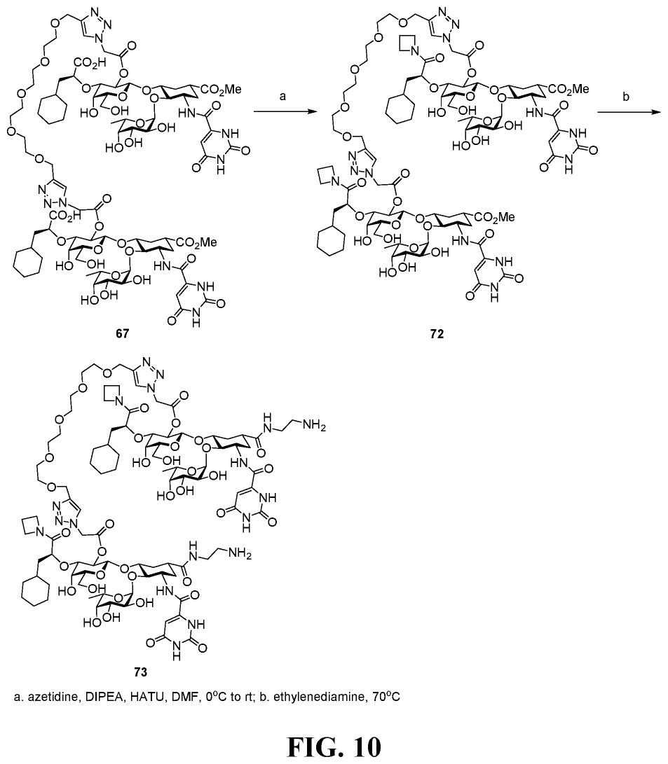

Compound 69: Compound 69 can be prepared in an analogous fashion to FIG. 9 by replacing compound 43 with PEG-8 bis propargyl ether in step a.

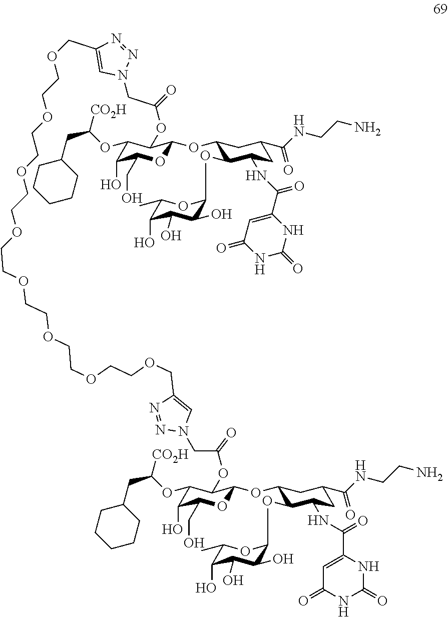

### Example 40

### Prophetic Synthesis of Multimeric Compound 70

Compound 70: Compound 70 can be prepared in an analogous fashion to FIG. 9 by replacing compound 43 with ethylene glycol bis propargyl ether in step a.

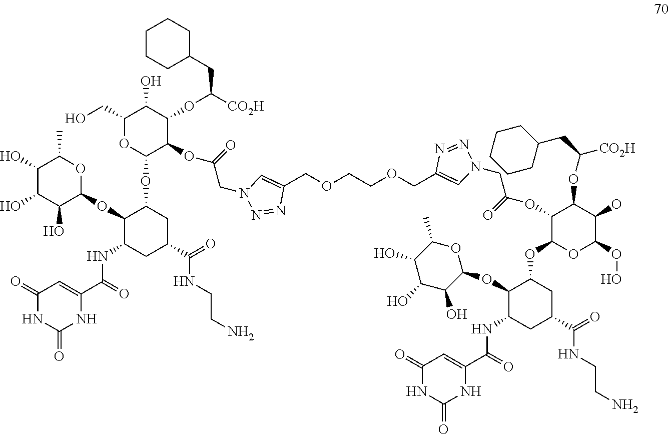 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5C[C@H](C(=O)NCCN)C[C@H](NC(=O)c6cc(=O)[nH]c(=O)[nH]6)[C@H]5O[C@@H]5O[C@@H](C)[C@@H](O)[C@@H](O)[C@@H]5O)O[C@H](OO)[C@H](O)[C@@H]4O[C@@H](CC4CCCCC4)C(=O)O)nn3)nn2)[C@@H](O)[C@H](O)[C@@H]1O]

### Example 41

### Prophetic Synthesis of Multimeric Compound 71

Compound 71: Compound 71 can be prepared in an analogous fashion to FIG. 9 using 3,3′-[[2,2-bis[(2-propyn-1-yloxy)methyl]-1,3-propanediyl]bis(oxy)]bis-1-propyne in place of compound 43 in step a.

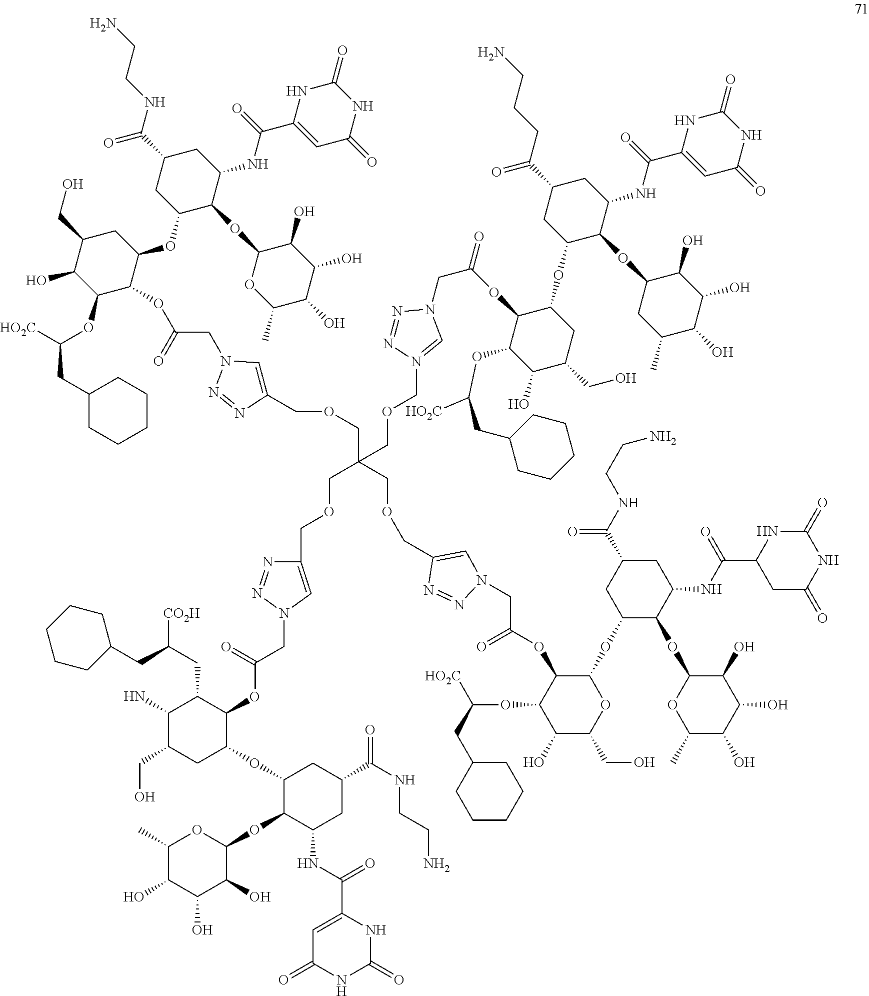

### Example 42

### Prophetic Synthesis of Multimeric Compound 72

Compound 72: Compound 67 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 72.

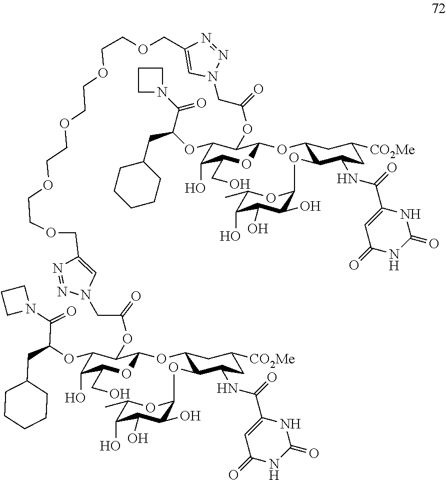

### Example 43

### Prophetic Synthesis of Multimeric Compound 73

Compound 73: Compound 72 is dissolved in ethylenediamine and stirred for 12 hrs at 70° C. The reaction mixture is concentrated under reduced pressure. The crude product is purified by C-18 column chromatography followed by lyophilization to afford compound 73.

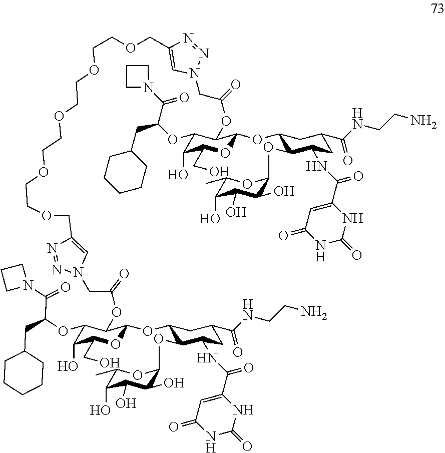

### Example 44

### Synthesis of Multimeric Compound 76

Compound 75: To a degassed solution of compound 74 (synthesis described in WO 2013/096926) (0.5 g, 0.36 mmole) in anhydrous DCM (10 mL) at 0° C. was added Pd(PPh₃)₄ (42 mg, 36.3 μmole, 0.1 eq), Bu₃SnH (110 μL, 0.4 μmole, 1.1 eq) and azidoacetic anhydride (0.14 g, 0.73 mmole, 2.0 eq). The resulting solution was stirred for 12 hrs under N2 atmosphere while temperature was gradually increased to room temperature. After the reaction was completed, the solution was diluted with DCM (20 mL), washed with distilled water, dried over Na₂SO₄, then concentrated. The crude product was purified by combi-flash (EtOAc/Hex, Hex only—3/2, v/v) to give compound 75 (0.33 g, 67%). MS: Calculated (C₈₁H₉₅N₄O₁₆, 1376.6), ES—Positive (1400.4, M+Na)).

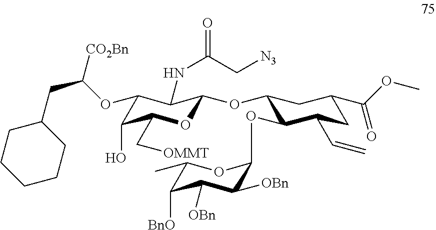

Compound 76: A solution of bispropargyl PEG-5 (compound 43, 27 mg, 0.1 mmole) and compound 75 (0.33 g, 0.24 mmole, 2.4 eq) in a mixed solution (MeOH/1,4 dioxane, 2/1, v/v, 12 mL) was degassed at room temperature. A solution of CuSO₄/THPTA in distilled water (0.04 M) (0.5 mL, 20 μmole, 0.2 eq) and sodium ascorbate (4.0 mg, 20 μmole, 0.2 eq) were added successively and the resulting solution was stirred 12 hrs at 70° C. The solution was cooled to room temperature and concentrated under reduced pressure. The crude product was purified by combi-flash (EtOAc/MeOH, EtOAc only—4/1, v/v) to give a compound 76 as a white foam (0.23 g, 70%).

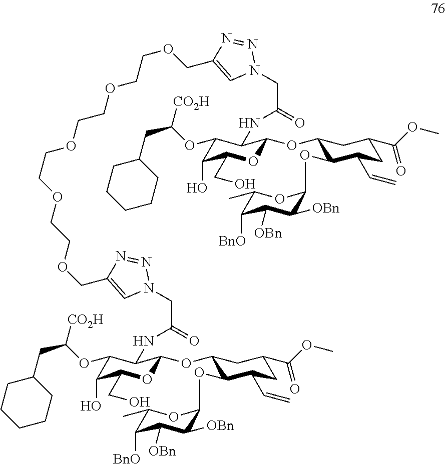

### Example 45

### Synthesis of Multimeric Compound 77

Compound 77: A solution of compound 76 (0.23 g, 0.76 μmole) in solution of MeOH/i-PrOH (2/1, v/v, 12 mL) was hydrogenated in the presence of Pd(OH)₂ (0.2 g) and 1 atm of H2 gas pressure for 24 hrs at room temperature. The solution was filtered through a Celite pad and the cake was washed with MeOH. The combined filtrate was concentrated under reduced pressure. The crude product was washed with hexane and dried under high vacuum to give compound 77 as a white solid (0.14 g, quantitative). MS: Calculated (C₈₀H₁₃₀N₈O₃₅, 1762.8), ES—positive (1785.4, M+Na), ES—Negative (1761.5, M-1, 879.8).

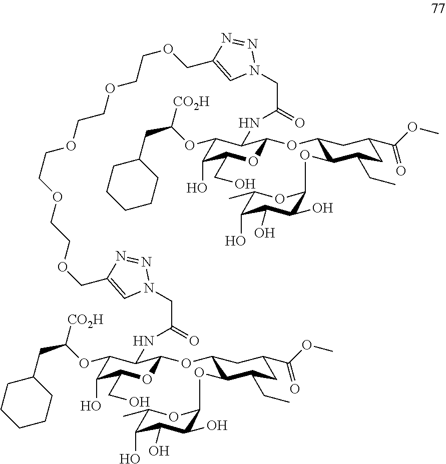

### Example 46

### Prophetic Synthesis of Multimeric Compound 78

Compound 78: Compound 77 (60 mg, 34.0 μmole) was dissolved in ethylenediamine (3 mL) and the homogeneous solution was stirred for 12 hrs at 70° C. The reaction mixture was concentrated under reduced pressure and the residue was dialyzed against distilled water with MWCO 500 dialysis tube. The crude product was further purified by C-18 column chromatography with water/MeOH (9/1-1/9, v/v) followed by lyophilization to give a compound 78 as a white solid (39 mg, 63%).

1H NMR (400 MHz, Deuterium Oxide) δ 8.00 (s, 2H), 5.26-5.14 (two d, J=16.0 Hz, 4H), 4.52 (d, J=4.0 Hz, 2H), 4.84 (dd, J=8.0 Hz, J=4.0 Hz, 2H), 4.66 (s, 4H), 4.54 (broad d, J=12 Hz, 2H), 3.97 (broad t, 2H), 3.91-3.78 (m, 6H), 3.77-3.58 (m, 28H), 3.57-3.46 (m, 4H), 3.42 (t, J=8.0 Hz, 6H), 3.24 (t, J=12.0 Hz, 2H), 3.02 (t, J=6.0 Hz, 4H), 2.67 (s, 2H), 2.32 (broad t, J=12 Hz, 2H), 2.22-2.06 (m, 2H), 1.96-1.74 (m, 4H), 1.73-1.39 (m, 18H), 1.38-1.21 (m, 6H), 1.20-0.99 (m, J=8.0 Hz, 14H), 0.98-0.73 (m, J=8.0 Hz, 10H).

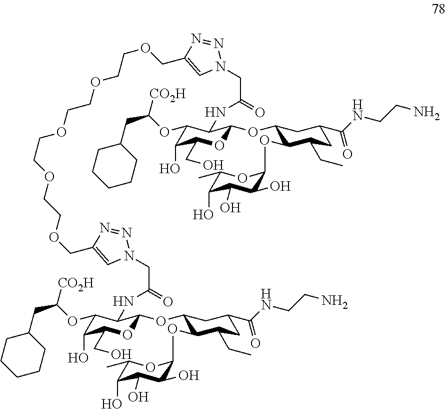

### Example 47

### Prophetic Synthesis of Multimeric Compound 79

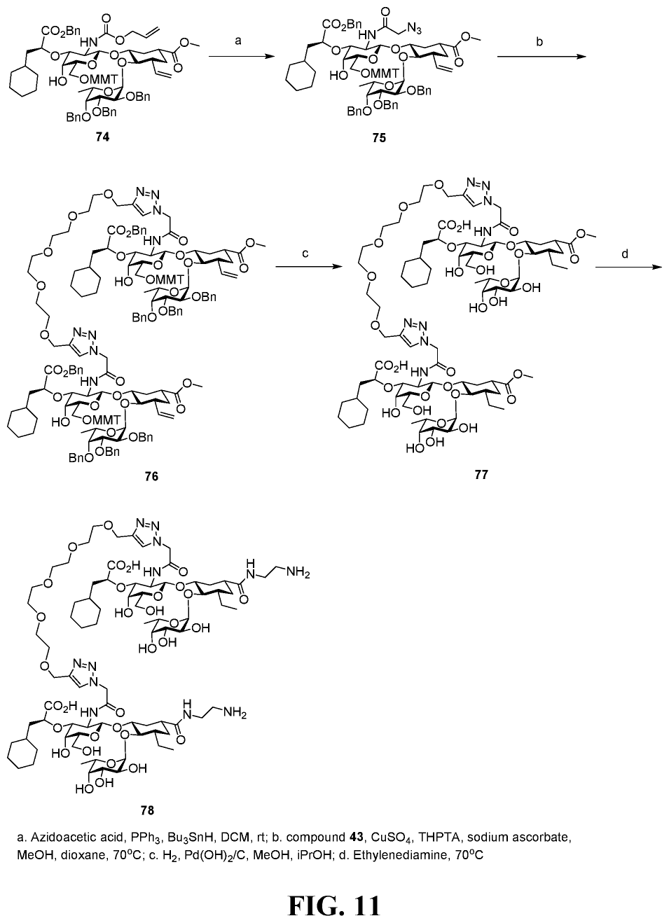

Compound 79: Compound 79 can be prepared in an analogous fashion to FIG. 11 using 3-azidopropionic anhydride (Yang, C. et. al. JACS, (2013) 135(21), 7791-7794) in place of azidoacetic anhydride in step a.

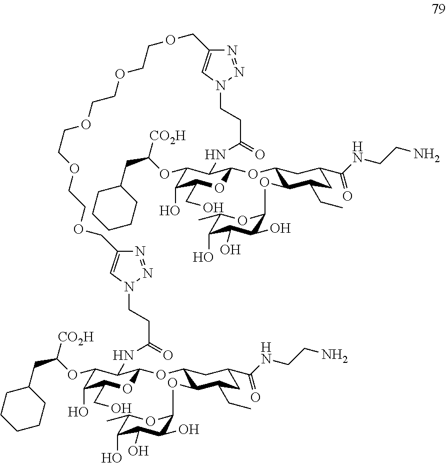

### Example 48

### Prophetic Synthesis of Multimeric Compound 80

Compound 80: Compound 80 can be prepared in an analogous fashion to FIG. 11 using 4-azidobutanoic anhydride (Yang, C. et. al. JACS, (2013) 135(21), 7791-7794) in place of azidoacetic anhydride in step a.

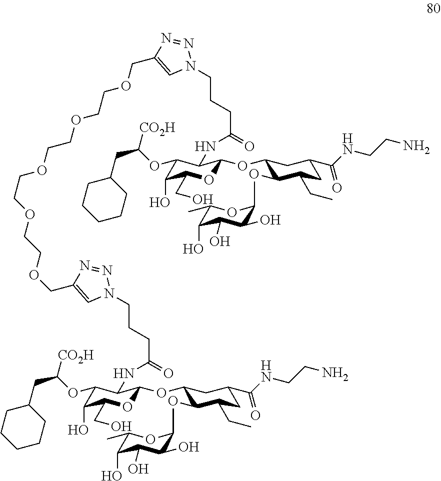

### Example 49

### Prophetic Synthesis of Multimeric Compound 81

Compound 81: Compound 81 can be prepared in an analogous fashion to FIG. 11 using 4-azidobutanoic anhydride (Yang, C. el. al. JACS, (2013) 135(21), 7791-7794) in place of azidoacetic anhydride in step a and using 1,2-bi(2-propynyloxy) ethane in place of compound 43 in step b.

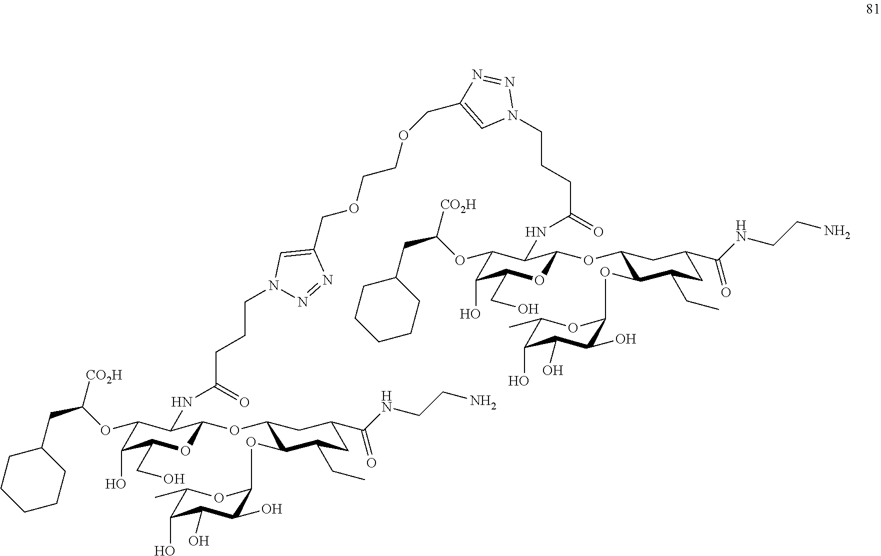

### Example 50

### Prophetic Synthesis of Multimeric Compound 82

Compound 82: Compound 82 can be prepared in an analogous fashion to FIG. 11 using 4,7,10,13,16,19,22,25,28,31-decaoxatetratriaconta-1,33-diyne in place of compound 43 in step b.

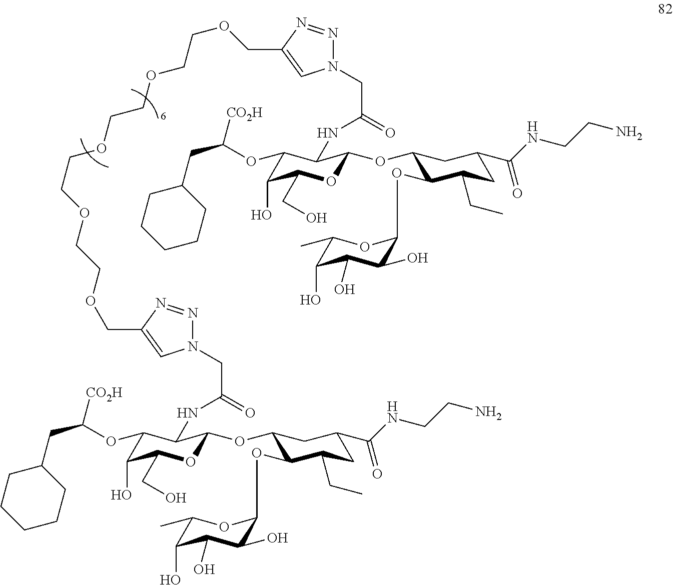

### Example 51

### Prophetic Synthesis of Multimeric Compound 83

Compound 83: Compound 83 can be prepared in an analogous fashion to FIG. 11 using 2-aminoethylether in place of ethylenediamine in step d.

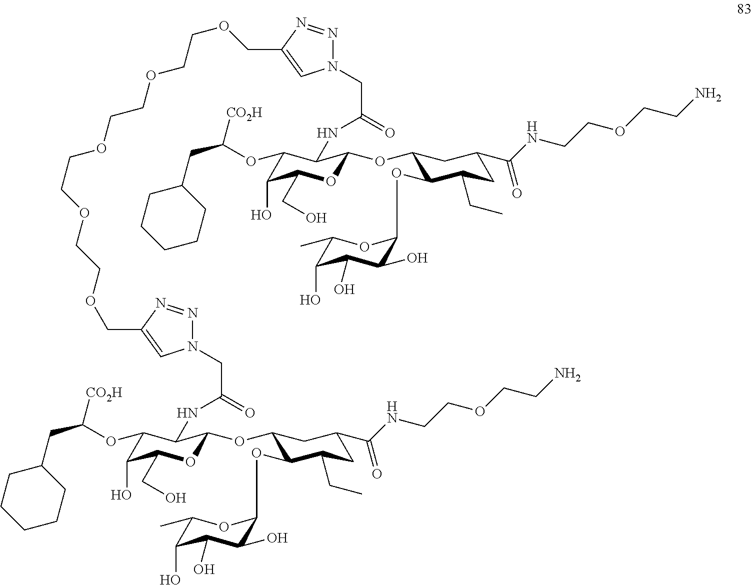

### Example 52

### Prophetic Synthesis of Multimeric Compound 84

Compound 84: Compound 84 can be prepared in an analogous fashion to FIG. 11 using 1,2-bi(2-propynyloxy) ethane in place of compound 43 in step b.

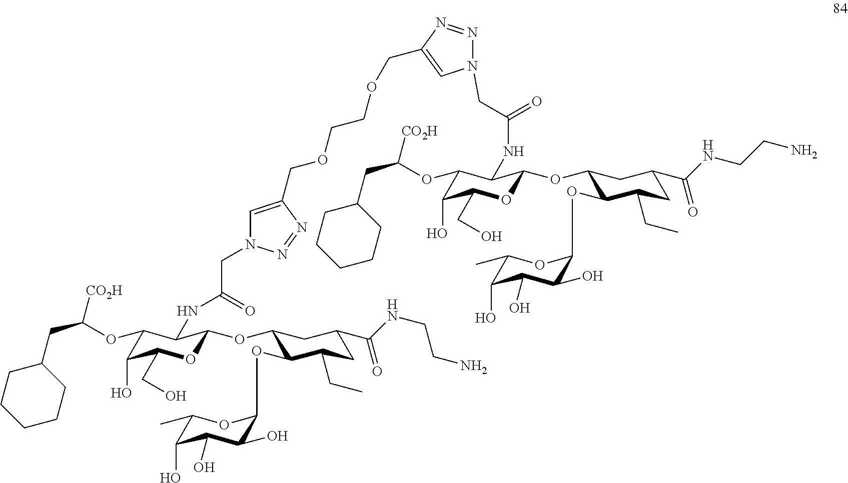

### Example 53

### Prophetic Synthesis of Multimeric Compound 85

Compound 85: Compound 85 can be prepared in an analogous fashion to FIG. 11 using PEG-8 dipropargyl ether in place of compound 43 in step b and 1,5-diaminopentane in place of ethylenediamine in step d.

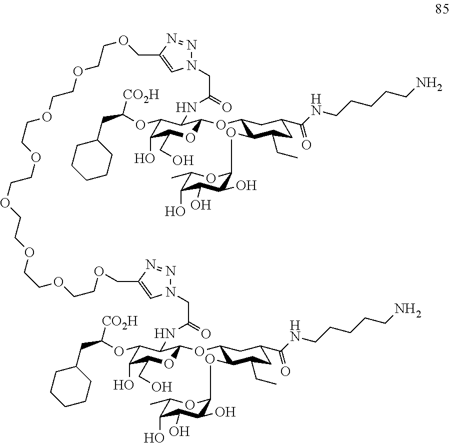

### Example 54

### Prophetic Synthesis of Multimeric Compound 86

Compound 86: Compound 77 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 86.

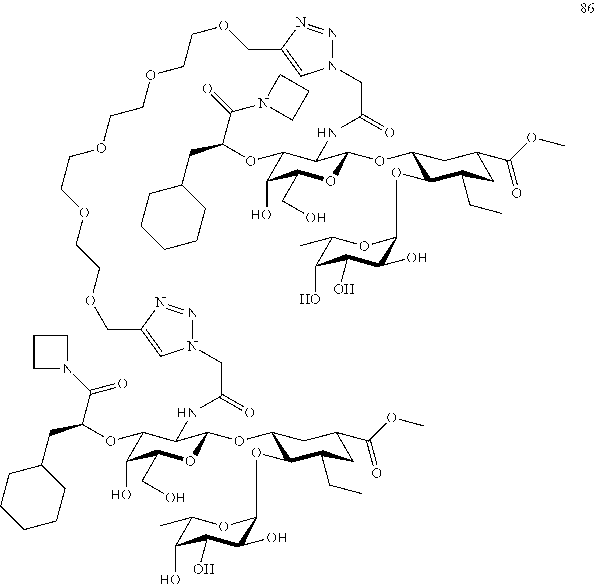

### Example 55

### Prophetic Synthesis of Multimeric Compound 87

Compound 87: Compound 86 is dissolved in ethylenediamine stirred for 12 hrs at 70° C. The reaction mixture was concentrated under reduced pressure. The residue was purified by C-18 column chromatography followed by lyophilization to give a compound 87.

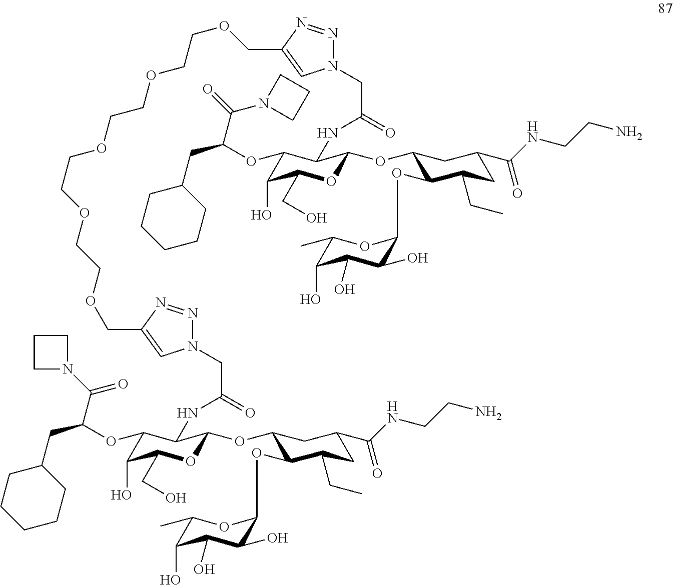

### Example 56

### Prophetic Synthesis of Multimeric Compound 88

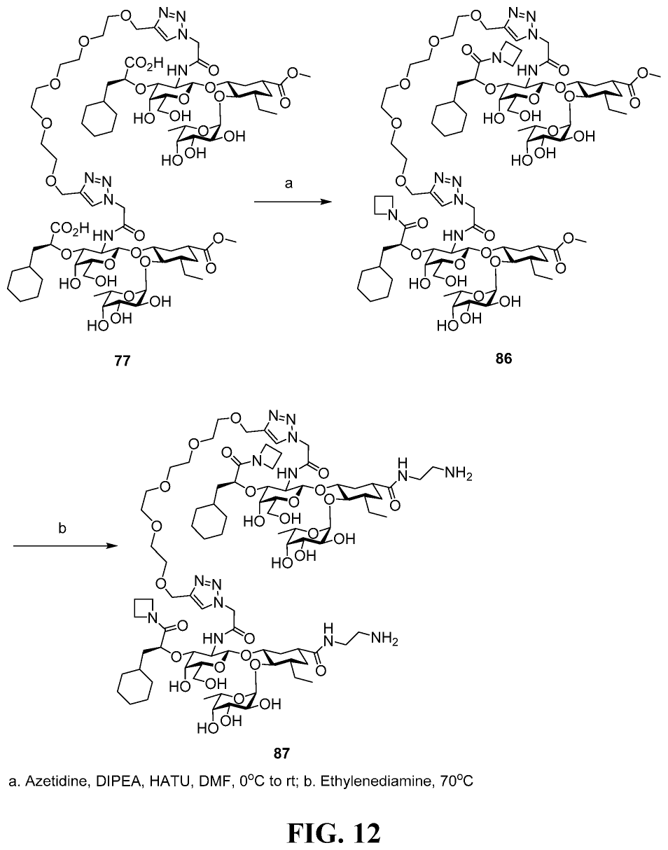

Compound 88: Compound 88 can be prepared in an analogous fashion to FIG. 12 using 2-aminoethylether in place of ethylenediamine in step b.

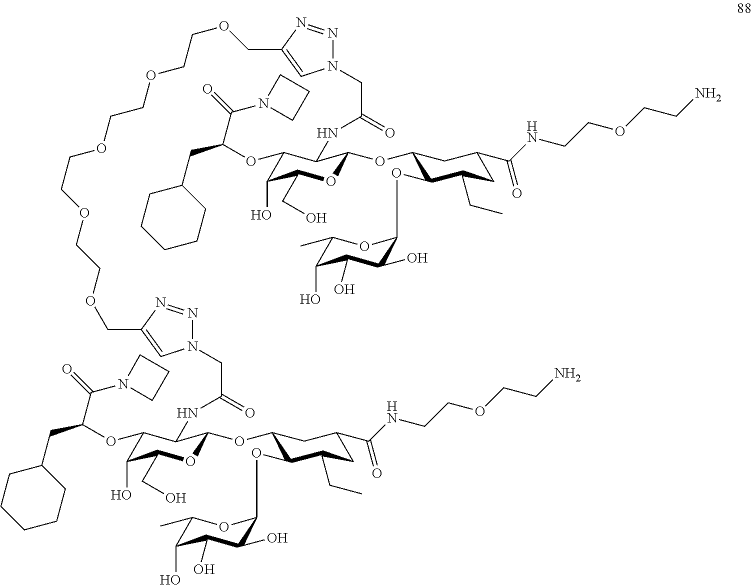

### Example 57

### Prophetic Synthesis of Multimeric Compound 89

Compound 89: Compound 89 can be prepared in an analogous fashion to FIG. 12 using dimethylamine in place of azetidine in step a and 2-aminoethylether in place of ethylenediamine in step b.

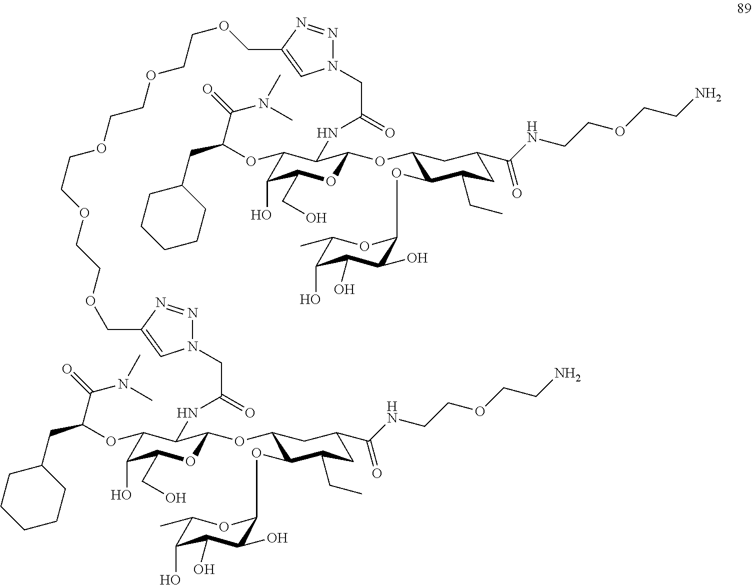

### Example 58

### Prophetic Synthesis of Multimeric Compound 90

Compound 90: Compound 90 can be prepared in an analogous fashion to FIG. 12 using piperidine in place of azetidine in step a.

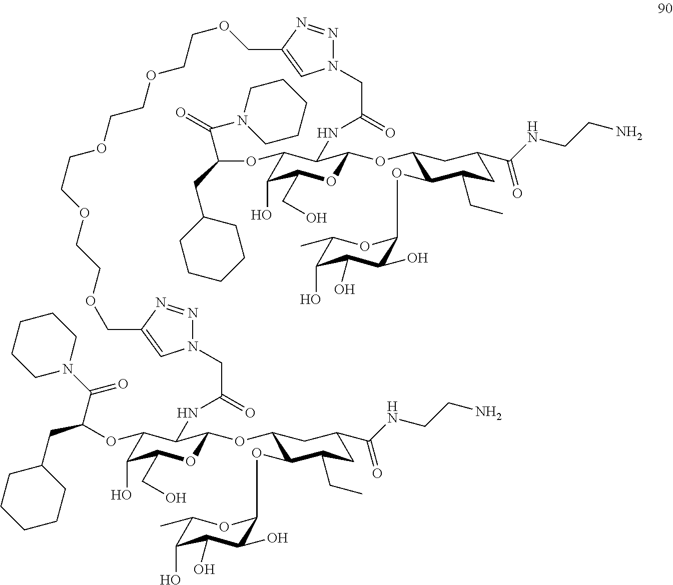

### Example 59

### Prophetic Synthesis of Multimeric Compound 91

Compound 91: Compound 91 can be prepared in an analogous fashion to FIGS. 11 and 12 using in PEG-9 bis-propargyl ether in place of compound 43 in step b of Scheme 11.

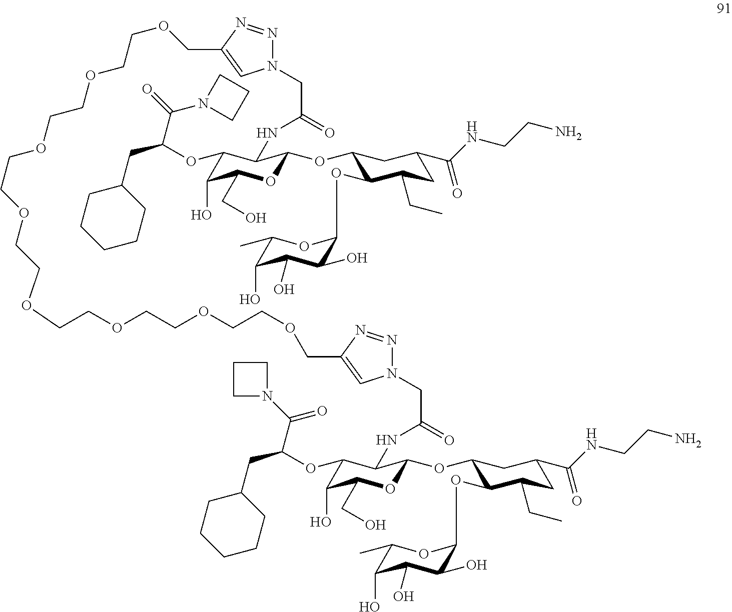

### Example 60

### Prophetic Synthesis of Multimeric Compound 92

Compound 92: Compound 92 can be prepared in an analogous fashion to FIGS. 11 and 12 using 1,2-bi(2-propynyloxy) ethane in place of compound 43 in step b in Scheme 11.

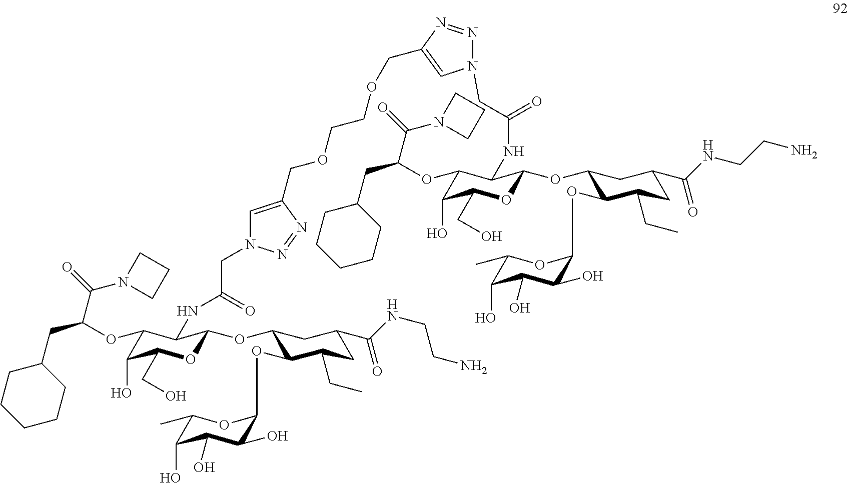

### Example 61

### Prophetic Synthesis of Multimeric Compound 93

Compound 93: Compound 93 can be prepared in an analogous fashion to FIGS. 11 and 12 using 1,2-bi(2-propynyloxy) ethane in place of compound 43 in step b in Scheme 11 and using 2-aminoethyl ether in place of ethylenediamine in step b of Scheme 12.

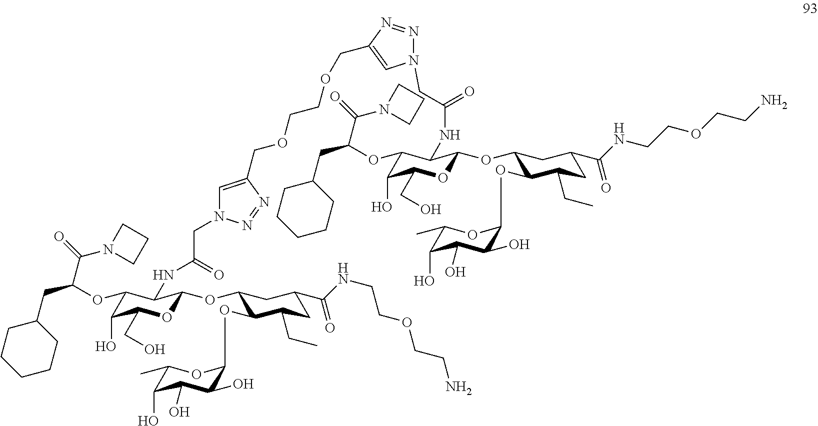

### Example 62

### Synthesis of Multimeric Compound 95

Compound 95: Compound 22 and compound 94 (5 eq) preparation described in WO/2016089872) is co-evaporated 3 times from methanol and stored under vacuum for 1 hour. The mixture is dissolved in methanol under an argon atmosphere and stirred for 1 hour at room temperature. Sodium triacetoxyborohydride (15 eq) is added and the reaction mixture is stirred overnight at room temperature. The solvent is removed and the residue is separated by C-18 reverse phase chromatography.

The purified material is dissolved in methanol at room temperature. The pH is adjusted to 12 with 1N NaOH. The reaction mixture is stirred at room temperature until completion. The pH is adjusted to 9. The solvent is removed under vacuum and the residue is separated by C-18 reverse phase chromatography to afford compound 95.

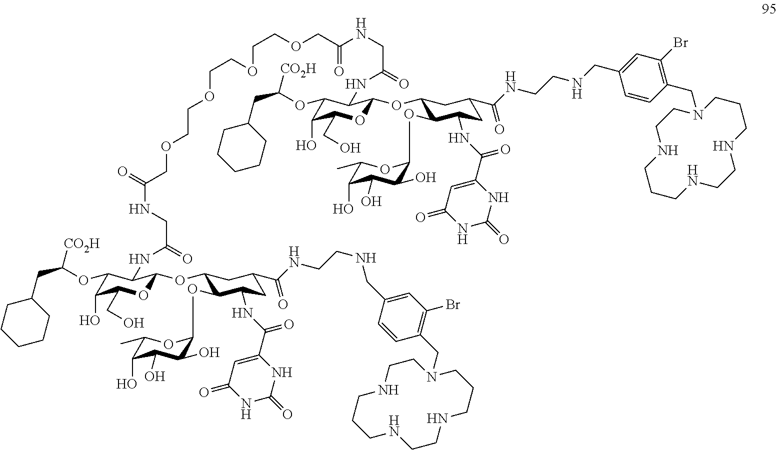

### Example 63

### Prophetic Synthesis of Multimeric Compound 96

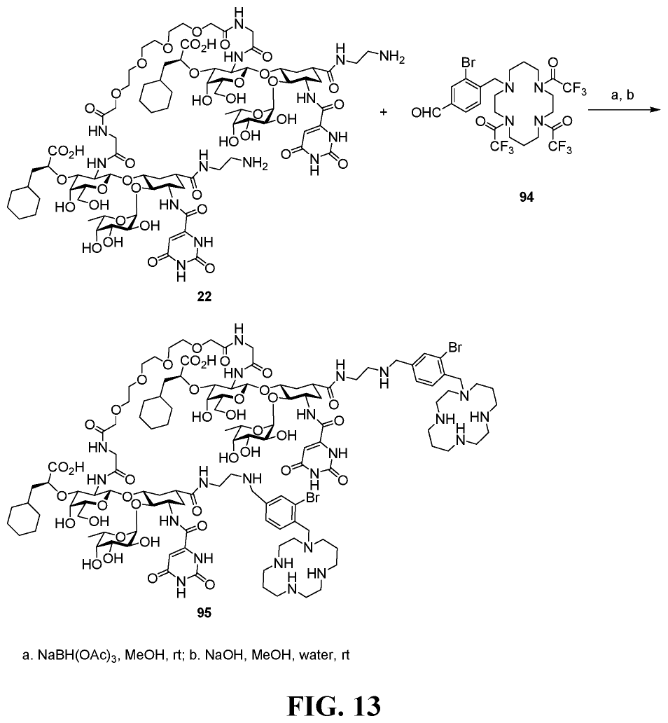

Compound 96: Compound 96 can be prepared in an analogous fashion to FIG. 13 by replacing compound 22 with compound 23 in step a.

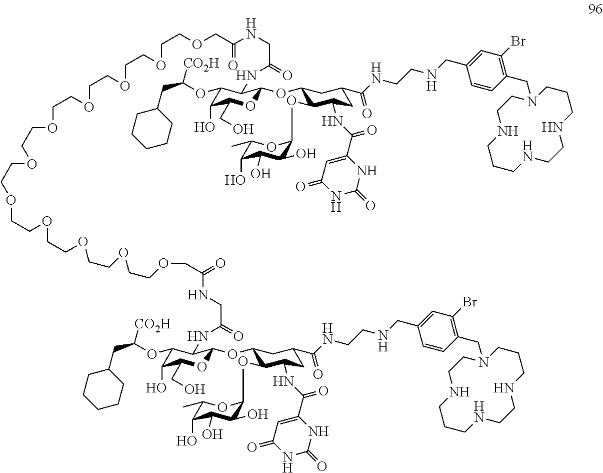

### Example 64

### Prophetic Synthesis of Multimeric Compound 97

Compound 97: Compound 97 can be prepared in an analogous fashion to FIG. 13 by replacing compound 22 with compound 24 in step a.

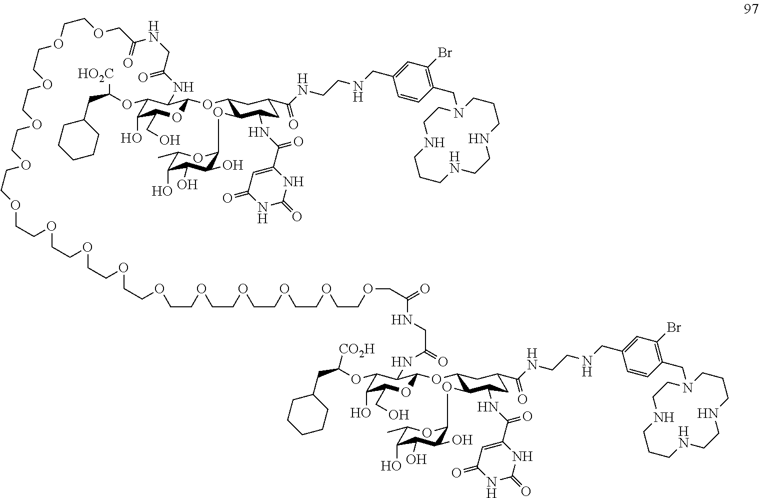

### Example 65

### Prophetic Synthesis of Multimeric Compound 98

Compound 98: Compound 98 can be prepared in an analogous fashion to FIG. 13 by replacing compound 22 with compound 25 in step a.

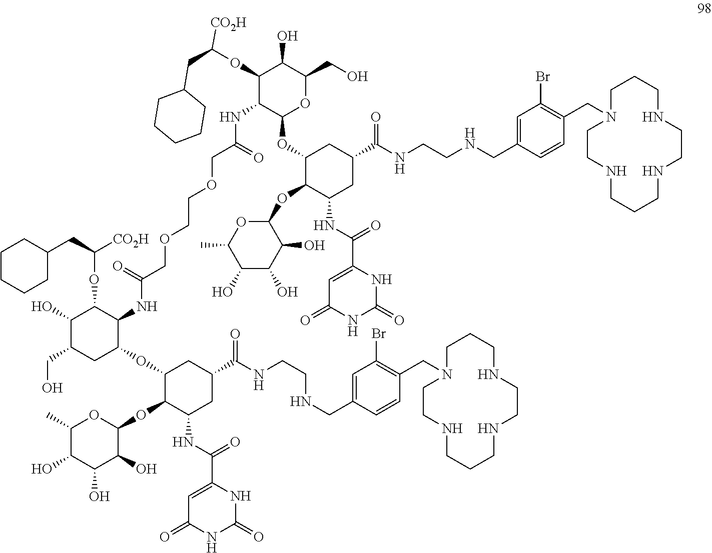 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2NC(=O)COCCOCC(=O)N[C@@H]2[C@@H](O[C@@H](CC3CCCCC3)C(=O)O)[C@@H](O)[C@@H](CO)C[C@H]2O[C@@H]2C[C@H](C(=O)NCCNCc3ccc(CN4CCCNCCNCCCNCC4)c(Br)c3)C[C@H](NC(=O)c3cc(=O)[nH]c(=O)[nH]3)[C@H]2O[C@@H]2O[C@@H](C)[C@@H](O)[C@@H](O)[C@@H]2O)[C@@H](O)[C@H](O)[C@@H]1O]

### Example 66

### Prophetic Synthesis of Multimeric Compound 99

Compound 99: Compound 99 can be prepared in an analogous fashion to FIG. 13 by replacing compound 22 with compound 26 in step a.

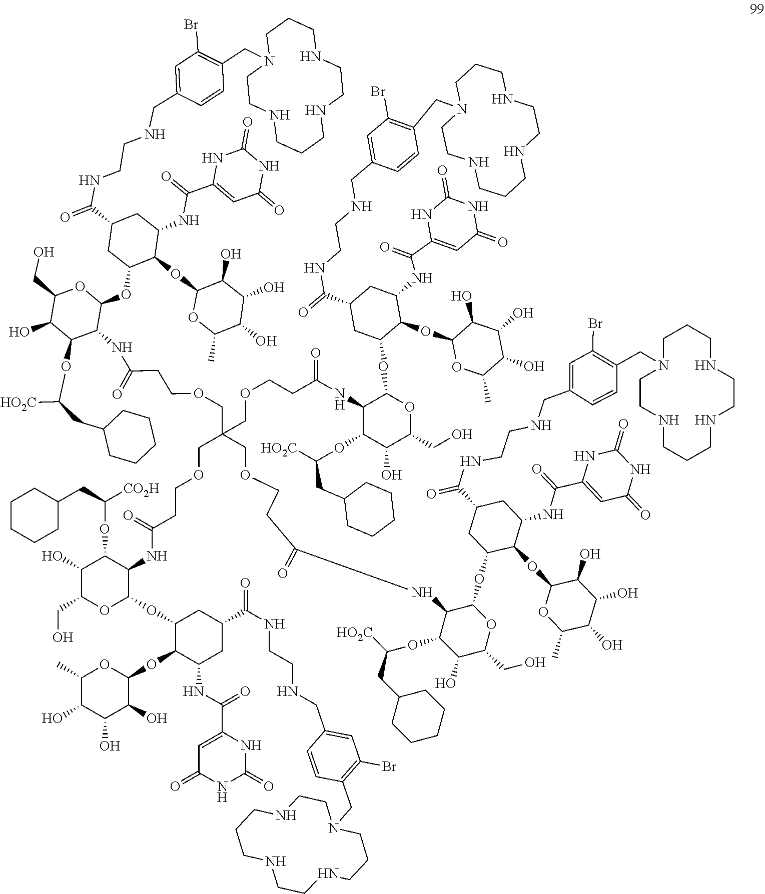

### Example 67

### Prophetic Synthesis of Multimeric Compound 100

Compound 100: Compound 100 can be prepared in an analogous fashion to FIG. 13 by replacing compound 22 with compound 27 in step a.

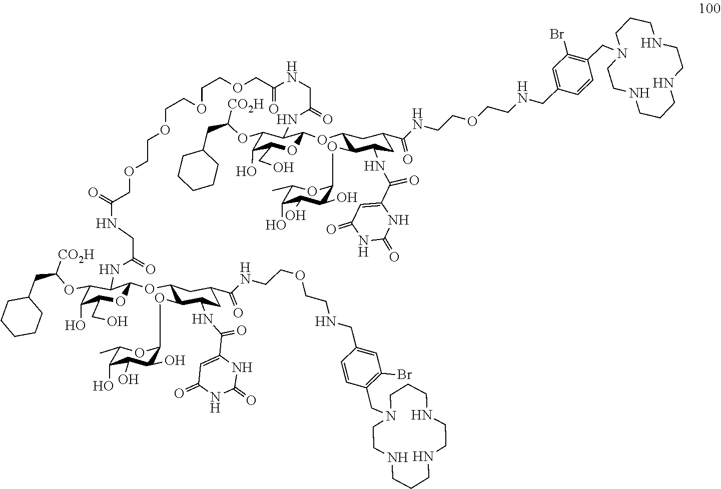

### Example 69

### Prophetic Synthesis of Multimeric Compound 102

Compound 101: Compound 101 can be prepared in an analogous fashion to FIG. 13 by replacing compound 22 with compound 28 in step a.

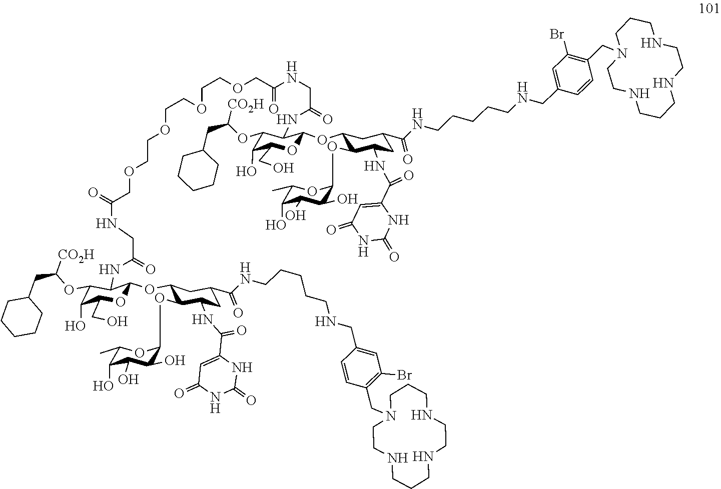

### Example 69

### Prophetic Synthesis of Multimeric Compound 102

Compound 102: Compound 102 can be prepared in an analogous fashion to FIG. 13 by replacing compound 22 with compound 29 in step a.

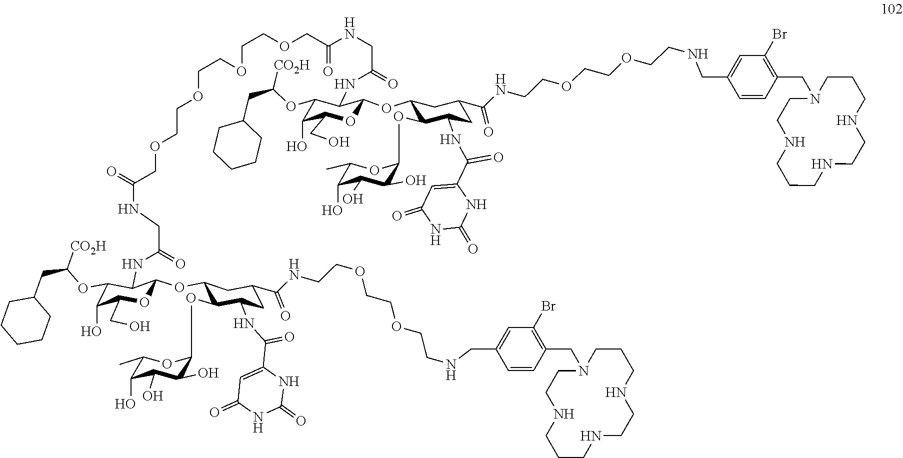

### Example 70

### Prophetic Synthesis of Multimeric Compound 103

Compound 103: Compound 103 can be prepared in an analogous fashion to FIG. 13 by replacing compound 22 with compound 30 in step a.

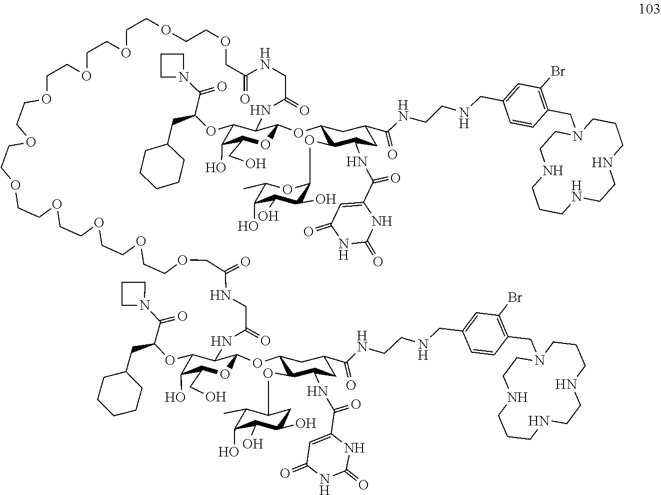

### Example 71

### Prophetic Synthesis of Multimeric Compound 104

Compound 104: Compound 104 can be prepared in an analogous fashion to FIG. 13 by replacing compound 22 with compound 31 in step a.

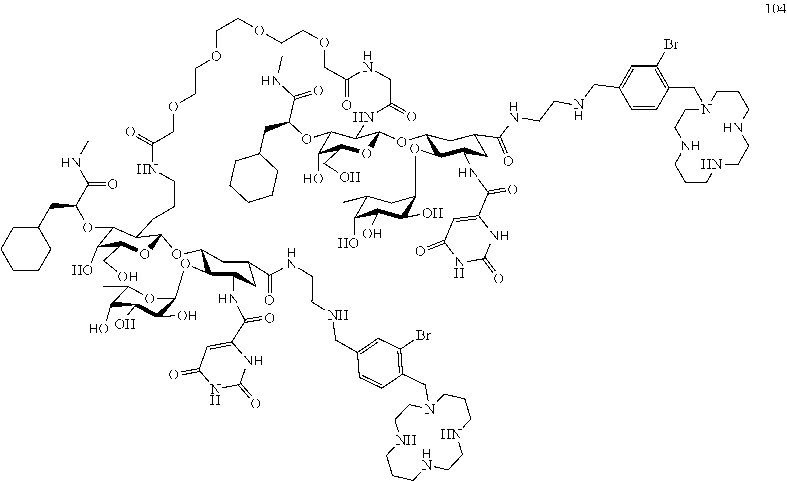

### Example 72

### Prophetic Synthesis of Multimeric Compound 105

Compound 105: Compound 105 can be prepared in an analogous fashion to FIG. 13 by replacing compound 22 with compound 32 in step a.

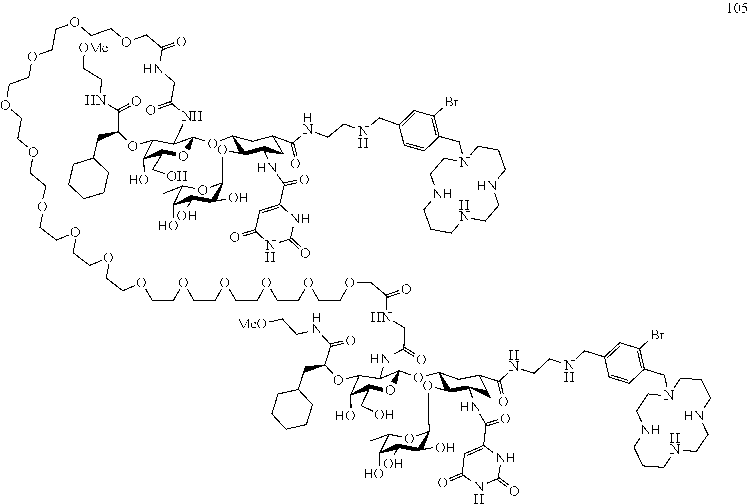

### Example 73

### Prophetic Synthesis of Multimeric Compound 106

Compound 106: Compound 106 can be prepared in an analogous fashion to FIG. 13 by replacing compound 22 with compound 33 in step a.

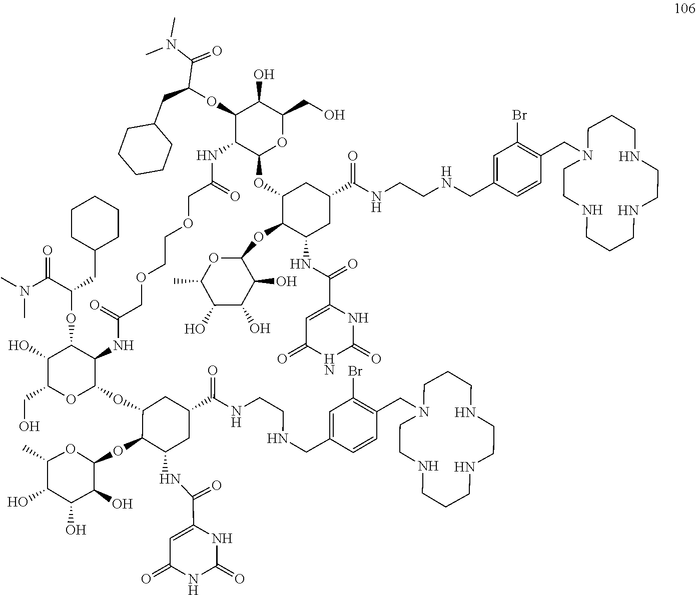 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 74

### Prophetic Synthesis of Multimeric Compound 107

Compound 107: Compound 107 can be prepared in an analogous fashion to FIG. 13 by replacing compound 22 with compound 34 in step a.

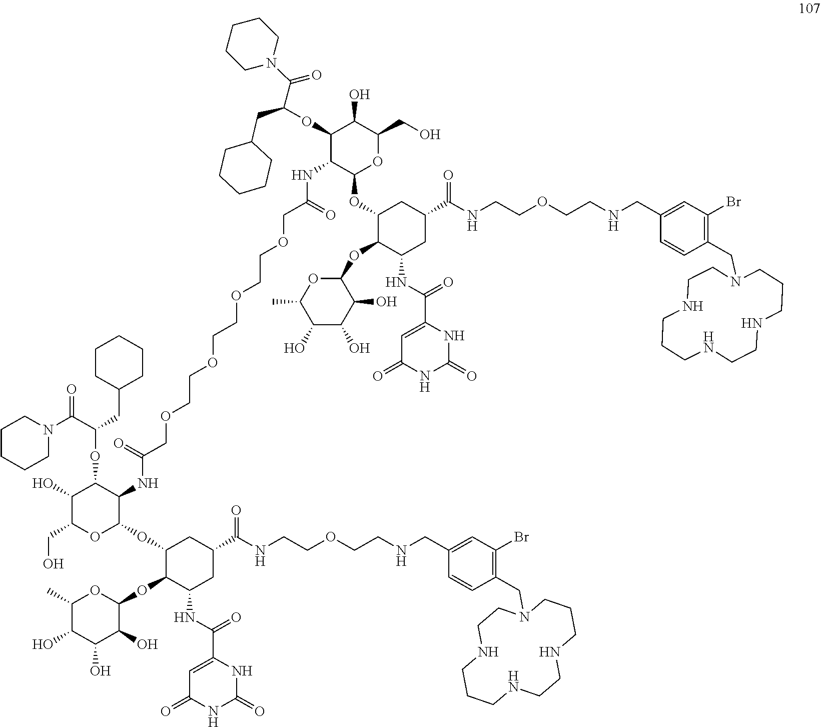 [SMILES: C[C@@H]1O[C@@H](O[C@@H]2[C@@H](NC(=O)c3cc(=O)[nH]c(=O)[nH]3)C[C@@H](C(=O)NCCOCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H](O)[C@H](O)[C@@H]1O]

### Example 75

### Prophetic Synthesis of Multimeric Compound 108

Compound 108: Compound 108 can be prepared in an analogous fashion to FIG. 13 by replacing compound 22 with compound 37 in step a.

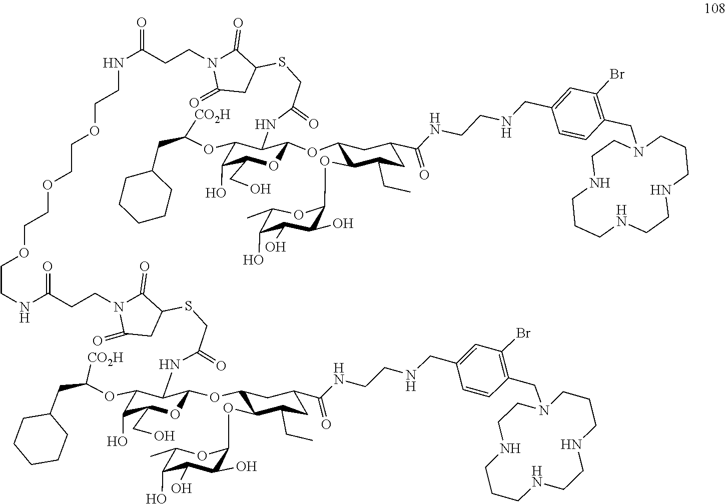

### Example 76

### Prophetic Synthesis of Multimeric Compound 109

Compound 109: Compound 109 can be prepared in an analogous fashion to FIG. 13 by replacing compound 22 with compound 38 in step a.

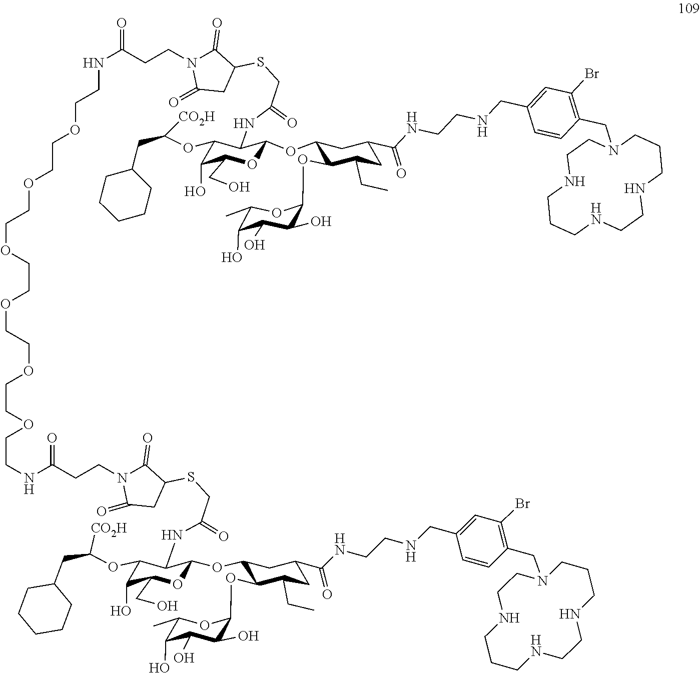

### Example 77

### Prophetic Synthesis of Multimeric Compound 110

Compound 110: Compound 110 can be prepared in an analogous fashion to FIG. 13 by replacing compound 22 with compound 39 in step a.

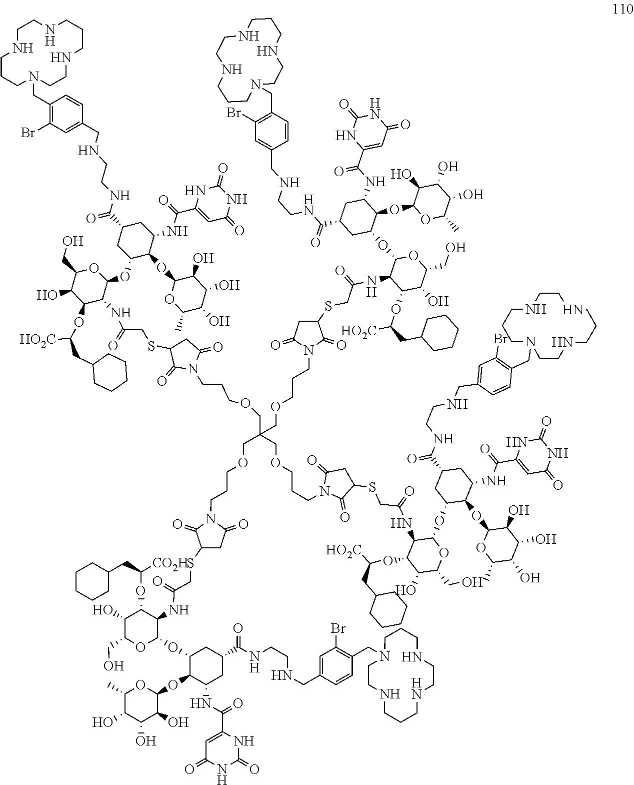

### Prophetic Synthesis of Multimeric Compound 111

Compound 111: Compound 111 can be prepared in an analogous fashion to FIG. 13 by replacing compound 22 with compound 40 in step a.

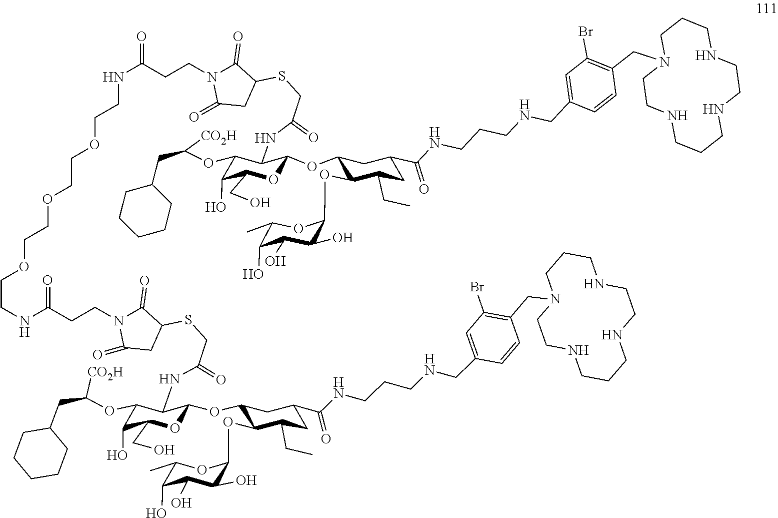

### Example 78

### Prophetic Synthesis of Multimeric Compound 112

Compound 112: Compound 112 can be prepared in an analogous fashion to FIG. 13 by replacing compound 22 with compound 46 in step a.

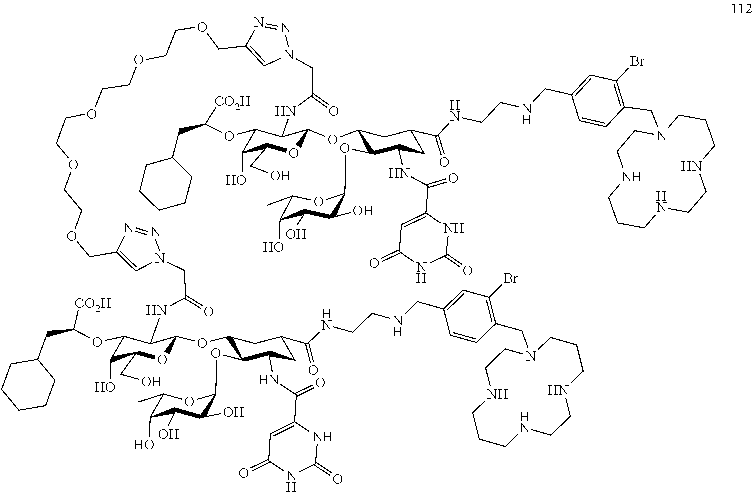

### Example 79

### Prophetic Synthesis of Multimeric Compound 113

Compound 113: Compound 113 can be prepared in an analogous fashion to FIG. 13 by replacing compound 22 with compound 47 in step a.

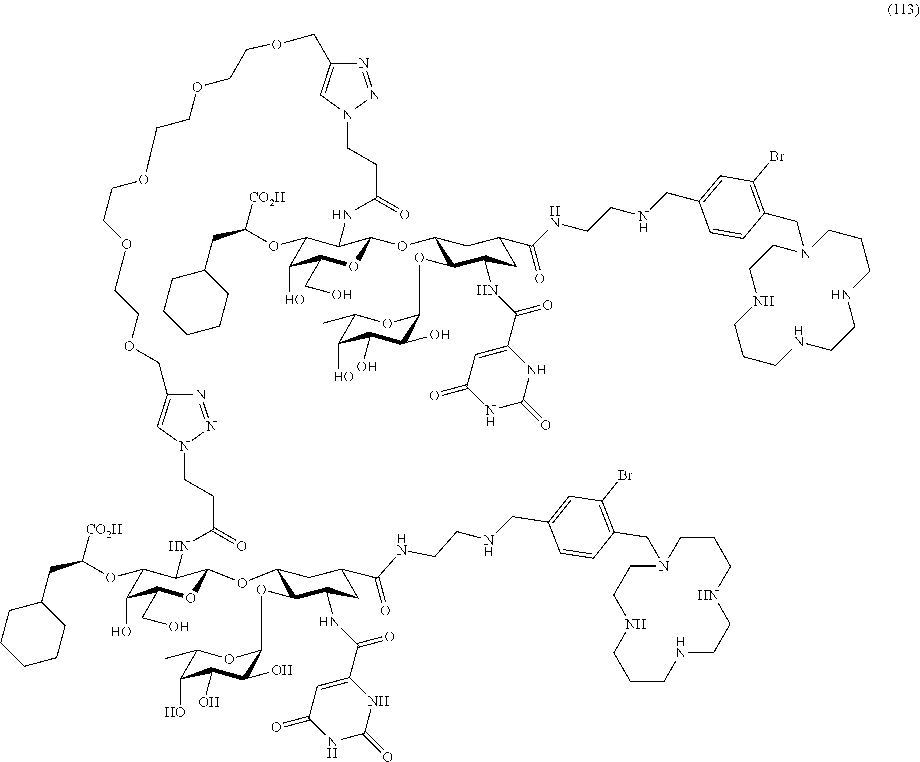

### Example 80

### Prophetic Synthesis of Multimeric Compound 114

Compound 114: Compound 114 can be prepared in an analogous fashion to FIG. 13 by replacing compound 22 with compound 48 in step a.

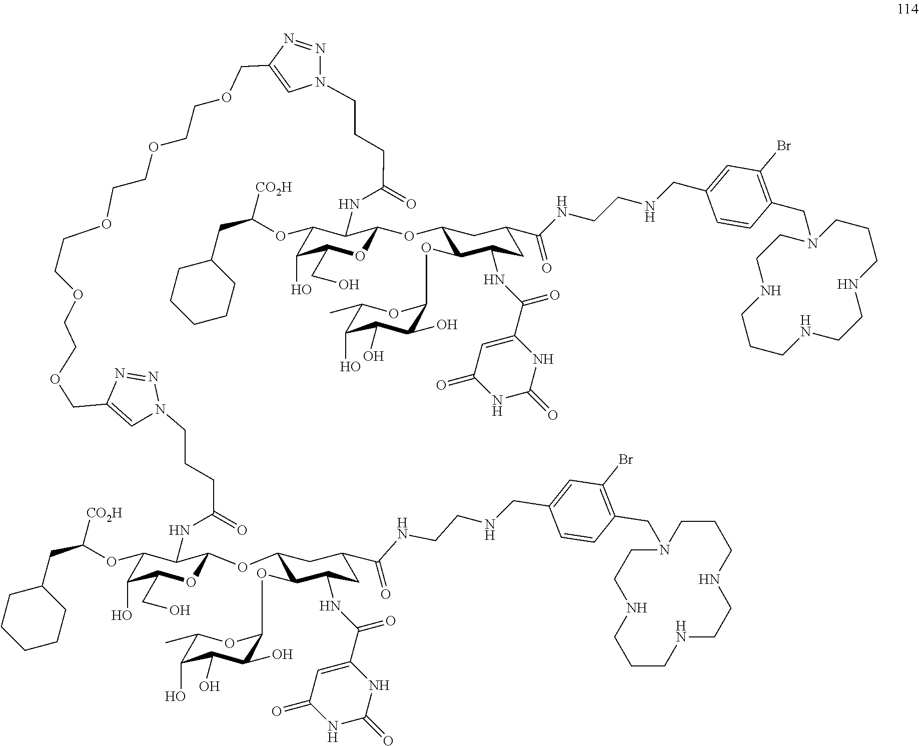

### Example 81

### Prophetic Synthesis of Multimeric Compound 115

Compound 115: Compound 115 can be prepared in an analogous fashion to FIG. 13 by replacing compound 22 with compound 49 in step a.

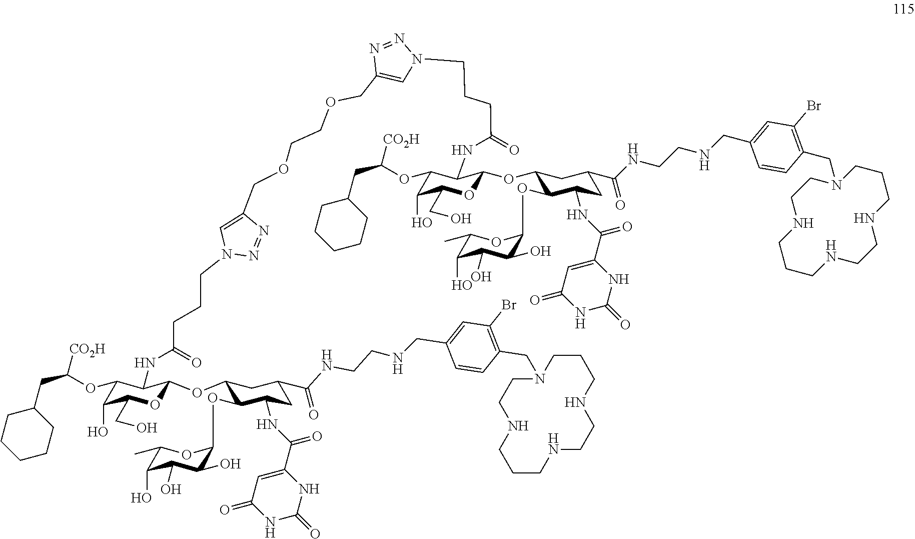

### Example 82

### Prophetic Synthesis of Multimeric Compound 116

Compound 116: Compound 116 can be prepared in an analogous fashion to FIG. 13 by replacing compound 22 with compound 50 in step a.

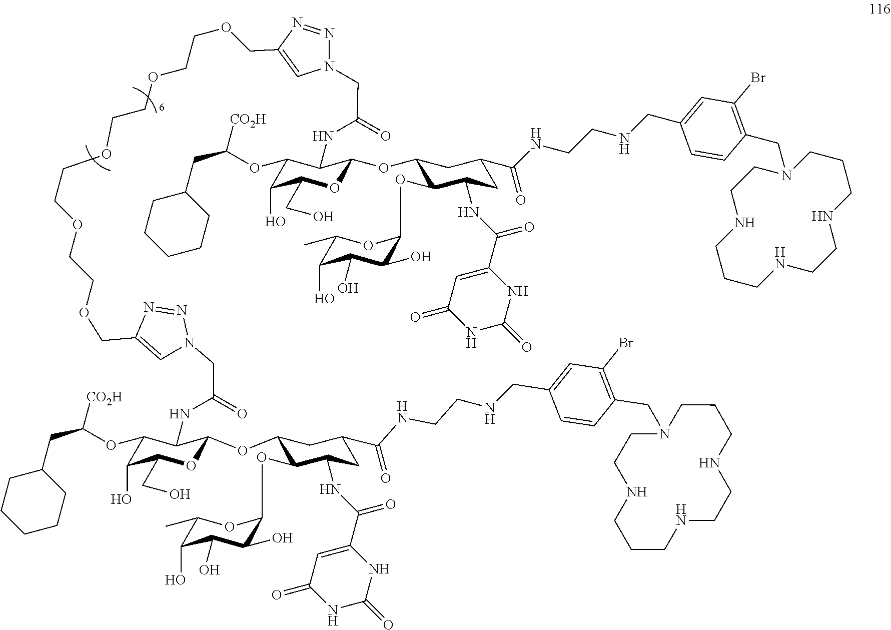

### Example 83

### Prophetic Synthesis of Multimeric Compound 117

Compound 117: Compound 117 can be prepared in an analogous fashion to FIG. 13 by replacing compound 22 with compound 51 in step a.

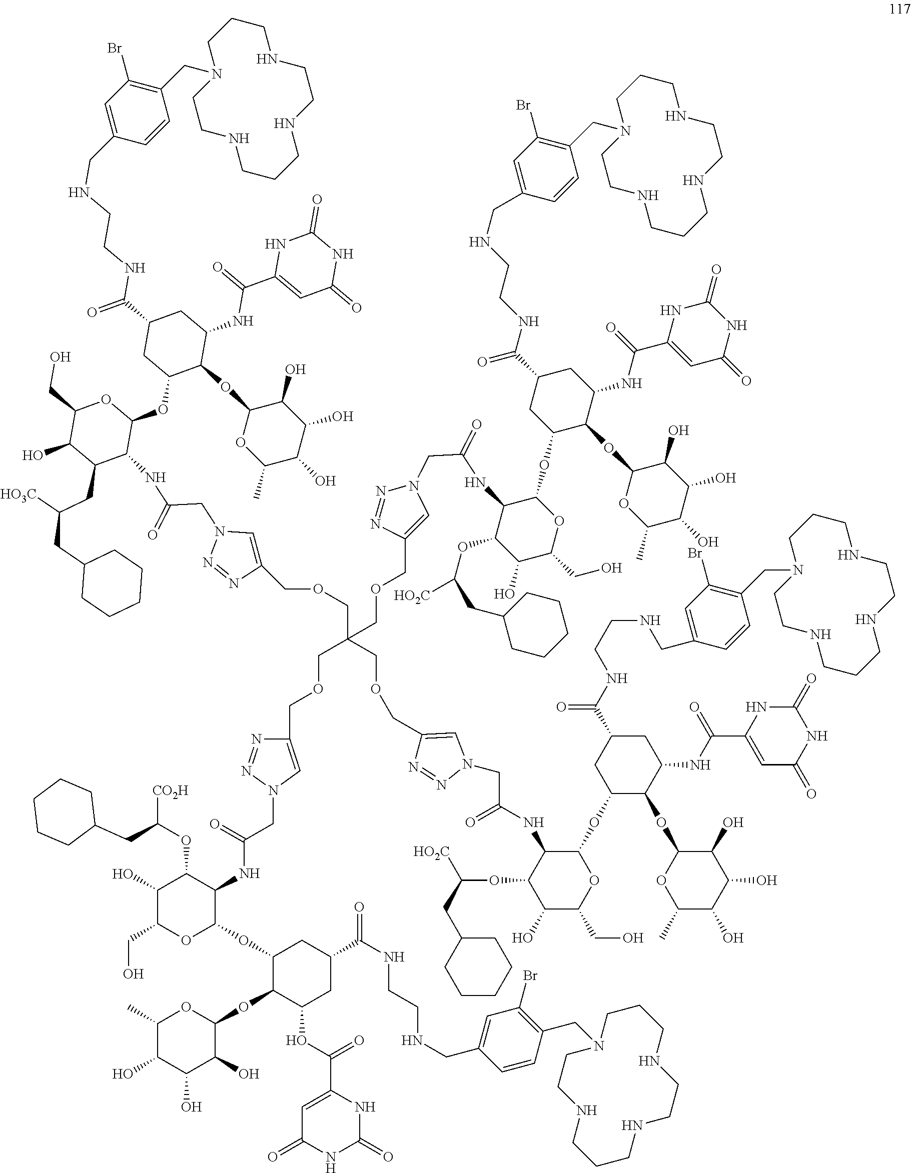

### Example 84

### Prophetic Synthesis of Multimeric Compound 118

Compound 118: Compound 118 can be prepared in an analogous fashion to FIG. 13 by replacing compound 22 with compound 52 in step a.

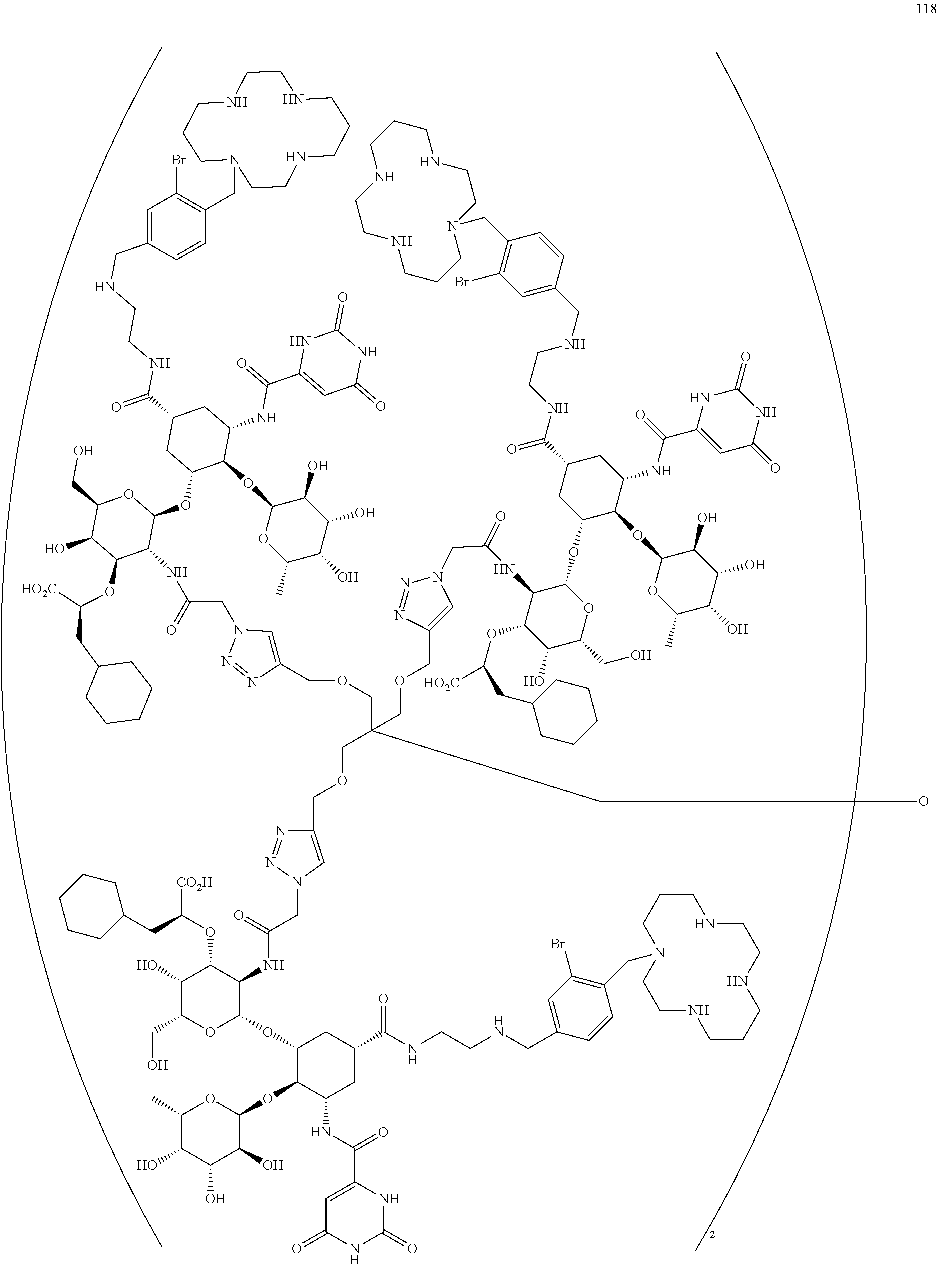

### Example 85

### Prophetic Synthesis of Multimeric Compound 119

Compound 119: Compound 119 can be prepared in an analogous fashion to FIG. 13 by replacing compound 22 with compound 53 in step a.

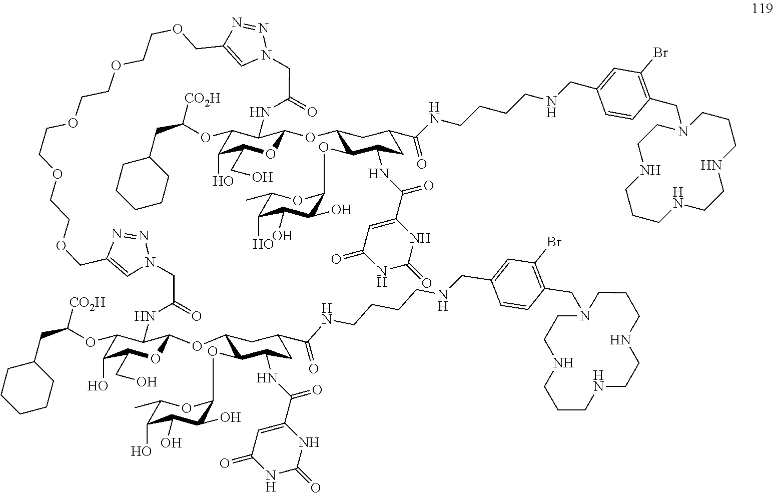

### Example 86

### Prophetic Synthesis of Multimeric Compound 120

Compound 120: Compound 120 can be prepared in an analogous fashion to FIG. 13 by replacing compound 22 with compound 54 in step a.

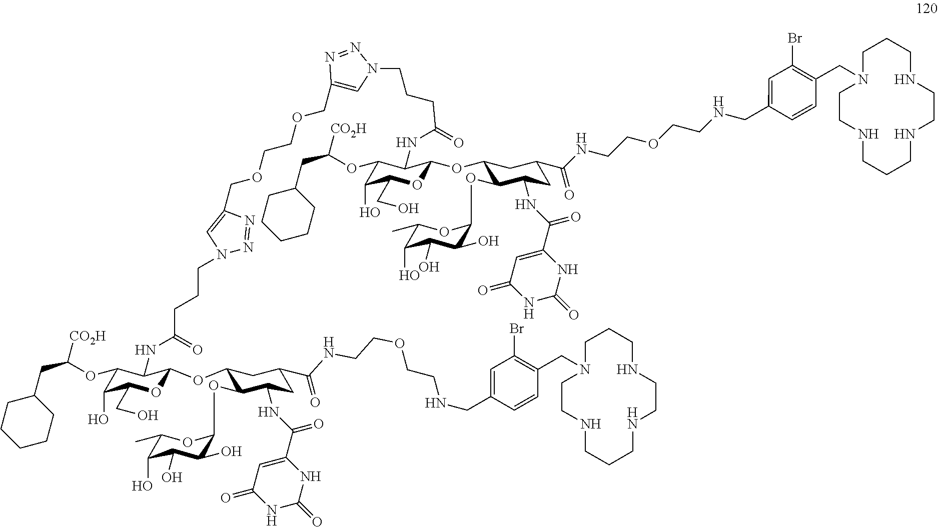

### Example 87

### Prophetic Synthesis of Multimeric Compound 121

Compound 121: Compound 121 can be prepared in an analogous fashion to FIG. 13 by replacing compound 22 with compound 56 in step a.

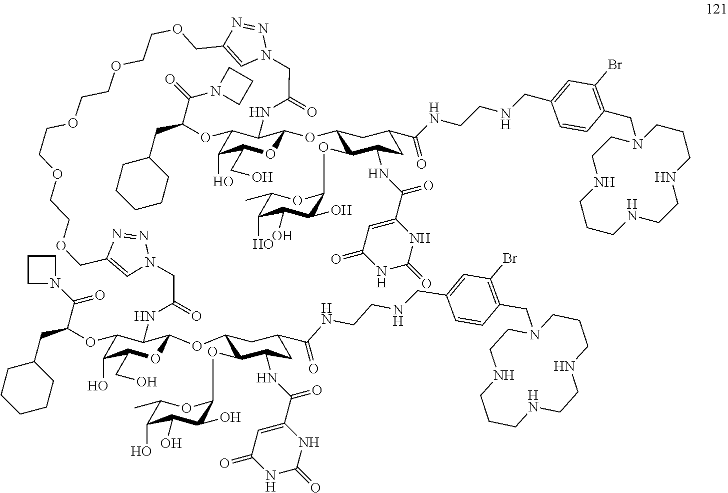

### Example 88

### Prophetic Synthesis of Multimeric Compound 122

Compound 122: Compound 122 can be prepared in an analogous fashion to FIG. 13 by replacing compound 22 with compound 57 in step a.

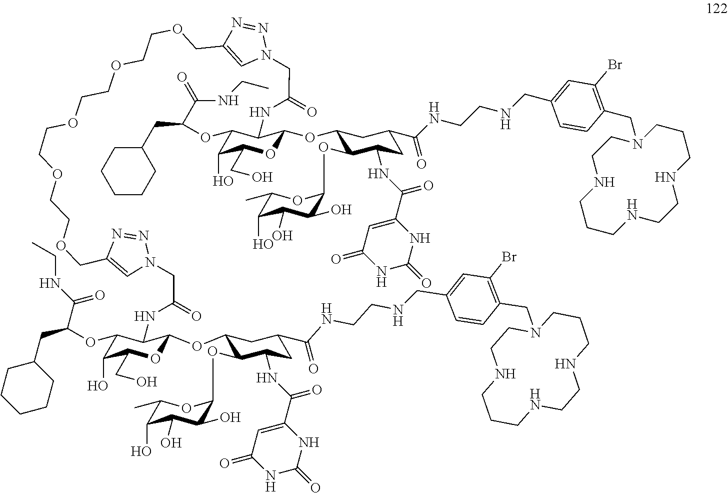

### Example 89

### Prophetic Synthesis of Multimeric Compound 123

Compound 123: Compound 123 can be prepared in an analogous fashion to FIG. 13 by replacing compound 22 with compound 58 in step a.

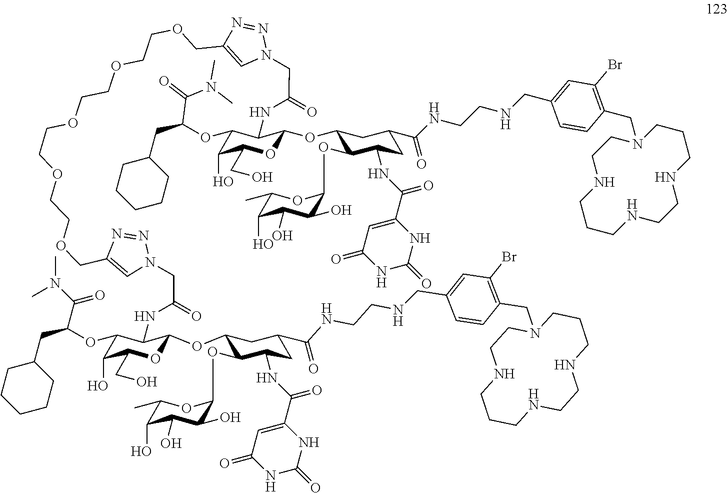

### Example 90

### Prophetic Synthesis of Multimeric Compound 124

Compound 124: Compound 124 can be prepared in an analogous fashion to FIG. 13 by replacing compound 22 with compound 59 in step a.

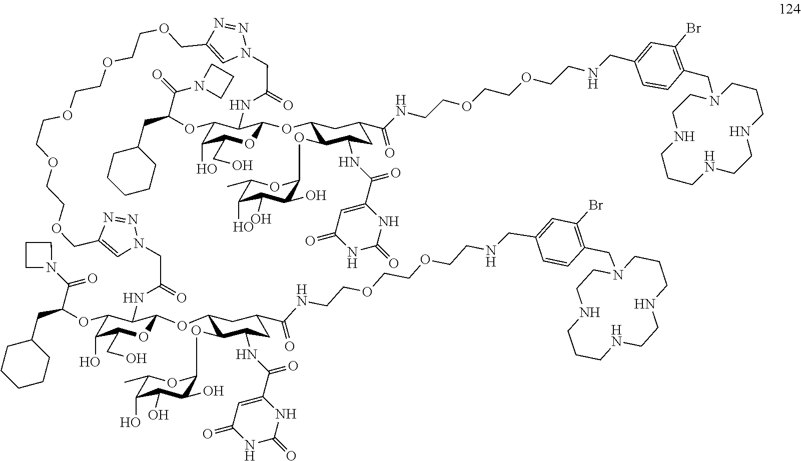

### Example 91

### Prophetic Synthesis of Multimeric Compound 125

Compound 125: Compound 125 can be prepared in an analogous fashion to FIG. 13 by replacing compound 22 with compound 68 in step a.

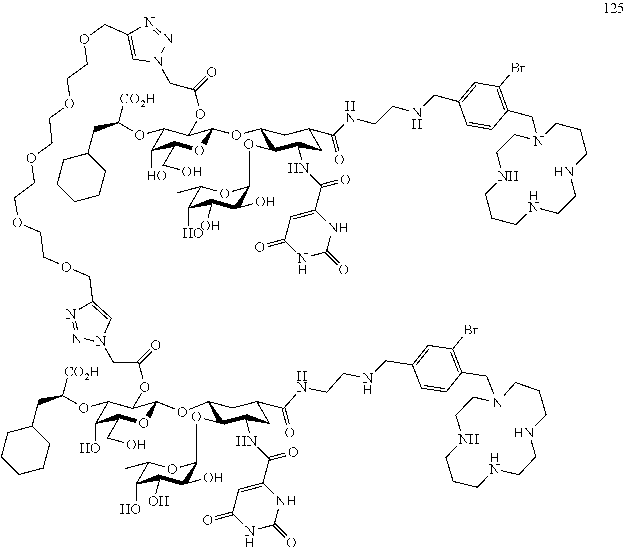

### Example 92

### Prophetic Synthesis of Multimeric Compound 126

Compound 126: Compound 126 can be prepared in an analogous fashion to FIG. 13 by replacing compound 22 with compound 69 in step a.

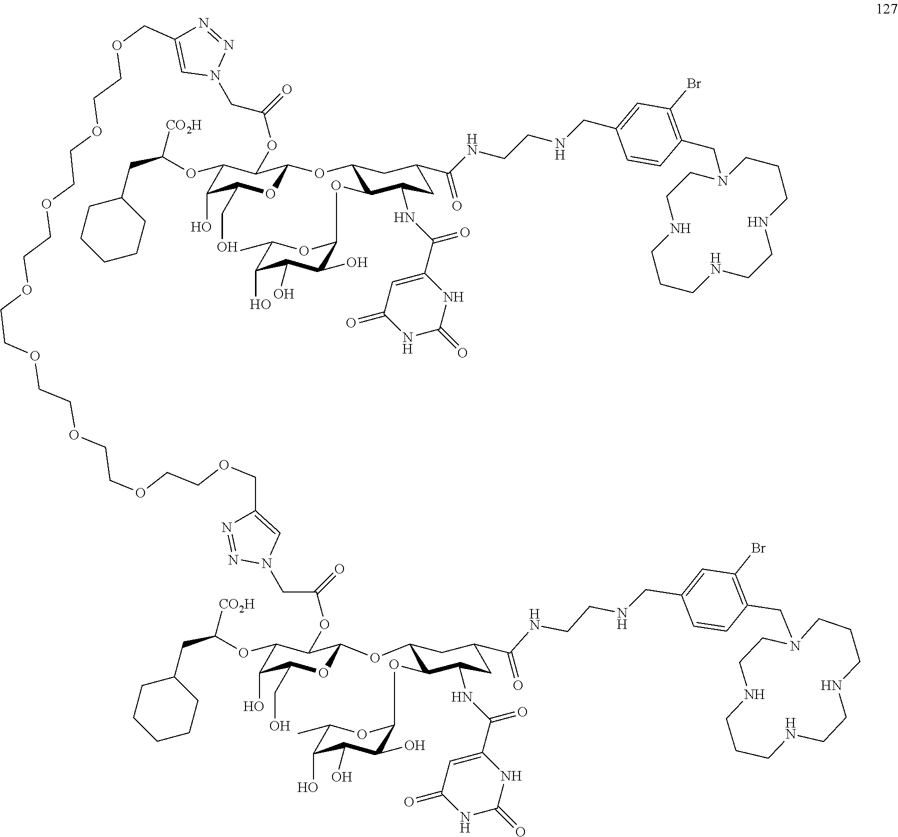

### Example 93

### Prophetic Synthesis of Multimeric Compound 127

Compound 127: Compound 127 can be prepared in an analogous fashion to FIG. 13 by replacing compound 22 with compound 70 in step a.

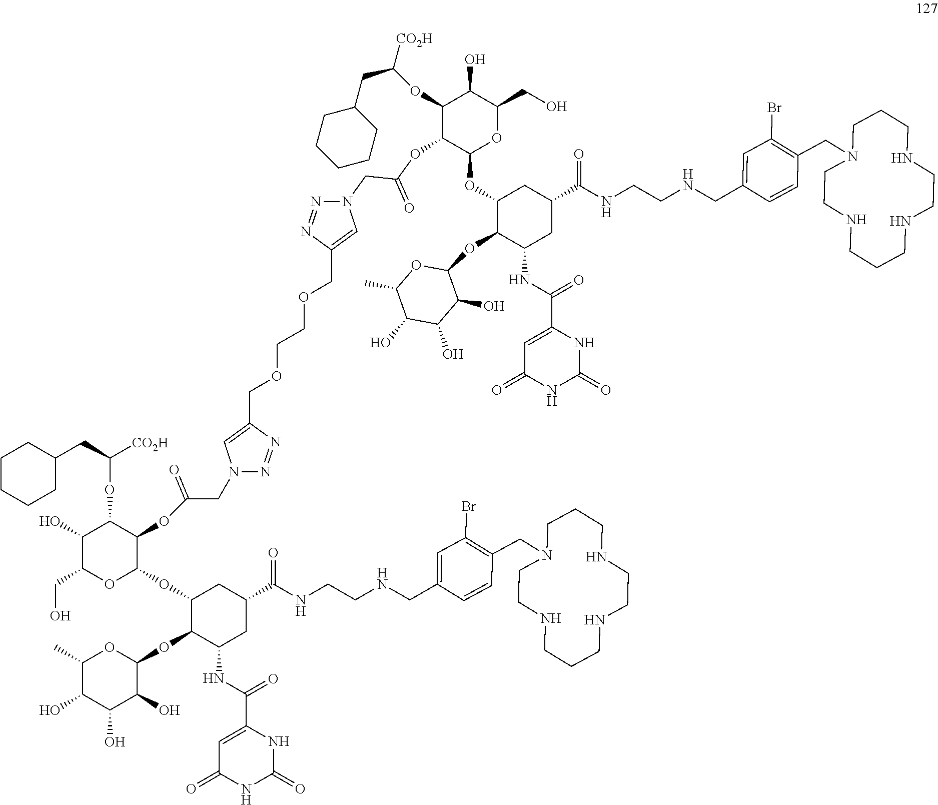 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5C[C@H](C(=O)NCCNCc6ccc(CN7CCCNCCNCCCNCC7)c(Br)c6)C[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 94

### Prophetic Synthesis of Multimeric Compound 128

Compound 128: Compound 128 can be prepared in an analogous fashion to FIG. 13 by replacing compound 22 with compound 71 in step a.

### Example 95

### Prophetic Synthesis of Multimeric Compound 129

Compound 129: Compound 129 can be prepared in an analogous fashion to FIG. 13 by replacing compound 22 with compound 73 in step a.

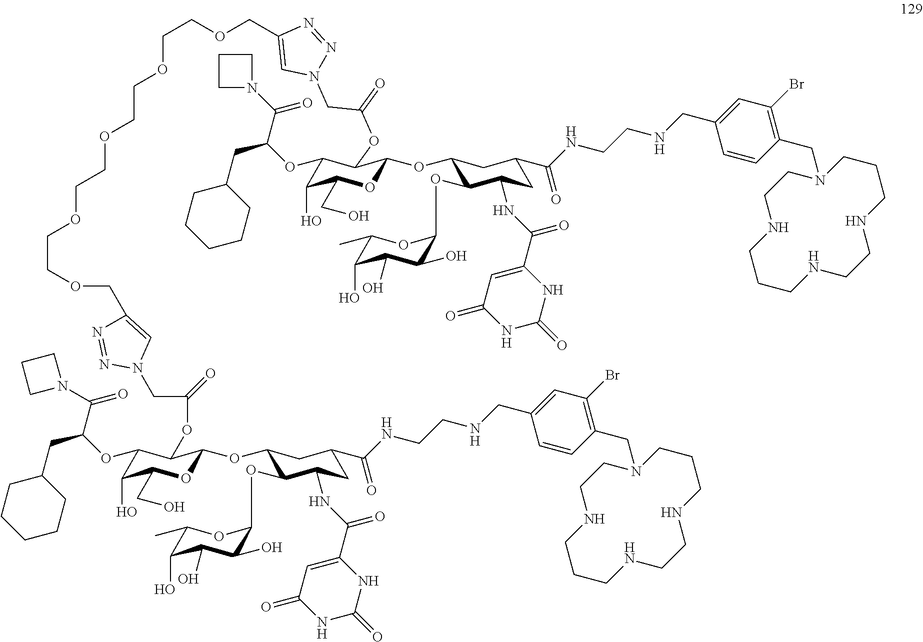

### Example 96

### Prophetic Synthesis of Multimeric Compound 130

Compound 130: Compound 130 can be prepared in an analogous fashion to FIG. 13 by replacing compound 22 with compound 78 in step a.

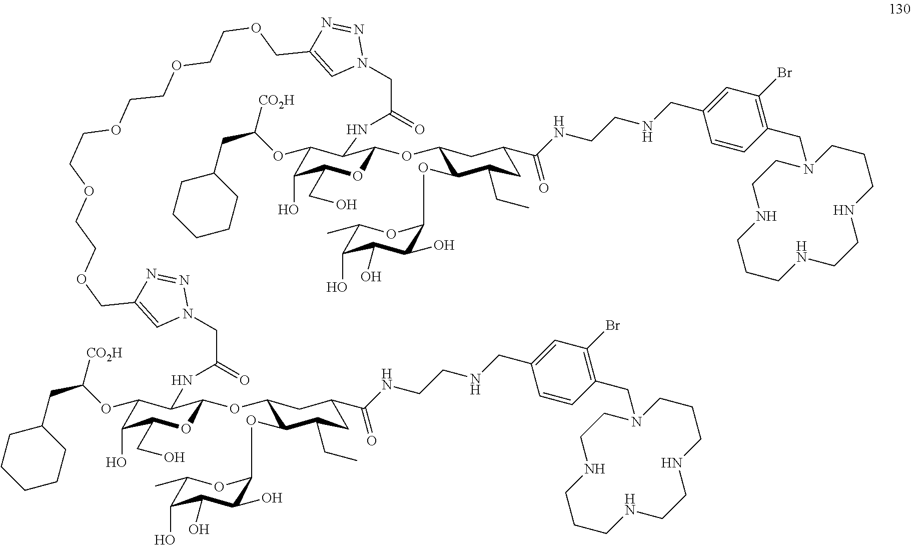

### Example 97

### Prophetic Synthesis of Multimeric Compound 131

Compound 131: Compound 131 can be prepared in an analogous fashion to FIG. 13 by replacing compound 22 with compound 79 in step a.

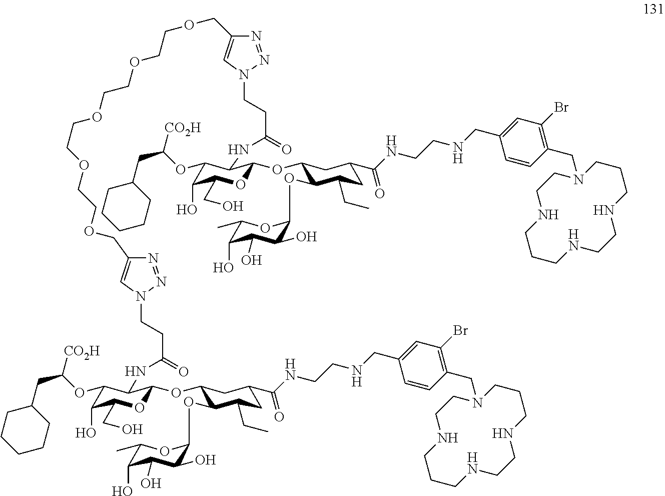

### Example 98

### Prophetic Synthesis of Multimeric Compound 132

Compound 132: Compound 132 can be prepared in an analogous fashion to FIG. 13 by replacing compound 22 with compound 80 in step a.

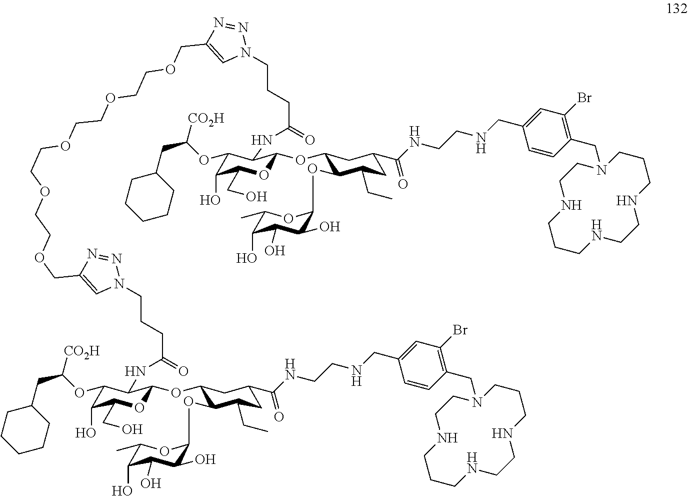

### Example 99

### Prophetic Synthesis of Multimeric Compound 133

Compound 133: Compound 133 can be prepared in an analogous fashion to FIG. 13 by replacing compound 22 with compound 81 in step a.

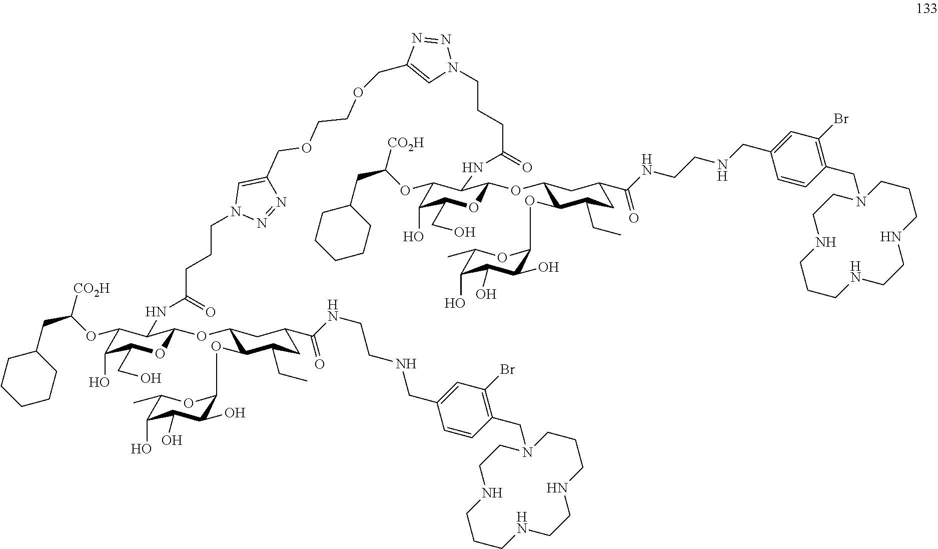

### Example 100

### Prophetic Synthesis of Multimeric Compound 134

Compound 134: Compound 134 can be prepared in an analogous fashion to FIG. 13 by replacing compound 22 with compound 82 in step a.

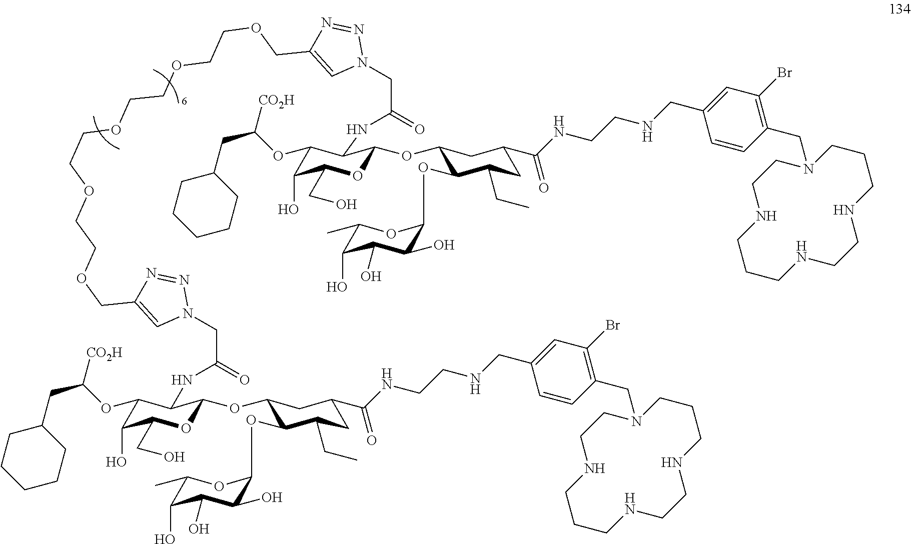

### Example 101

### Prophetic Synthesis of Multimeric Compound 135

Compound 135: Compound 135 can be prepared in an analogous fashion to FIG. 13 by replacing compound 22 with compound 83 in step a.

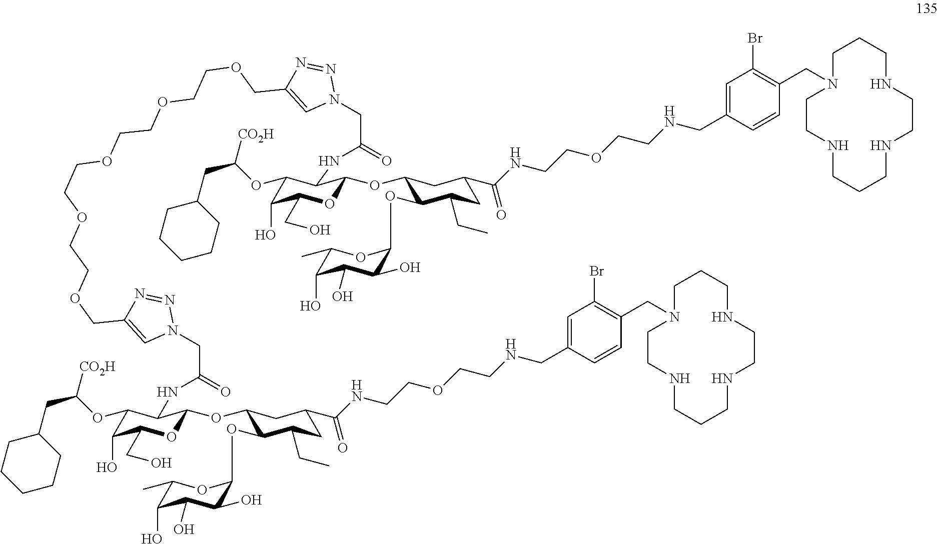

### Example 102

### Prophetic Synthesis of Multimeric Compound 136

Compound 136: Compound 136 can be prepared in an analogous fashion to FIG. 13 by replacing compound 22 with compound 84 in step a.

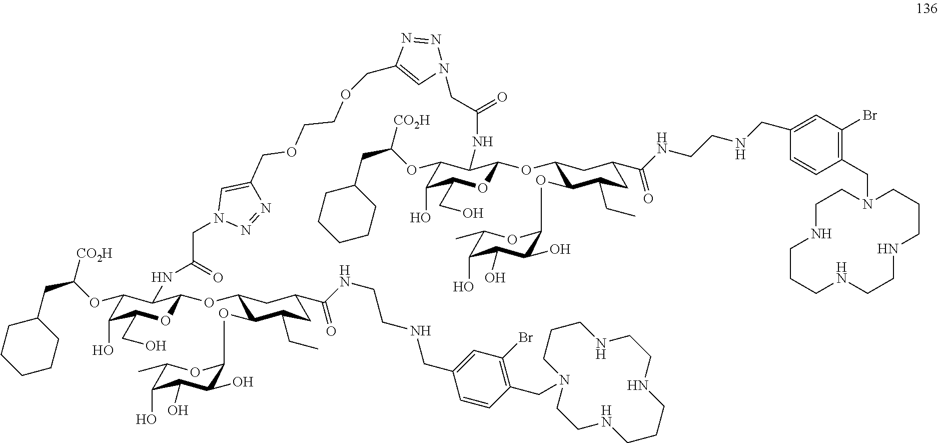

### Example 103

### Prophetic Synthesis of Multimeric Compound 137

Compound 137: Compound 137 can be prepared in an analogous fashion to FIG. 13 by replacing compound 22 with compound 85 in step a.

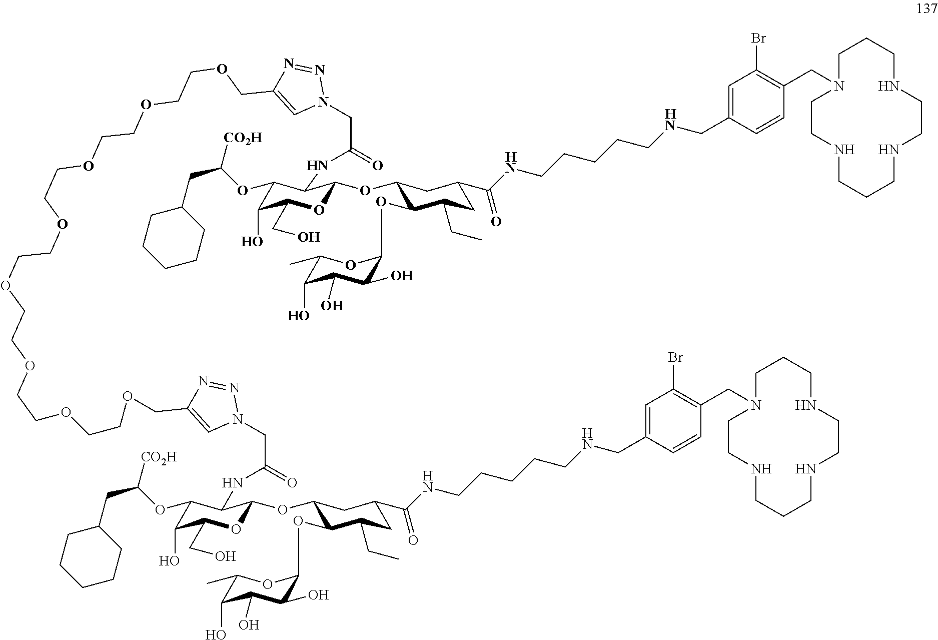

### Example 104

### Prophetic Synthesis of Multimeric Compound 138

Compound 138: Compound 138 can be prepared in an analogous fashion to FIG. 13 by replacing compound 22 with compound 87 in step a.

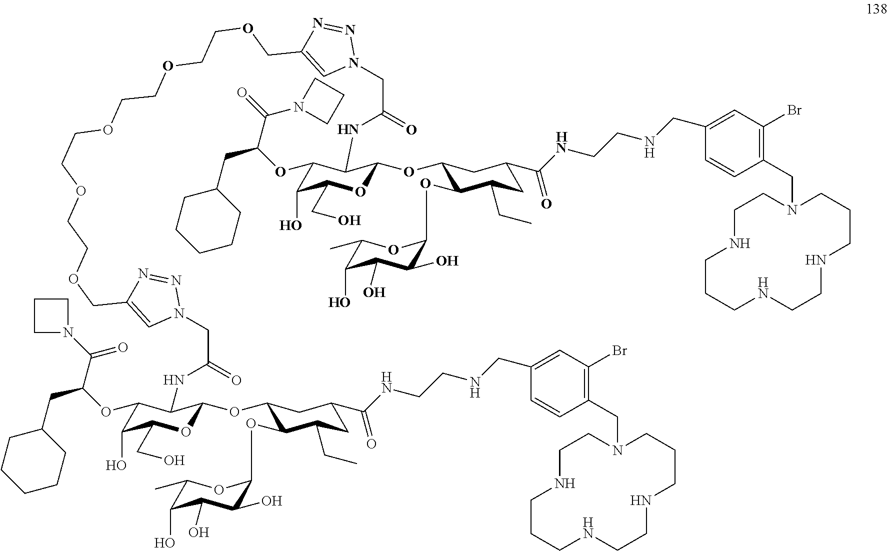

### Example 105

### Prophetic Synthesis of Multimeric Compound 139

Compound 139: Compound 139 can be prepared in an analogous fashion to FIG. 13 by replacing compound 22 with compound 88 in step a.

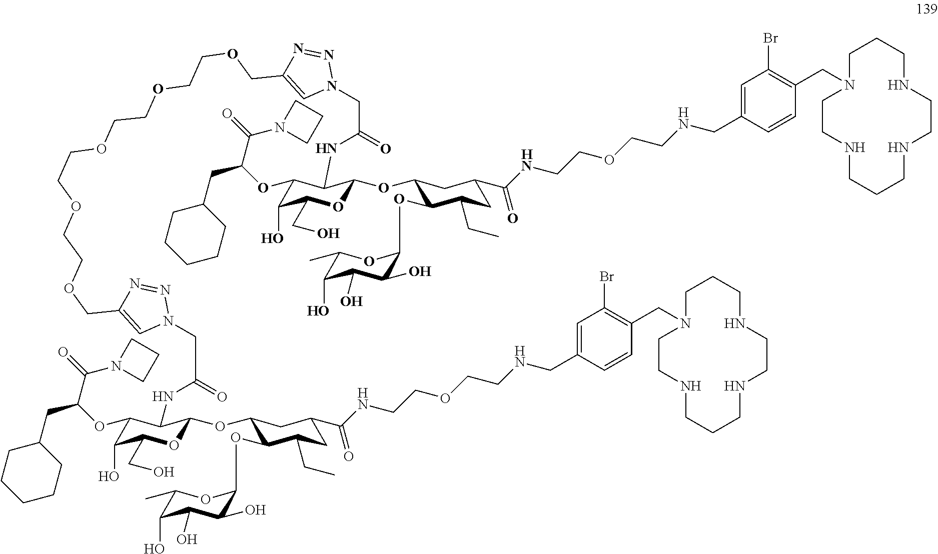

### Example 106

### Prophetic Synthesis of Multimeric Compound 140

Compound 140: Compound 140 can be prepared in an analogous fashion to FIG. 13 by replacing compound 22 with compound 89 in step a.

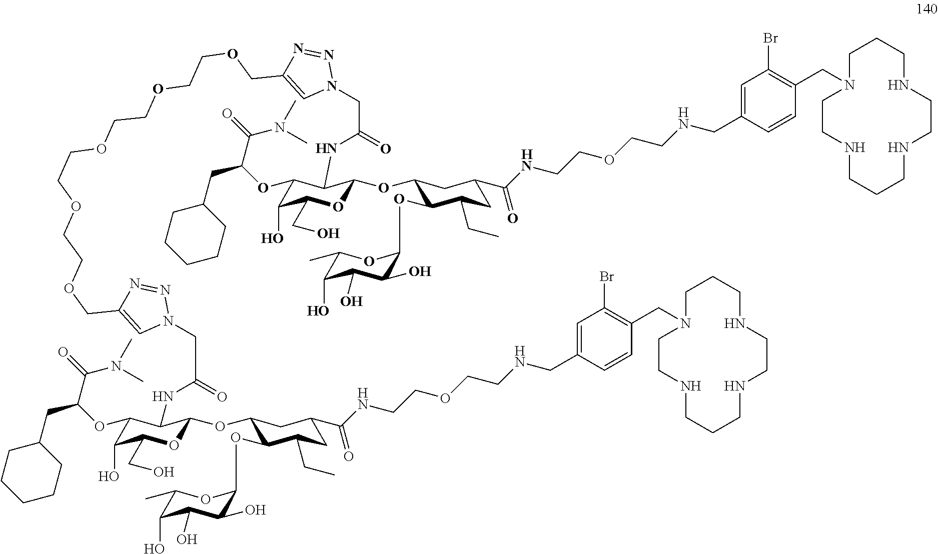

### Example 107

### Prophetic Synthesis of Multimeric Compound 141

Compound 141: Compound 141 can be prepared in an analogous fashion to FIG. 13 by replacing compound 22 with compound 90 in step a.

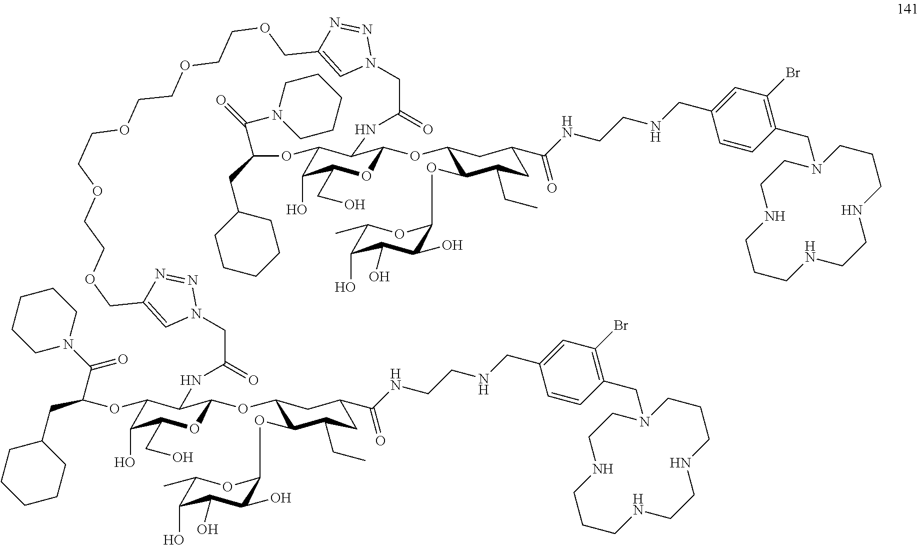

### Example 108

### Prophetic Synthesis of Multimeric Compound 142

Compound 142: Compound 142 can be prepared in an analogous fashion to FIG. 13 by replacing compound 22 with compound 91 in step a.

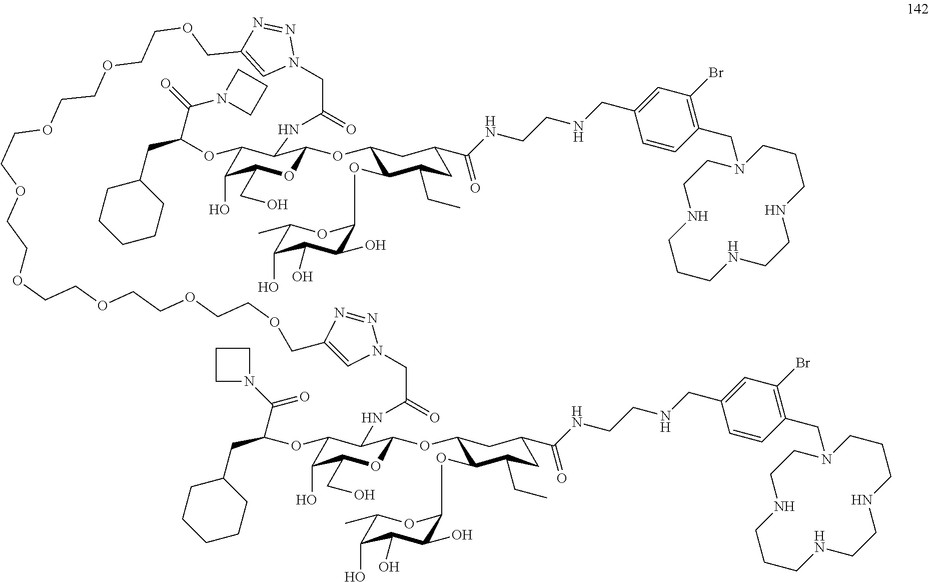

### Example 109

### Prophetic Synthesis of Multimeric Compound 143

Compound 143: Compound 143 can be prepared in an analogous fashion to FIG. 13 by replacing compound 22 with compound 92 in step a.

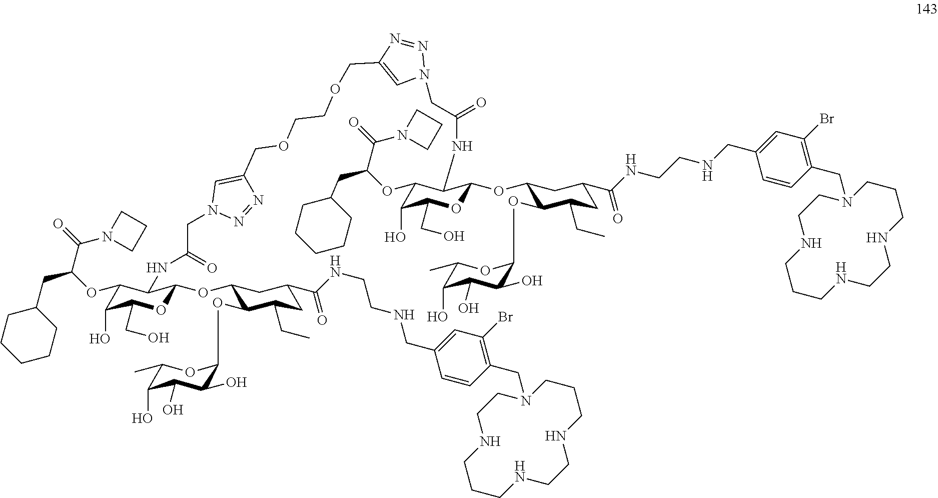

### Example 110

### Prophetic Synthesis of Multimeric Compound 144

Compound 144: Compound 144 can be prepared in an analogous fashion to FIG. 13 by replacing compound 22 with compound 93 in step a.

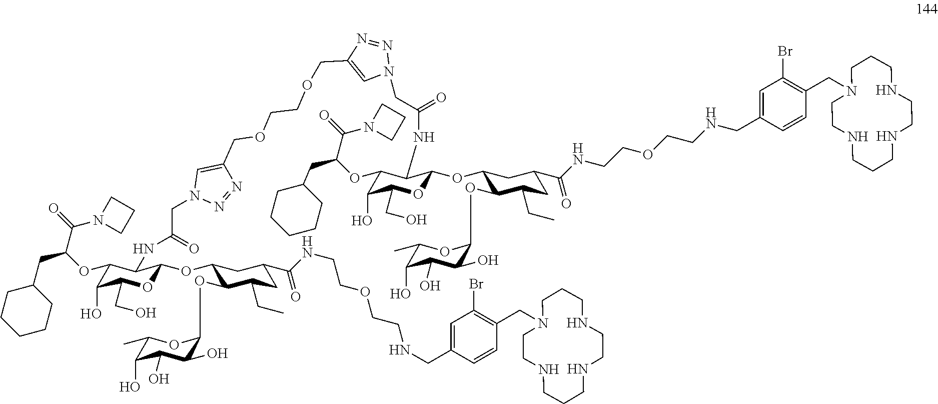

### Example 111

### Prophetic Synthesis of Multimeric Compound 146

Compound 315: To a solution of compound 314 (1 gm, 3.89 mol) (preparation described in WO 2007/028050) and benzyl trichloroacetaimidate (1.1 ml, 5.83 mmol) in anhydrous dichloromethane (10 ml) was added trimethylsilyl trifluoromethanesulfonate (70 uL, 0.4 mmol). The mixture was stirred at ambient temperature for 12 h. After this period the reaction was diluted with dichloromethane, washed with saturated NaHCO₃, dried over MgSO₄ and concentrated. The residue was purified by column chromatography to give compound 315 (0.8 gm, 60%).

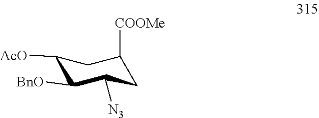

Compound 316: To a solution of compound 315 (800 mg, 2.3 mmol) in anhydrous methanol (1 ml) and anhydrous methyl acetate (5 ml) was added 0.5M sodium methoxide solution in methanol (9.2 ml). The mixture was stirred at 40° C. for 4 h. The reaction was quenched with acetic acid and concentrated. The residue was purified by column chromatography to afford compound 316 as mixture of epimers at the methyl ester with 75% equatorial and 25% axial epimer (242 mg, 35%).

¹H NMR (400 MHz, Chloroform-d) δ 7.48-7.32 (m, 6H), 4.97 (d, J=11.1 Hz, 1H), 4.72 (dd, J=11.1, 5.7 Hz, 1H), 3.77-3.65 (m, 6H), 3.22-3.15 (m, 1H), 2.92-2.82 (m, 1H), 2.39 (dddd, J=15.7, 10.6, 5.1, 2.7 Hz, 2H), 1.60 (dtd, J=13.9, 11.2, 5.4 Hz, 3H). MS: Calculated for C₁₅H₁₉N₃O₄=305.3, Found ES—positive m/z=306.1 (M+Na⁺).

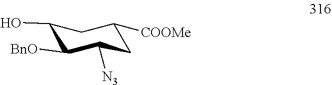

Compound 318: A solution of compound 317 (5 gm, 11.8 mmol) (preparation described in WO 2009/139719) in anhydrous methanol (20 ml) was treated with 0.5 M solution of sodium methoxide in methanol (5 ml) for 3 h. Solvent was removed in vacuo and the residue was co-evaporated with toluene (20 ml) three times. The residue was dissolved in pyridine (20 ml) followed by addition of benzoyl chloride (4.1 ml, 35.4 mmol) over 10 minutes. The reaction mixture was stirred at ambient temperature under an atmosphere of argon for 22 h. The reaction mixture was concentrated to dryness, dissolved in dichloromethane, washed with cold 1N hydrochloric acid and cold water, dried over MgSO₄, filtered, and concentrated. The residue was purified by column chromatography to give compound 318. MS: Calculated for C₃₃H₂₇N₃O₇S=609.2, Found ES—positive m/z=610.2 (M+Na⁺).

Compound 319: A mixture of compound 318 (2.4 gm, 3.93 mmol), diphenyl sulfoxide (1.5 gm, 7.3 mmol) and 2,6-di-tert-butyl pyridine (1.8 gm, 7.8 mmol) was dissolved in anhydrous dichloromethane (10 ml) at room temperature. The reaction mixture was cooled to −60° C. Triflic anhydride (0.62 ml, 3.67 mmol) was added dropwise and the mixture was stirred for 15 minutes at the same temperature. A solution of compound 316 (0.8 gm, 2.6 mmol) in anhydrous dichloromethane (10 ml) was added dropwise to the reaction mixture. The mixture was allowed to warm to 0° C. over 2 h. The reaction mixture was diluted with dichloromethane, transferred to a separatory funnel and washed with saturated sodium bicarbonate solution followed by brine. The organic phase was dried over MgSO₄, filtered, and concentrated. The residue was separated by column chromatography to afford compound 319 as a white solid (1.2 gm, 57%). MS: Calculated for C₄₂H₄₀N₆O₁₁=804.3, Found ES—positive m/z=805.3 (M+Na⁺).

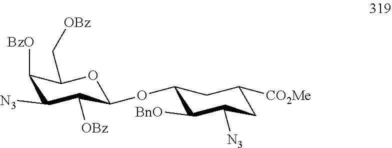

Compound 320: To a solution of compound 319 (1.2 gm 2.067 mmol) and 2-fluorophenyl acetylene (1.2 ml, 10.3 mmol) in methanol (30 ml) was added a stock solution of copper sulfate and tris(3-hydroxypropyltriazolylmethyl) amine in water (2.58 ml). The reaction was initiated by addition of an aqueous solution of sodium ascorbate (0.9 gm, 4.5 mmol) and the mixture was stirred at ambient temperature for 16 hours. The mixture was co-evaporated with dry silica gel and purified by column chromatography to afford compound 320 as a white solid (1.2 gm, 77%).

Stock solution of Copper Sulfate/THPTA—(100 mg of copper sulfate pentahydrate and 200 mg of tris(3-hydroxypropyltriazolylmethyl)amine were dissolved in 10 ml of water).

¹H NMR (400 MHz, Chloroform-d) δ 8.07-8.00 (m, 2H), 7.96 (ddd, J=9.8, 8.2, 1.3 Hz, 4H), 7.79 (d, J=5.4 Hz, 2H), 7.65-7.53 (m, 5H), 7.43 (ddt, J=22.4, 10.7, 5.0 Hz, 7H), 7.25-7.01 (m, 9H), 6.92 (td, J=7.6, 7.1, 2.2 Hz, 1H), 6.13-6.02 (m, 2H), 5.58 (dd, J=11.6, 3.2 Hz, 1H), 5.15 (d, J=7.5 Hz, 1H), 4.98 (d, J=10.3 Hz, 1H), 4.68 (dd, J=11.2, 5.7 Hz, 1H), 4.52 (dq, J=22.1, 6.6, 5.6 Hz, 2H), 4.35 (dd, J=11.1, 7.6 Hz, 1H), 4.28-4.18 (m, 1H), 4.11 (d, J=10.3 Hz, 1H), 3.87 (t, J=9.1 Hz, 1H), 3.71 (s, 3H), 2.95 (s, 1H), 2.62-2.43 (m, 3H), 1.55 (dt, J=12.7, 6.1 Hz, 1H). MS: Calculated for C₅₈H₅₀N₆O₁₁=1044.4, Found ES—positive m/z=1045.5 (M+Na⁺).

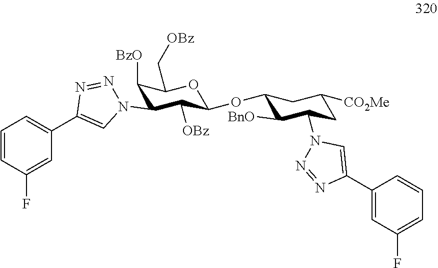

Compound 145: To a solution of compound 320 (1.2 gm, 1.1 mmol) in iso-propanol (40 ml) was added Na-metal (80 mg, 3.4 mmol) at ambient temperature and the mixture was stirred for 12 hours at 50° C. 10% aqueous sodium hydroxide (2 ml) was added to the reaction mixture and stirring continued for another 6 hours at 50° C. The reaction mixture was cooled to room temperature and neutralized with 50% aqueous hydrochloric acid. To the mixture was added 10% Pd(OH)₂ on carbon (0.6 gm) and the reaction mixture was stirred under an atmosphere of hydrogen for 12 hours. The reaction mixture was filtered through a Celite pad and concentrated. The residue was separated by HPLC to give compound 145 as a white solid (0.5 gm, 70%). HPLC Conditions—Waters preparative HPLC system was used with ELSD & PDA detectors. Kinetex XB-C18, 100 A, 5 uM, 250×21.2 mm column (from Phenomenex) was used with 0.2% formic acid in water as solvent A and acetonitrile as solvent B at a flow rate of 20 mL/min.

¹H NMR (400 MHz, DMSO-d6) δ 8.77 (s, 1H), 8.68 (s, 1H), 7.77-7.60 (m, 5H), 7.49 (tdd, J=8.3, 6.1, 2.6 Hz, 3H), 7.15 (tt, J=8.6, 3.2 Hz, 3H), 4.83 (dd, J=10.9, 3.1 Hz, 1H), 4.63 (d, J=7.5 Hz, 1H), 4.53-4.41 (m, 1H), 4.10 (dd, J=10.9, 7.5 Hz, 1H), 3.92 (d, J=3.2 Hz, 1H), 3.74 (h, J=6.0, 5.6 Hz, 3H), 3.65-3.24 (m, 5H), 2.37 (d, J=13.4 Hz, 1H), 2.24-2.04 (m, 2H), 1.93 (q, J=12.5 Hz, 1H), 1.46 (t, J=12.1 Hz, 1H). MS: Calculated for C₂₉H₃₀F₂N₆O₈=628.2, Found ES—positive m % z=629.2 (M+Na⁺).

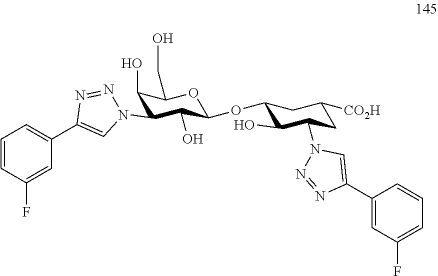

Compound 146: To a solution of compound 145 (3 eq) in anhydrous DMF was added HATU (3.3 eq) and DIPEA (5 eq). The mixture was stirred at ambient temperature for 15 minutes followed by addition of compound 22 (1 eq). The mixture was stirred at ambient temperature for 12 h. The solvent was removed in vacuo and the residue was purified by HPLC to afford compound 146.

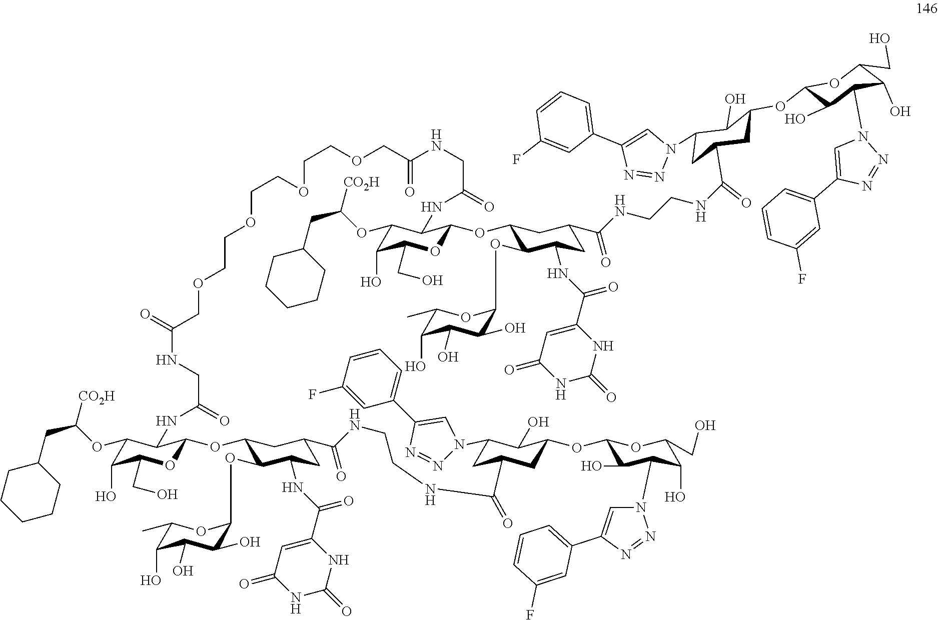

### Example 112

### Prophetic Synthesis of Multimeric Compound 147

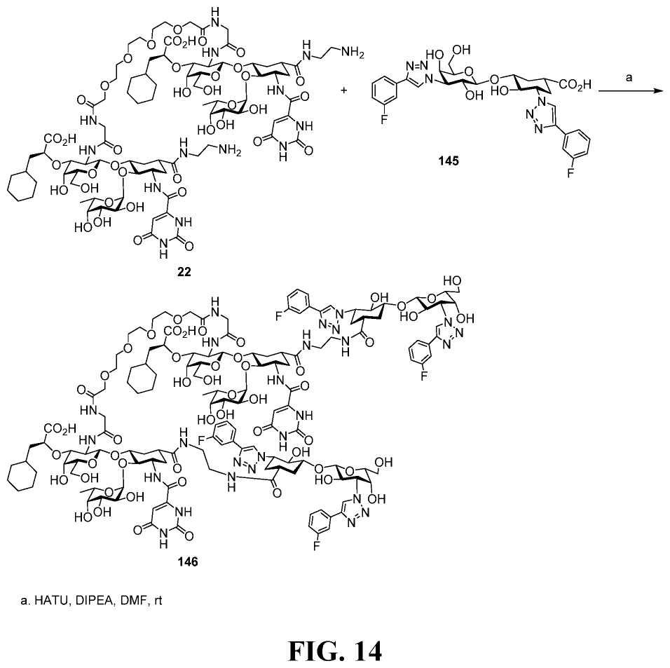

Compound 147: Compound 147 can be prepared in an analogous fashion to FIG. 14 by replacing compound 22 with compound 23.

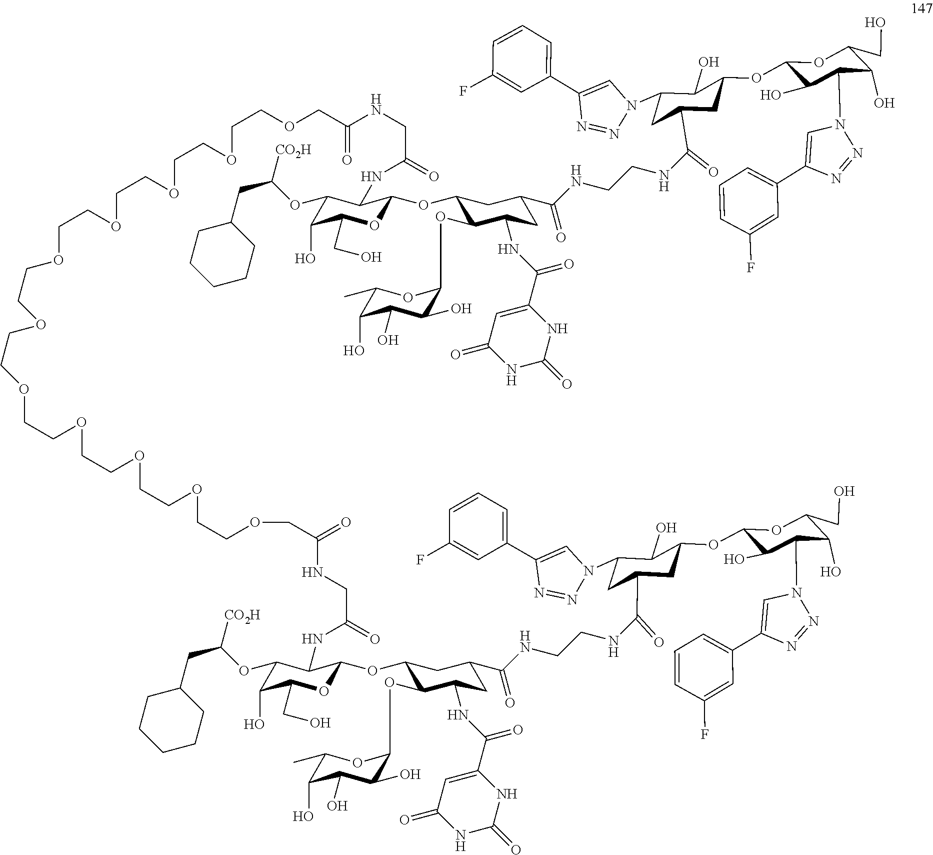

### Example 113

### Prophetic Synthesis of Multimeric Compound 148

Compound 148: Compound 148 can be prepared in an analogous fashion to FIG. 14 by replacing compound 22 with compound 24.

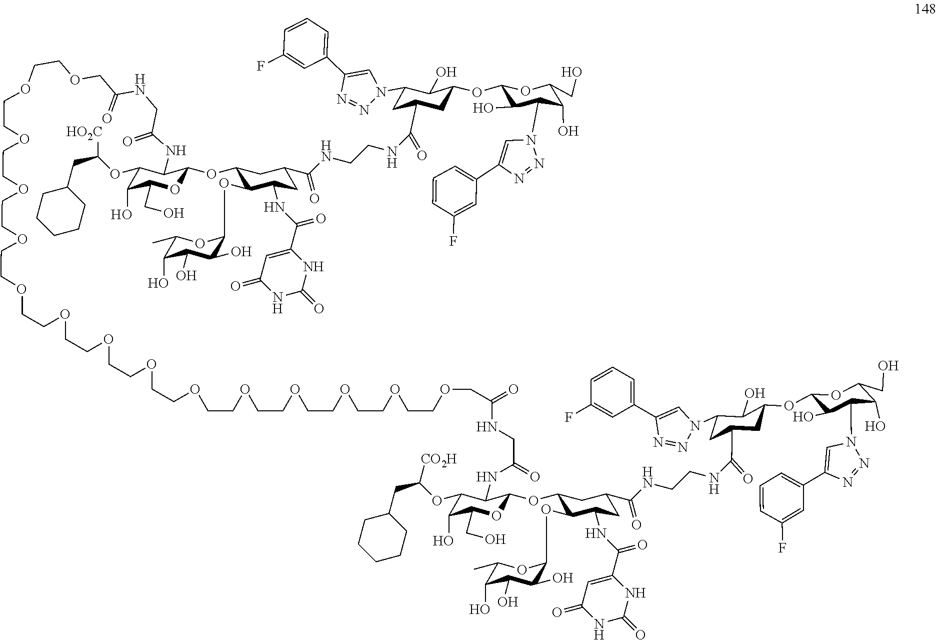

### Example 114

### Prophetic Synthesis of Multimeric Compound 149

Compound 149: Compound 149 can be prepared in an analogous fashion to FIG. 14 by replacing compound 22 with compound 25.

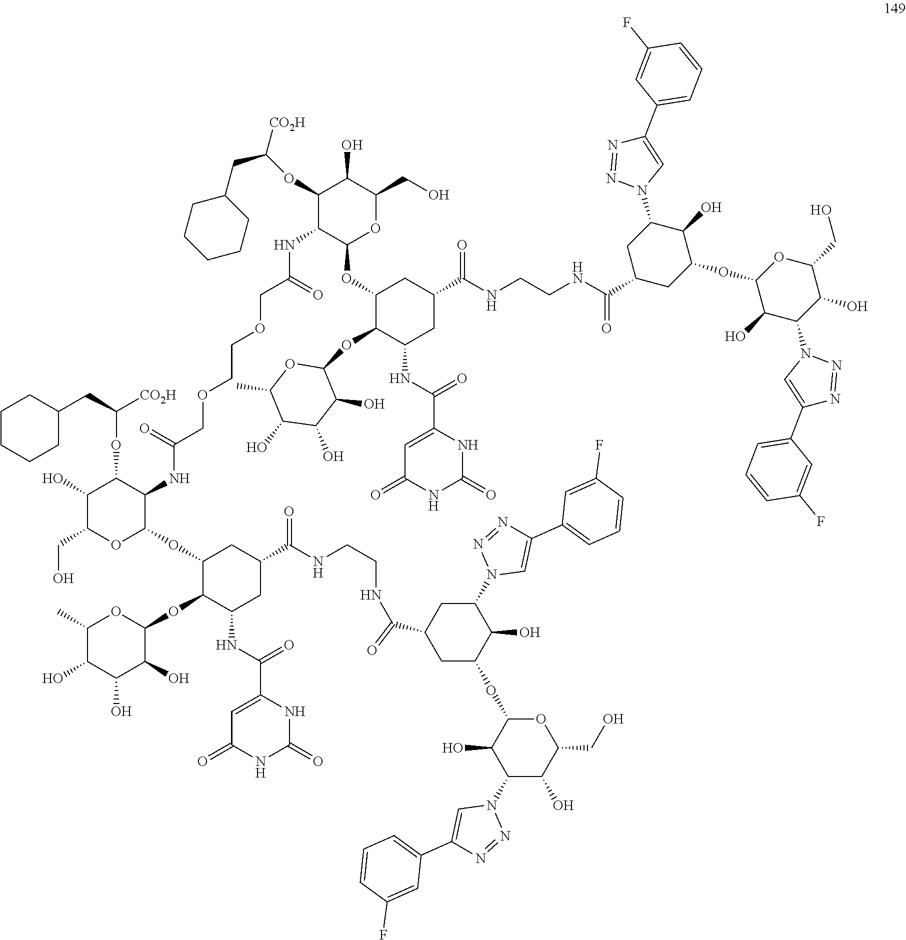

### Example 115

### Prophetic Synthesis of Multimeric Compound 150

Compound 150: Compound 150 can be prepared in an analogous fashion to FIG. 14 by replacing compound 22 with compound 26.

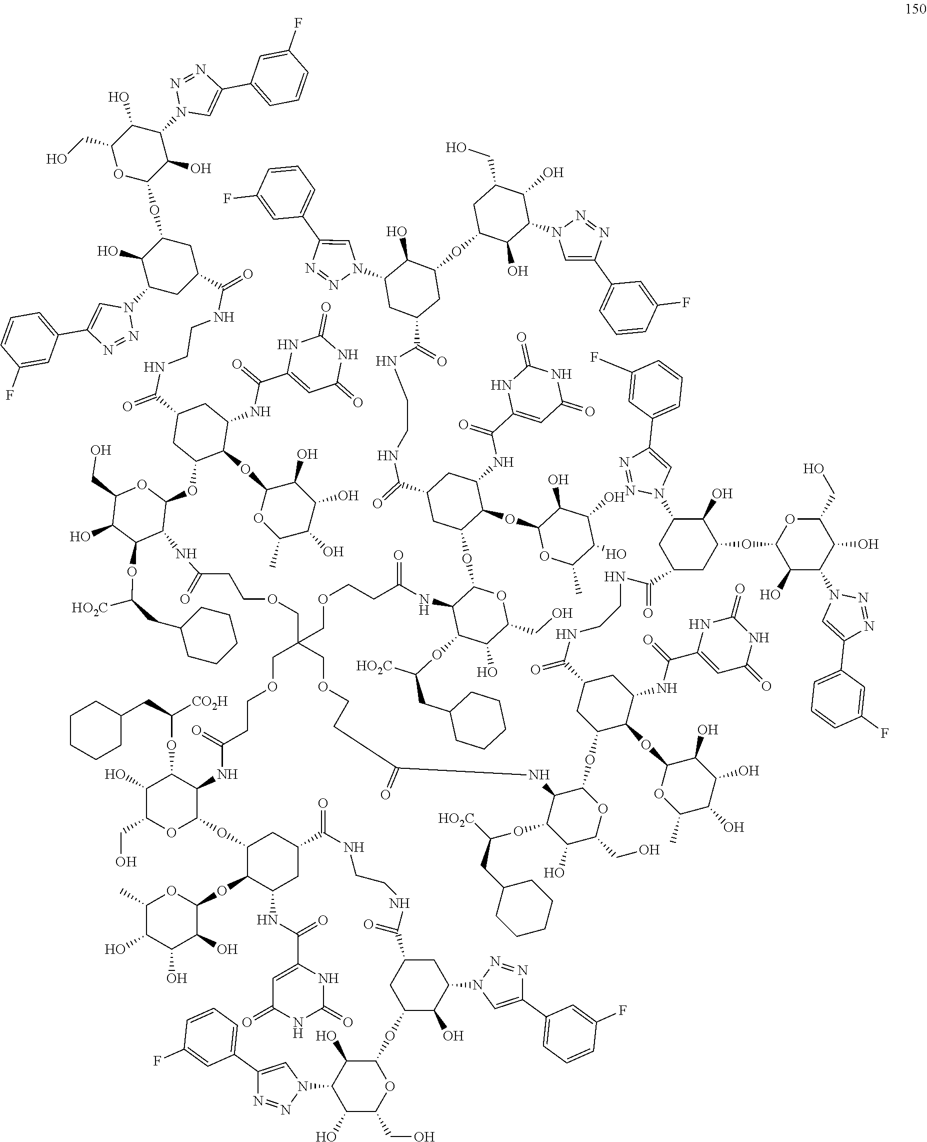

### Example 116

### Prophetic Synthesis of Multimeric Compound 151

Compound 151: Compound 151 can be prepared in an analogous fashion to FIG. 14 by replacing compound 22 with compound 27.

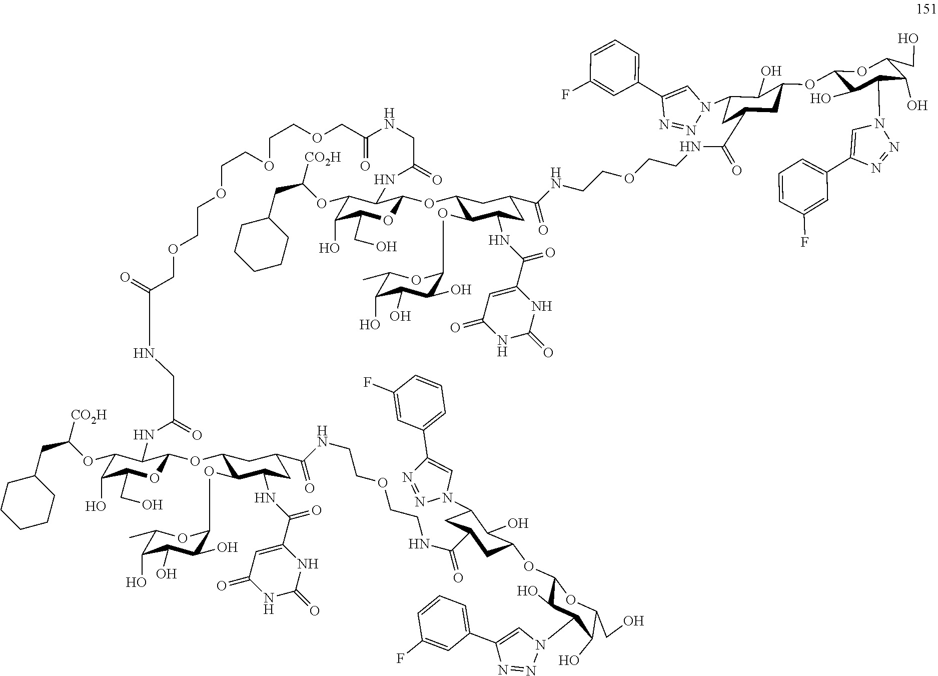

### Example 117

### Prophetic Synthesis of Multimeric Compound 152

Compound 152: Compound 152 can be prepared in an analogous fashion to FIG. 14 by replacing compound 22 with compound 28.

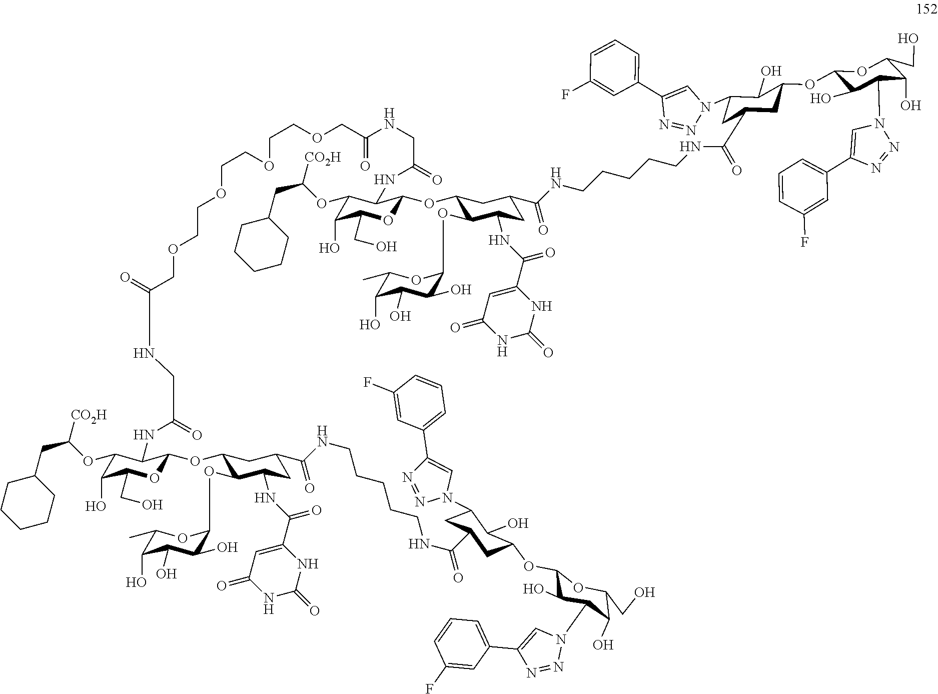

### Example 118

### Prophetic Synthesis of Multimeric Compound 153

Compound 153: Compound 153 can be prepared in an analogous fashion to FIG. 14 by replacing compound 22 with compound 29.

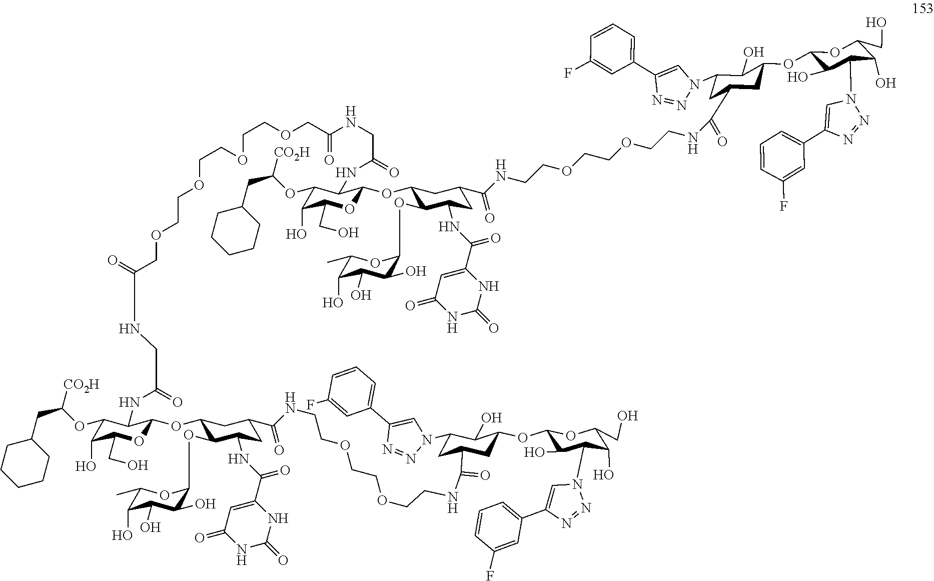

### Example 119

### Prophetic Synthesis of Multimeric Compound 154

Compound 154: Compound 154 can be prepared in an analogous fashion to FIG. 14 by replacing compound 22 with compound 30.

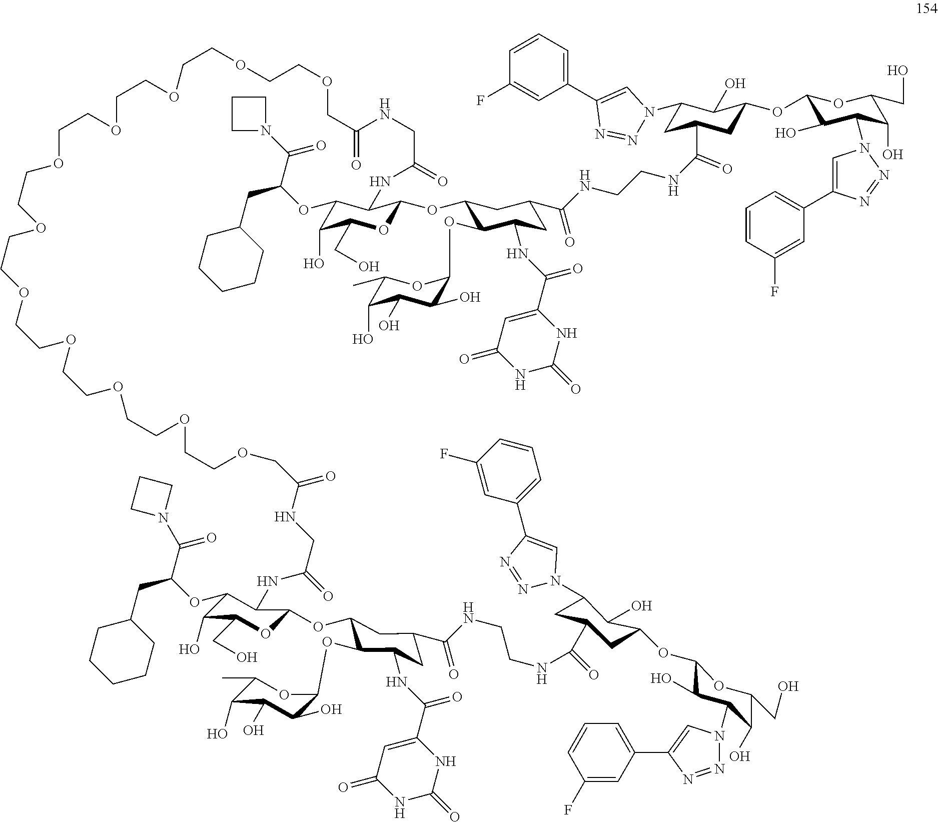

### Example 120

### Prophetic Synthesis of Multimeric Compound 155

Compound 155: Compound 155 can be prepared in an analogous fashion to FIG. 14 by replacing compound 22 with compound 31.

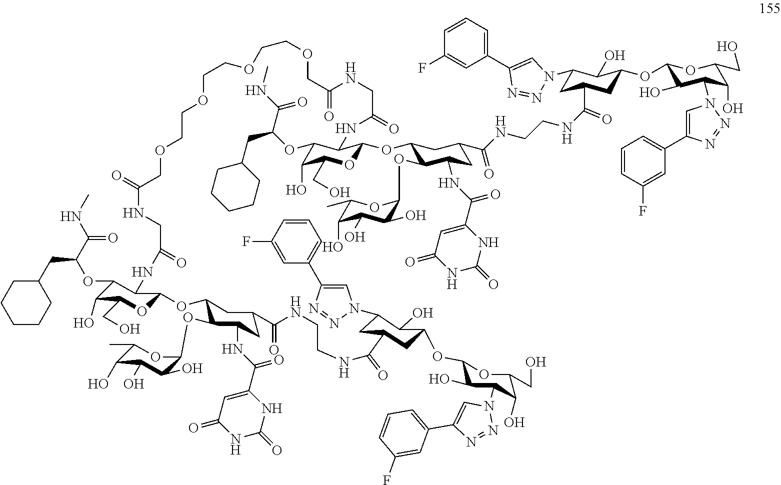

### Example 121

### Prophetic Synthesis of Multimeric Compound 156

Compound 156: Compound 156 can be prepared in an analogous fashion to FIG. 14 by replacing compound 22 with compound 32.

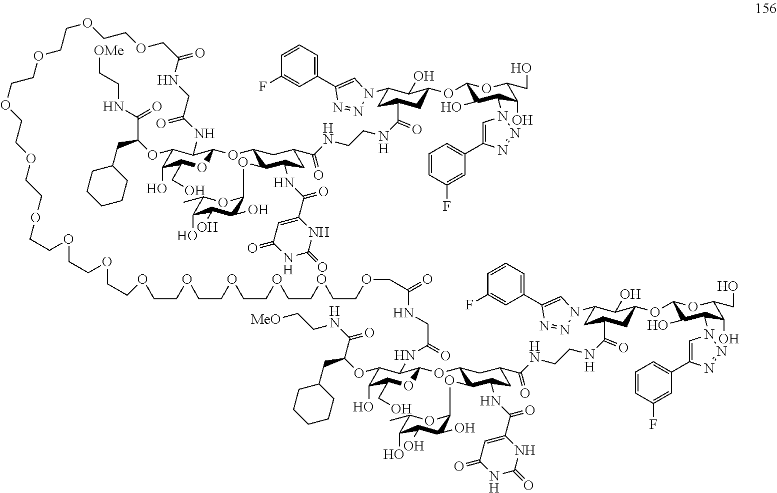

### Example 122

### Prophetic Synthesis of Multimeric Compound 157

Compound 157: Compound 157 can be prepared in an analogous fashion to FIG. 14 by replacing compound 22 with compound 33.

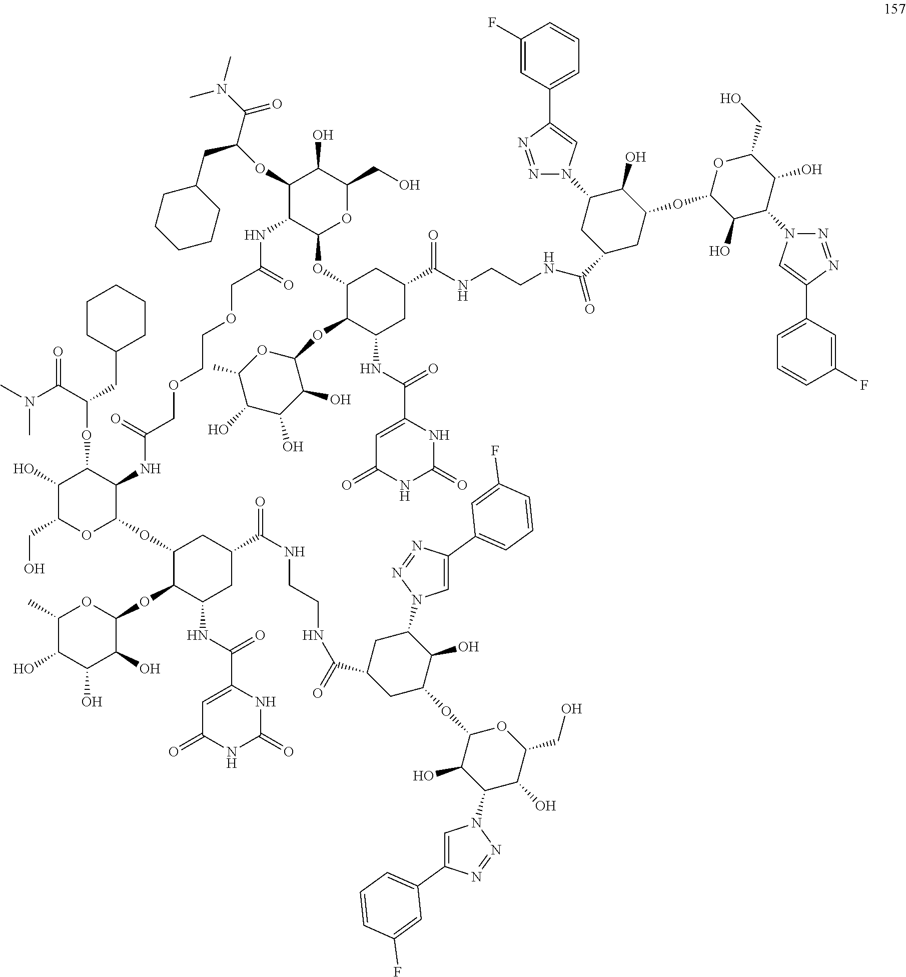

### Example 123

### Prophetic Synthesis of Multimeric Compound 158

Compound 158: Compound 158 can be prepared in an analogous fashion to FIG. 14 by replacing compound 22 with compound 34.

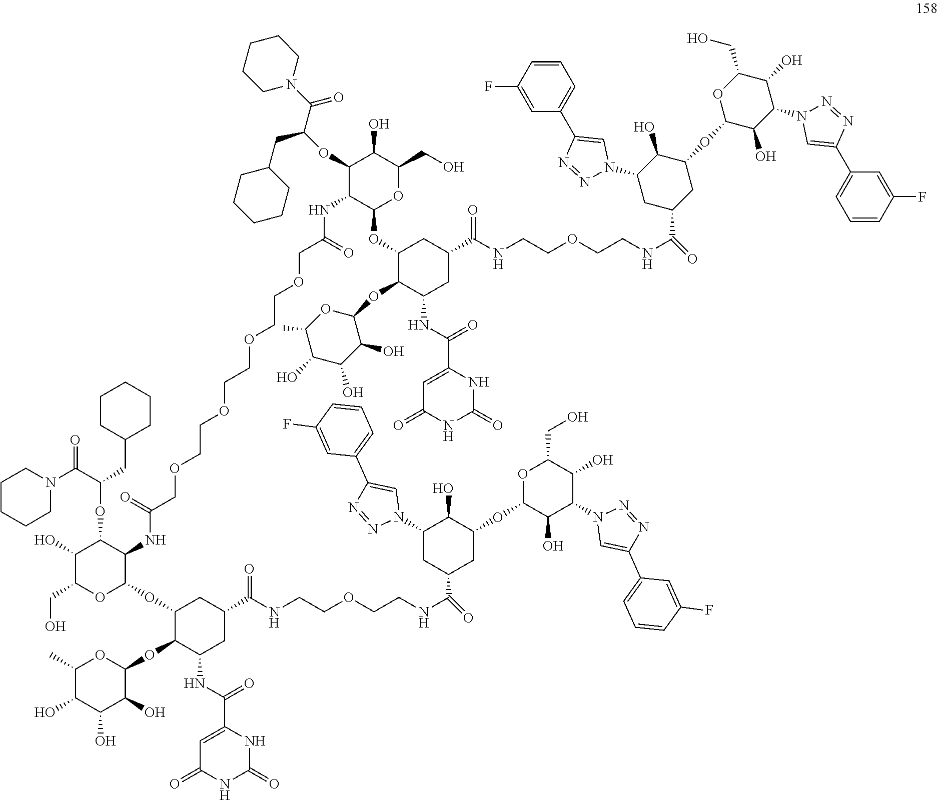

### Example 124

### Prophetic Synthesis of Multimeric Compound 159

Compound 159: Compound 159 can be prepared in an analogous fashion to FIG. 14 by replacing compound 22 with compound 37.

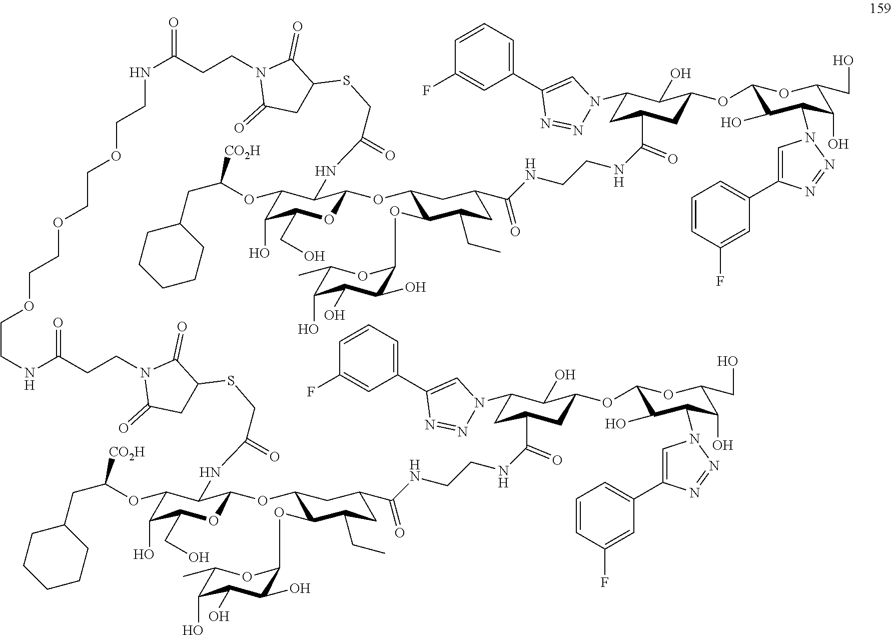

### Example 125

### Prophetic Synthesis of Multimeric Compound 160

Compound 160: Compound 160 can be prepared in an analogous fashion to FIG. 14 by replacing compound 22 with compound 38.

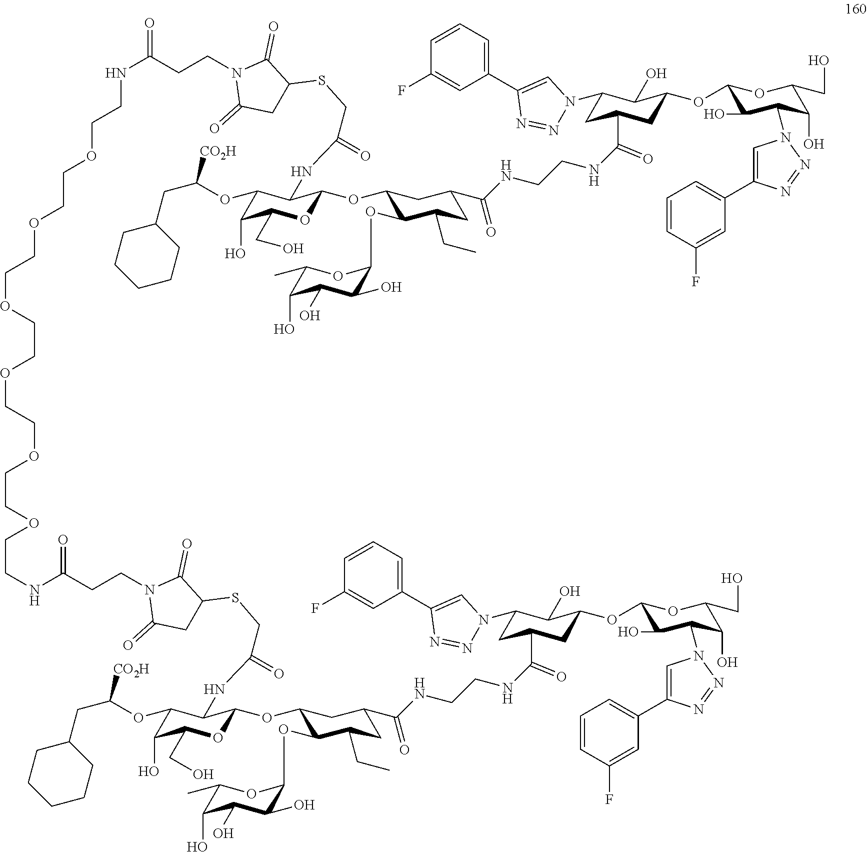

### Example 126

### Prophetic Synthesis of Multimeric Compound 161

Compound 161: Compound 161 can be prepared in an analogous fashion to FIG. 14 by replacing compound 22 with compound 39.

### Example 127

### Prophetic Synthesis of Multimeric Compound 162

Compound 162: Compound 162 can be prepared in an analogous fashion to FIG. 14 by replacing compound 22 with compound 40.

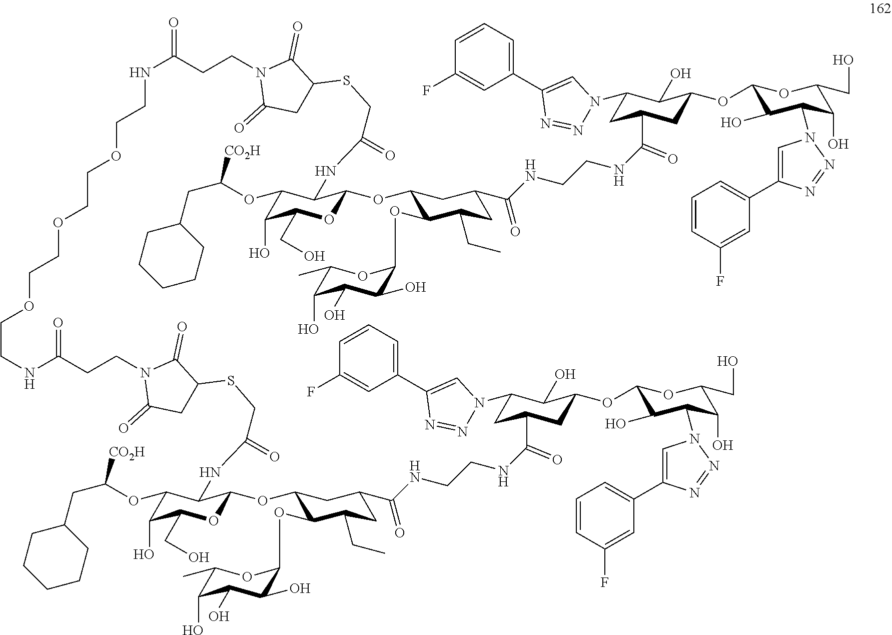

### Example 128

### Prophetic Synthesis of Multimeric Compound 163

Compound 163: Compound 163 can be prepared in an analogous fashion to FIG. 14 by replacing compound 22 with compound 46.

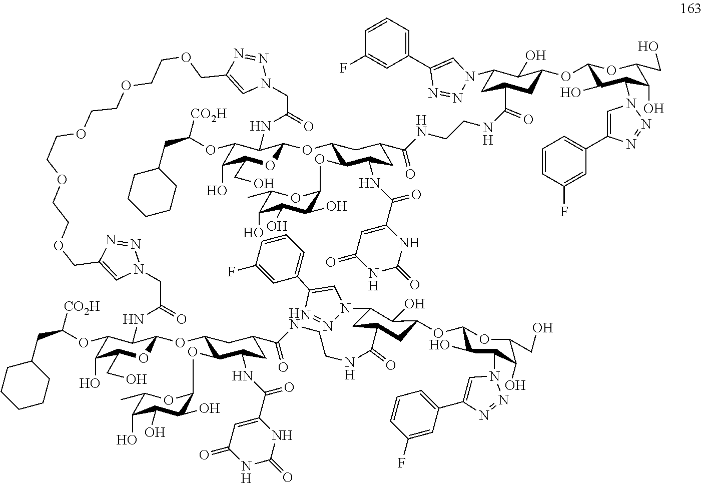

### Example 129

### Prophetic Synthesis of Multimeric Compound 164

Compound 164: Compound 164 can be prepared in an analogous fashion to FIG. 14 by replacing compound 22 with compound 47.

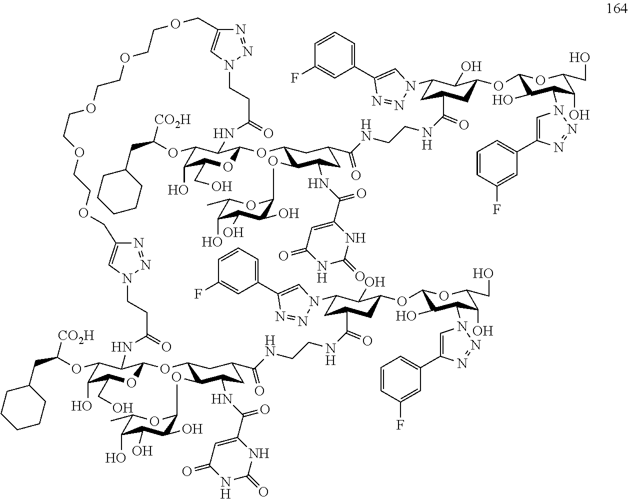

### Example 130

### Prophetic Synthesis of Multimeric Compound 165

Compound 165: Compound 165 can be prepared in an analogous fashion to FIG. 13 by replacing compound 22 with compound 48.

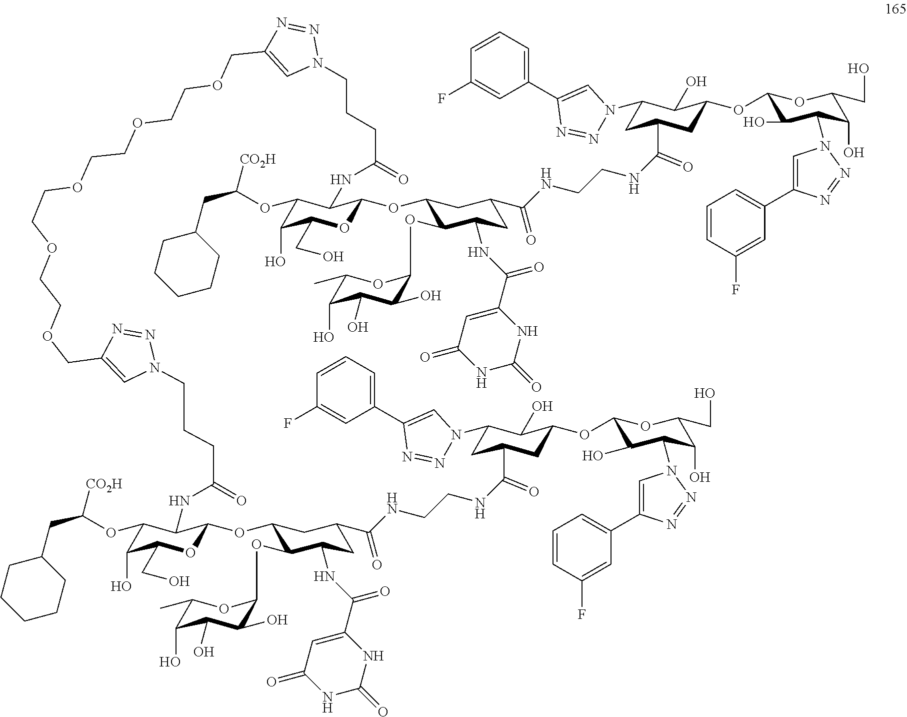

### Example 131

### Prophetic Synthesis of Multimeric Compound 166

Compound 166: Compound 166 can be prepared in an analogous fashion to FIG. 14 by replacing compound 22 with compound 49.

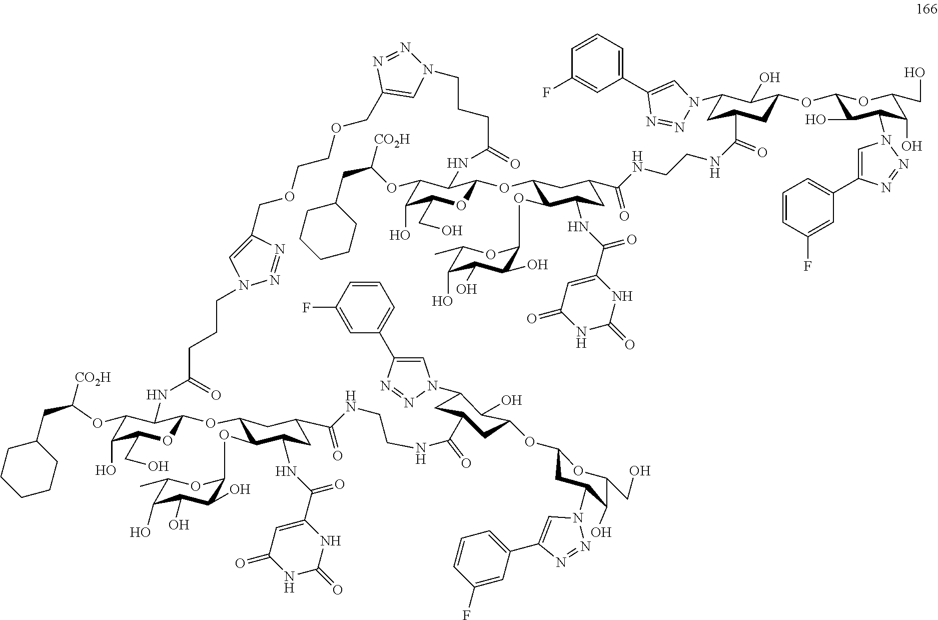

### Example 132

### Prophetic Synthesis of Multimeric Compound 167

Compound 167: Compound 167 can be prepared in an analogous fashion to FIG. 14 by replacing compound 22 with compound 50.

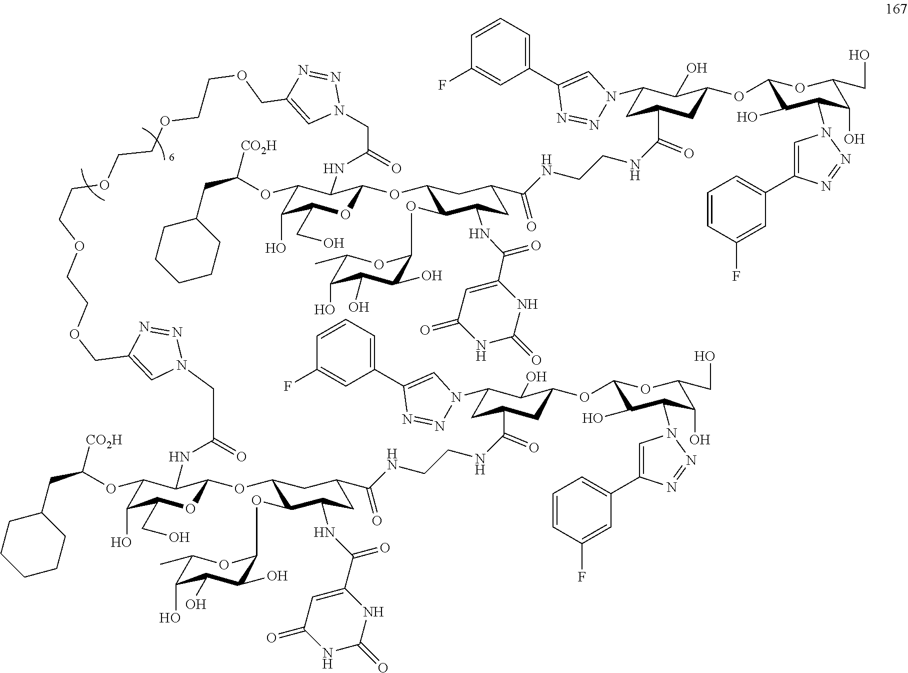

### Example 133

### Prophetic Synthesis of Multimeric Compound 168

Compound 168: Compound 168 can be prepared in an analogous fashion to FIG. 14 by replacing compound 22 with compound 51.

### Example 134

### Prophetic Synthesis of Multimeric Compound 169

Compound 169: Compound 169 can be prepared in an analogous fashion to FIG. 14 by replacing compound 22 with compound 52.

### Example 135

### Prophetic Synthesis of Multimeric Compound 170

Compound 170: Compound 170 can be prepared in an analogous fashion to FIG. 14 by replacing compound 22 with compound 53.

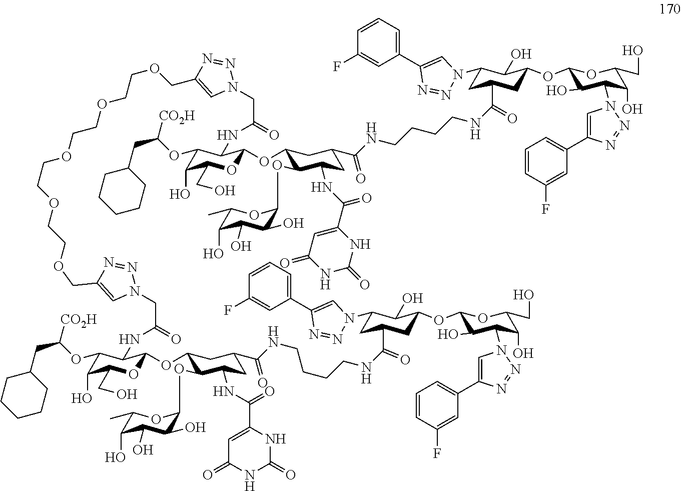

### Example 136

### Prophetic Synthesis of Multimeric Compound 171

Compound 171: Compound 171 can be prepared in an analogous fashion to FIG. 14 by replacing compound 22 with compound 54.

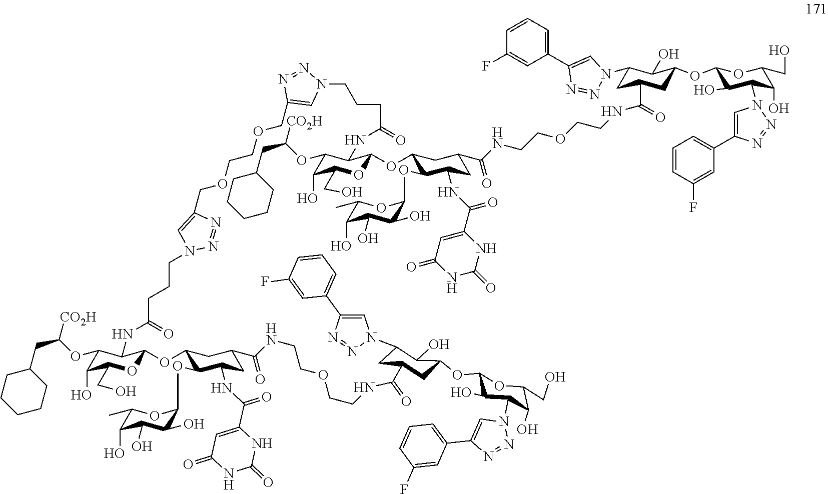

### Example 137

### Prophetic Synthesis of Multimeric Compound 172

Compound 172: Compound 172 can be prepared in an analogous fashion to FIG. 14 by replacing compound 22 with compound 56.

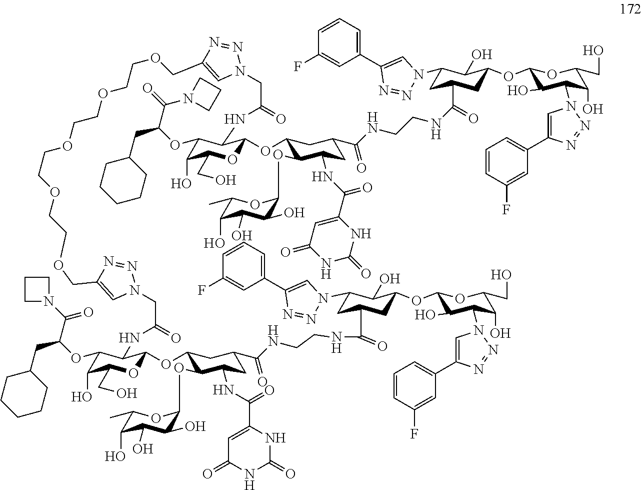

### Example 138

### Prophetic Synthesis of Multimeric Compound 173

Compound 173: Compound 173 can be prepared in an analogous fashion to FIG. 14 by replacing compound 22 with compound 57.

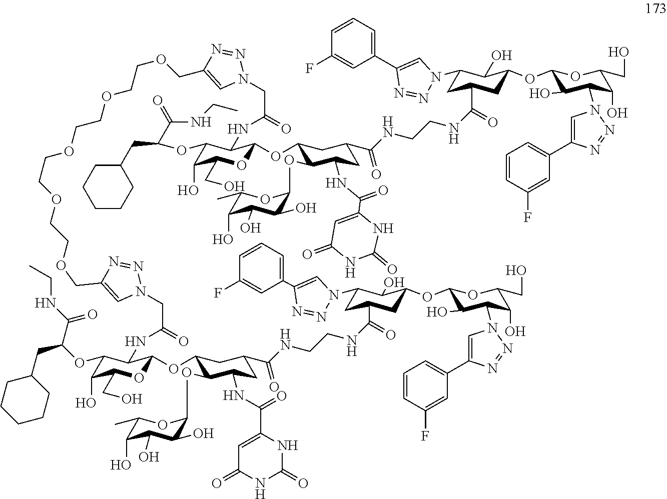

### Example 139

### Prophetic Synthesis of Multimeric Compound 174

Compound 174: Compound 174 can be prepared in an analogous fashion to FIG. 14 by replacing compound 22 with compound 58.

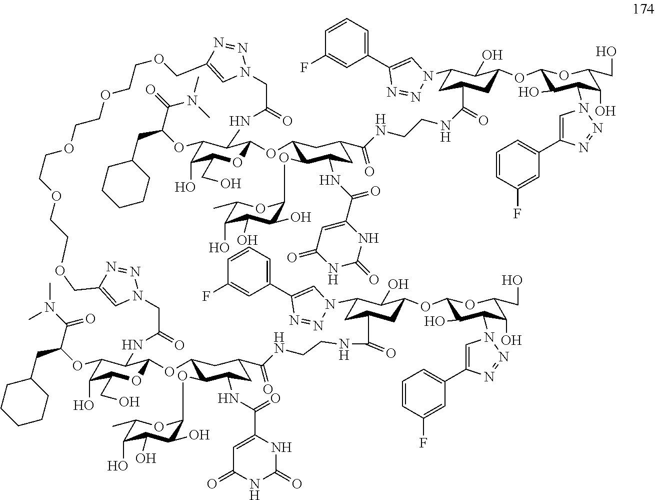

### Example 140

### Prophetic Synthesis of Multimeric Compound 175

Compound 175: Compound 175 can be prepared in an analogous fashion to FIG. 14 by replacing compound 22 with compound 59.

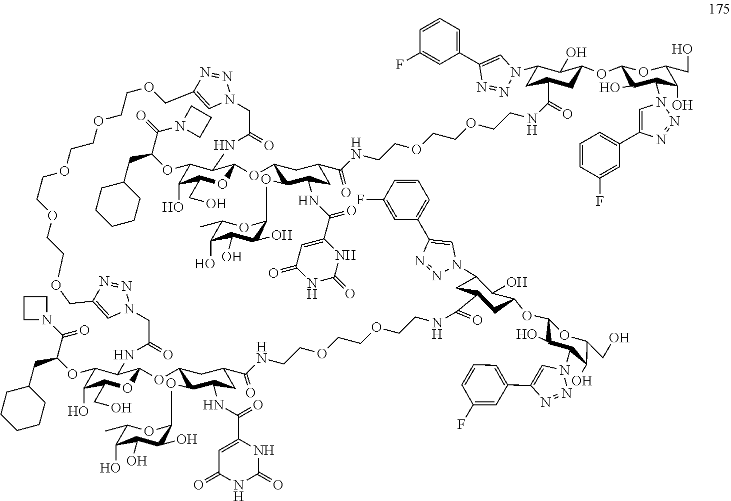

### Example 141

### Prophetic Synthesis of Multimeric Compound 176

Compound 176: Compound 176 can be prepared in an analogous fashion to FIG. 14 by replacing compound 22 with compound 68.

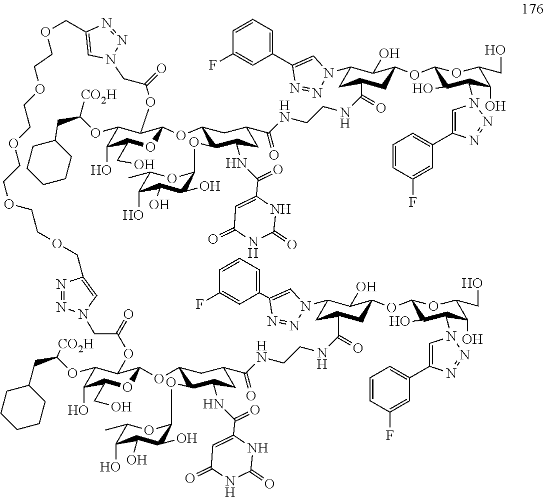

### Example 142

### Prophetic Synthesis of Multimeric Compound 177

Compound 177: Compound 177 can be prepared in an analogous fashion to FIG. 14 by replacing compound 22 with compound 69.

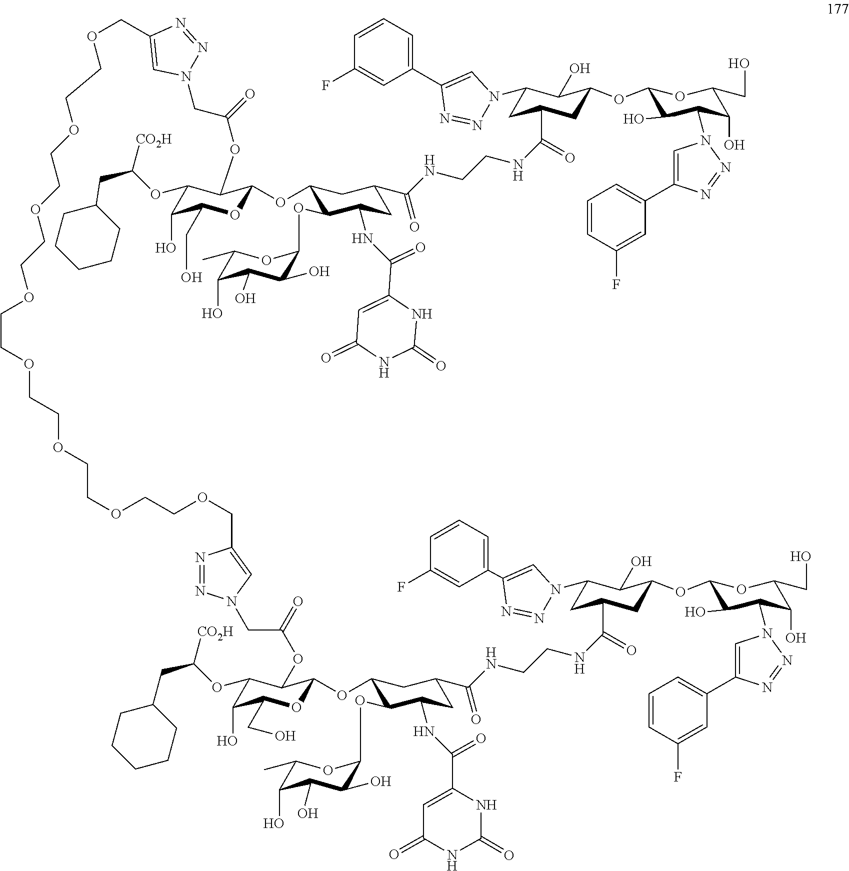

### Example 143

### Prophetic Synthesis of Multimeric Compound 178

Compound 178: Compound 178 can be prepared in an analogous fashion to FIG. 14 by replacing compound 22 with compound 70.

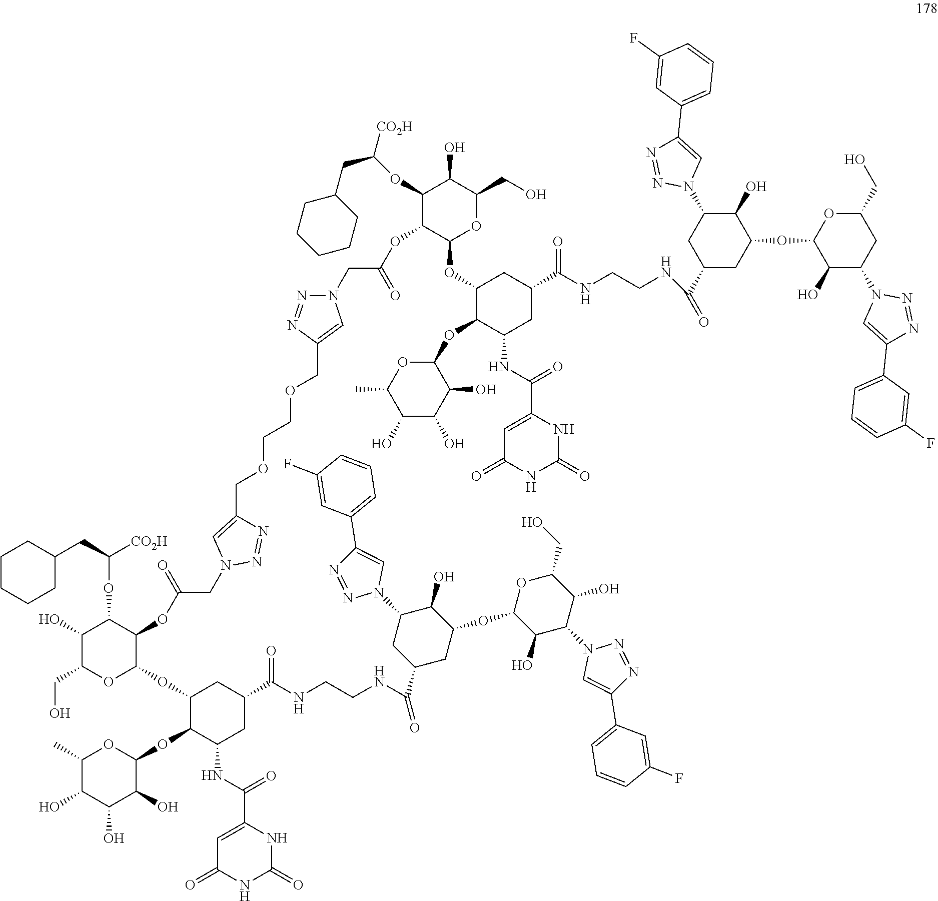

### Example 144

### Prophetic Synthesis of Multimeric Compound 179

Compound 179: Compound 179 can be prepared in an analogous fashion to FIG. 14 by replacing compound 22 with compound 71.

### Example 145

### Prophetic Synthesis of Multimeric Compound 180

Compound 180: Compound 180 can be prepared in an analogous fashion to FIG. 14 by replacing compound 22 with compound 73.

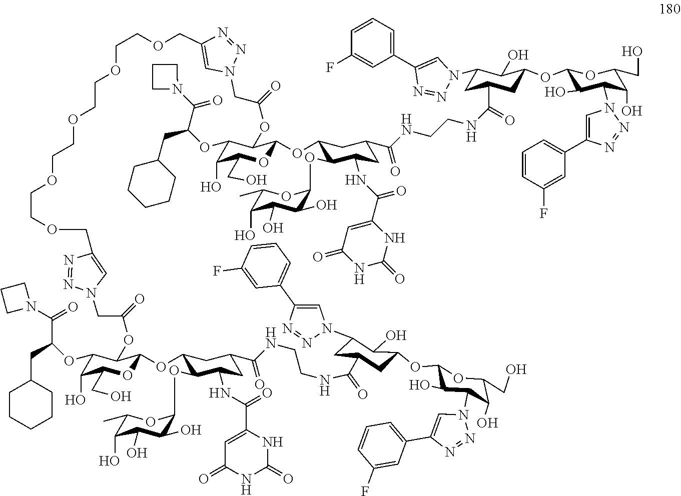

### Example 146

### Prophetic Synthesis of Multimeric Compound 181

Compound 181: Compound 181 can be prepared in an analogous fashion to FIG. 14 by replacing compound 22 with compound 78.

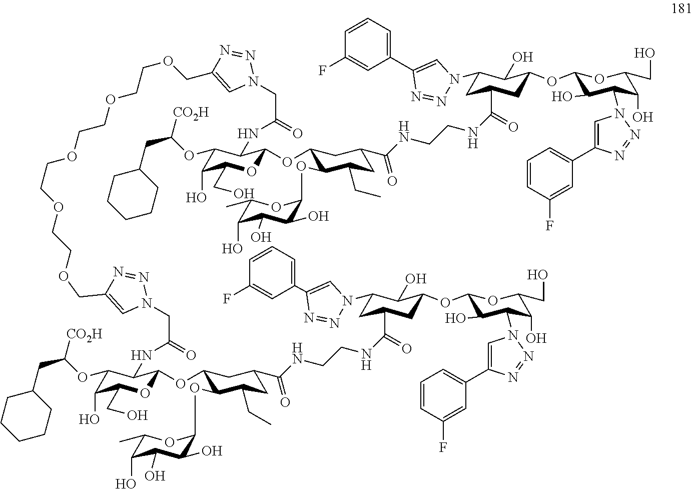

### Example 147

### Prophetic Synthesis of Multimeric Compound 182

Compound 182: Compound 182 can be prepared in an analogous fashion to FIG. 14 by replacing compound 22 with compound 79.

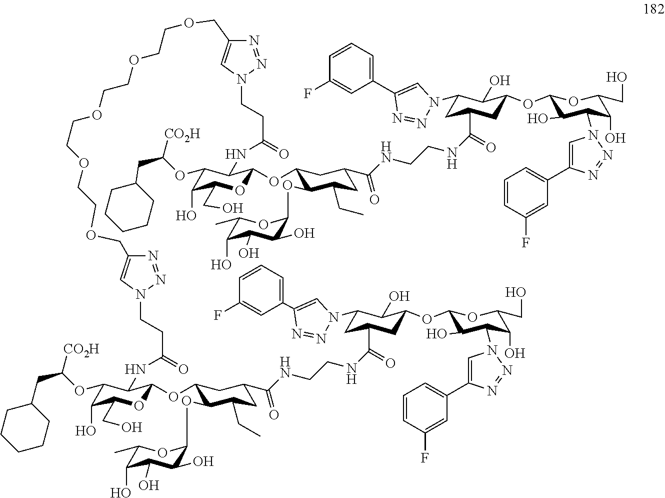

### Example 148

### Prophetic Synthesis of Multimeric Compound 183

Compound 183: Compound 183 can be prepared in an analogous fashion to FIG. 14 by replacing compound 22 with compound 80.

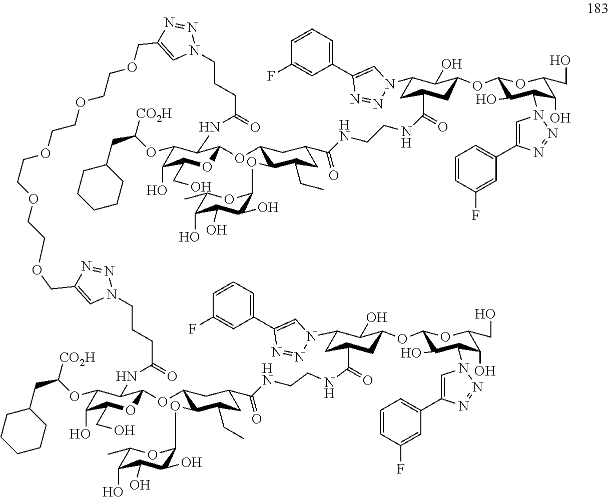

### Example 149

### Prophetic Synthesis of Multimeric Compound 184

Compound 184: Compound 184 can be prepared in an analogous fashion to FIG. 14 by replacing compound 22 with compound 81.

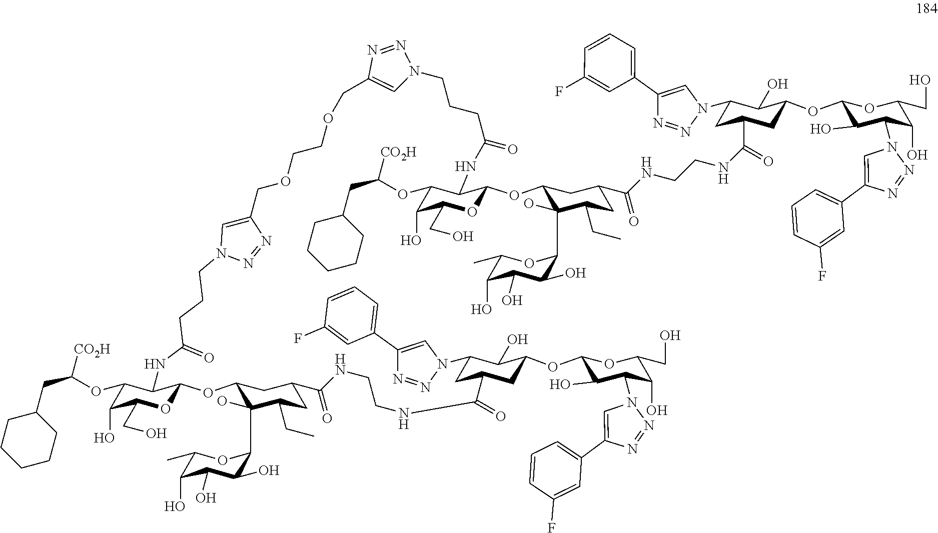

### Example 150

### Prophetic Synthesis of Multimeric Compound 185

Compound 185: Compound 185 can be prepared in an analogous fashion to FIG. 14 by replacing compound 22 with compound 82.

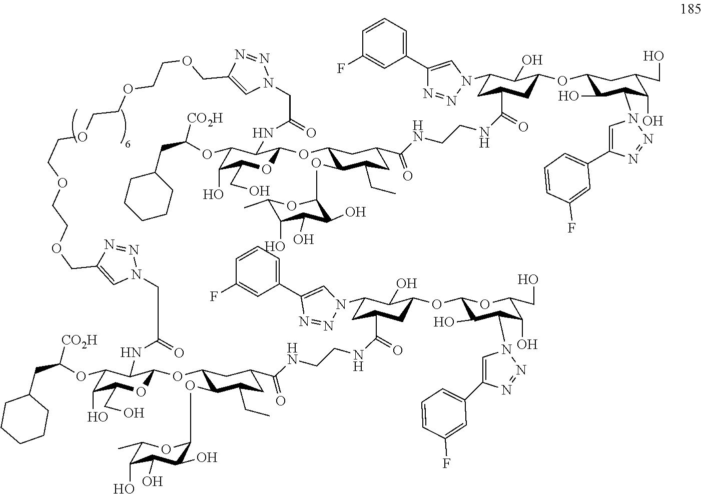

### Example 151

### Prophetic Synthesis of Multimeric Compound 186

Compound 186: Compound 186 can be prepared in an analogous fashion to FIG. 14 by replacing compound 22 with compound 83.

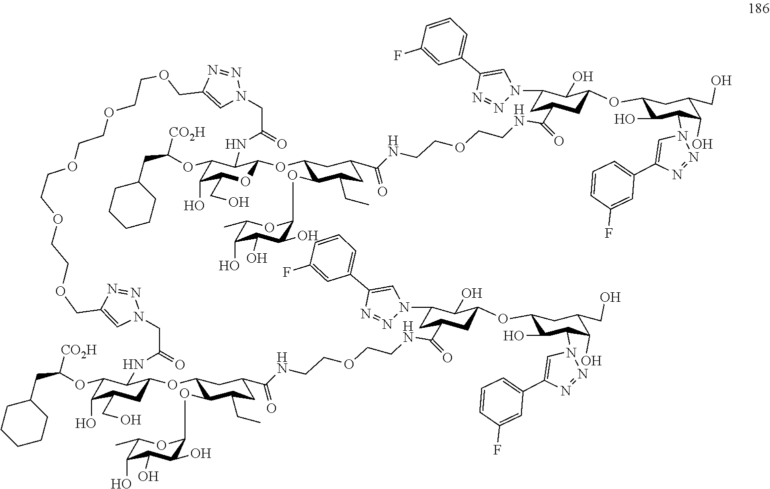

### Example 152

### Prophetic Synthesis of Multimeric Compound 187

Compound 187: Compound 187 can be prepared in an analogous fashion to FIG. 14 by replacing compound 22 with compound 84.

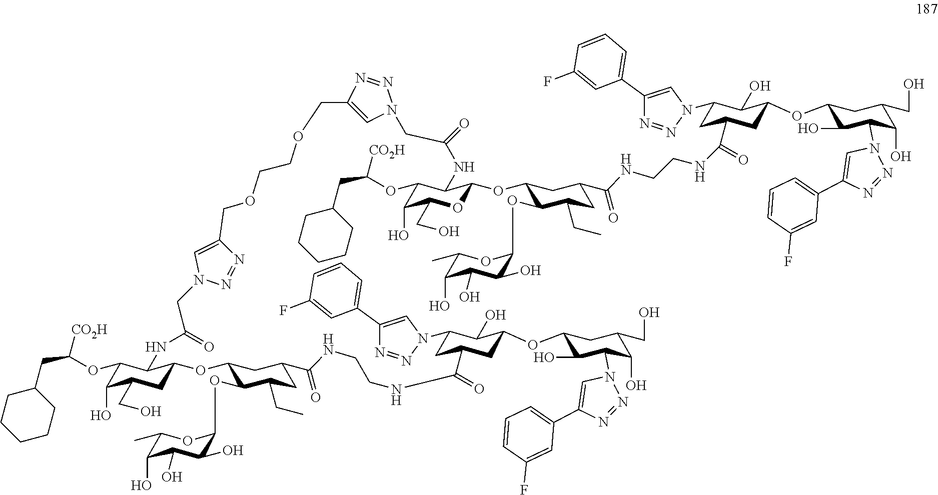

### Example 153

### Prophetic Synthesis of Multimeric Compound 188

Compound 188: Compound 188 can be prepared in an analogous fashion to FIG. 14 by replacing compound 22 with compound 85.

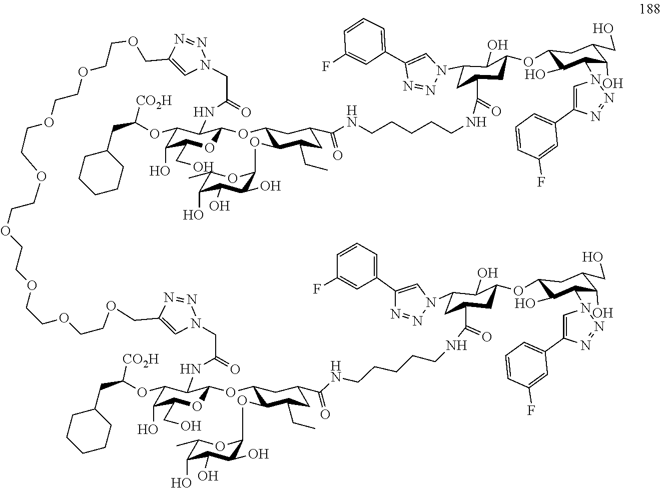

### Example 154

### Prophetic Synthesis of Multimeric Compound 189

Compound 189: Compound 189 can be prepared in an analogous fashion to FIG. 14 by replacing compound 22 with compound 87.

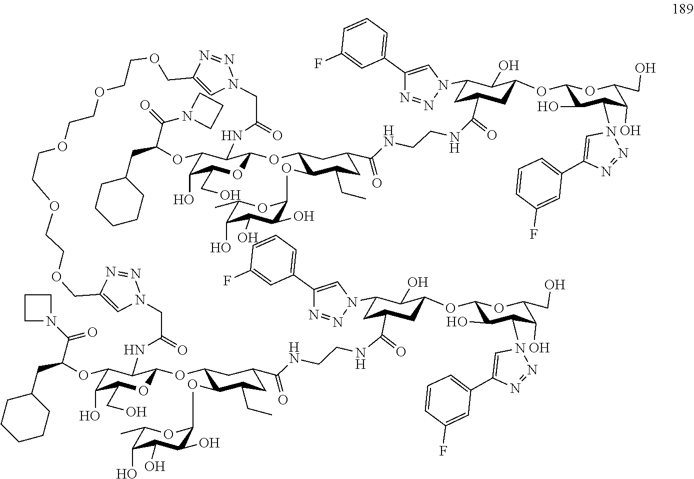

### Example 155

### Prophetic Synthesis of Multimeric Compound 190

Compound 190: Compound 190 can be prepared in an analogous fashion to FIG. 14 by replacing compound 22 with compound 88.

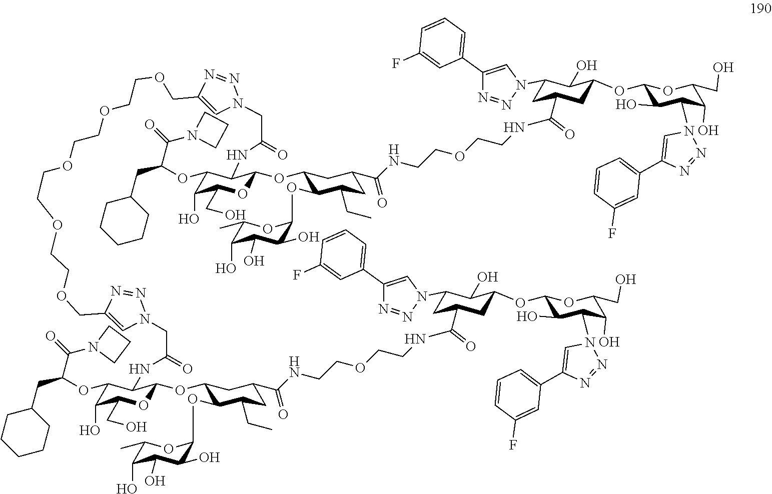

### Example 156

### Prophetic Synthesis of Multimeric Compound 191

Compound 191: Compound 191 can be prepared in an analogous fashion to FIG. 14 by replacing compound 22 with compound 89.

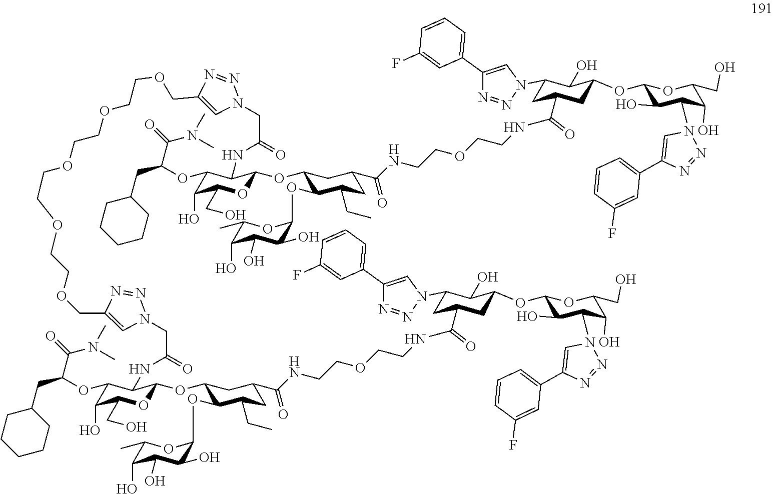

### Example 157

### Prophetic Synthesis of Multimeric Compound 192

Compound 192: Compound 192 can be prepared in an analogous fashion to FIG. 14 by replacing compound 22 with compound 90.

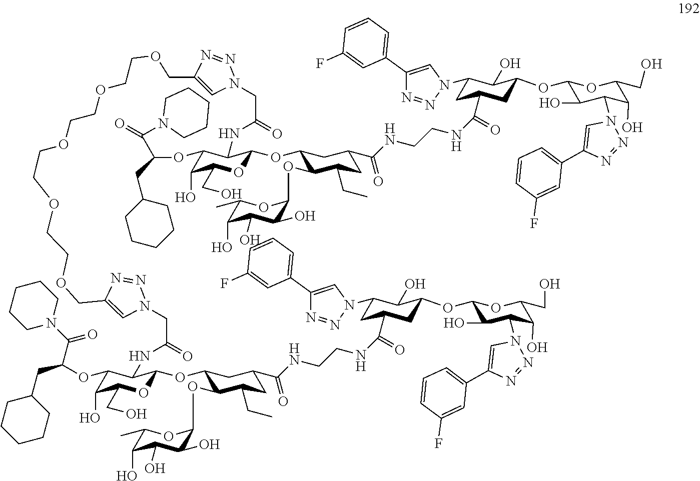

### Example 158

### Prophetic Synthesis of Multimeric Compound 193

Compound 193: Compound 193 can be prepared in an analogous fashion to FIG. 14 by replacing compound 22 with compound 91.

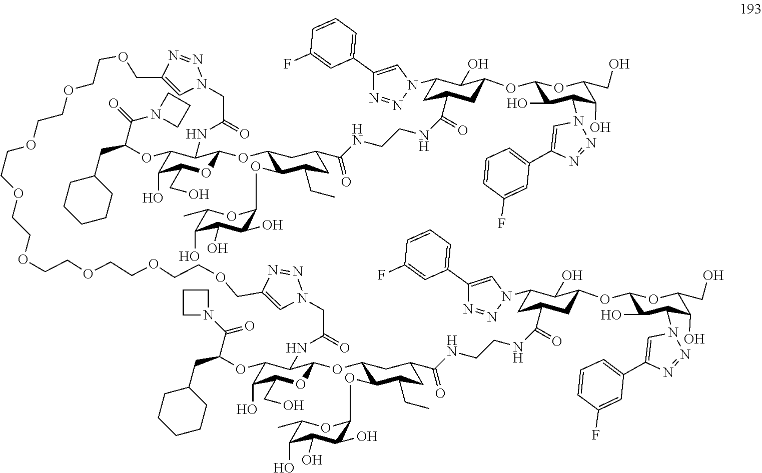

### Example 159

### Prophetic Synthesis of Multimeric Compound 194

Compound 194: Compound 194 can be prepared in an analogous fashion to FIG. 14 by replacing compound 22 with compound 92.

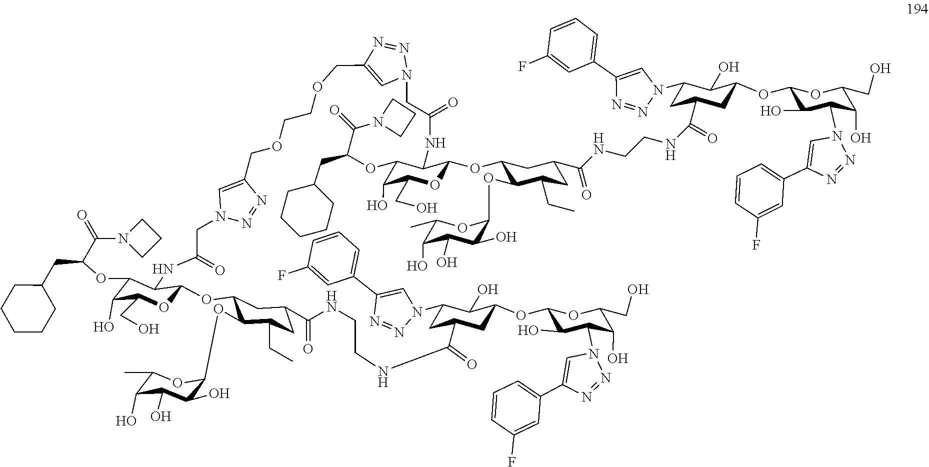

### Example 160

### Prophetic Synthesis of Multimeric Compound 195

Compound 195: Compound 195 can be prepared in an analogous fashion to FIG. 14 by replacing compound 22 with compound 93.

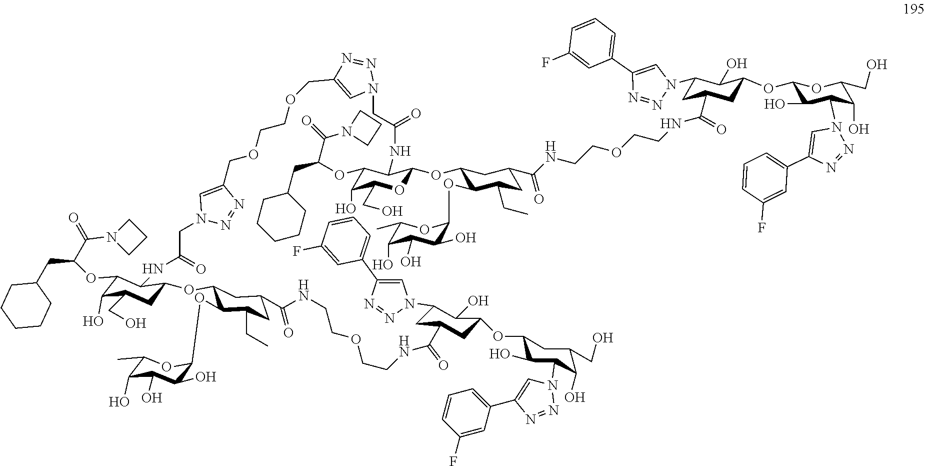

### Example 161

### Prophetic Synthesis of Multimeric Compound 197

Compound 197: To a solution of compound 22 (1 eq) in anhydrous DMSO was acetic acid NHS ester (compound 196) (5 eq). The mixture was stirred at ambient temperature for 12 hours. The solvent was removed in vacuo and the residue was purified by HPLC to afford compound 197.

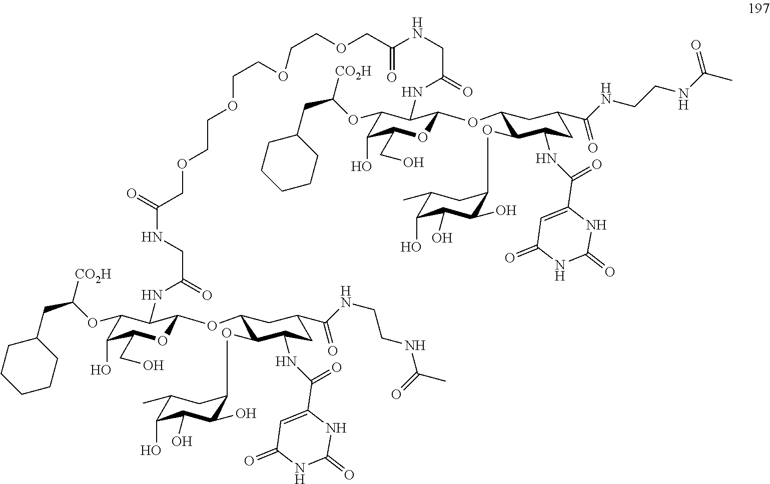

### Example 162

### Prophetic Synthesis of Multimeric Compound 198

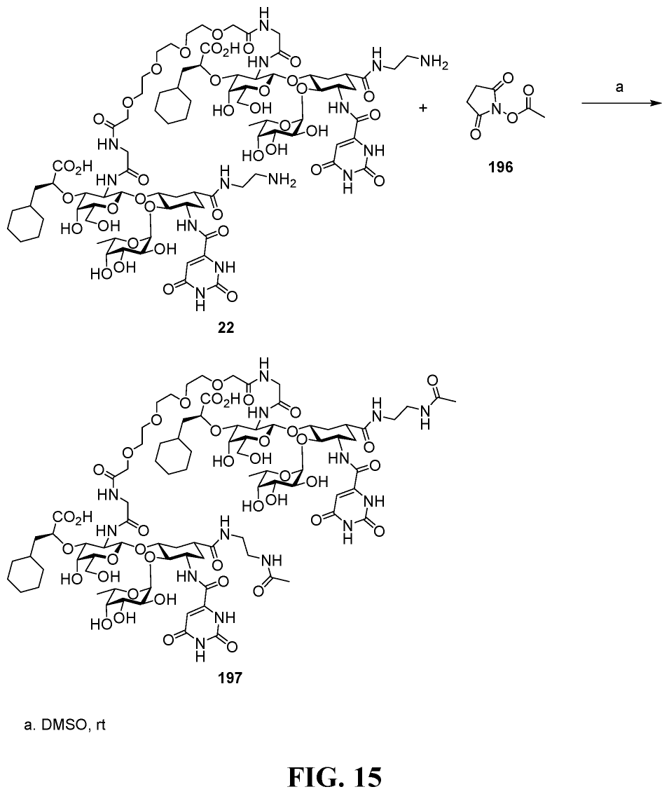

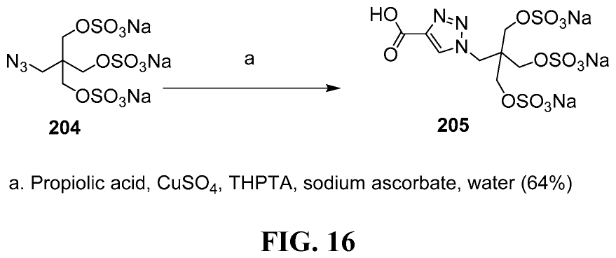

Compound 198: Compound 198 can be prepared in an analogous fashion to FIG. 15 by replacing compound 196 with NHS-methoxyacetate.

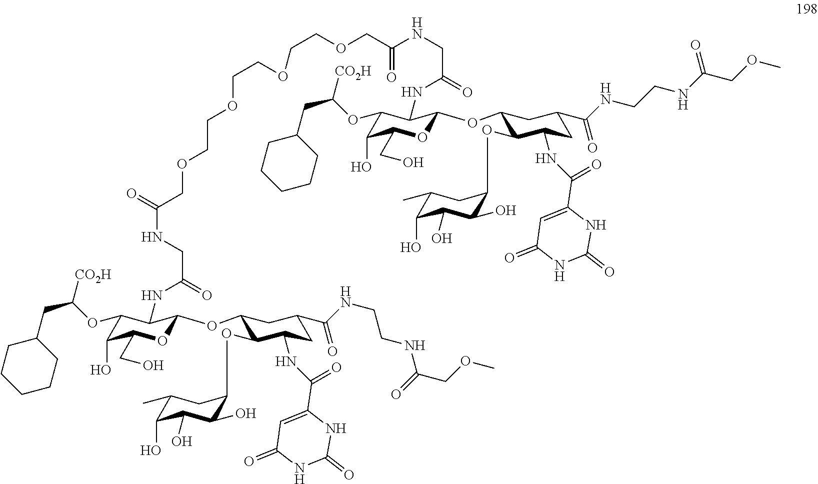

### Example 163

### Prophetic Synthesis of Multimeric Compound 199

Compound 199: Compound 199 can be prepared in an analogous fashion to FIG. 15 by replacing compound 196 with PEG-12 propionic acid NHS ester.

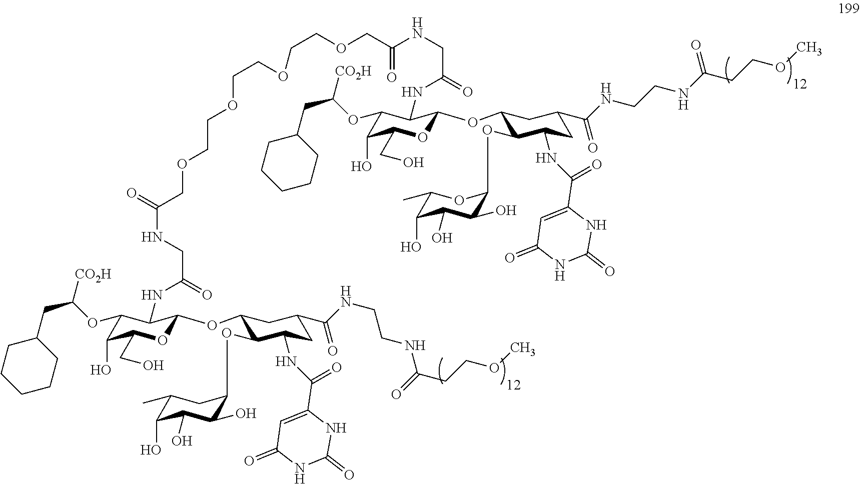

### Example 164

### Prophetic Synthesis of Multimeric Compound 200

Compound 200: Compound 200 can be prepared in an analogous fashion to FIG. 15 by replacing compound 22 with compound 78.

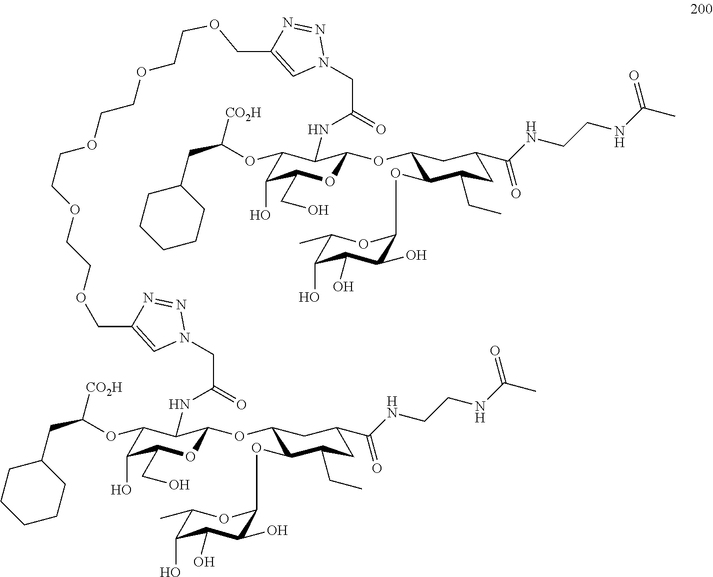

### Example 165

### Prophetic Synthesis of Multimeric Compound 201

Compound 201: Compound 201 can be prepared in an analogous fashion to FIG. 15 by replacing compound 22 with compound 78 and replacing compound 196 with NHS-methoxyacetate.

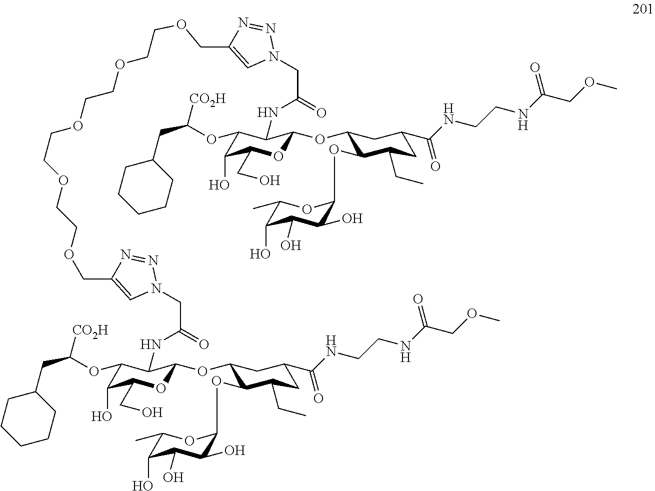

### Example 166

### Prophetic Synthesis of Multimeric Compound 202

Compound 202: Compound 202 can be prepared in an analogous fashion to FIG. 15 by replacing compound 22 with compound 78 and replacing compound 196 with PEG-12 propionic acid NHTS ester.

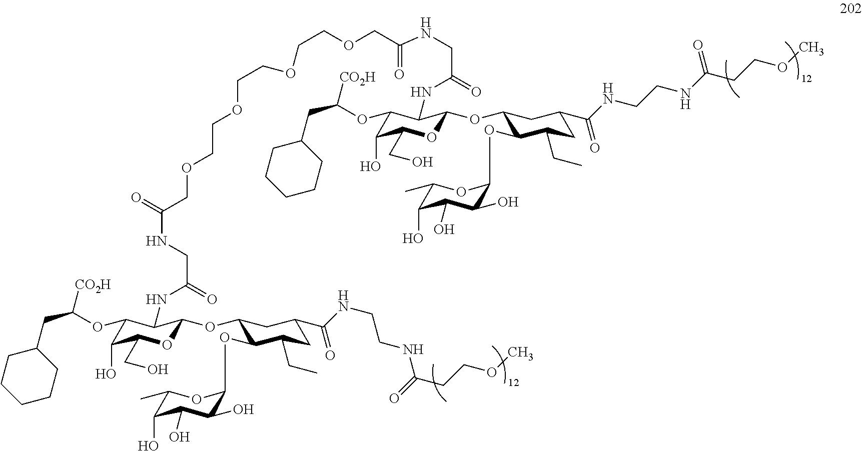

### Prophetic Synthesis of Multimeric Compound 203

Compound 203: Compound 203 can be prepared in an analogous fashion to FIG. 15 by replacing compound 22 with compound 78.

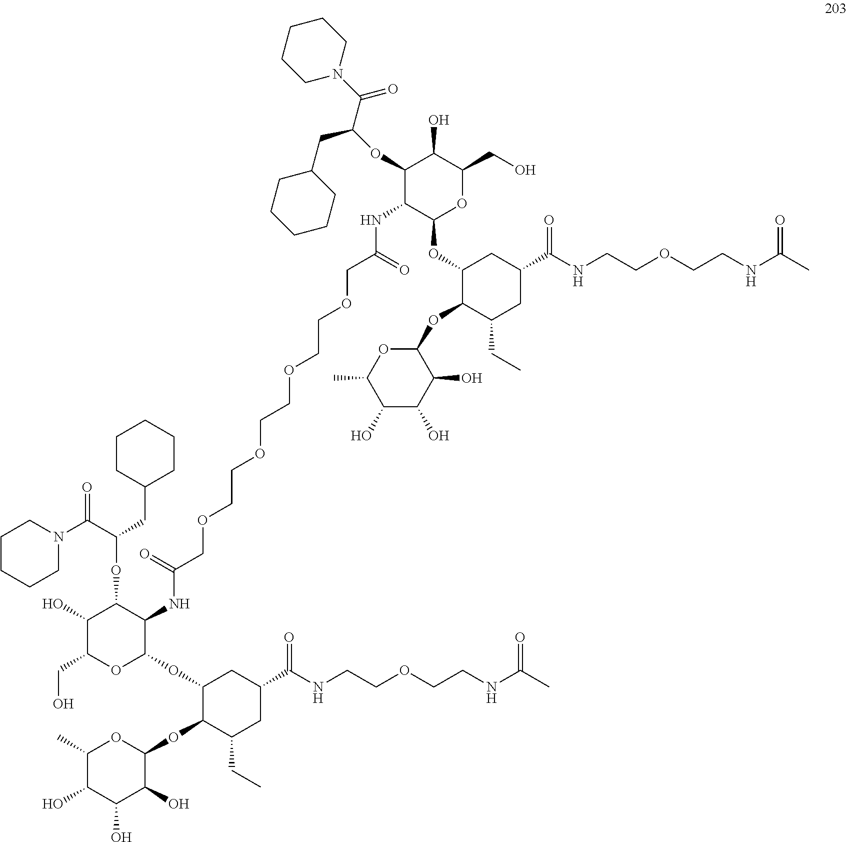 [SMILES: CC[C@H]1C[C@@H](C(=O)NCCOCCNC(C)=O)C[C@@H](O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCNC(C)=O)C[C@H](CC)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H]1O[C@@H]1O[C@@H](C)[C@@H](O)[C@@H](O)[C@@H]1O]

### Example 167

### Synthesis of Multimeric Compound 206

Compound 205: A solution of compound 204 (synthesis described in Mead, G. et. al., Bioconj. Chem., 2015, 25, 1444-1452) (0.25 g, 0.53 mmole) and propiolic acid (0.33 mL, 5.30 mmole, 10 eq) in distilled water (1.5 mL) was degassed. A solution of CuSO₄/THPTA in distilled water (0.04 M) (1.3 mL, 53 μmole, 0.1 eq) and sodium ascorbate (21 mg, 0.11 mmole, 0.2 eq) were added successively and the resulting solution was stirred 3 hrs at room temperature. The reaction mixture was concentrated under reduced pressure and partially purified by C-18 column chromatography (water/MeOH, water only—5/5, v/v). The resulting material was further purified by C-18 column chromatography eluting with water to afford compound 205 (0.16 g, 0.34 mmole, 64%). MS: (Calculated for C₈H₁₀₃N₃Na₃O₁₄S3, 537.34), ES—Negative (513.5, M-Na-1).

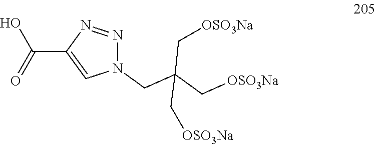

Compound 206: To a solution of compound 205 (7.5 mg, 14 μmole), DIPEA (2.4 μL, 14 μmole) and a catalytic amount of DMAP in DMF/DMSO (3/1, v/v, 0.15 mL) at 0° C. was added EDCI (1.6 mg, 8.22 μmole). The solution was stirred for 20 min. This solution was slowly added to a solution of compound 78 (5.0 mg, 2.7 μmole) in DMF/DMSO (3/1, v/v, 0.2 mL) cooled at 0° C. The resulting solution was stirred 12 hrs allowing the reaction temperature to increase to room temperature. The reaction mixture was purified directly by HPLC. The product portions were collected, concentrated under reduced pressure, then lyophilized to give compound 206 as a white solid (0.4 mg, 1.15 μmole, 1.1%). MS: Calculated (C₉₈H₁₅₄N₁₈Na₆O₅₉S₆, 2856.7), ES—Negative (907.7, M/3; 881.0, M-1SO₃/3; 854.1 M-2SO₃/3; 685.8 M+1Na/4; 680.5 M/4); Fraction of RT=10.65 min, 1399.4, M+7Na-1SO₃/2; 959.3 M+7Na/3; M+7Na-1SO₃/3; 724.8, M+8Na/4; 549. M+1Na/5; 460.9 M+2Na/6; 401. M+4Na/7).

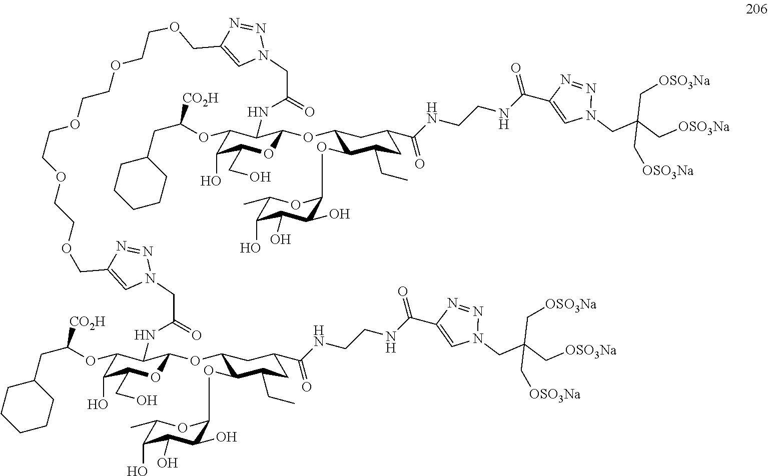

### Example 168

### Prophetic Synthesis of Multimeric Compound 207

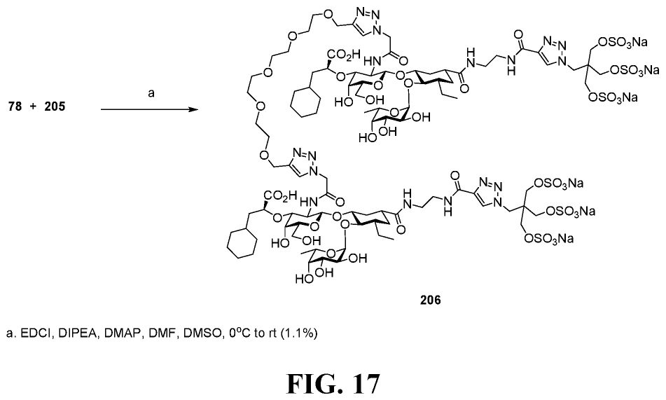

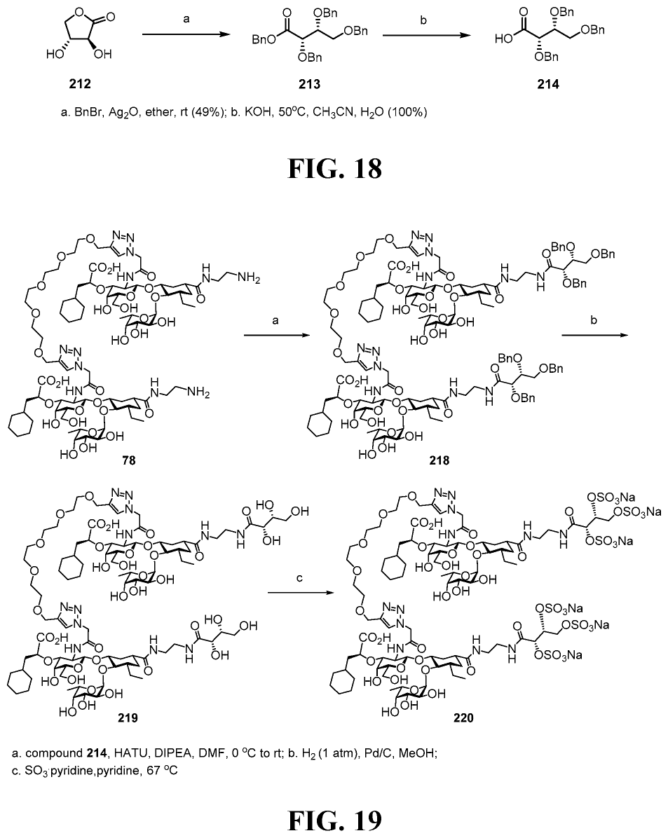

Compound 207: Compound 207 can be prepared in an analogous fashion to FIG. 17 by replacing compound 78 with compound 22.

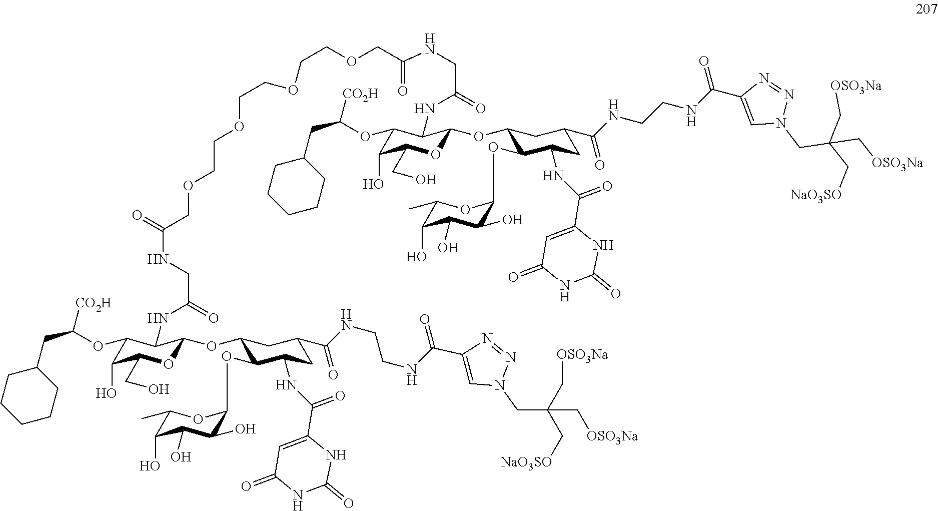

### Example 169

### Prophetic Synthesis of Multimeric Compound 208

Compound 208: Compound 208 can be prepared in an analogous fashion to FIG. 17 using compound 83 in place of compound 78.

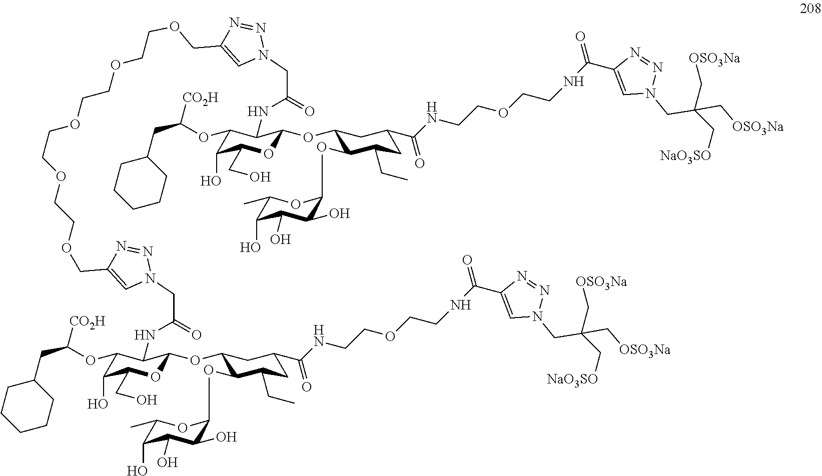

### Example 170

### Prophetic Synthesis of Multimeric Compound 209

Compound 209: Compound 209 can be prepared in an analogous fashion to FIG. 17 using compound 87 in place of compound 78.

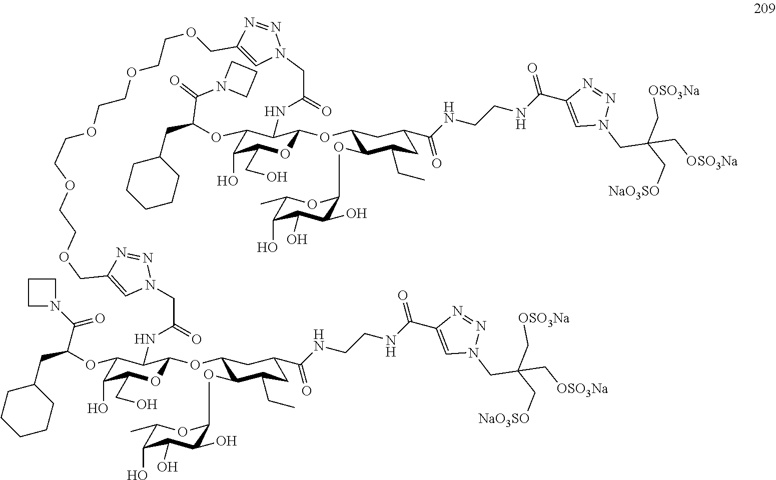

### Example 171

### Prophetic Synthesis of Multimeric Compound 210

Compound 210: Compound 210 can be prepared in an analogous fashion to FIG. 17 using compound 93 in place of compound 78.

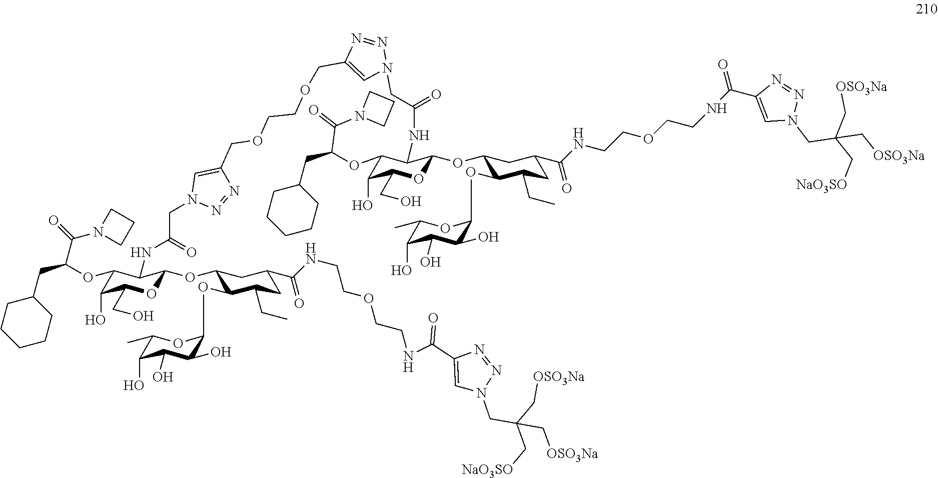

### Example 172

### Prophetic Synthesis of Multimeric Compound 211

Compound 211: Compound 211 can be prepared in an analogous fashion to FIG. 17 using compound 37 in place of compound 78.

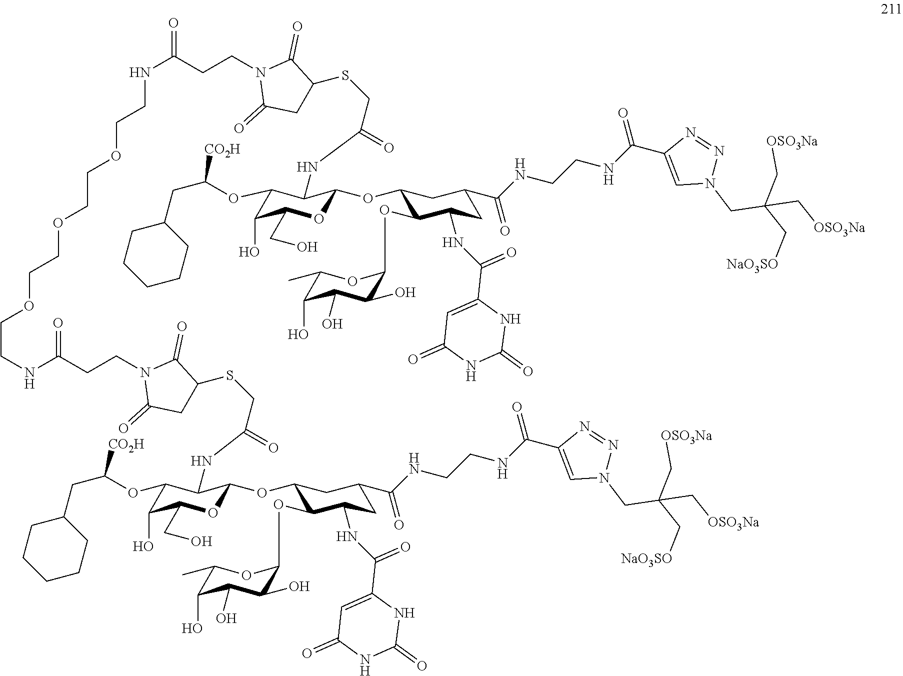

### Example 173

### Synthesis of Multimeric Compound 218

Compound 213: Prepared according to Bioorg. Med. Chem. Lett. 1995, 5, 2321-2324 starting with D-threonolactone.

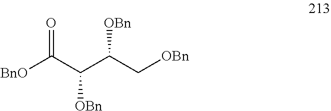 [SMILES: O=C(OCc1ccccc1)[C@@H](OCc1ccccc1)[C@@H](COCc1ccccc1)OCc1ccccc1]

Compound 214: Compound 213 (500 mg, 1 mmol) was dissolved in 9 mL acetonitrile. Potassium hydroxide (1 mL of a 2M solution) was added and the reaction mixture was stirred at 50° C. for 12 hours. The reaction mixture was partitioned between dichloromethane and water. The phases were separated and the aqueous phase was extracted 3 times with dichloromethane. The aqueous phase was acidified with 1N HCl until pH˜1 and extracted 3 times with dichloromethane. The combined dichloromethane extracts from after acidification of the aqueous phase were concentrated in vacuo to give compound 214 as a yellow oil (406 mg). LCMS (C-18; 5-95 H2O/MeCN) UV (peak at 4.973 mi), positive mode: m/z=407 [M+H]⁺; negative mode: m/z=405 [M−H]⁻ C₂₅H₂₆O₅ (406).

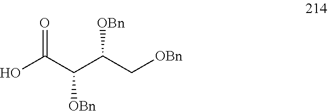 [SMILES: O=C(O)[C@@H](OCc1ccccc1)[C@@H](COCc1ccccc1)OCc1ccccc1]

Compound 215: Prepared in an analogous fashion to compound 214 using L-erythronolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻ C₂₅H₂₆O₅ (406).

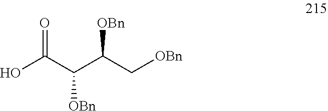 [SMILES: O=C(O)[C@@H](OCc1ccccc1)[C@H](COCc1ccccc1)OCc1ccccc1]

Compound 216: Prepared in an analogous fashion to compound 214 using L-threonolactone as the starting material. LCMS (C-18; 5-95 H₂O/MeCN): ELSD (5.08 min), UV (peak at 4.958 min), positive mode: m-z=407 [M+H]⁺; negative mode: m-z=405 [M−H]⁻ C₂₅H₂₆O₅ (406).

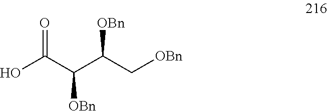 [SMILES: O=C(O)[C@H](OCc1ccccc1)[C@H](COCc1ccccc1)OCc1ccccc1]

Compound 217: Prepared in an analogous fashion to compound 214 using D-erythronolactone as the starting material. LCMS (C-18; 5-95 H₂O/MeCN): ELSD (5.08 min), UV (peak at 4.958 min), positive mode: m-z=407 [M+H]⁺; negative mode: m/z=405 [M−H]⁻ C₂₅H₂₆O₅ (406).

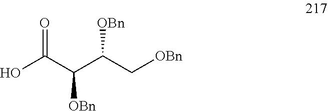 [SMILES: O=C(O)[C@H](OCc1ccccc1)[C@@H](COCc1ccccc1)OCc1ccccc1]

Compound 218: To a solution of compound 214 (3 eq) in anhydrous DMF was added HATU (3.3 eq) and DIPEA (5 eq). The mixture was stirred at ambient temperature for 15 minutes followed by addition of compound 78 (1 eq). The mixture was stirred at ambient temperature for 12 h. The solvent was removed in vacuo and the residue was purified by HPLC to afford compound 218.

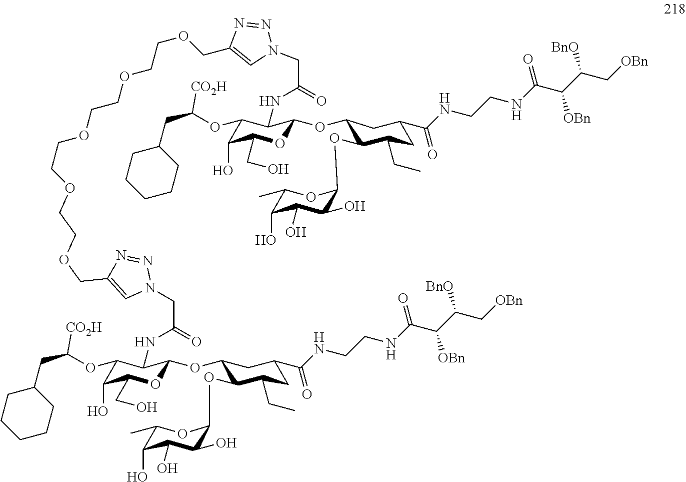

### Example 174

### Prophetic Synthesis of Multimeric Compound 219

Compound 219: Compound 218 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 219.

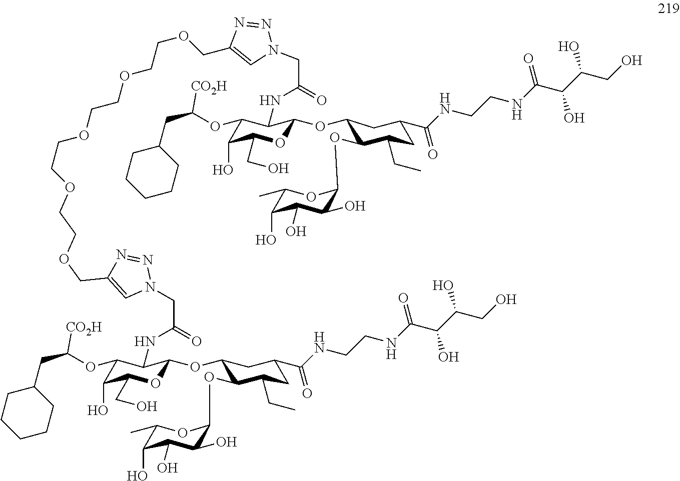

### Example 175

### Synthesis of Multimeric Compound 220

Compound 220: A solution of the sulfur trioxide pyridine complex (100 eq) and compound 219 (1 eq) in pyridine was stirred at 67° C. for 1 h. The reaction mixture was concentrated under vacuum. The resulting solid was dissolved in water and cooled to 0° C. A 1N solution of NaOH was then added slowly until pH-10 and the latter was freeze dried. The resulting residue was purified by Gel Permeation (water as eluent). The collected fractions were lyophilised to give compound 220.

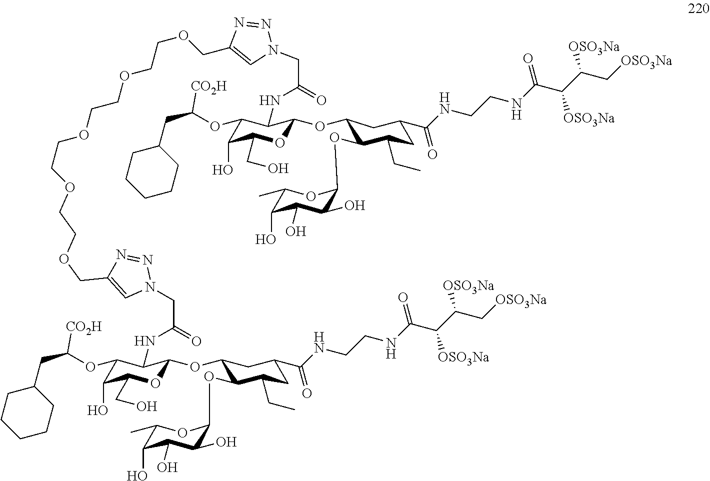

### Example 176

### Prophetic Synthesis of Multimeric Compound 221

Compound 221: Compound 221 can be prepared in an analogous fashion to FIG. 19 by replacing compound 214 with compound 215.

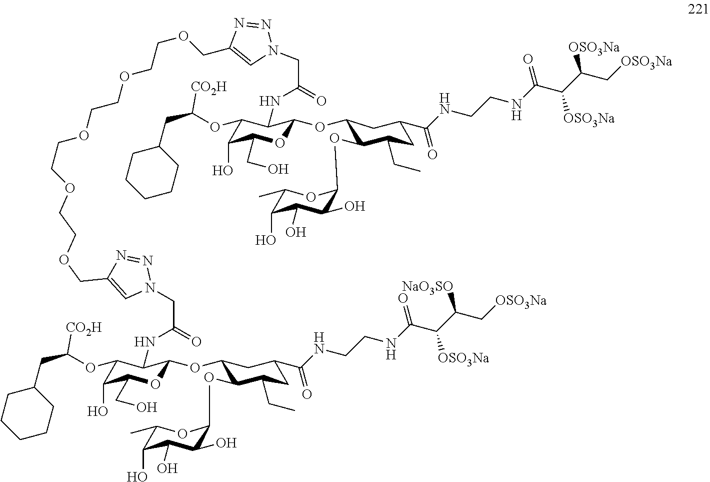

### Example 177

### Prophetic Synthesis of Multimeric Compound 222

Compound 222: Compound 222 can be prepared in an analogous fashion to FIG. 19 by replacing compound 214 with compound 216.

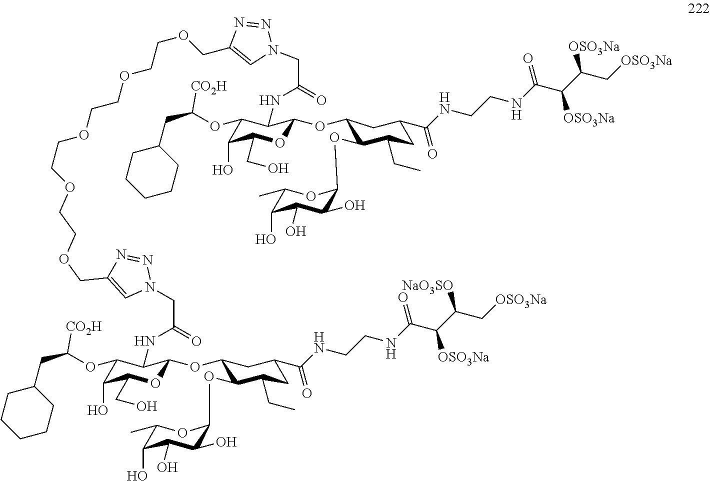

### Example 178

### Prophetic Synthesis of Multimeric Compound 223

Compound 223: Compound 223 can be prepared in an analogous fashion to FIG. 19 by replacing compound 214 with compound 217.

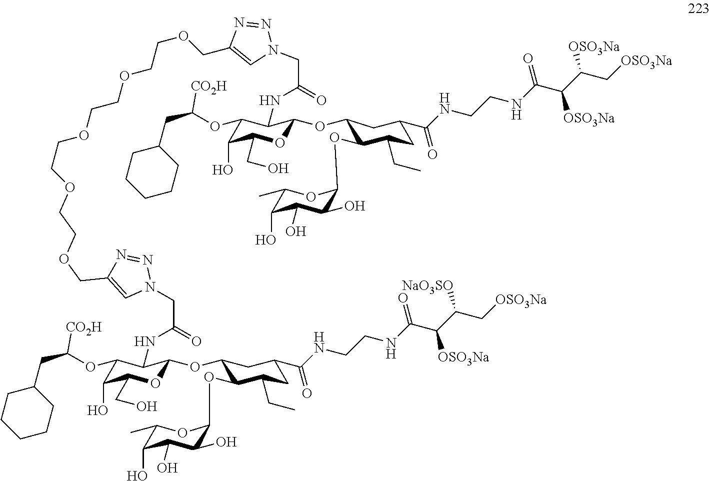

### Example 179

### Synthesis of Multimeric Compound 224

Compound 224: To a solution of compound 78 in anhydrous DMSO was added a drop of DIPEA and the solution was stirred at room temperature until a homogeneous solution was obtained. A solution of succinic anhydride (2.2 eq) in anhydrous DMSO was added and the resulting solution was stirred at room temperature overnight. The solution was lyophilized to dryness and the crude product was purified by HPLC to give compound 224.

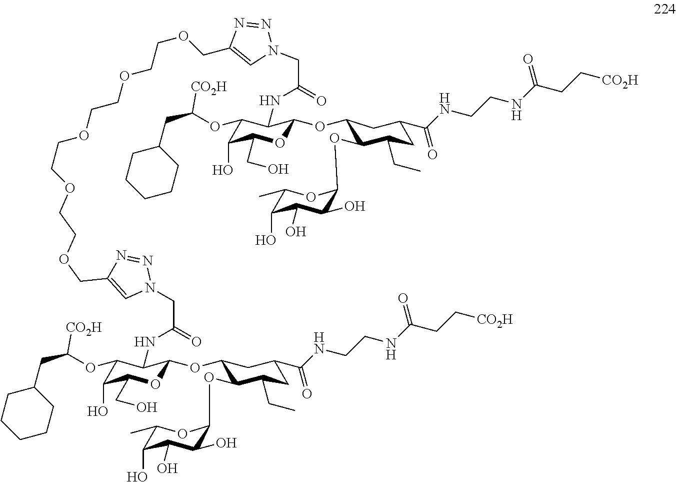

### Example 180

### Prophetic Synthesis of Multimeric Compound 225

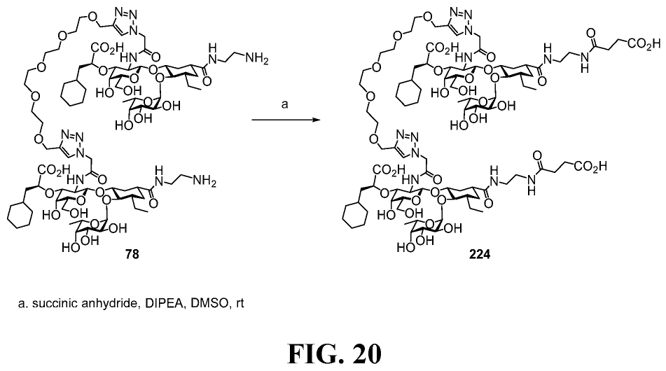

Compound 225: Compound 225 can be prepared in an analogous fashion to FIG. 20 substituting glutaric anhydride for succinic anhydride.

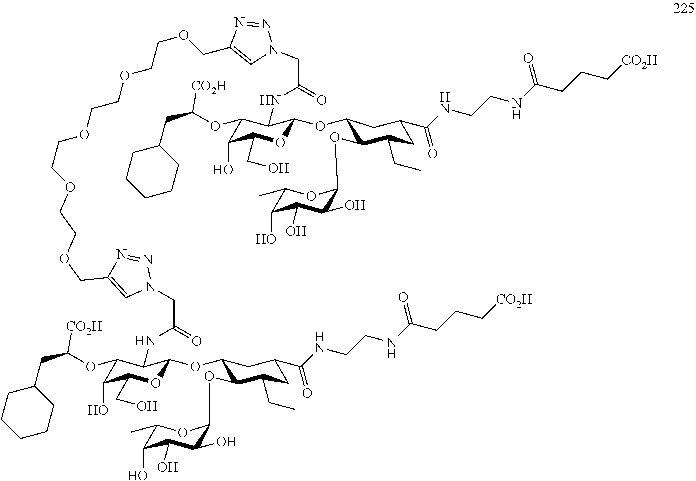

### Example 181

### Prophetic Synthesis of Multimeric Compound 226

Compound 226: Compound 226 can be prepared in an analogous fashion to FIG. 20 substituting compound 87 for compound 78.

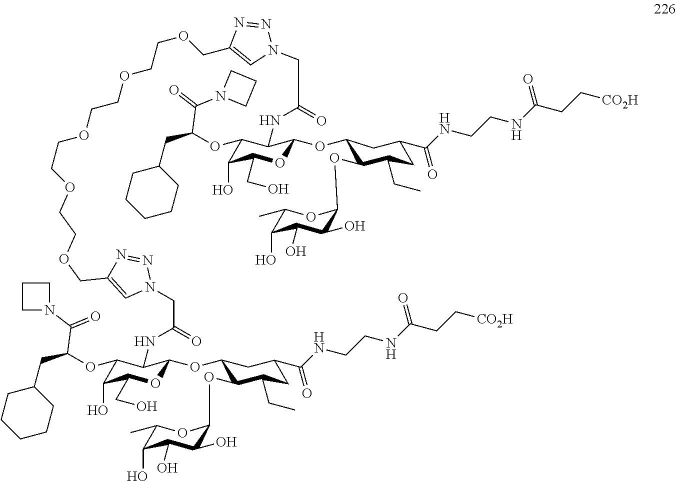

### Example 182

### Prophetic Synthesis of Multimeric Compound 227

Compound 227: Compound 227 can be prepared in an analogous fashion to FIG. 20 substituting phthalic anhydride for succinic anhydride.

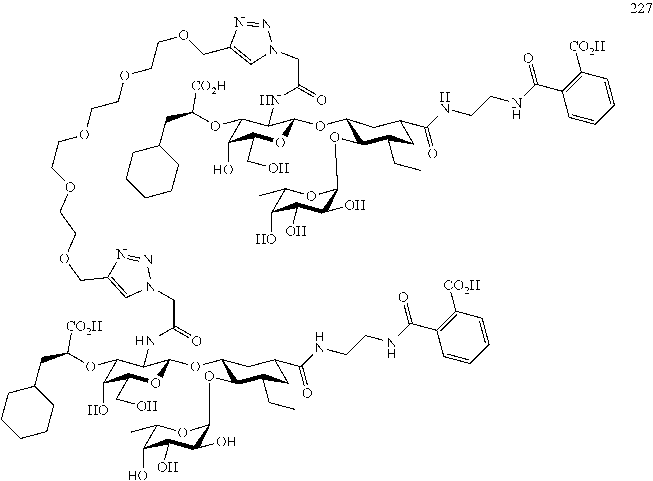

### Example 183

### Prophetic Synthesis of Multimeric Compound 228

Compound 228: Compound 228 can be prepared in an analogous fashion to FIG. 20 using compound 83 in place of compound 78.

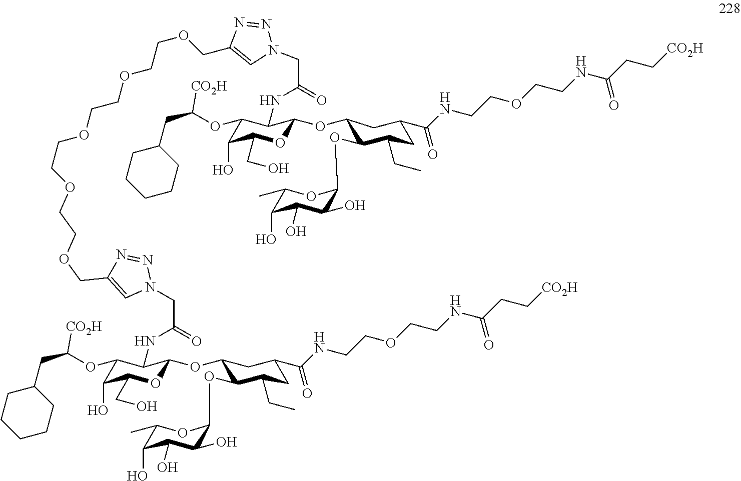

### Example 184

### Prophetic Synthesis of Multimeric Compound 229

Compound 229: Compound 229 can be prepared in an analogous fashion to FIG. 20 using compound 87 in place of compound 78.

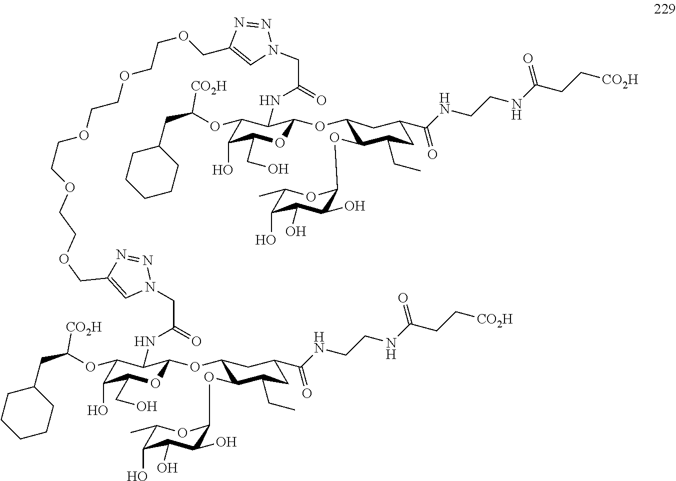

### Example 185

### Prophetic Synthesis of Multimeric Compound 245

Compound 231: A mixture of compound 230 (preparation described in Schwizer, et. al., Chem. Eur. J., 2012, 18, 1342) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 231.

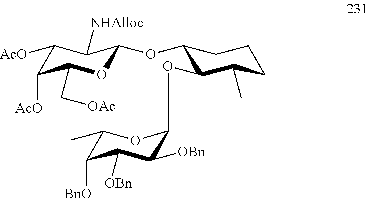

Compound 232: Compound 231 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 232.

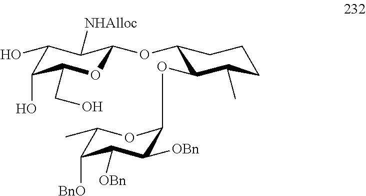

Compound 233: To a solution of compound 232 in dichloromethane cooled on an ice bath is added DABCO (1.5 eq) followed by monomethoxytrityl chloride (1.2 eq). The reaction mixture is stirred overnight allowing to warm to room temperature. The reaction mixture is concentrated and the residue is purified by flash chromatography to afford compound 233.

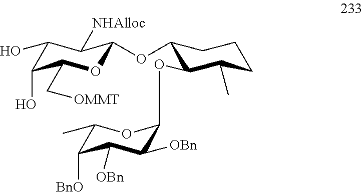

Compound 234: To a solution of compound 233 in methanol is added dibutyltin oxide (1.1 eq). The reaction mixture is refluxed for 3 hours then concentrated. The residue is suspended in DME. To this suspension is added compound 6 (preparation described in Thoma el. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 234.

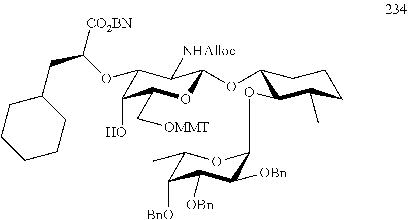

Compound 235: To a degassed solution of compound 234 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 235.

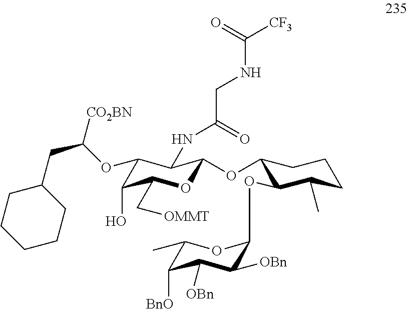

Compound 236: Compound 235 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 236.

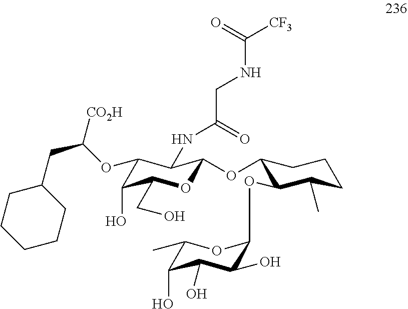

Compound 237: Compound 236 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 237.

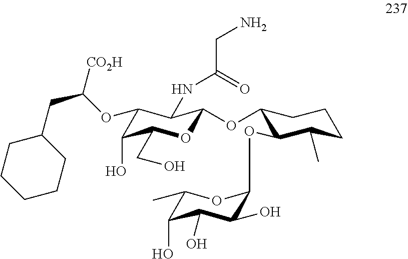

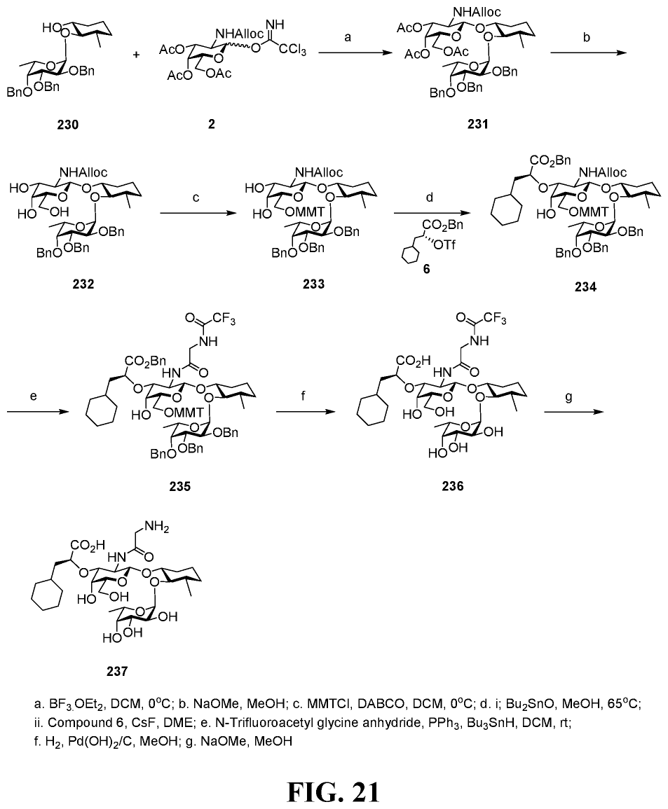

Compound 238: Compound 238 can be prepared in an analogous fashion to FIG. 21 by substituting (acetylthio)acetyl chloride for N-trifluoroacetyl glycine anhydride in step e.

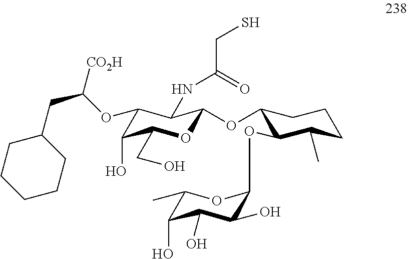

Compound 239: Compound 239 can be prepared in an analogous fashion to FIG. 21 by substituting the vinylcyclohexyl analog of compound 230 (preparation described in Schwizer, et. al., Chem. Eur. J., 2012, 18, 1342) for compound 230 in step a.

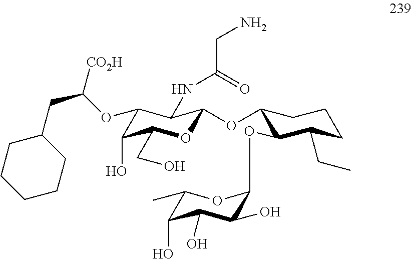

Compound 240: Compound 236 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 240.

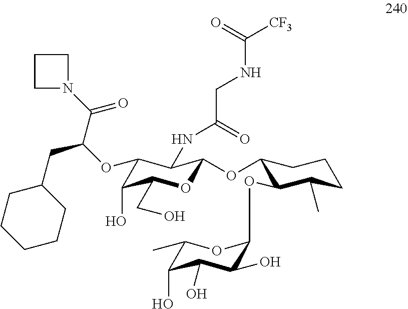

Compound 241: Compound 240 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 241.

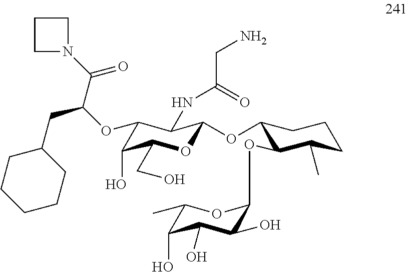

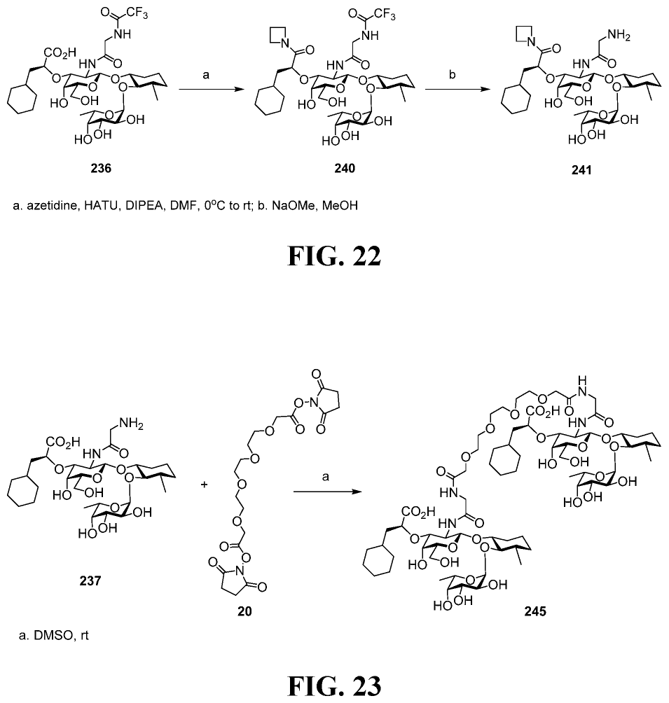

Compound 242: Compound 242 can be prepared in an analogous fashion to FIG. 22 by using methylamine in place of azetidine in step a.

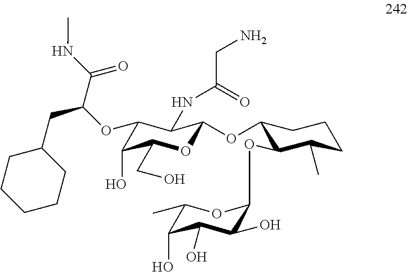

Compound 243: Compound 243 can be prepared in an analogous fashion to FIG. 22 by using dimethylamine in place of azetidine in step a.

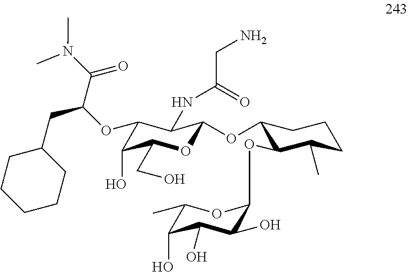

Compound 244: Compound 244 can be prepared in an analogous fashion to FIG. 22 by using the ethylcyclohexyl analog of compound 236 in place of compound 236 in step a.

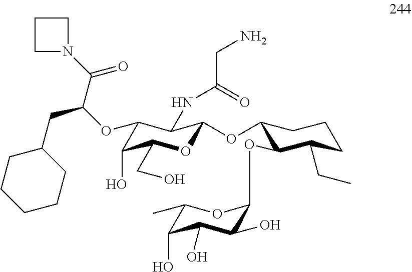

Compound 245: A solution of compound 20 (0.4 eq) in DMSO is added to a solution of compound 237 (1 eq) and DIPEA (10 eq) in anhydrous DMSO at room temperature. The resulting solution is stirred overnight. The reaction mixture is separated by reverse phase chromatography and the product lyophilized to give compound 245.

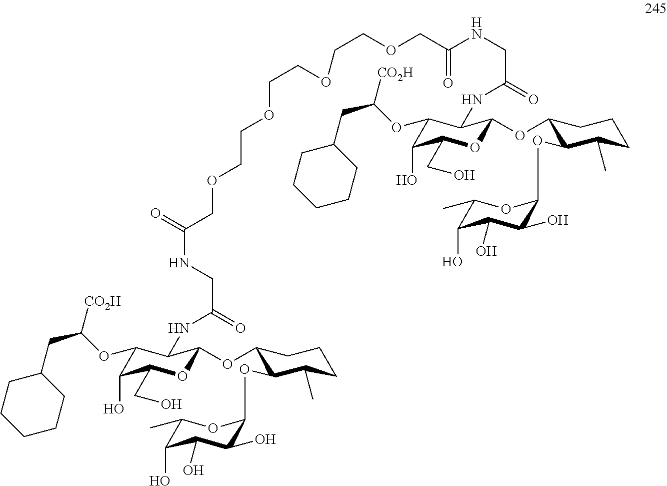

### Example 186

### Prophetic Synthesis of Multimeric Compound 246

Compound 246: Compound 246 can be prepared in an analogous fashion to FIG. 23 by replacing compound 20 with PEG-11 diacetic acid di-NHS ester.

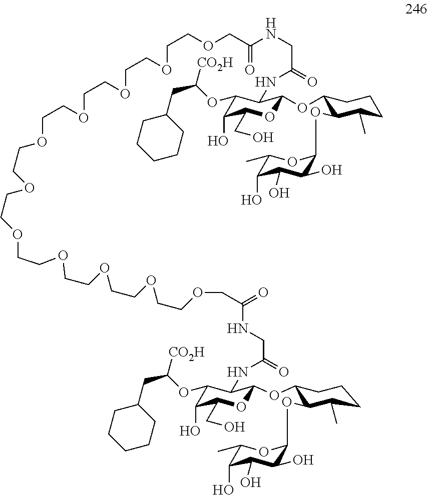

### Example 187

### Prophetic Synthesis of Multimeric Compound 247

Compound 247: Compound 247 can be prepared in an analogous fashion to FIG. 23 by replacing compound 20 with PEG-15 diacetic acid di-NHS ester.

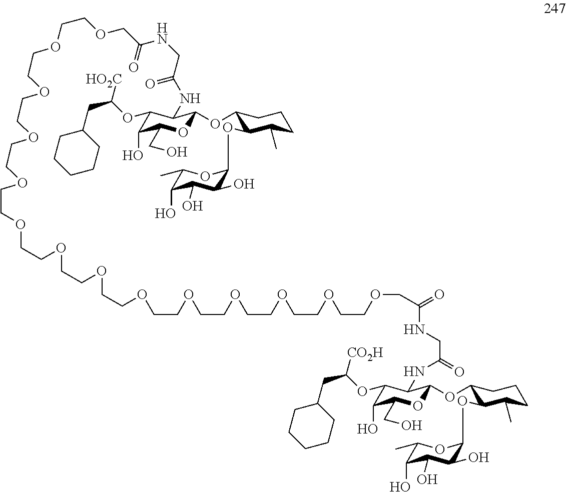

### Example 188

### Prophetic Synthesis of Multimeric Compound 248

Compound 248: Compound 248 can be prepared in an analogous fashion to FIG. 23 by replacing compound 20 with ethylene glycol diacetic acid di-NHS ester.

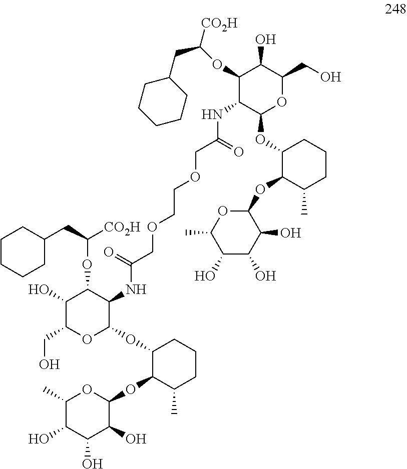 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 189

### Prophetic Synthesis of Multimeric Compound 249

Compound 249: Compound 249 can be prepared in an analogous fashion to FIG. 23 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester.

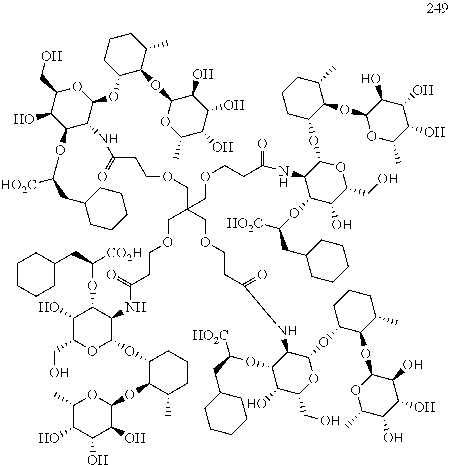

### Example 190

### Prophetic Synthesis of Multimeric Compound 250

Compound 250: Compound 250 can be prepared in an analogous fashion to FIG. 23 by replacing compound 237 with compound 239.

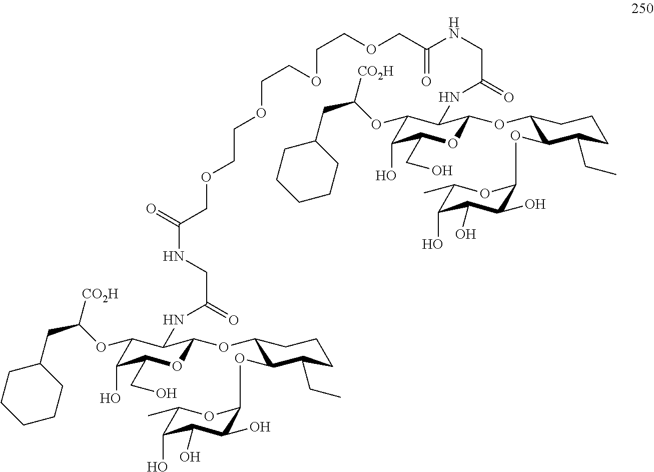

### Example 191

### Prophetic Synthesis of Multimeric Compound 251

Compound 251: Compound 251 can be prepared in an analogous fashion to FIG. 23 by replacing compound 237 with compound 241 and compound 20 with PEG-11 diacetic acid di-NHS ester.

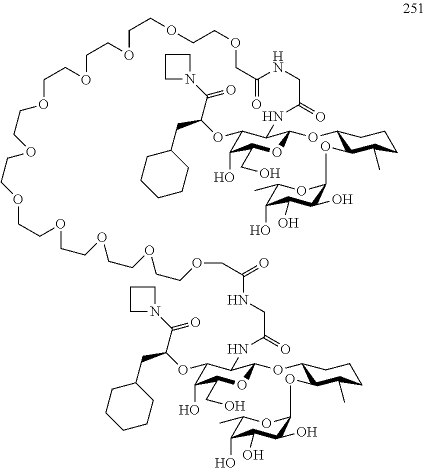

### Example 192

### Prophetic Synthesis of Multimeric Compound 252

Compound 252: Compound 252 can be prepared in an analogous fashion to FIG. 23 by replacing compound 237 with compound 242.

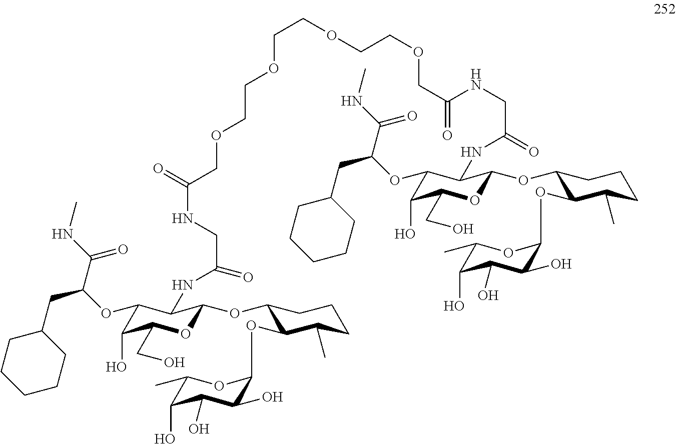

### Example 193

### Prophetic Synthesis of Multimeric Compound 253

Compound 253: Compound 253 can be prepared in an analogous fashion to FIG. 23 by replacing compound 237 with compound 243 and compound 20 with ethylene glycol diacetic acid di-NHS ester.

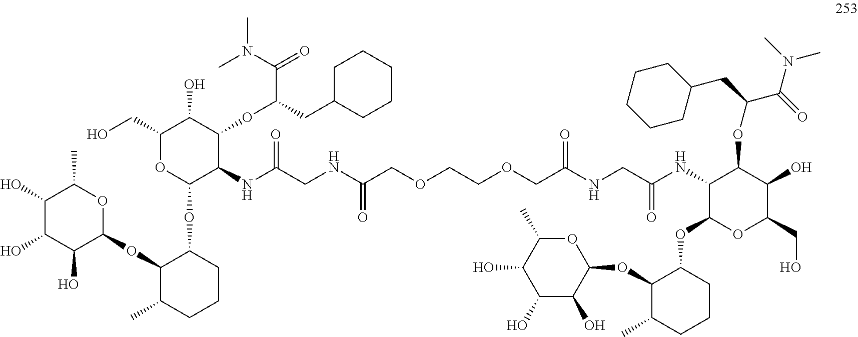 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N(C)C)[C@H]2NC(=O)CNC(=O)COCCOCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 194

### Prophetic Synthesis of Multimeric Compound 254

Compound 254: Compound 254 can be prepared in an analogous fashion to FIG. 23 by replacing compound 237 with compound 244 and compound 20 with PEG-11 diacetic acid di-NHS ester.

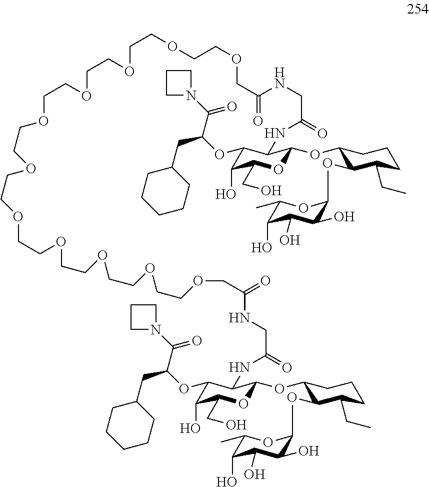

### Example 195

### Prophetic Synthesis of Multimeric Compound 255

Compound 255: Compound 255 can be prepared in an analogous fashion to FIG. 23 by replacing compound 237 with compound 241 and compound 20 with 1,1′-[oxybis[(1-oxo-2,1-ethanediyl)oxy]]bis-2,5-pyrrolidinedione.

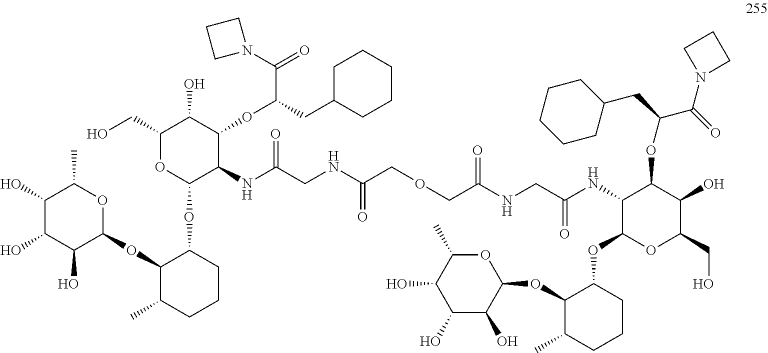 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 196

### Prophetic Synthesis of Multimeric Compound 256

Compound 256: Compound 256 can be prepared in an analogous fashion to FIG. 23 by replacing compound 237 with compound 244 and compound 20 with 1,1′-[oxybis[(1-oxo-2,1-ethanediyl)oxy]]bis-2,5-pyrrolidinedione.

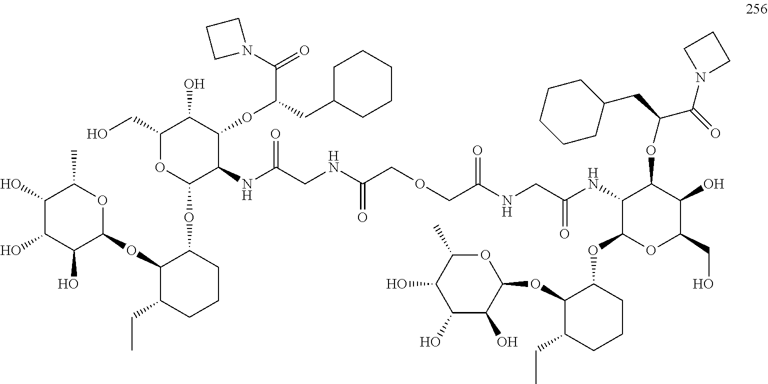 [SMILES: CC[C@H]1CCC[C@@H](O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](CC)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H]1O[C@@H]1O[C@@H](C)[C@@H](O)[C@@H](O)[C@@H]1O]

### Example 197

### Prophetic Synthesis of Multimeric Compound 257

Compound 257: To a solution of compound 238 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 257.

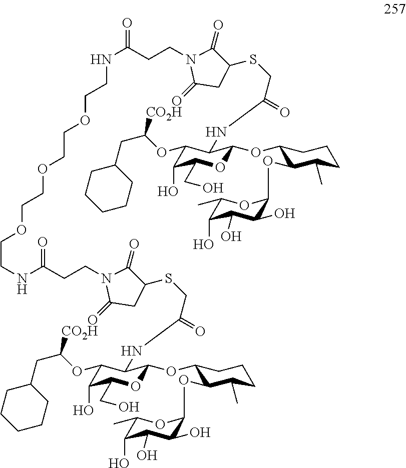

### Example 198

### Prophetic Synthesis of Multimeric Compound 258

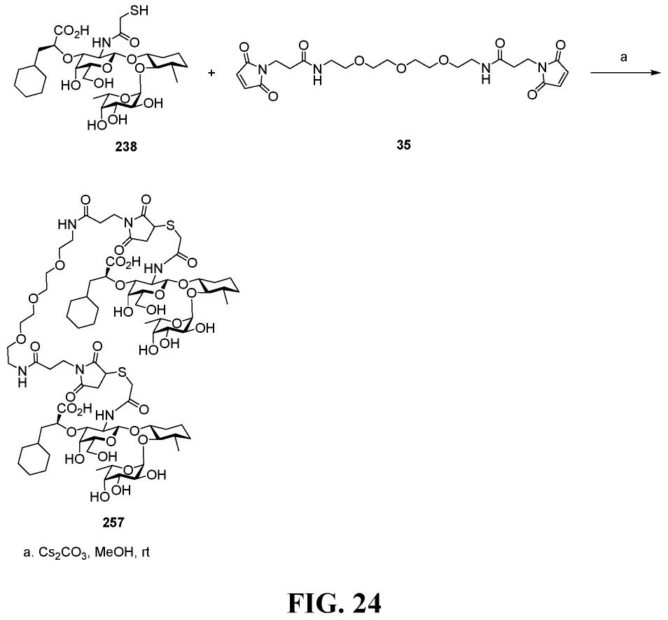

Compound 258: Compound 258 can be prepared in an analogous fashion to FIG. 24 by substituting PEG-6-bis maleimidoylpropionamide for compound 35.

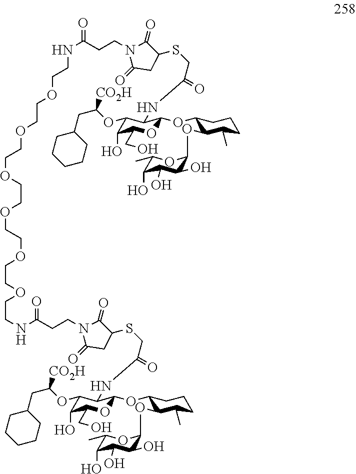

### Example 199

### Prophetic Synthesis of Multimeric Compound 259

Compound 259: Compound 259 can be prepared in an analogous fashion to FIG. 24 by substituting compound 35 for, 1,1′-[[2,2-bis[[3-(2,5-dihydro-2,5-dioxo-1H-pyrrol-1-yl)propoxy]methyl]-1,3-propanediyl]bis(oxy-3,1-propanediyl)]bis-1H-pyrrole-2,5-dione.

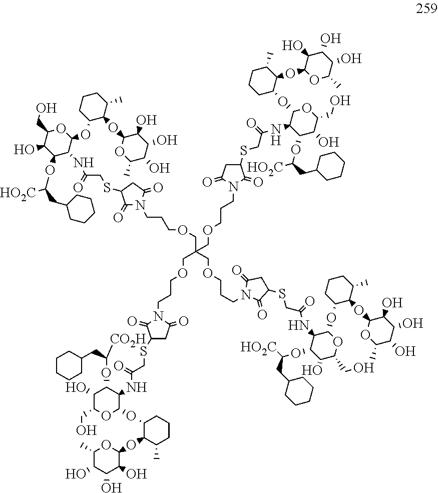

### Example 200

### Prophetic Synthesis of Multimeric Compound 261

Compound 260: To a degassed solution of compound 234 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 260.

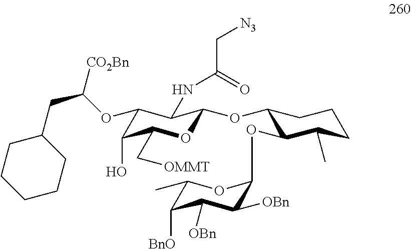

Compound 261: A solution of bis-propagyl PEG-5 (compound 43) and compound 260 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 261.

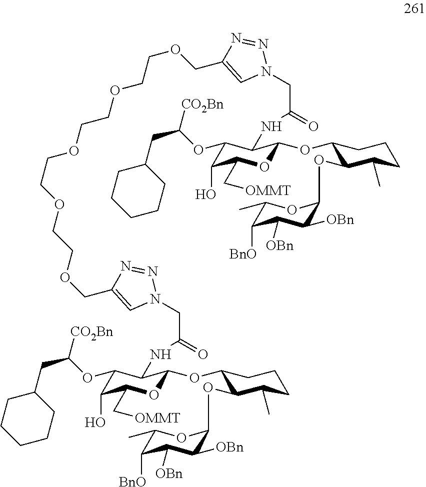

### Example 201

### Prophetic Synthesis of Multimeric Compound 262

Compound 262: Compound 261 is dissolved in MeOH and hydrogenated in the presence of Pd(OH)₂ (20 wt %) at 1 atm of H2 gas pressure for 24 hrs at room temperature. The solution is filtered through a Celite pad. The filtrate is concentrated to give compound 262.

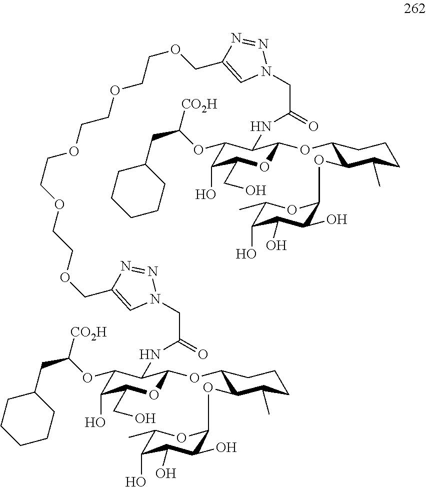

### Example 202

### Prophetic Synthesis of Multimeric Compound 263

Compound 263: Compound 262 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 263.

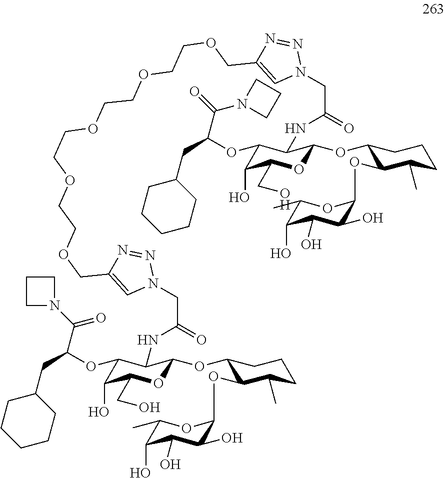

### Example 203

### Prophetic Synthesis of Multimeric Compound 264

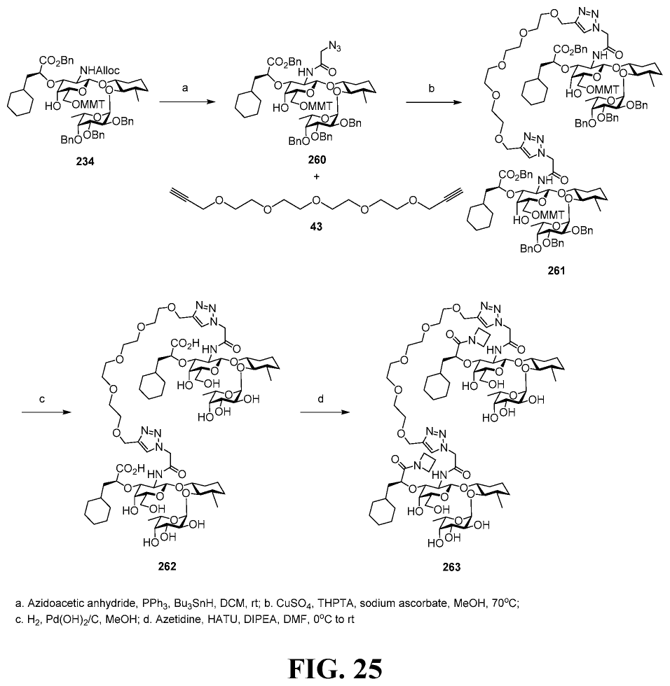

Compound 264: Compound 264 can be prepared in an analogous fashion to FIG. 25 using 4,7,10,13,16,19,22,25,28,31-decaoxatetratriaconta-1,33-diyne in place of compound 43 in step b.

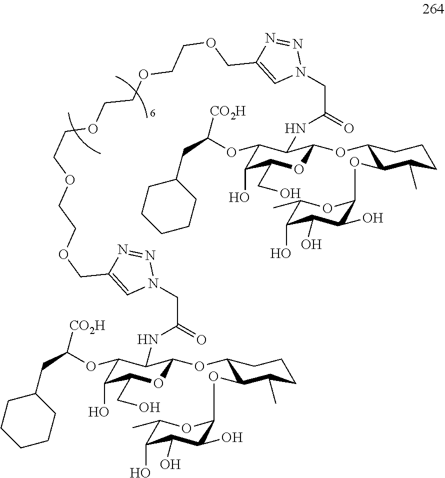

### Example 204

### Prophetic Synthesis of Multimeric Compound 265

Compound 265: Compound 265 can be prepared in an analogous fashion to FIG. 25 using 3,3′-[[2,2-bis[(2-propyn-1-yloxy)methyl]-1,3-propanediyl]bis(oxy)]bis-1-propyne in place of compound 43 in step b.

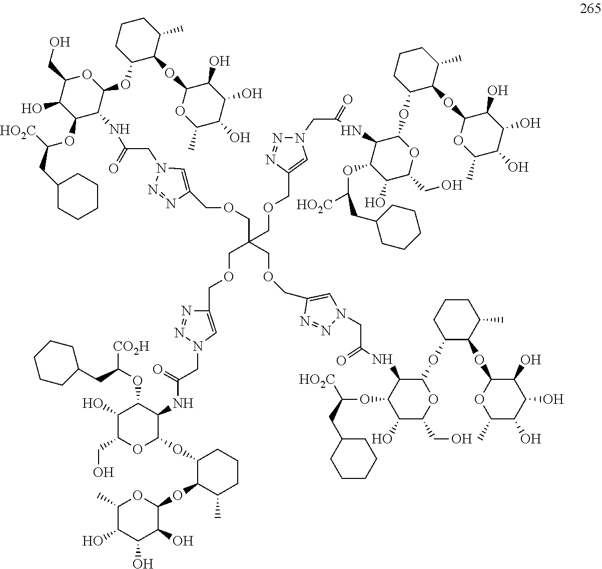

### Example 205

### Prophetic Synthesis of Multimeric Compound 266

Compound 266: Compound 266 can be prepared in an analogous fashion to FIG. 25 using 3,3′-[oxybis[[2,2-bis[(2-propyn-1-yloxy)methyl]-3,1-propanediyl]oxy]]bis-1-propyne in place of compound 43 in step b.

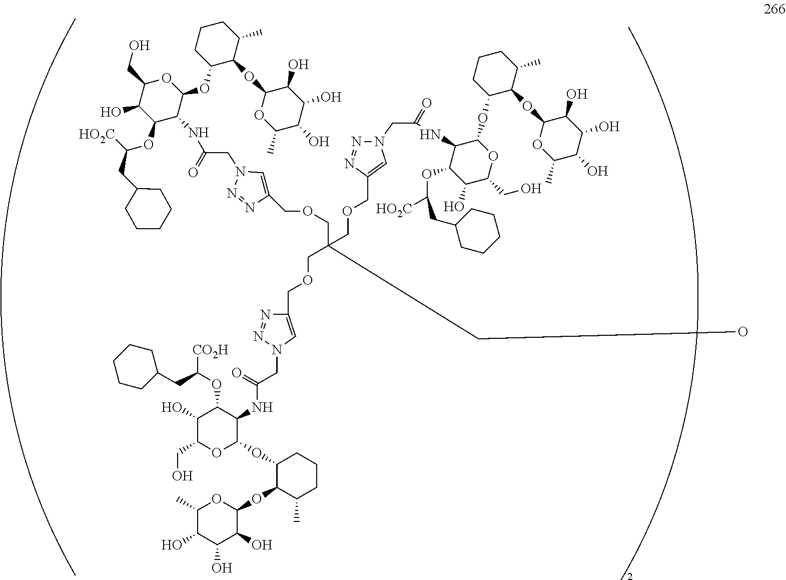

### Example 206

### Prophetic Synthesis of Multimeric Compound 267

Compound 267: Compound 267 can be prepared in an analogous fashion to FIG. 25 using ethylamine in place of azetidine in step d.

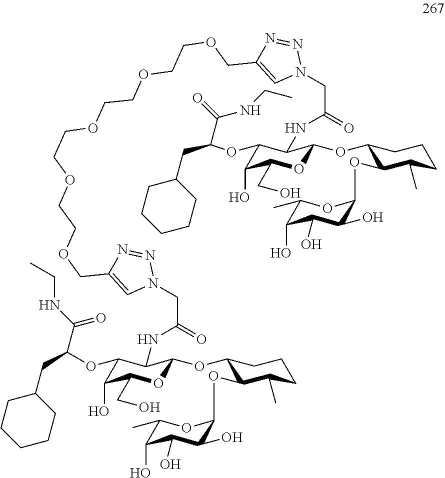

### Example 207

### Prophetic Synthesis of Multimeric Compound 268

Compound 268: Compound 268 can be prepared in an analogous fashion to FIG. 25 using dimethylamine in place of azetidine in step d.

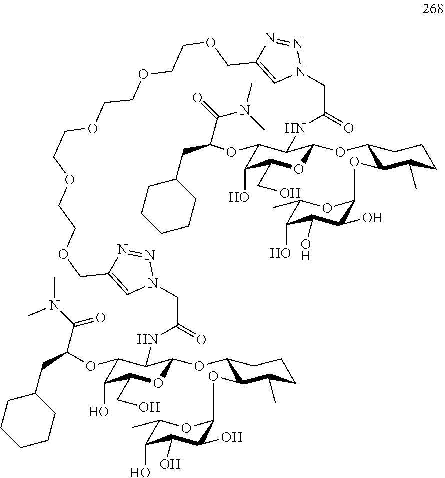

### Example 208

### Prophetic Synthesis of Multimeric Compound 269

Compound 269: Compound 269 can be prepared in an analogous fashion to FIG. 25 using the analog of compound 234 prepared from vinylcyclohexane in place of compound 234 in step a.

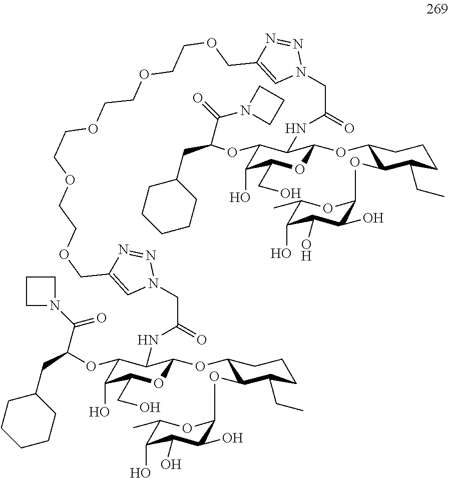

### Example 209

### Prophetic Synthesis of Multimeric Compound 270

Compound 270: Compound 270 can be prepared in an analogous fashion to FIG. 25 using propargyl ether in place of compound 43 in step b.

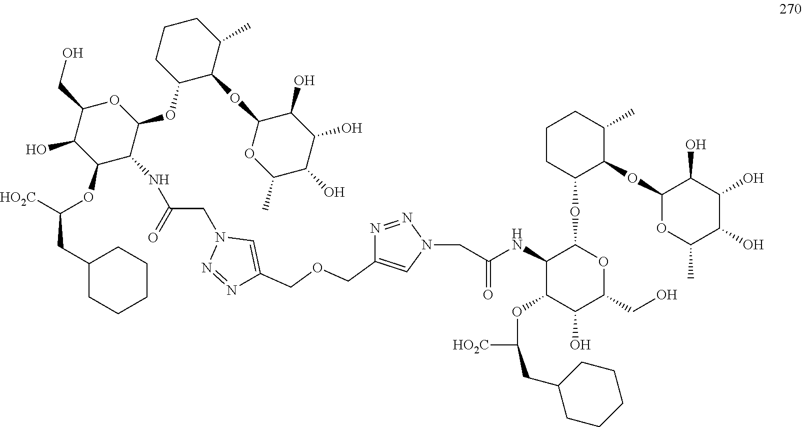 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 210

### Prophetic Synthesis of Multimeric Compound 271

Compound 271: Compound 271 can be prepared in an analogous fashion to FIG. 25 using propargyl ether in place of compound 43 in step b.

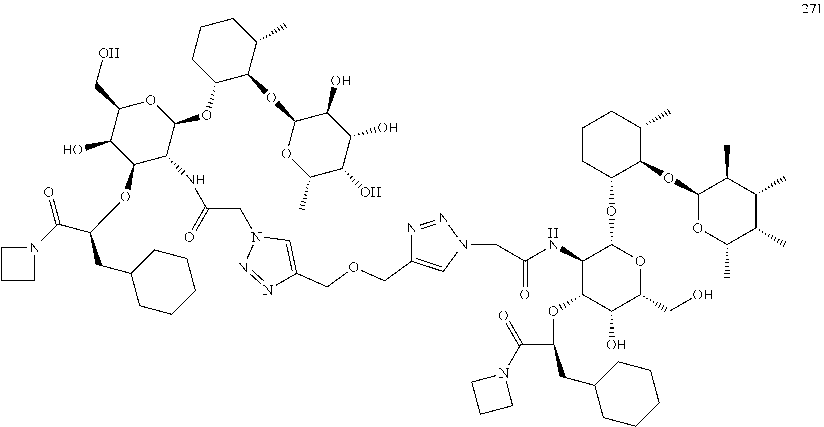 [SMILES: C[C@H]1[C@H](C)[C@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)O[C@@H](C)[C@H]1C]

### Example 211

### Prophetic Synthesis of Multimeric Compound 274

Compound 272: Activated powdered 4 Å molecular sieves are added to a solution of compound 230 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 272.

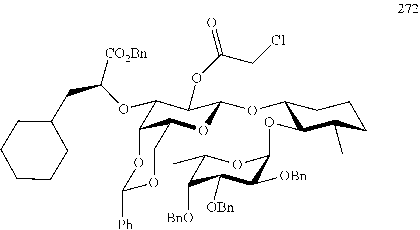

Compound 273: Compound 272 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 273.

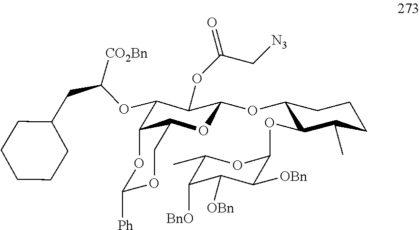

Compound 274: A solution of bispropagyl PEG-5 (compound 43) and compound 273 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 274.

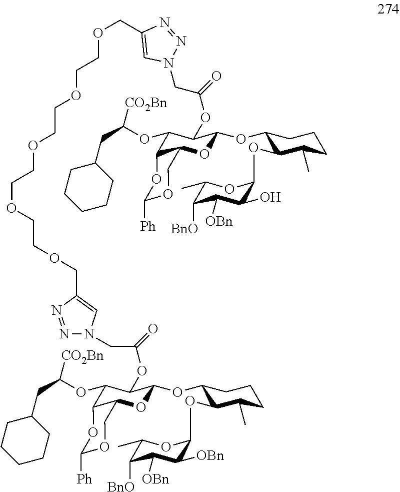

### Example 212

### Prophetic Synthesis of Multimeric Compound 275

Compound 275: To a solution of compound 274 in dioxane/water (4/1) is added Pd(OH)₂/C. The reaction mixture is stirred vigorously overnight under a hydrogen atmosphere. The reaction mixture is filtered through Celite and concentrated. The residue is purified by C-18 reverse phase column chromatography to afford compound 275.

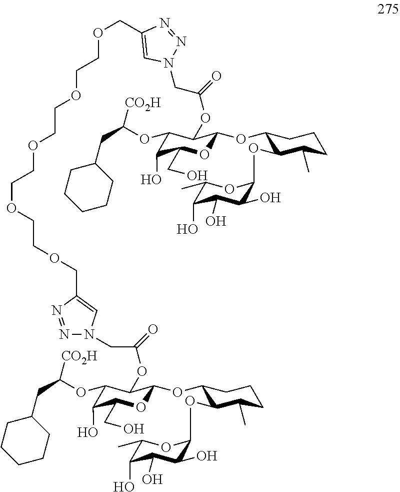

### Example 213

### Prophetic Synthesis of Multimeric Compound 276

Compound 276: Compound 275 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 276.

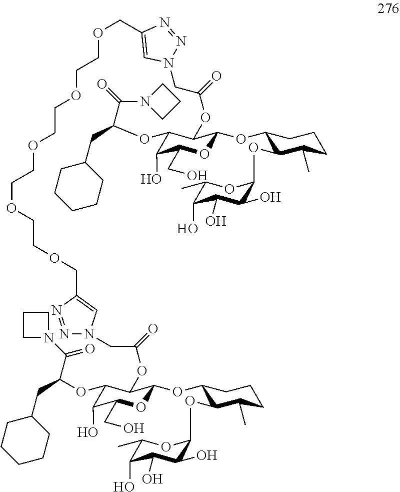

### Example 214

### Prophetic Synthesis of Multimeric Compound 277

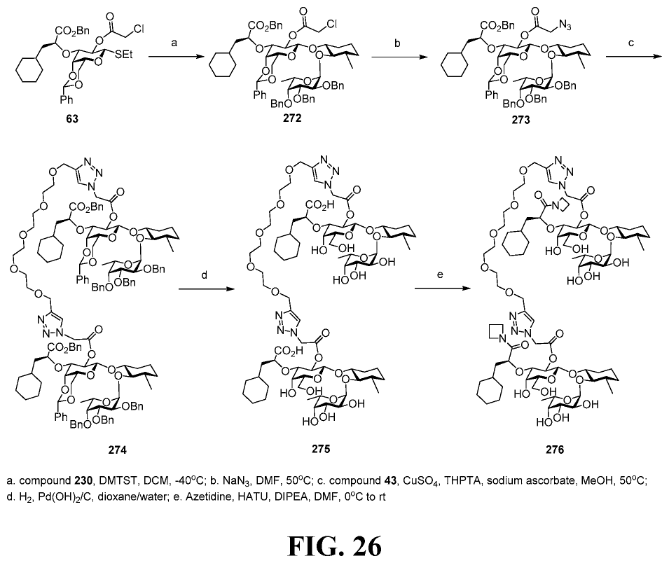

Compound 277: Compound 277 can be prepared in an analogous fashion to FIG. 26 by replacing compound 43 with PEG-8 bis propargyl ether in step c.

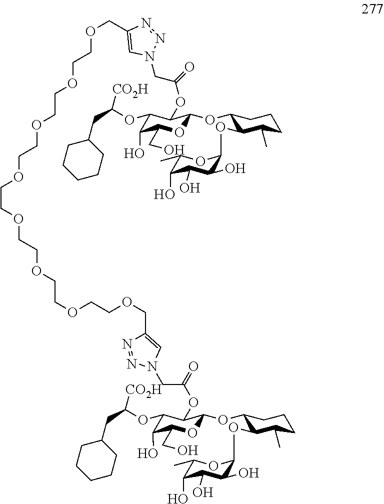

### Example 215

### Prophetic Synthesis of Multimeric Compound 278

Compound 278: Compound 278 can be prepared in an analogous fashion to FIG. 26 by replacing compound 43 with ethylene glycol bis propargyl ether in step c.

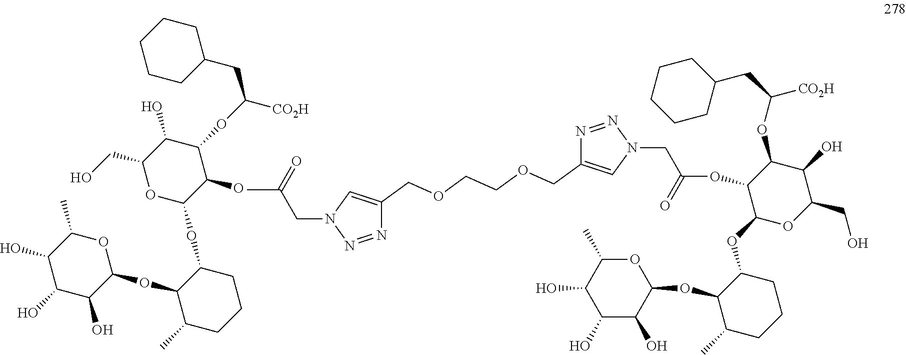

### Example 216

### Prophetic Synthesis of Multimeric Compound 279

Compound 279: Compound 279 can be prepared in an analogous fashion to FIG. 26 using 3,3′-[[2,2-bis[(2-propyn-1-yloxy)methyl]-1,3-propanediyl]bis(oxy)]bis-1-propyne in place of compound 43 in step c.

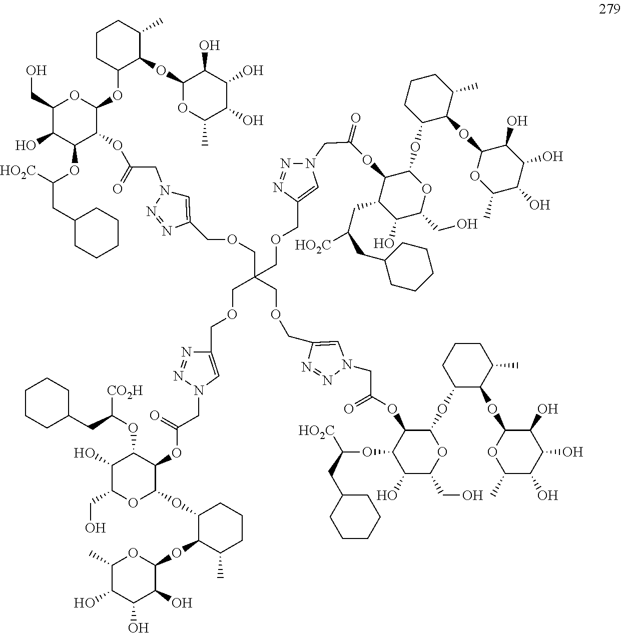

### Example 217

### Prophetic Synthesis of Multimeric Compound 280

Compound 280: Compound 280 can be prepared in an analogous fashion to FIG. 26 using propargyl ether in place of compound 43 in step c.

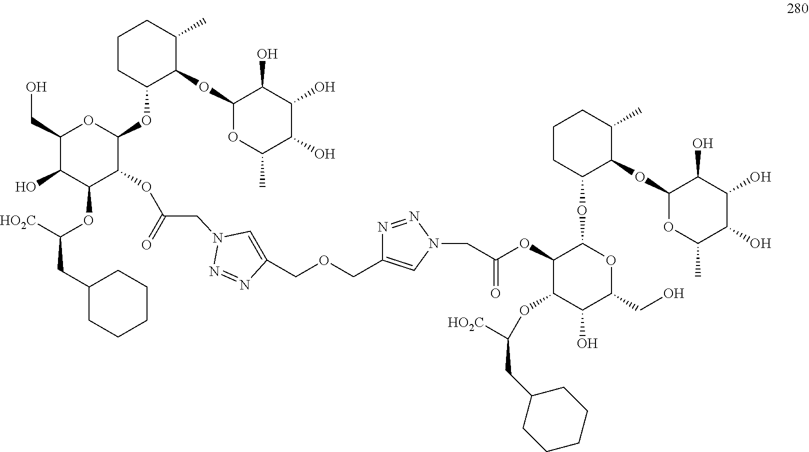 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2OC(=O)Cn2cc(COCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 218

### Prophetic Synthesis of Multimeric Compound 281

Compound 281: Compound 281 can be prepared in an analogous fashion to FIG. 26 using propargyl ether in place of compound 36 in step c.

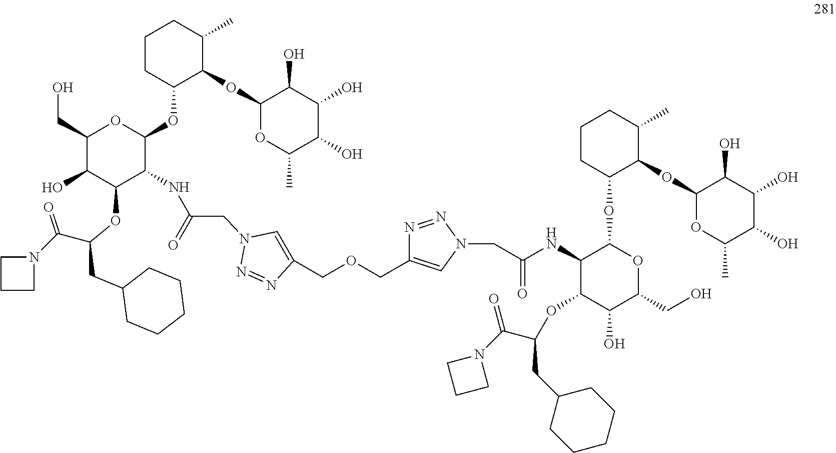 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 219

### Prophetic Synthesis of Multimeric Compound 282

Compound 282: Compound 282 can be prepared in an analogous fashion to FIG. 26 by replacing compound 43 with ethylene glycol bis propargyl ether in step c.

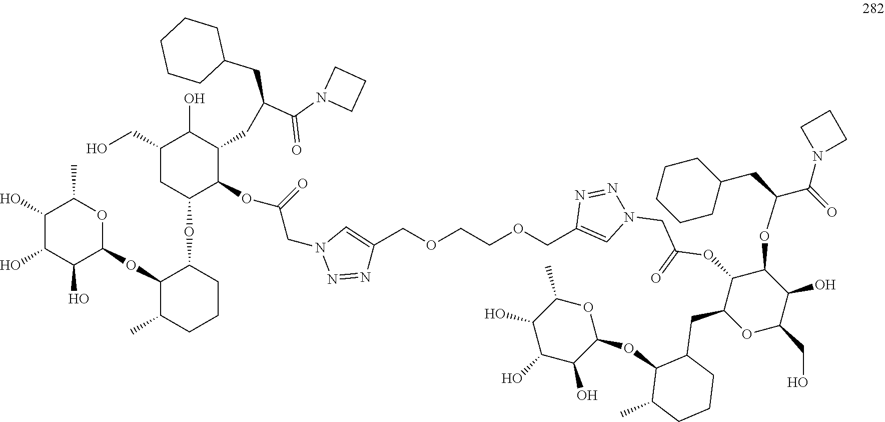 [SMILES: C[C@@H]1O[C@@H](O[C@@H]2[C@@H](C)CCC[C@H]2O[C@@H]2C[C@H](CO)C(O)[C@H](C[C@@H](CC3CCCCC3)C(=O)N3CCC3)[C@H]2OC(=O)Cn2cc(COCCOCc3cn(CC(=O)O[C@@H]4[C@@H](O[C@@H](CC5CCCCC5)C(=O)N5CCC5)[C@@H](O)[C@@H](CO)O[C@H]4CC4CCC[C@H](C)[C@H]4O[C@@H]4O[C@@H](C)[C@@H](O)[C@@H](O)[C@@H]4O)nn3)nn2)[C@@H](O)[C@H](O)[C@@H]1O]

### Example 220

### Prophetic Synthesis of Multimeric Compound 294

Compound 284: A mixture of compound 283 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 284.

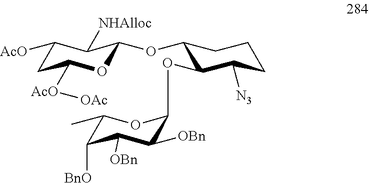

Compound 285: Compound 284 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 285.

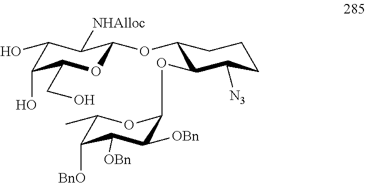

Compound 286: To a solution of compound 285 in dichloromethane cooled on an ice bath is added DABCO (1.5 eq) followed by monomethyoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 286.

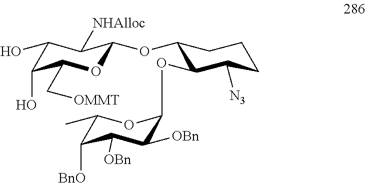

Compound 287: To a solution of compound 286 in methanol is added dibutyltin oxide (1.1 eq). The reaction mixture is refluxed for 3 hours then concentrated. The residue is suspended in DME. To this suspension is added compound 6 (preparation described in Thoma et. al. J. Med Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 287.

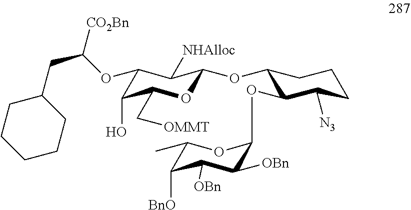

Compound 288: To a degassed solution of compound 287 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 288.

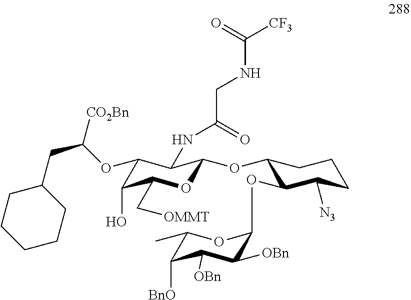

Compound 289: To a stirred solution of compound 288 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 289.

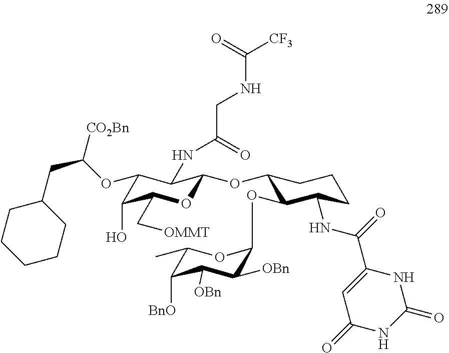

Compound 290: Compound 289 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 290.

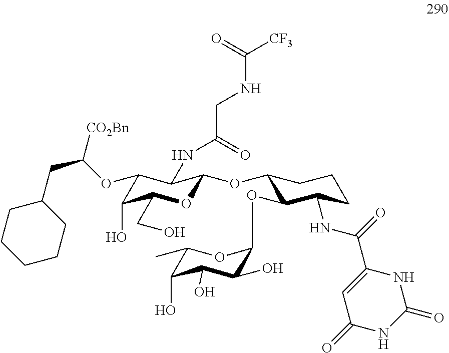

Compound 291: Compound 290 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 291.

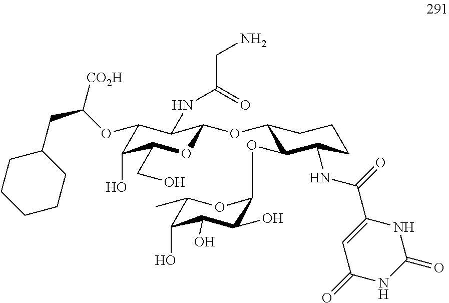

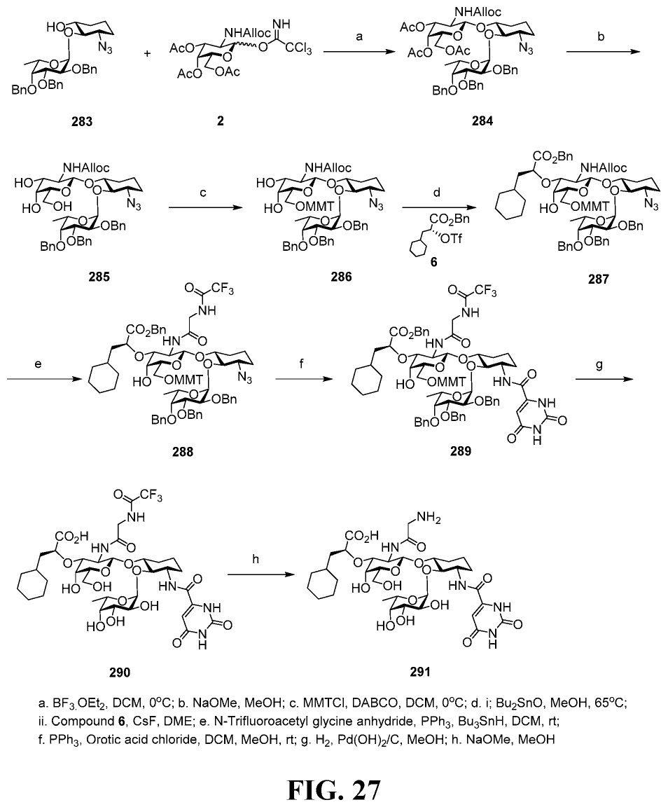

Compound 292: Compound 292 can be prepared in an analogous fashion to FIG. 27 by replacing orotic acid chloride with acetyl chloride in step f.

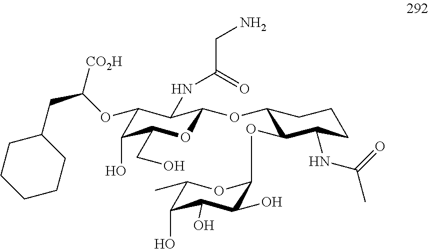

Compound 293: Compound 293 can be prepared in an analogous fashion to FIG. 27 by replacing orotic acid chloride with benzoyl chloride in step f.

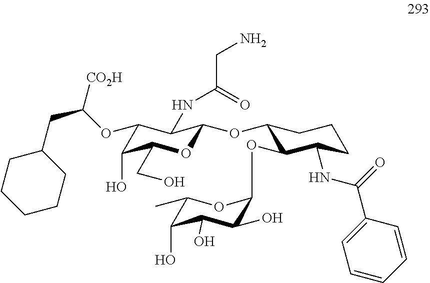

Compound 294: A solution of compound 291 (0.4 eq) in DMSO is added to a solution of compound 20 (1 eq) and DIPEA (10 eq) in anhydrous DMSO at room temperature. The resulting solution is stirred overnight. The reaction mixture is separated by reverse phase chromatography and the product lyophilized to give compound 294.

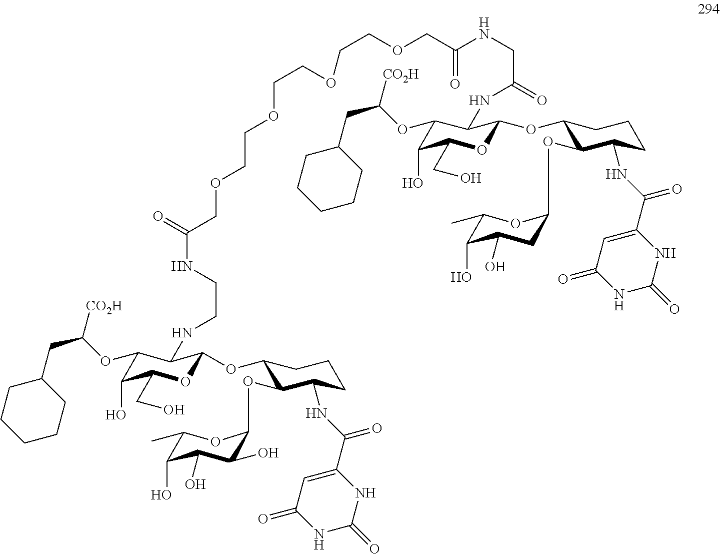

### Example 221

### Prophetic Synthesis of Multimeric Compound 295

Compound 295: Compound 294 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 295.

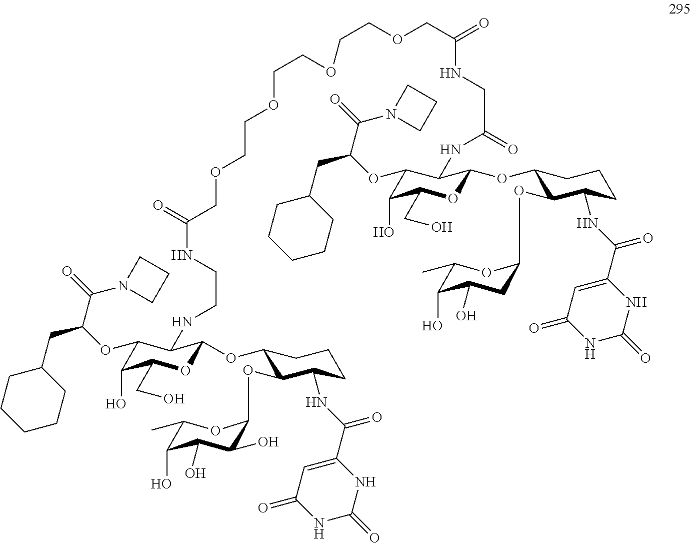

### Example 222

### Prophetic Synthesis of Multimeric Compound 296

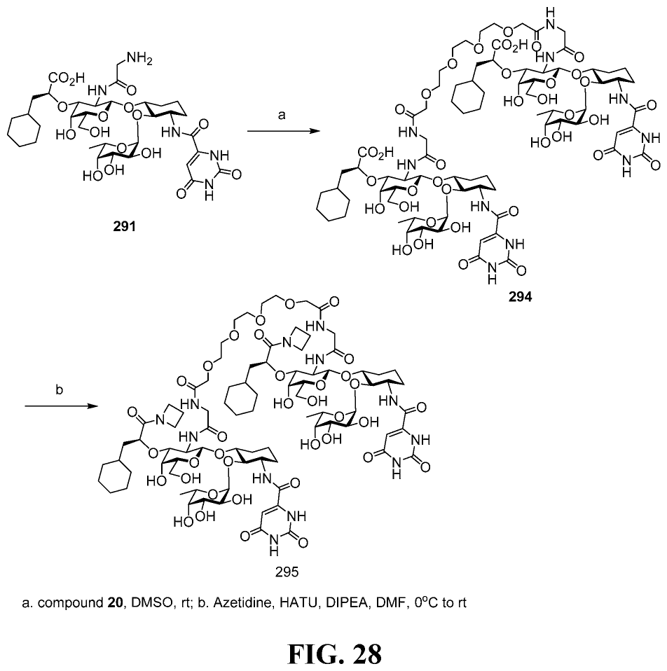

Compound 296: Compound 296 can be prepared in an analogous fashion to FIG. 28 by replacing compound 20 with ethylene glycol diacetic acid di-NHS ester in step a.

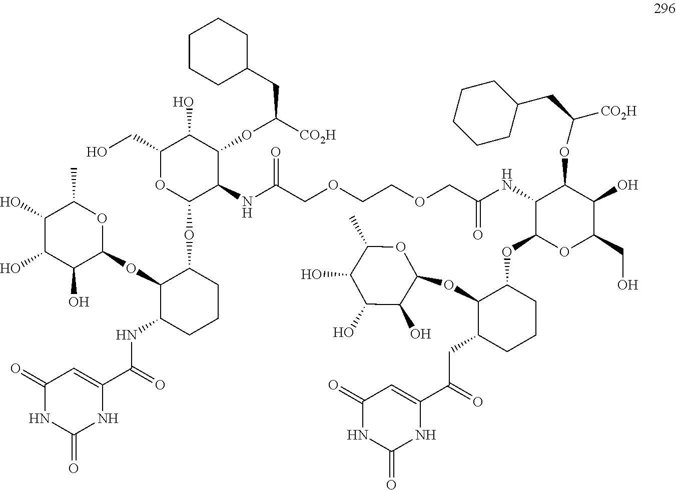 [SMILES: C[C@@H]1O[C@@H](O[C@@H]2[C@@H](CC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 223

### Prophetic Synthesis of Multimeric Compound 297

Compound 297: Compound 297 can be prepared in an analogous fashion to FIG. 28 by replacing compound 20 with ethylene glycol diacetic acid di-NHS ester in step a.

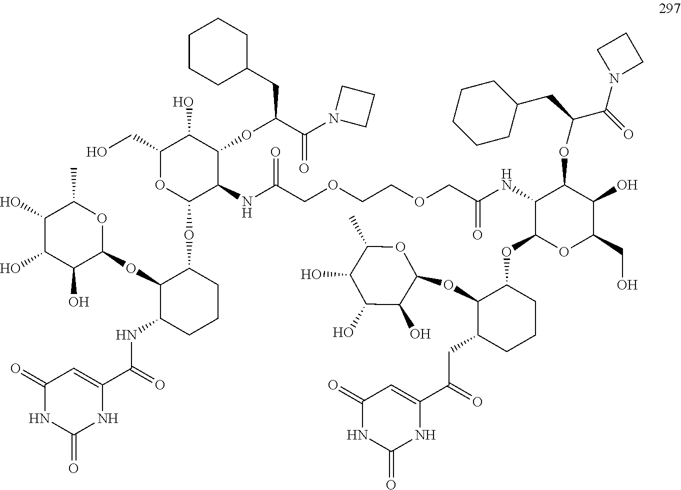 [SMILES: C[C@@H]1O[C@@H](O[C@@H]2[C@@H](CC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 224

### Prophetic Synthesis of Multimeric Compound 298

Compound 298: Compound 298 can be prepared in an analogous fashion to FIG. 28 by replacing compound 291 with compound 292 and compound 20 with ethylene glycol diacetic acid di-NHS ester in step a.

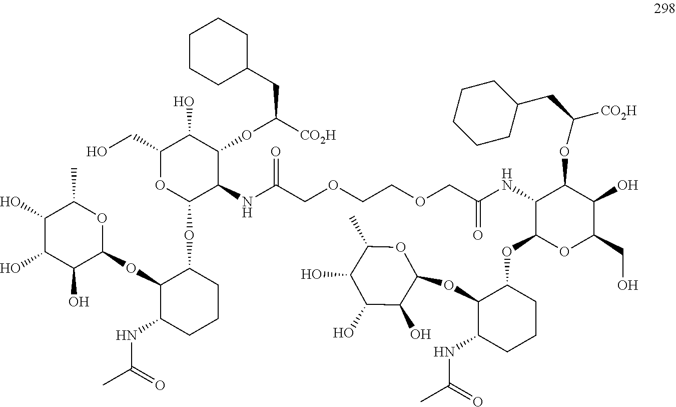 [SMILES: CC(=O)N[C@H]1CCC[C@@H](O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(C)=O)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H]1O[C@@H]1O[C@@H](C)[C@@H](O)[C@@H](O)[C@@H]1O]

### Example 225

### Prophetic Synthesis of Multimeric Compound 299

Compound 299: Compound 299 can be prepared in an analogous fashion to FIG. 28 by replacing compound 291 with compound 292 and compound 20 with ethylene glycol diacetic acid di-NHS ester in step a.

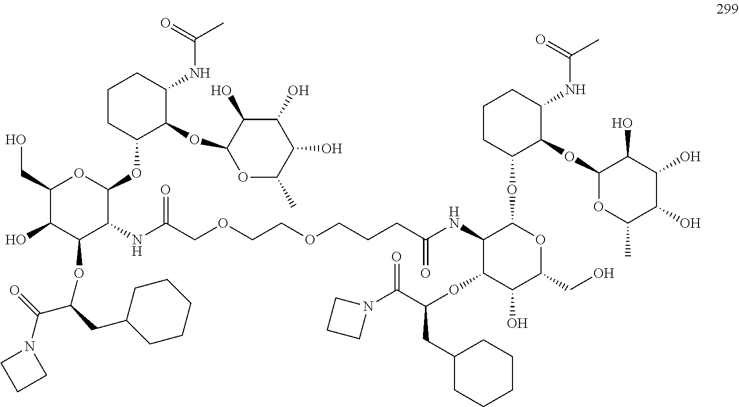 [SMILES: CC(=O)N[C@H]1CCC[C@@H](O[C@@H]2O[C@H](CO)[C@H](O)[C@H](O[C@@H](CC3CCCCC3)C(=O)N3CCC3)[C@H]2NC(=O)CCCOCCOCC(=O)N[C@H]2[C@H](O[C@@H]3CCC[C@H](NC(C)=O)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H]1O[C@@H]1O[C@@H](C)[C@@H](O)[C@@H](O)[C@@H]1O]

### Example 226

### Prophetic Synthesis of Multimeric Compound 300

Compound 300: Compound 300 can be prepared in an analogous fashion to FIG. 28 by replacing compound 291 with compound 293 and compound 20 with ethylene glycol diacetic acid di-NHS ester in step a.

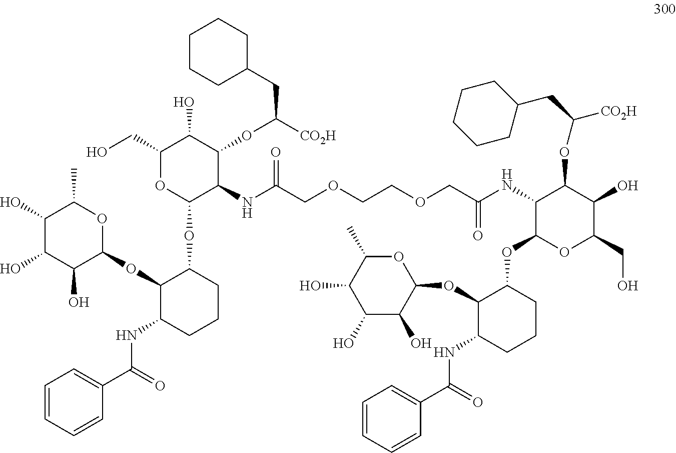 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 227

### Prophetic Synthesis of Multimeric Compound 301

Compound 301: Compound 301 can be prepared in an analogous fashion to FIG. 28 by replacing compound 291 with compound 293 and compound 20 with ethylene glycol diacetic acid di-NHS ester in step a.

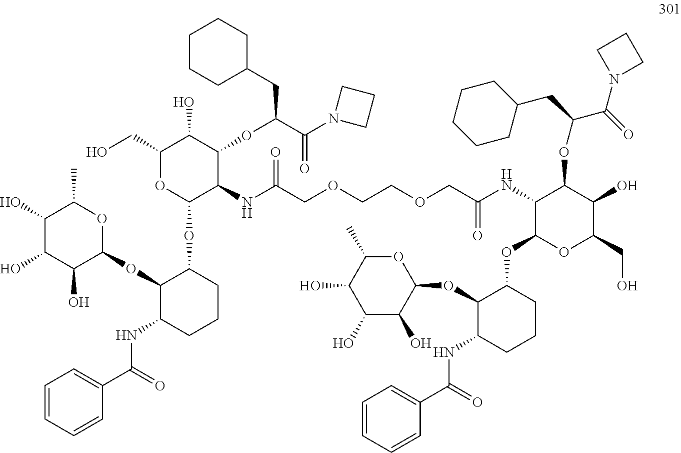 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 228

### Prophetic Synthesis of Multimeric Compound 302

Compound 302: Compound 302 can be prepared in an analogous fashion to FIG. 28 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester in step a.

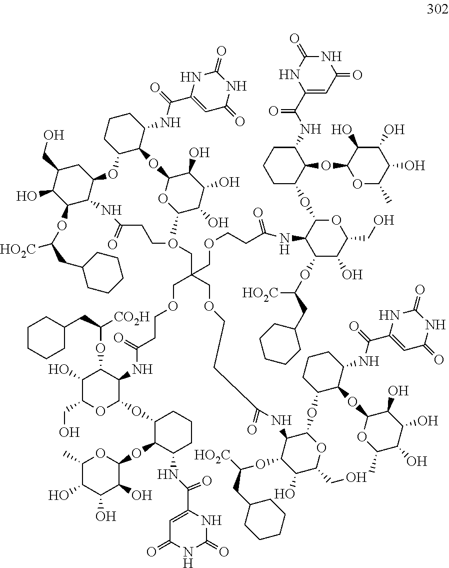

### Example 229

### Prophetic Synthesis of Multimeric Compound 305

Compound 303: To a stirred solution of compound 287 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 303.

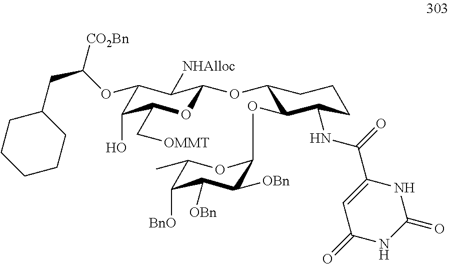

Compound 304: To a degassed solution of compound 303 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N2 atmosphere at room temperature. The reaction mixture is diluted with DCM, washed with water, dried over Na₂SO₄, then concentrated. The crude product is purified by column chromatography to give compound 304.

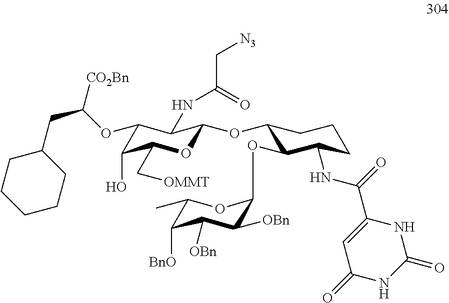

Compound 305: A solution of bispropagyl PEG-5 (compound 43) and compound 304 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is cooled to room temperature and concentrated under reduced pressure. The crude product is purified by chromatography to give compound 305.

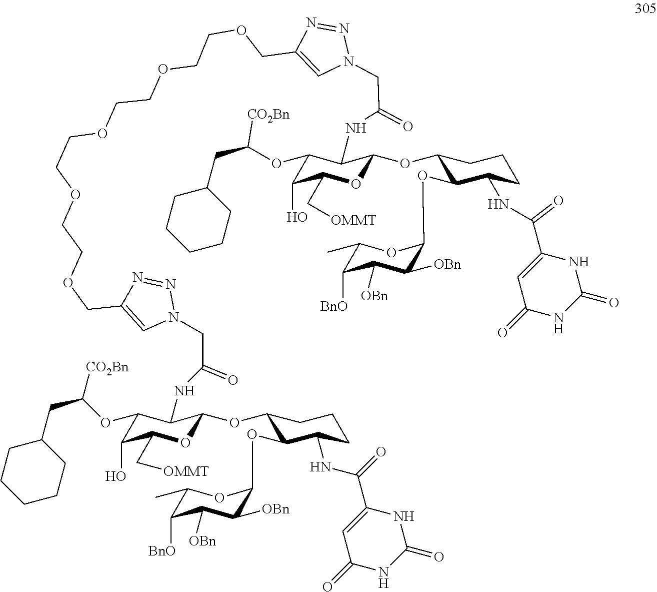

### Example 230

### Prophetic Synthesis of Multimeric Compound 306

Compound 306: Compound 305 is dissolved in MeOH and hydrogenated in the presence of Pd(OH)₂ (20 wt %) at 1 atm of H2 gas pressure for 24 hrs at room temperature. The solution is filtered through a Celite pad. The filtrate is concentrated to give compound 306.

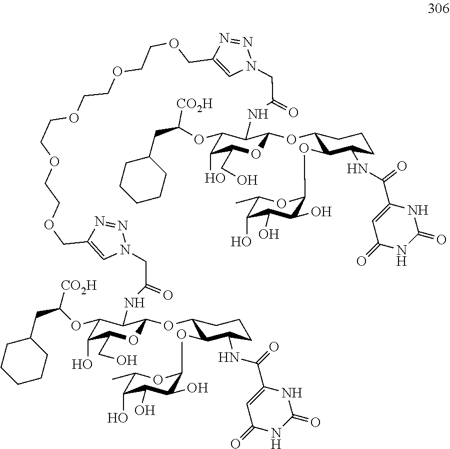

### Example 231

### Prophetic Synthesis of Multimeric Compound 307

Compound 307: Compound 306 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 307.

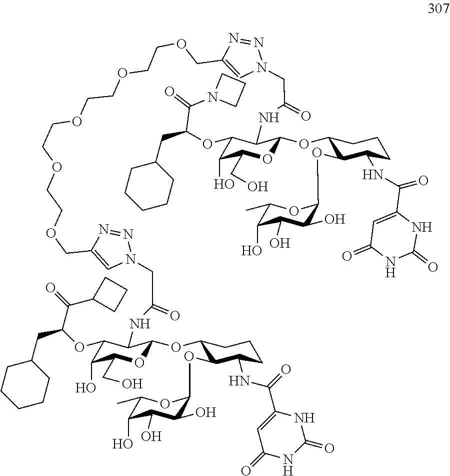

### Example 232

### Prophetic Synthesis of Multimeric Compound 308

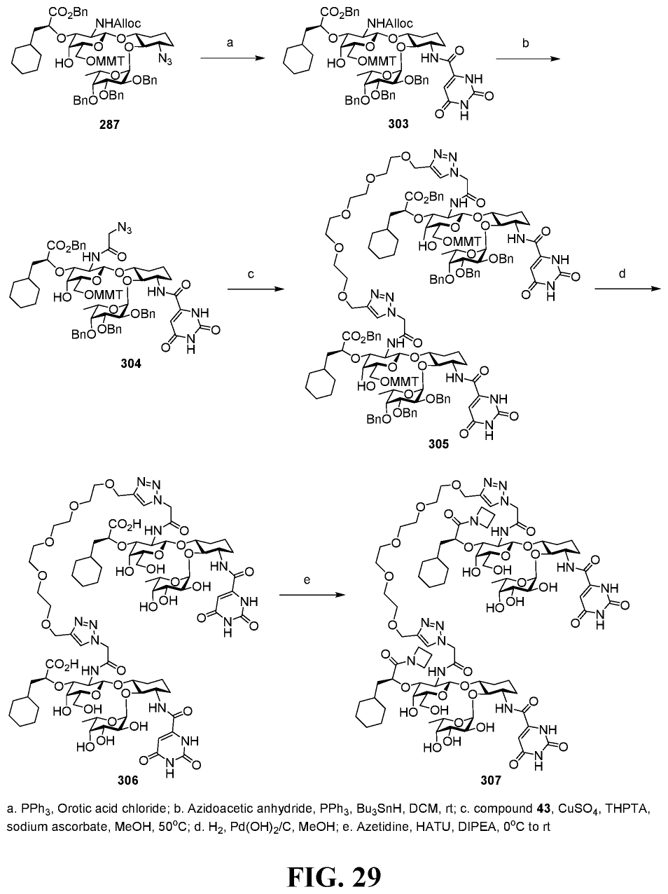

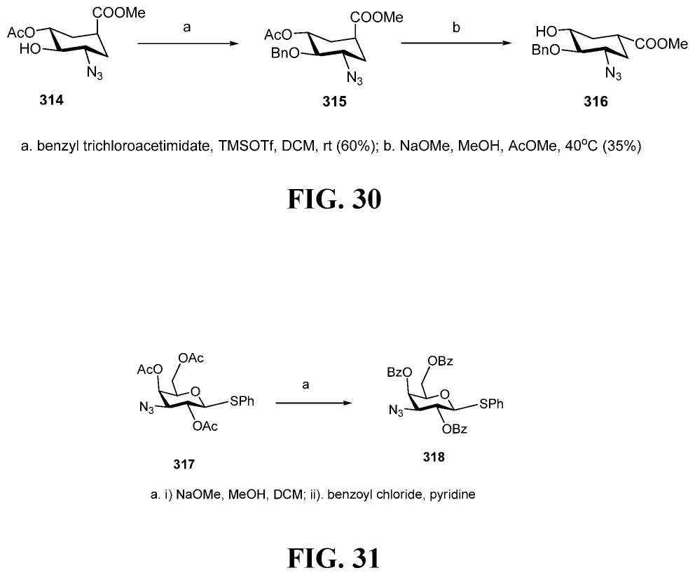

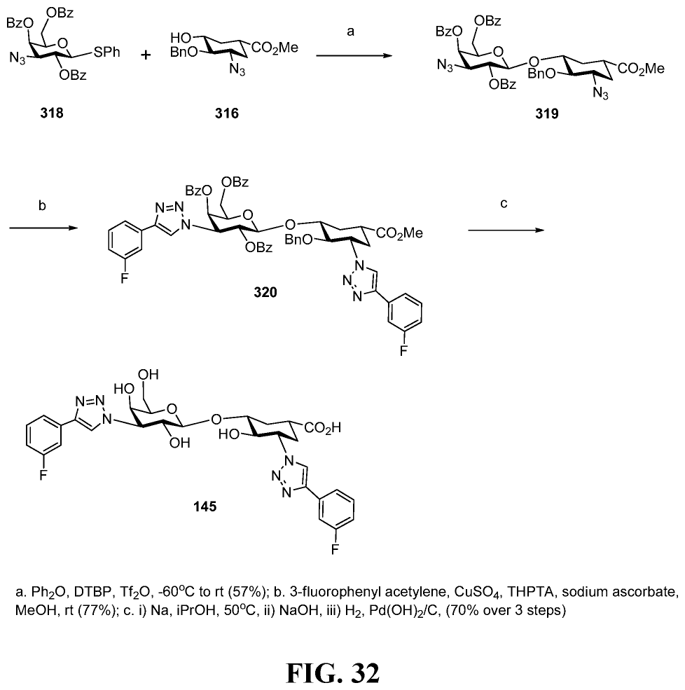

Compound 308: Compound 308 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

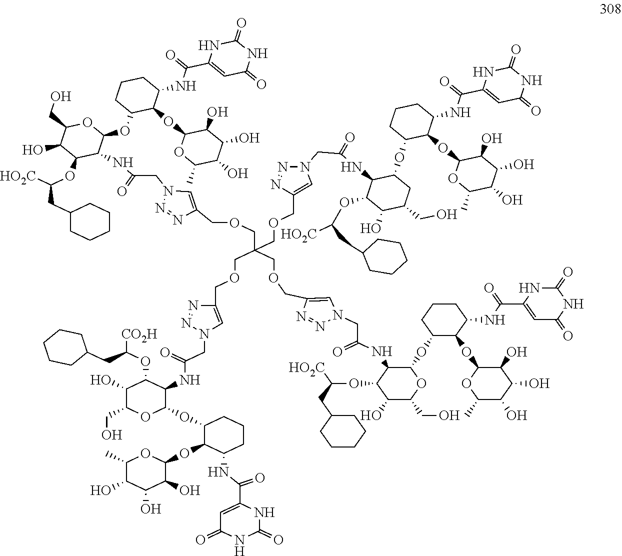

### Example 233

### Prophetic Synthesis of Multimeric Compound 309

Compound 309: Compound 309 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

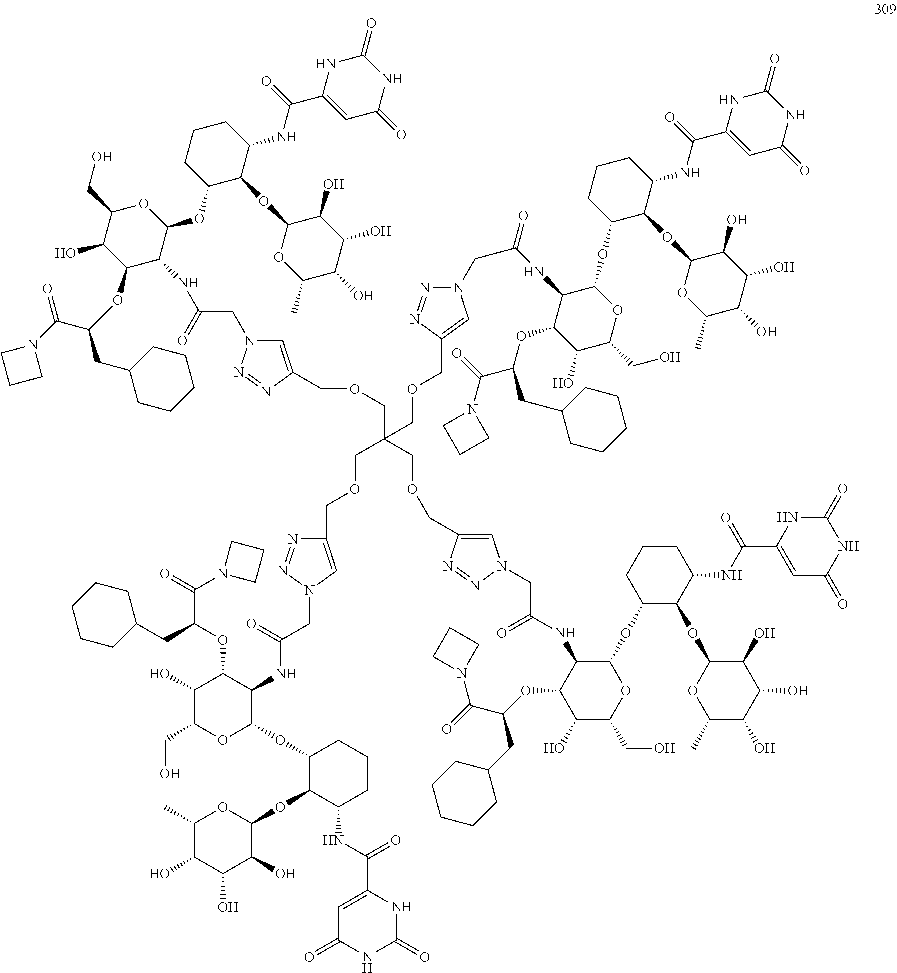

### Example 234

### Prophetic Synthesis of Multimeric Compound 310

Compound 310: Compound 310 can be prepared in an analogous fashion to FIG. 29 by replacing compound 43 with bis-propargyl ethylene glycol in step c.

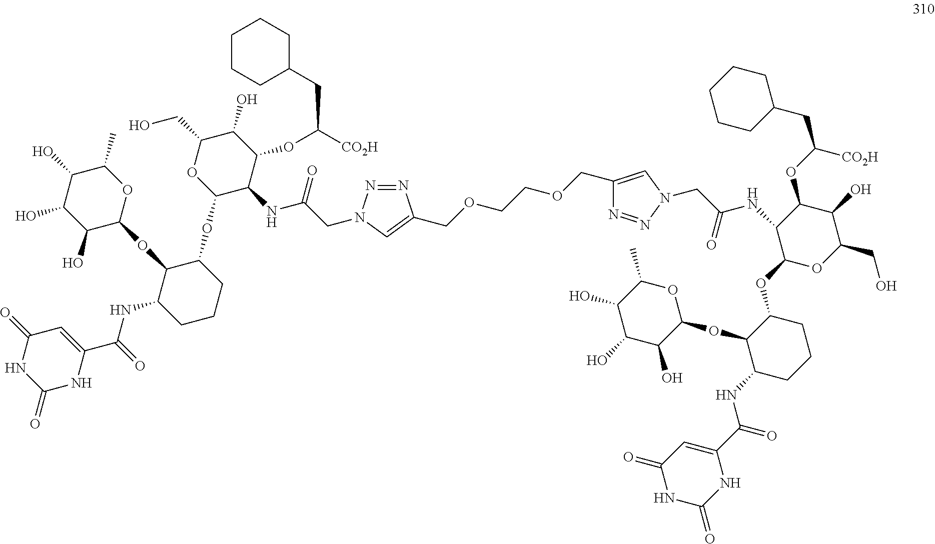 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 235

### Prophetic Synthesis of Multimeric Compound 311

Compound 311: Compound 311 can be prepared in an analogous fashion to FIG. 29 by replacing compound 43 with bis-propargyl ethylene glycol in step c.

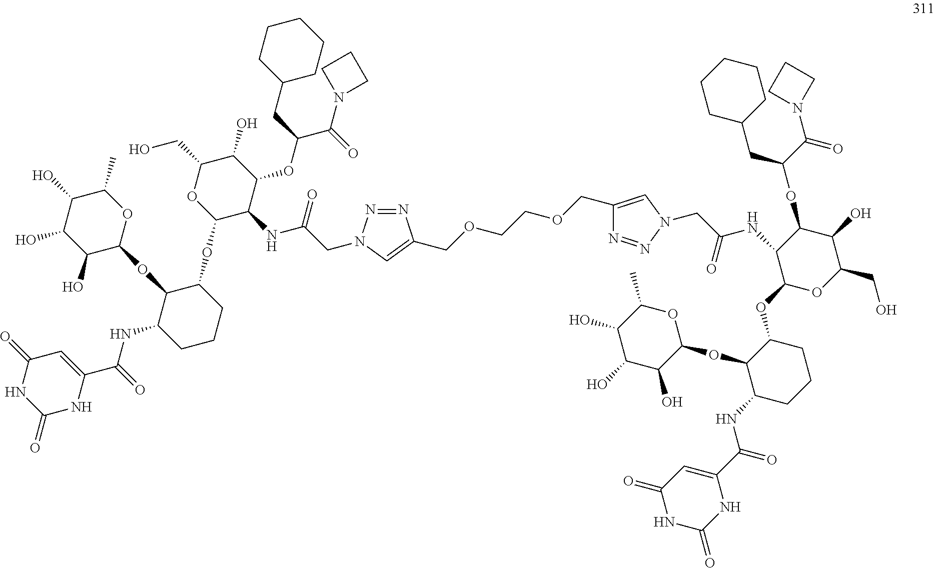 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 236

### Prophetic Synthesis of Multimeric Compound 312

Compound 312: Compound 312 can be prepared in an analogous fashion to FIG. 29 by replacing compound 43 with propargyl ether in step c.

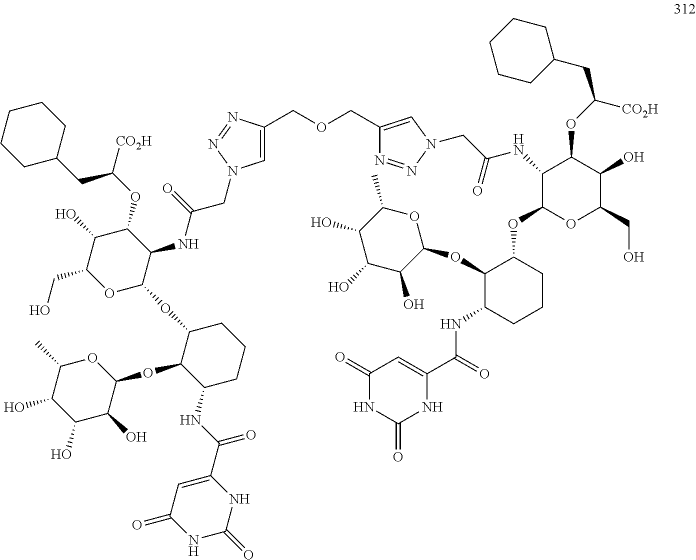 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 237

### Prophetic Synthesis of Multimeric Compound 313

Compound 313: Compound 313 can be prepared in an analogous fashion to FIG. 29 by replacing compound 43 with propargyl ether in step c.

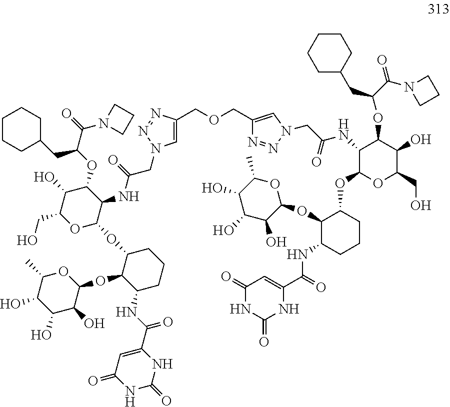 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 238

### Synthesis of Building Block 332

Compound 321: Compound 317 (1.1 g, 2.60 mmoles) was dissolved in methanol (25 mL) at room temperature. Sodium methoxide (0.1 mL, 25% sol. in MeOH) was added and the reaction mixture was stirred at room temperature for 2 hours. The reaction mixture neutralized by the addition of Amberlyst acidic resin, filtered and concentrated to give crude 321, which was used for the next step without further purification. LCMS (ESI): m/z calculated for C₁₂H₁₅N₃O₄S: 297.3, found 298.1 (M+1); 320.1 (M+Na).

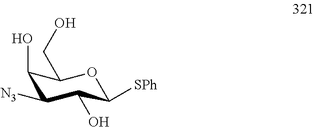

Compound 322: Crude compound 321 (2.60 mmoles), 3,4,5-trifluorophenyl-1-acetylene (2.5 equiv), THPTA (0.11 equiv), and copper (II) sulfate (0.1) were dissolved in methanol (15 mL) at room temperature. Sodium ascorbate (2.4 equiv) dissolved in water was added and the reaction mixture was stirred overnight at room temperature. The resultant precipitate was collected by filtration, washed with hexanes and water, and dried to give compound 322 as a pale yellow solid (1.2 g, 100% yield for 2 steps). LCMS (ESI): m/z calculated for C₂₀H₁₈F₃N₃O₄S: 453.1, found 454.2 (M+1); 476.2 (M+Na).

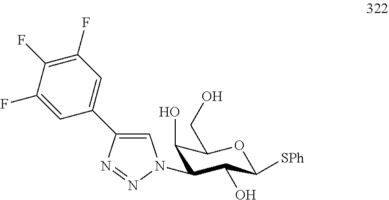

Compound 323: Compound 322 (1.2 g, 2.65 mmoles) was dissolved in DMF (15 mL) and cooled on an ice bath. Sodium hydride (60% oil dispersion, 477 mg, 11.93 mmoles) was added and the mixture stirred for 30 minutes. Benzyl bromide (1.42 mL, 11.93 mmoles) was added and the reaction was warmed to room temperature and stirred overnight. The reaction mixture was quenched by the addition of aqueous saturated ammonium chloride solution, transferred to a separatory funnel and extracted 3 times with ether. The combined organic phases were dried over magnesium sulfate, filtered, and concentrated. The residue was purified by flash chromatography to afford compound 323 (1.8 g, 94% yield). LCMS (ESI): m/z calculated for C₄₁H₃₆F₃N₃O₄S: 723.2, found 724.3 (M+1); 746.3 (M+Na).

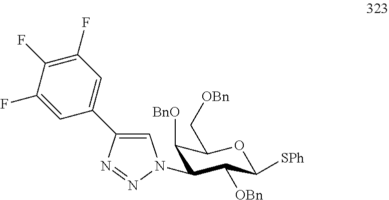

Compound 324: Compound 323 (1.8 g, 2.49 mmol) was dissolved in acetone (20 mL) and water (2 mL) and cooled on an ice bath. Trichloroisocyanuric acid (637 mg, 2.74 mmoles) was added and the reaction mixture stirred on the ice bath for 3 h. The acetone was removed in vacuo and the residue was diluted with DCM, transferred to a separatory funnel, and washed with saturated aqueous NaHCO₃. The organic phase was concentrated and the residue was purified by flash chromatography to afford compound 324 (1.5 g, 95%). LCMS (ESI): m/z calculated for C₃₅H₃₂F₃N₃O₅: 631.2, found 632.2 (M+1); 654.2 (M+Na).

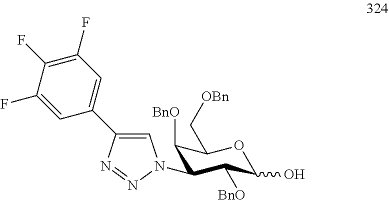

Compound 325: Compound 324 (1.0 g, 1.58 mmoles) was dissolved in DCM (20 mL) and cooled on an ice bath. Dess-Martin periodinane (1.0 g, 2.37 mmoles) was added and mixture was allowed to warm to room temperature and stirred overnight. The reaction mixture quenched by the addition of aqueous saturated NaHCO₃, transferred to a separatory funnel, and extracted 2 times with DCM. The combined organic phases were dried over sodium sulfate, filtered, and concentrated. The residue was purified by flash chromatography to afford compound 325 (520 mg, 52% yield). LCMS (ESI): m/z calculated for C₃₅H₃₀F₃N₃O₅: 629.2, found 652.2 (M+Na); 662.2 (M+MeOH+1); 684.2 (M+MeOH+Na).

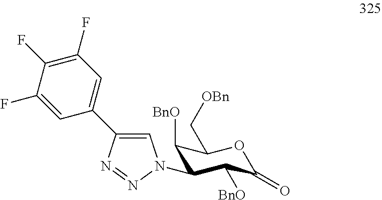

Compound 326: Methyl bromoacetate (253 mg, 1.65 mmoles) dissolved in 0.5 mL of THF was added dropwise to a solution of lithium bis(trimethylsilyl)amide (1.0 M in THF, 1.65 mL, 1.65 mmoles) cooled at −78 C. The reaction mixture was stirred for 30 minutes at −78 C. Compound 325 (260 mg, 0.41 mmoles) dissolved in THF (2.0 mL) was then added. The reaction mixture was stirred at −78 C for 30 minutes. The reaction was quenched by the addition of aqueous saturated NH₄Cl and warmed to rt. The reaction mixture was transferred to a separatory funnel and extracted 3 times with ethyl acetate. The combined organic phases were dried over sodium sulfate, filtered and concentrated. The residue was separated by flash chromatography to afford compound 326 (183 mg, 64% yield).

¹H NMR (400 MHz, Chloroform-d) δ 7.38-7.22 (m, 9H), 7.15-7.11 (m, 3H), 7.09 (dd, J=8.4, 6.6 Hz, 1H), 7.06-7.00 (m, 2H), 6.98-6.93 (m, 2H), 5.11 (dd, J=11.3, 3.2 Hz, 1H), 4.60 (d, J=11.8 Hz, 1H), 4.57-4.49 (m, 2H), 4.49-4.42 (m, 2H), 4.35 (d, J=11.8 Hz, 1H), 4.14 (d, J=3.2 Hz, 1H), 4.05 (s, 1H), 4.02 (d, J=7.0 Hz, 1H), 3.84 (d, J=11.0 Hz, 1H), 3.81 (s, 3H), 3.70 (dd, J=9.5, 7.7 Hz, 1H), 3.62 (dd, J=9.4, 6.0 Hz, 1H). LCMS (ESI): m/z calculated for C₃₈H₃₄F₃N₃O₇: 701.2, found 702.3 (M+1); 724.3 (M+Na).

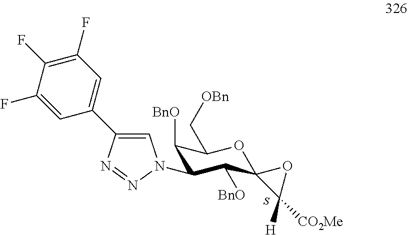

Compound 327: Compound 326 (5.0 g, 7.13 mmol) was azeotroped with toluene two times under reduced pressure, and then dried under high vacuum for 2 hours. It was then dissolved in anhydrous CH₂Cl₂ (125 mL) and cooled on an ice bath while stirring under an atmosphere of argon. Tributyltin hydride (15.1 mL, 56.1 mmol) was added dropwise and the solution was allowed to stir for 25 minutes on the ice bath. Trimethylsilyl triflate (2.1 mL, 11.6 mmol) dissolved in 20 mL of anhydrous CH₂Cl₂ was then added dropwise over the course of 5 minutes. The reaction was slowly warmed to ambient temperature and stirred for 16 hours. The reaction mixture was then diluted with CH₂C₁₂ (50 mL), transferred to a separatory funnel, and washed with saturated aqueous NaHCO₃ (50 mL). The aqueous phase was separated and extracted with CH₂Cl₂ (50 mL×2). The combined organic phases were washed with saturated aqueous NaHCO₃ (50 mL), dried over Na₂SO₄, filtered, and concentrated. The residue was purified by flash chromatography (hexanes to 40% EtOAc in hexanes, gradient) to afford compound 327 (2.65 g, 48%).

¹H-NMR (400 MHz, CDCl3): δ 7.65 (s, 1H), 7.36-7.22 (m, 8H), 7.16-7.06 (m, 7H), 6.96-6.90 (m, 2H), 5.03 (dd, J=10.7, 3.2 Hz, 1H), 4.72 (d, J=2.3 Hz, 1H), 4.51 (dt, J=22.6, 11.4 Hz, 3H), 4.41 (d, J=10.9 Hz, 1H), 4.32 (dd, J=10.7, 9.2 Hz, 1H), 4.07 (d, J=3.1 Hz, 1H), 3.94 (d, J=10.9 Hz, 1H), 3.92-3.84 (m, 3H), 3.78-3.71 (m, 4H), 3.65 (dd, J=9.1, 5.5 Hz, 1H), 0.24 (s, 9H). LCMS (ESI): m/z (M+Na) calculated for C₄₁H₄₄F₃N₃O₇SiNa: 798.87, found 798.2.

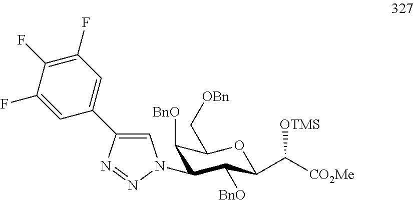

Compound 328: To a solution of compound 327 (2.65 g, 3.4 mmol) in anhydrous MeOH (40 mL) was added Pd(OH)₂ (0.27 g, 200% by wt). The mixture was cooled on an ice bath and stirred for 30 minutes. Triethylsilane (22 mL, 137 mmol) was added dropwise. The solution was allowed to slowly warm to ambient temperature and stirred for 16 hours. The reaction mixture was filtered through a bed of Celite and concentrated. The residue was purified by flash chromatography (hexanes to 100% EtOAc, gradient) to afford compound 328 (1.09 g, 73%).

¹H-NMR (400 MHz, CD₃OD): δ 8.57 (s, 1H), 7.77-7.53 (m, 2H), 4.91-4.82 (m, 1H), 4.66-4.59 (m, 1H), 4.55 (dd, J=10.8, 9.4 Hz, 1H), 4.13 (d, J=2.8 Hz, 1H), 3.86 (dd, J=9.4, 2.1 Hz, 1H), 3.81 (s, 3H), 3.77-3.74 (m, 1H), 3.71-3.68 (m, 2H). LCMS (ESI): m/z (M+Na) calculated for C₁₇H₁₈F₃N₃O₇Na: 456.33, found 456.0.

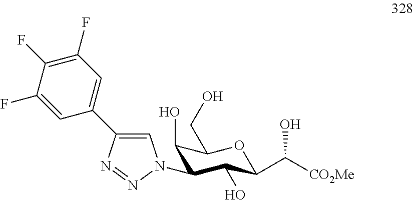

Compound 329: Compound 328 (1.09 g, 2.5 mmol) and CSA (0.115 g, 0.49 mmol) were suspended in anhydrous MeCN (80 mL) under an argon atmosphere. Benzaldehyde dimethyl acetal (0.45 mL, 2.99 mmol) was added dropwise. The reaction mixture was allowed to stir for 16 hours at ambient temperature, during which time it became a homogenous solution. The reaction mixture was then neutralized with a few drops of Et₃N, and concentrated. The residue was purified via flash chromatography (CH₂Cl₂ to 10% MeOH in CH₂C₁₂, gradient) to afford compound 329 (978 mg, 75%).

¹H NMR (400 MHz, DMSO-d6): δ 8.84 (s, 1H), 7.95-7.73 (m, 2H), 7.33 (qdt, J=8.4, 5.6, 2.7 Hz, 5H), 5.51 (t, J=3.8 Hz, 2H), 5.47 (d, J=6.8 Hz, 1H), 5.14 (dd, J=10.8, 3.6 Hz, 1H), 4.54 (dd, J=6.7, 2.2 Hz, 1H), 4.47 (ddd, J=10.8, 9.3, 7.5 Hz, 1H), 4.40 (d, J=4.0 Hz, 1H), 4.09-3.99 (m, 2H), 3.85 (dd, J=9.3, 2.2 Hz, 1H), 3.81-3.76 (m, 1H), 3.71 (s, 3H). LCMS (ESI): m/z (M+Na) calculated for C₂₄H₂₂F₃N₃O₇Na: 544.43, found 544.1.

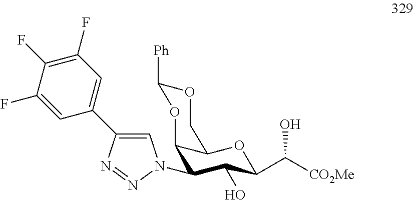

Compound 330: Compound 329 (25.2 mg, 0.048 mmol) was azeotroped with toluene 2 times under reduced pressure, dried under high vacuum for 2 hours, then dissolved in anhydrous DMF (2 mL) and cooled on an ice bath. Benzyl bromide (6 uL, 0.05 mmol) dissolved in 0.5 mL of anhydrous DMF was added and the reaction and was stirred under an atmosphere of argon for 30 minutes at 0° C. Sodium hydride (2 mg, 0.05 mmol, 60%) was added and the reaction was allowed to gradually warm to ambient temperature while stirring for 16 hours. The reaction mixture was diluted with EtOAc (20 mL), transferred to a separatory funnel, and washed with H₂O (10 mL). The aqueous phase was separated and extracted with EtOAc (10 mL×3). The combined organic phases were washed with H₂O (10 mL×3), dried over Na₂SO₄, filtered, and concentrated. The residue was purified via preparative TLC (5% MeOH in CH₂Cl₂) to afford compound 330 (6.3 mg, 21%). LCMS (ESI): m/z (M+Na) calculated for C₃₁H₂₈F₃N₃O₇Na: 634.55, found 634.1.

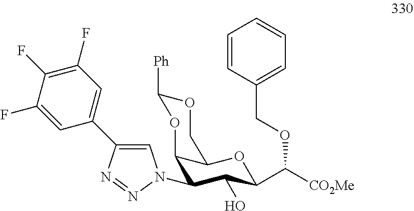

Compound 331: Compound 330 (6.3 mg, 0.01 mmol) was dissolved in anhydrous MeOH (1 mL) containing CSA (0.26 mg, 0.001 mmol). The reaction mixture was heated to 76° C. in a screw-cap scintillation vial while stirring. After 2 hours, an additional 0.13 mg of CSA in 0.5 mL of MeOH was added. The reaction mixture was stirred at 76° C. for 16 hours. The reaction mixture concentrated under reduced pressure. The residue was purified via preparative TLC (10° % MeOH in CH₂Cl₂) to afford compound 331 (4.2 mg, 80%).

¹H NMR (400 MHz, DMSO-d6) δ 8.80 (s, 1H), 7.94-7.86 (m, 2H), 7.48-7.42 (m, 2H), 7.38 (t, J=7.4 Hz, 2H), 7.36-7.28 (m, 1H), 5.46 (d, J=7.7 Hz, 1H), 5.28 (d, 1=6.0 Hz, 1H), 4.85 (dd, =10.7, 2.9 Hz, 1H), 4.67 (d, J=11.0 Hz, 1H), 4.62-4.58 (m, 1H), 4.54 (d, J=11.1 Hz, 1H), 4.44 (d, J=2.5 Hz, 1H), 4.36 (q, J=9.5 Hz, 1H), 3.95-3.90 (m, 1H), 3.78 (dd, J=9.3, 2.5 Hz, 1H), 3.71 (s, 3H), 3.61-3.54 (m, 1H), 3.52-3.43 (m, 1H), 3.43-3.38 (m, 1H). LCMS (ESI): m/z (M+Na) calculated for C₂₄H₂₄F₃N₃O₇Na: 546.45, found 546.0.

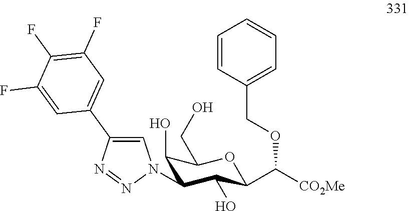

Compound 332: To a solution of compound 331 (3.5 mg, 0.007 mmoles) in methanol (0.5 mL) was added 1.0 M NaOH solution (0.1 mL). The reaction mixture was stirred overnight at room temperature then neutralized with acidic resin, filtered and concentrated. The residue was purified by reverse phase chromatography using a C-8 matrix to afford 3.0 mg compound 332 (90%).

¹H NMR (400 MHz, Deuterium Oxide) δ 8.39 (s, 1H), 8.37 (s, 2H), 7.54-7.45 (m, 1H), 7.43 (d, J=7.4 Hz, 2H), 7.35 (dt, J=14.3, 7.2 Hz, 3H), 4.86 (dd, J=11.0, 2.9 Hz, 1H), 4.76 (d, J=11.0 Hz, 1H), 4.40-4.30 (m, 2H), 4.16 (d, J=1.9 Hz, 1H), 4.04 (d, J=3.0 Hz, 1H), 3.81 (d, J=9.6 Hz, 1H), 3.73 (d, J=3.9 Hz, 0H), 3.67 (d, J=7.6 Hz, 1H), 3.56 (dd, J=11.7, 3.9 Hz, 1H). LCMS (ESI): m/z+Na calculated for C₂₃H₂₂F₃N₃O₇: 509.1, found 508.2 (M−H).

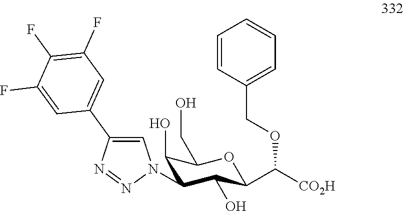

### Example 239

### Prophetic Synthesis of Building Block 333

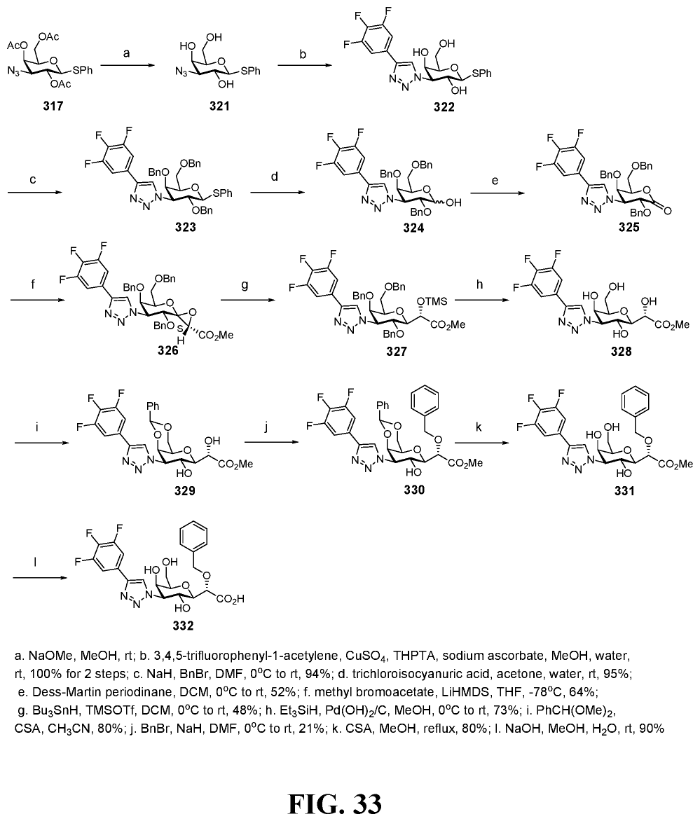

Compound 333: Compound 333 can be prepared in an analogous fashion to FIG. 33 by replacing benzyl bromide with 4-chlorobenzyl bromide in step j.

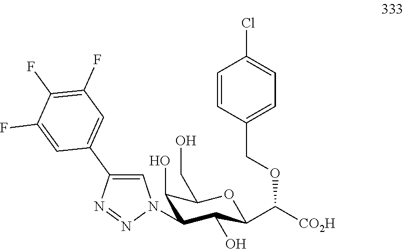

### Example 240

### Prophetic Synthesis of Building Block 334

Compound 334: Compound 334 can be prepared in an analogous fashion to FIG. 33 by replacing benz y bromide with 4-methanesulfonylbenzoyl bromide in step j.

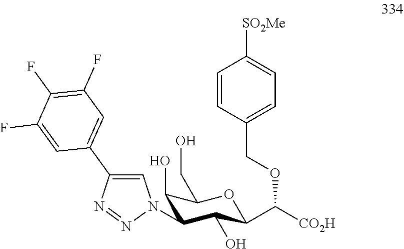

### Example 241

### Prophetic Synthesis of Building Block 335

Compound 335: Compound 335 can be prepared in an analogous fashion to FIG. 33 by replacing benzyl bromide with 3-picolyl bromide in step j.

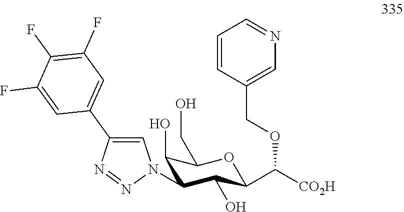

### Example 242

### Prophetic Synthesis of Multimeric Compound 336

Compound 336: Compound 336 can be prepared in an analogous fashion to FIG. 14 by replacing compound 145 with compound 332.

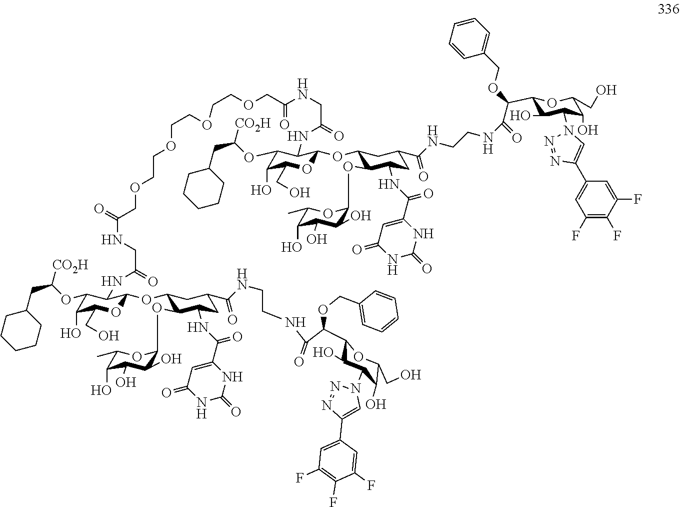

### Example 243

### Prophetic Synthesis of Multimeric Compound 337

Compound 337: Compound 337 can be prepared in an analogous fashion to FIG. 14 by replacing compound 145 with compound 333.

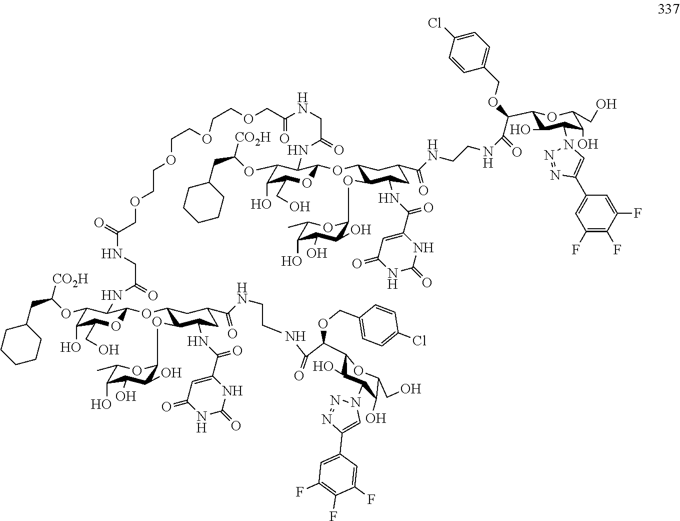

### Example 244

### Prophetic Synthesis of Multimeric Compound 338

Compound 338: Compound 338 can be prepared in an analogous fashion to FIG. 14 by replacing compound 145 with compound 334.

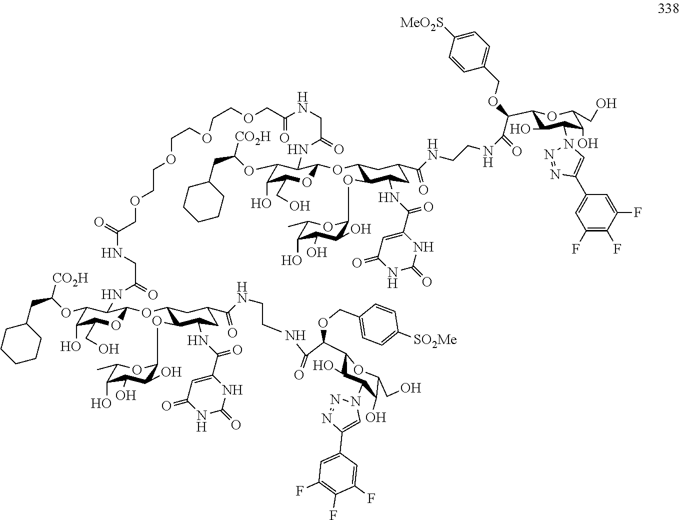

### Example 245

### Prophetic Synthesis of Multimeric Compound 339

Compound 339: Compound 339 can be prepared in an analogous fashion to FIG. 14 by replacing compound 145 with compound 335.

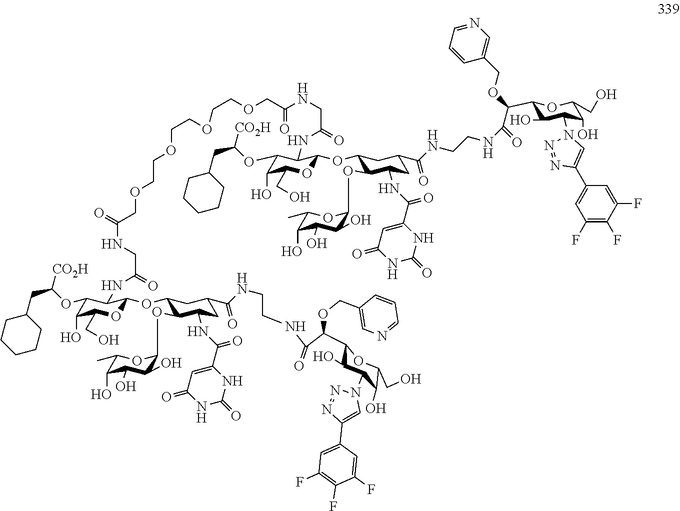

### Example 246

### Prophetic Synthesis of Multimeric Compound 340

Compound 340: Compound 340 can be prepared in an analogous fashion to FIG. 14 by replacing compound 22 with compound 40 and replacing compound 145 with compound 333.

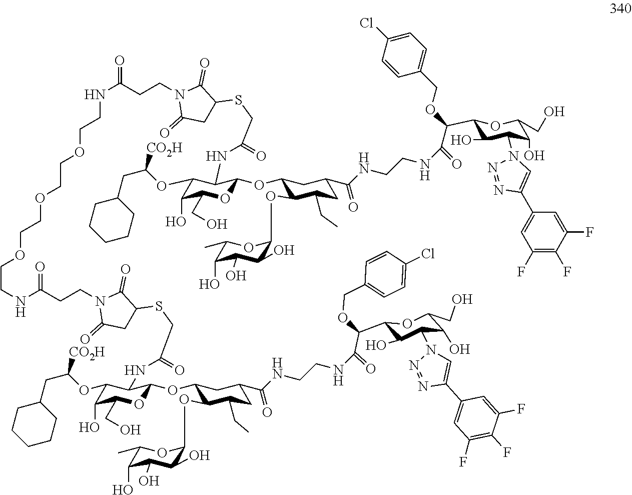

### Example 247

### Prophetic Synthesis of Multimeric Compound 341

Compound 341: Compound 341 can be prepared in an analogous fashion to FIG. 14 by replacing compound 22 with compound 78 and replacing compound 145 with compound 333.

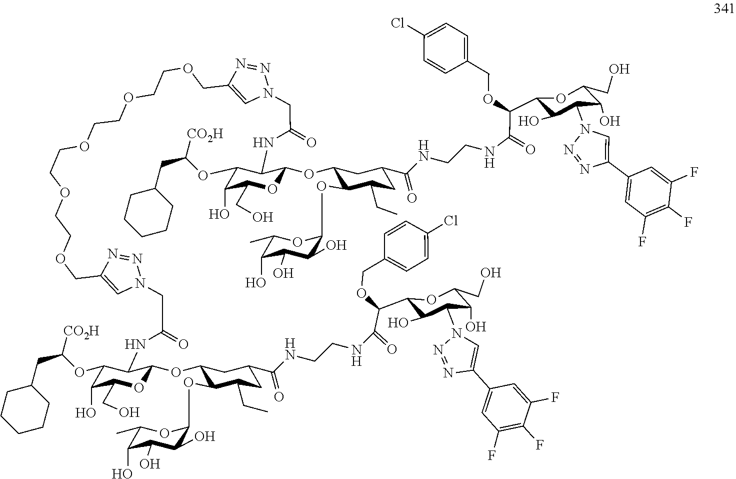

### Example 248

### Prophetic Synthesis of Multimeric Compound 342

Compound 342: Compound 342 can be prepared in an analogous fashion to FIG. 14 by replacing compound 22 with compound 87 and replacing compound 145 with compound 333.

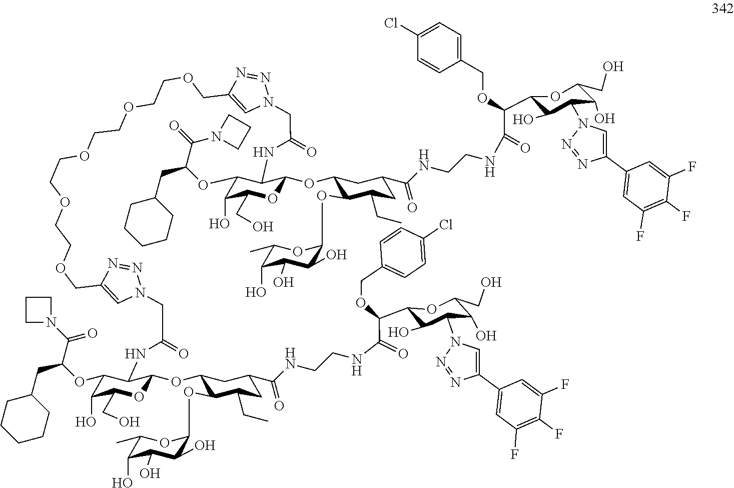

### Example 249

### Prophetic Synthesis of Multimeric Compound 343

Compound 343: Compound 342 can be prepared in an analogous fashion to FIG. 14 by replacing compound 22 with compound 88 and replacing compound 145 with compound 333.

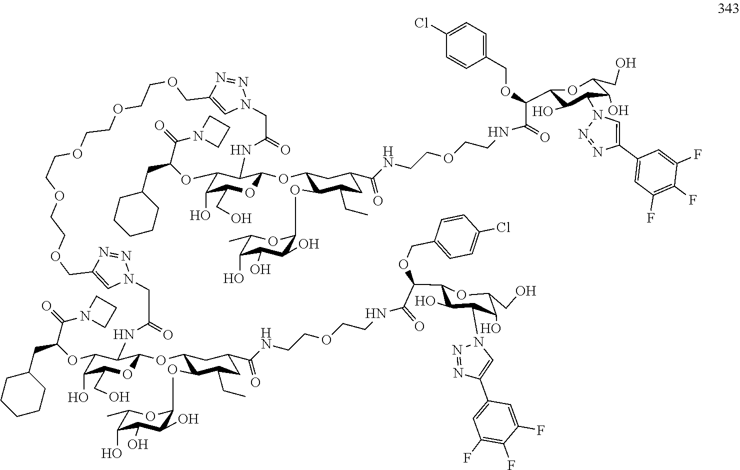

### Example 250

### E-Selectin Activity—Binding Assay

The inhibition assay to screen and characterize antagonists of E-selectin is a competitive binding assay, from which IC₅₀ values may be determined. E-selectin/Ig chimera are immobilized in 96 well microtiter plates by incubation at 37° C. for 2 hours. To reduce nonspecific binding, bovine serum albumin is added to each well and incubated at room temperature for 2 hours. The plate is washed and serial dilutions of the test compounds are added to the wells in the presence of conjugates of biotinylated, sLea polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramethylbenzidine (TMB) is added. After 3 minutes, the enzyme reaction is stopped by the addition of H₃PO₄, and the absorbance of light at a wavelength of 450 nm is determined. The concentration of test compound required to inhibit binding by 50% is determined.

E-Selectin Antagonist Activity

| Compound | IC50 (nM) |
|---|---|
| Compound 206 | 1.6 |

### Example 251

### Galectin-3 Activity—ELISA Assay

Galectin-3 antagonists can be evaluated for their ability to inhibit binding of galectin-3 to a Galβ1-3GlcNAc carbohydrate structure. The detailed protocol is as follows. A 1 ug/mL suspension of a Galβ1-3GlcNAcβ1-3Galβ1-4GlcNAcβ-PAA-biotin polymer (Glycotech, catalog number 01-096) is prepared. A 100 uL aliquot of the polymer is added to the wells of a 96-well streptavidin-coated plate (R&D Systems, catalog number CP004). A 100 uL aliquot of 1×Tris Buffered Saline (TBS, Sigma, catalog number T5912—10×) is added to control wells. The polymer is allowed to bind to the streptavidin-coated wells for 1.5 hours at room temperature. The contents of the wells are discarded and 200 uL of 1×TBS containing 1% bovine serum albumin (BSA) is added to each well as a blocking reagent and the plate is kept at room temperature for 30 minutes. The wells are washed three times with 1×TBS containing 0.1% BSA. A serial dilution of test compounds is prepared in a separate V-bottom plate (Corning, catalog number 3897). A 75 uL aliquot of the highest concentration of the compound to be tested is added to the first well in a column of the V-bottom plate then 15 ul is serially transferred into 60 uL 1×TBS through the remaining wells in the column to generate a 1 to 5 serial dilution. A 60 uL aliquot of 2 ug/mL galectin-3 (IBL, catalog number IBATGP0414) is added to each well in the V-bottom plate. A 100 uL aliquot of the galectin-3/test compound mixture is transferred from the V-bottom plate into the assay plate containing the Galβ1-3GlcNAc polymer. Four sets of control wells in the assay plate are prepared in duplicate containing 1) both Galβ1-3GlcNAc polymer and galectin-3, 2) neither the polymer nor galectin-3, 3) galectin-3 only, no polymer, or 4) polymer only, no galectin-3. The plate is gently rocked for 1.5 hours at room temperature. The wells are washed four times with TBS/0.1% BSA. A 100 uL aliquot of anti-galectin-3 antibody conjugated to horse radish peroxidase (R&D Systems, from DGAL30 kit) is added to each well and the plate is kept at room temperature for 1 hour. The wells are washed four times with TBS/0.1% BSA. A 100 uL aliquot of TMB substrate solution is added to each well. The TMB substrate solution is prepared by making a 1:1 mixture of TMB Peroxidase Substrate (KPL, catalog number 5120-0048) and Peroxidase Substrate Solution B (KPL, catalog number 5120-0037). The plate is kept at room temperature for 10 to 20 minutes. The color development is stopped by adding 100 uL 10% phosphoric acid (RICCA Chemical Co., catalog number 5850-16). The absorbance at 450 nm (A₄₅₀) is measured using a FlexStation 3 plate reader (Molecular Devices). Plots of A₄₅₀ versus test compound concentration and IC₅₀ determinations are made using GraphPad Prism 6.

### Example 252

### CXCR4 Assay—Inhibition of Cyclic Amp

The CXCR4-cAMP assay measures the ability of a glycomimetic CXCR4 antagonist to inhibit the binding of CXCL12 (SDF-1α) to CHO cells that have been genetically engineered to express CXCR4 on the cell surface. Assay kits may be purchased from DiscoveRx (95-0081E2CP2M; cAMP Hunter eXpress CXCR4 CHO-K1). The Gi-coupled receptor antagonist response protocol described in the kit instruction manual can be followed. GPCRs, such as CXCR4, are typically coupled to one of the 3 G-proteins: Gs, Gi, or Gq. In the CHO cells supplied with the kit, CXCR4 is coupled to Gi. After activation of CXCR4 by ligand binding (CXCL12), Gi dissociates from the CXCR4 complex, becomes activated, and binds to adenylyl cyclase, thus inactivating it, resulting in decreased levels of intracellular cAMP. Intracellular cAMP is usually low, so the decrease of the low level of cAMP by a Gi-coupled receptor will be difficult to detect. Forskolin is added to the CHO cells to directly activate adenylyl cyclase (bypassing all GPCRs), thus raising the level of cAMP in the cell, so that a Gi response can be more easily observed. CXCL12 interaction with CXCR4 decreases the intracellular level of cAMP and inhibition of CXCL12 interaction with CXCR4 by a CXCR4 antagonist increases the intracellular cAMP level, which is measured by luminescence.

### Example 253

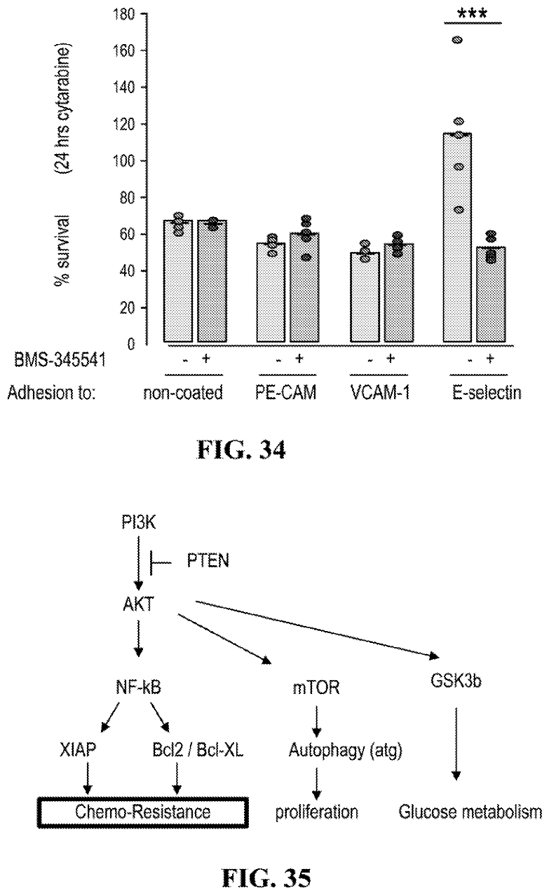

Acute myelogenous leukemia (AML) cells may express the carbohydrate structures that contain the E-selectin ligand. When these AML cells circulate through the BM microvasculature, they will adhere to E-selectin, which in turn activates the NfkB pathway, causing chemoresistance. See FIG. 34 and FIG. 35; Winkler I. G. et al., Blood 128: 2823 (2016), which is incorporated by reference in its entirety.

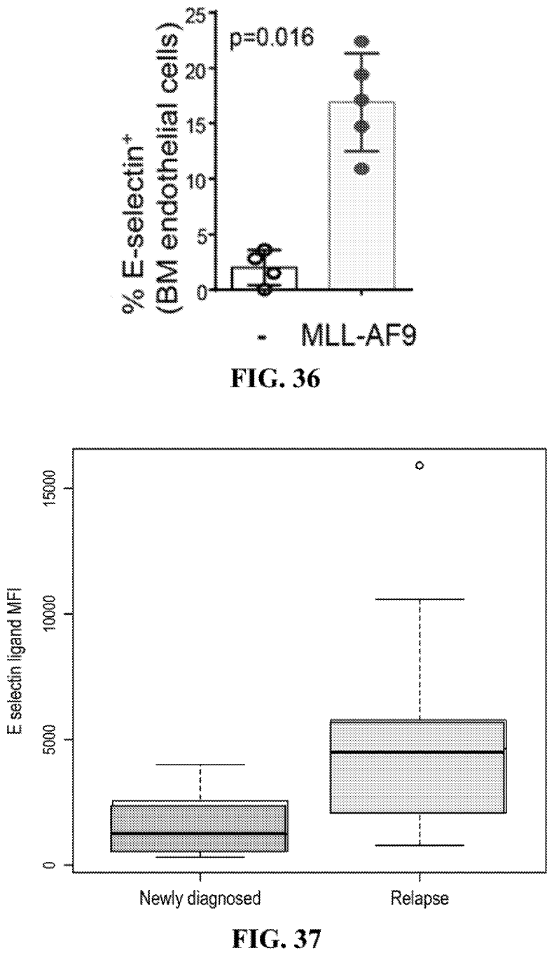

The result is that AML patients undergoing chemotherapy treatment will have those AML cells expressing high levels of the E-selectin ligands adhered to E-selectin in the microvasculature of these protective microdomains in the BM. These bound AML cells are chemoresistant and will be a source of surviving AML cells during relapse. This mechanism predicts that AML cells from relapsed patients should express higher levels of the E-selectin ligands. Indeed, mice engrafted with murine AML cells from the MLL-AF9 cell line showed higher expression of E-selectin on the surface of bone marrow endothelial cells than control animals (FIG. 36). As shown in FIG. 37, the AML cells from relapsed patients do, indeed, express significantly higher levels of the E-selectin ligand compared to AML cells from newly diagnosed patients.

In order to treat AML patients effectively, there is a need for understanding the mechanisms of leukemic cell chemotherapy evasion. Drugs, e.g., E-selectin inhibitors, that can be used alone, or in combination with chemotherapy, to treat relapsed/refractory AML are also desired. It may also be useful to identify patient subpopulations that are more or less likely to build chemoresistance, and protein or gene biomarkers, e.g., those involved in E-selectin ligand biosynthesis or metabolism, that may serve as effective biomarkers for identifying such patient subpopulations.

E-selectin antagonists, such as the compound of Formula I, which interrupt leukemic cell homing to the vascular niche and increase susceptibility to cytotoxic therapies, can be potent adjuncts to therapeutics.

Recent data demonstrated a correlation between leukemic cell surface levels of E-selectin ligands and response to the compound of Formula I, linking E-selecting ligand expression to E-selectin antagonist response (DeAngelo et al. 2018).

Multiple genes involved in the glycan synthesis of E-selectin ligands are highly expressed in pediatric AML. These genes provide novel therapeutic targets for overcoming drug resistance induced by the tumor microenvironment and lend support for the use of E-selectin ligand glycosylation genes as predictive biomarkers.

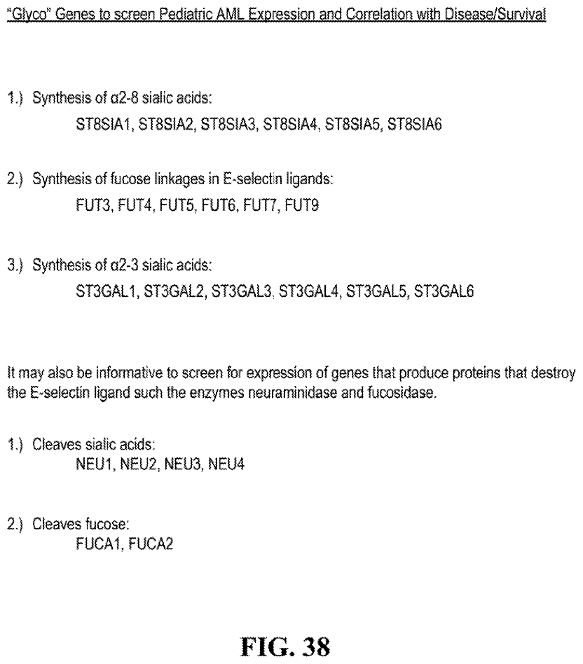

24 different genes (FIG. 38) that code for enzymes that either build carbohydrate chains (glycosyltransferases) or enzymes that destroy carbohydrate chains (glycosidases) may be analyzed for expression of the E-selectin ligand.

High coverage single strand mRNA sequencing may be performed on clinical samples from pediatric AML patients (0 to 30 years old). The data from this analysis may then be screened for expression of the 24 different genes listed in FIG. 38.

We questioned whether transcriptome profiling of E-selectin ligand forming glycosylation genes can be used to identify elevated E-selectin ligand expression in patients with cancers such as acute myeloid leukemia (AML), and subsequently which patients might benefit from and respond to E-selectin antagonists.

RNA-seq data from patients treated in COG AAML1031 (N=1,074) was available for evaluation. We examined transcriptome expression of 24 genes that code for enzymes involved in glycosylation of E-selectin ligands. All analyses were performed in R (v 3.5.2). Cox proportional hazards models were generated using the survival package (v 2.44-1.1). Multidimensional flow cytometry (MDF) was used to detect cell surface E-selectin ligand expression by two techniques: direct binding of an E-sel/hIg, PE labeled chimera, and the anti-sLe^{x} antibody HECA-452.

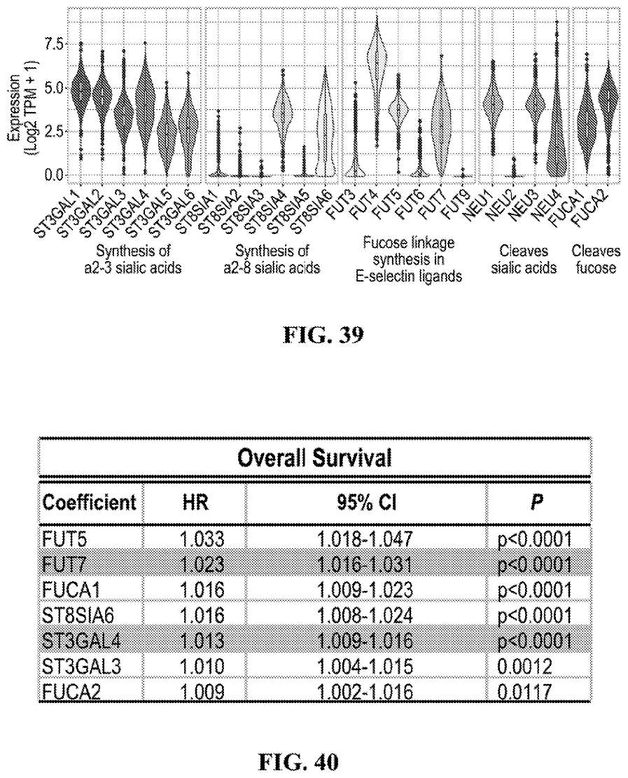

Seven of the 24 genes examined had minimal expression (mean <1 TPM) and were excluded from further analysis. The remaining 17 were variably expressed (FIG. 39). To assess association of expression with outcome, univariate Cox models for overall survival (OS) were generated, using gene expression as a continuous coefficient (N=1,061). Of the 17 genes, 7 were significantly associated with increased risk (p<0.05, FIG. 40).

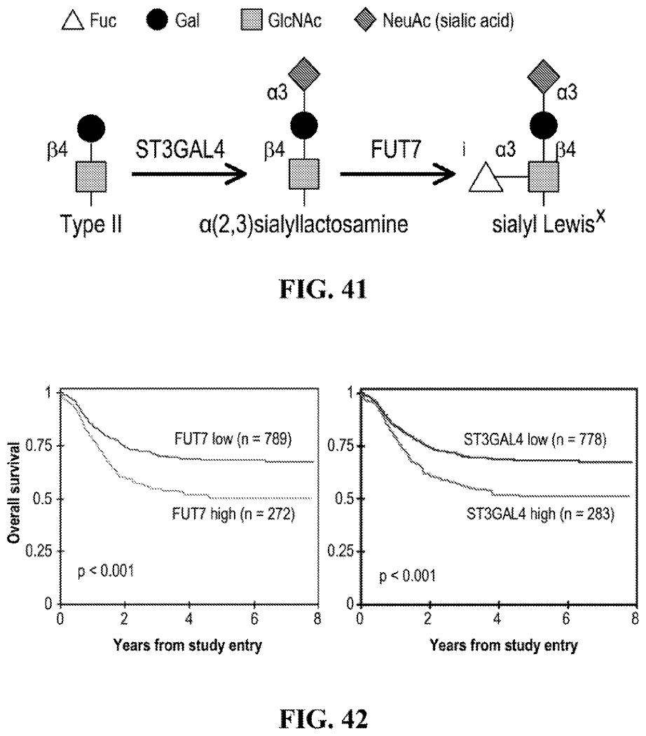

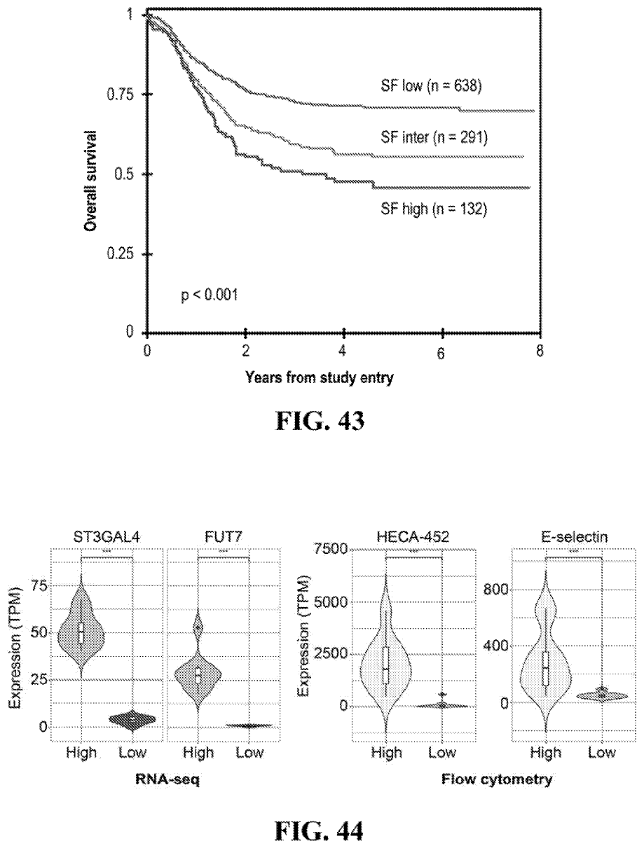

ST3GAL4 and FUT7 were targeted for further evaluation, as they directly synthesize sLe^{x} (FIG. 41) and were significantly associated with adverse outcome (HR=1.013, p<0.0001, and HR=1.023, p<0.0001, respectively). Patients highly expressing FUT7 (highest quartile of expression) had significantly worse outcome than lower expressors (lowest 3 quartiles of expression), with a 5-year OS of 50.3% vs. 68.3% (p<0.0001, FIG. 42). Similarly, those with high ST3GAL4 expression had a 5-year OS of 51.3%, compared to 68.1% for low expressors (p<0.0001, FIG. 42). A subset of patients highly expressed both genes (ST3GAL4 and FUT7 high; SF^{high}, N=132). Compared to patients that did not highly express either gene (SFI^{low}), these individuals had particularly adverse survival (45.8% OS vs 71.0% OS, p<0.0001). Patients with high expression of only one of the two genes (SF^{inter}) had a 5-year OS of 55.5%, illustrating what may be a compounding unfavorable impact conferred on survival by these genes (FIG. 43). Further investigation of clinical characteristics within these 3 groups revealed that 71.5% of infants <1 year old were SFI^{low}, with only 4.66% in SF^{high}. In addition, CBF-AML was greatly underrepresented in SF^{low}, with 97% of both t(8;21) and inv(16) patients in SF^{low}, and 0% in SF^{high}.

### Example 254

To verify surface protein expression of the two genes, leukemic specimens from SF^{high} patients (N=10) and SF^{low} patients (N=10) underwent cell surface expression evaluation of glycosylated E-selectin ligands using two MDF assays. SF^{low} patients had low or undetectable levels of cell surface E-selectin ligands by both assays, whereas SF^{high} patients had significantly higher expression of E-selectin ligands (p<0.001, FIG. 44). This suggests a strong correlation between transcriptome measurements of E-selectin ligand glycosylation genes and cell surface glycosylation levels of E-selectin ligands.

### Example 255

Expression levels of ST3GAL4 and FUT7, are associated with poor outcome. Additionally, high expression of these genes is detectable at the transcript level and associated with cell surface E-selectin ligand expression (Leonti et al. 2019).

Transcriptome profiling of E-selectin ligand-forming glycosylation genes was extended with an emphasis on ST3GAL4 and FUT7 in different cancers and in adult AML. Initially, expression levels of ST3GAL4 and FUT7 in 10,258 samples covering 33 cancer types from the TCGA PanCanAtlas were investigated. ST3GAL4 and FUT7 were consistently expressed in most of the cancers evaluated. The cancer types that expressed ST3GAL4 most highly were melanoma (uvual and skin), kidney chromphobe adrenocortical carcinoma and bladder urothelial carcinoma, while FUT7 was expressed most highly by AML, diffuse large B cell lymphoma, thymoma, testicular germ cell tumors, and head and neck squamous cell carcinoma.

Of particular interest was the identification of adult AML for the highest expression of FUT7 with high expression levels of ST3GAL4 (mean log 2 gene expression=8.1 and 9.4, respectively). Augmented expression of FUT7 was also observed in an analysis of 39 AML cell lines among the 1,457 cell lines comprising the Cancer Cell Line Encyclopedia RNAseq data set. The prognostic significance of FUT7 and ST3GAL4 in adult AML was further assessed using the TCGA-LAML RNAseq dataset for differential expression and associations with overall survival (OS).

The observed expression may then be correlated with the clinical outcome of overall survival (OS).

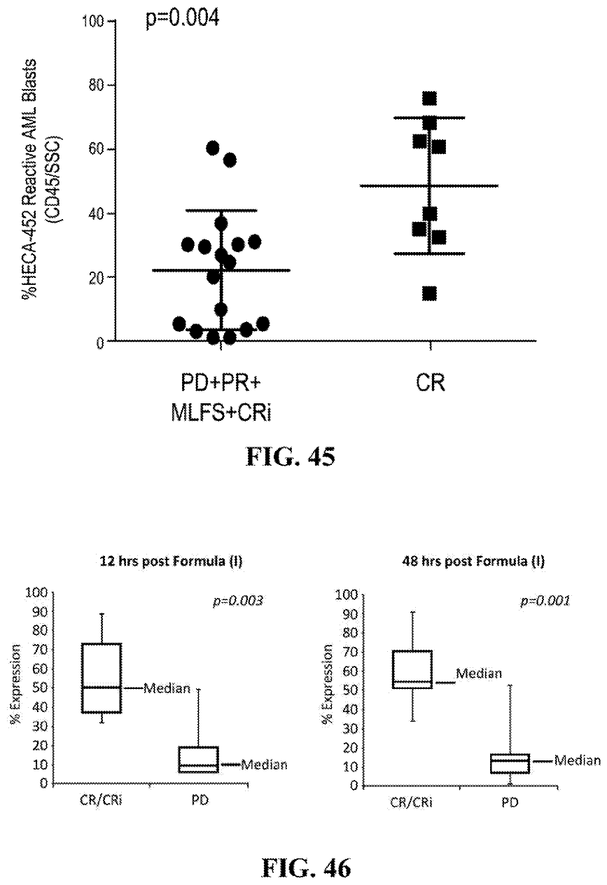

Treatment of AML relapsed/refractory patients with the compound of Formula I and chemotherapy were evaluated for response. Those patients with higher percentages of AML blasts expressing E-selectin ligands either in the BM (FIG. 45) or in the peripheral blood (FIG. 46) were more likely to have a complete response compared with those patients with lower percentages of blasts expressing E-selectin ligand.

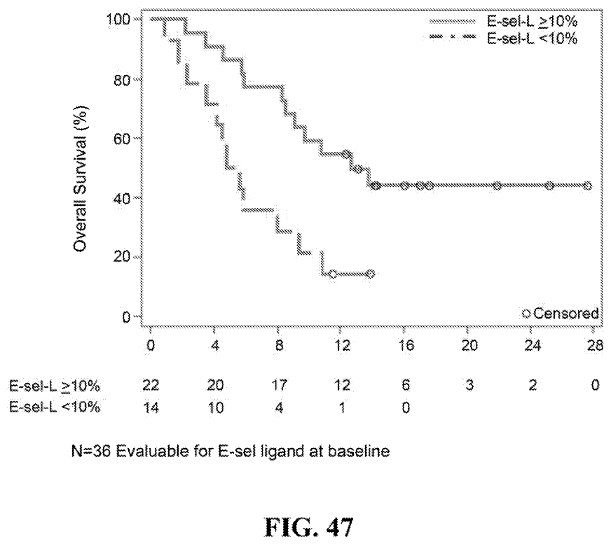

The association was also observed to contribute to better overall survival (OS). As shown in FIG. 47, treatment with the compound of Formula I has a much greater effect on extending overall survival to those patients whose AML blasts express higher levels of the E-selectin ligand as determined by binding to the anti-sialyl Le^{a/x} antibody, HECA-452. Patients with less than 10% blasts expressing the E-selectin ligand (“low expressers”) have an OS of 5.2 months. Patients with greater than 10% of blasts expressing the E-selectin ligand (“high expressers”) have an OS of 12.7 months. The clear benefit (highly significant, P=0.0056) of treatment with the compound of Formula I is observed in patients expressing the E-selectin ligand, as their E-selectin-mediated chemoresistance is broken by treatment with the E-selectin antagonist, the compound of Formula I. It is hypothesized that those patients with lower percentages (<10%) of blasts expressing E-selectin were probably chemoresistant (relapsed/refractory) by a different mechanism not involving E-selectin and therefore, the compound of Formula I showed less efficacy with a significantly lower OS (5.2 months).

### Example 256

The data set of the present disclosure included 151 RNAseq profiles of bone marrow samples from adult patients with AML, and within this data set the status of the FMS-like tyrosine kinase 3 (FLT3) proto-oncogene was considered.

Mutational alterations of FLT3 are associated with higher risk of relapse and shorter OS compared with wild-type FLT3. ST3GAL4 and FUT7 were both identified as being upregulated (fold-change=1.73 and 1.40, respectively) in the mutated FLT3 subset (n=46) as compared to wild type FLT-3 (p=0.000033 and 0.046, respectively). Notably in the FLT3-ITD mutated subset expression of FUT7 was significantly associated with a poor prognosis and decreased OS (Hazard Ratio=0.223, p=0.015).

Mutations in FLT3 tyrosine kinase are detected in about 1/3 of patients newly diagnosed for acute myelogenous leukemia (AML). About 3/4 of these mutations are internal tandem duplications (FLT3-ITD) and the rest (1/4) have missense mutations within the tyrosine kinase domain activation loop (TKD) (See Thiede C. et al., Blood 99: 4326-4335 (2002), which is incorporated by reference in its entirety). Both mutations cause constitutive kinase activation and are associated with aggressive proliferative disease and poor survival (See Yamamoto Y. et al., Blood 97: 2434-2439 (2001), which is incorporated by reference in its entirety). In particular, the FLT3-ITD mutation is a strong risk factor for relapse after treatment (See Schnittger S. et al., Blood 100: 59-66 (2002), which is incorporated by reference in its entirety).

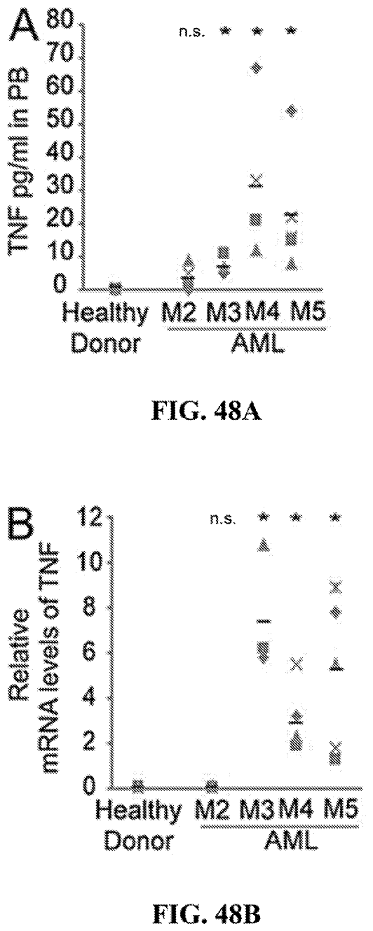

Patients expressing various subtypes of AML blast cells (M2, M3, M4, and M5) are known to contain high levels of TNFα circulating in their peripheral blood (See Volk A. et al., J. Exp. Med. 211: 1093-1108 (2014), which is incorporated by reference in its entirety; FIG. 48A, as well as high levels of TNFα mRNA expression in AML leukemic cells (LC) (See id., FIG. 48B). It has been suggested that secreted TNFα generates a proinflammatory environment which may provide a more favorable tumor microenvironment. It is well known that TNFα stimulates the expression of E-selectin, which is a marker for endothelial activation and inflammation. (See id.).

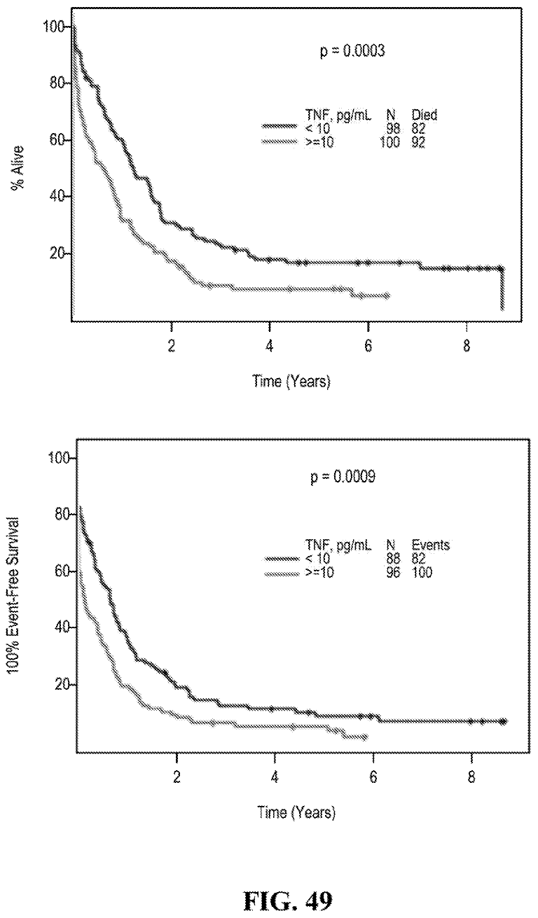

Cytokines TNFα, IL-1, IL-6, IL-10 and endostatin were measured in AML patients, and only TNFα levels correlated with poor survival. (See Tsimberidou A. M. et al., Cancer, 113: 1605-1613 (2008), which is incorporated by reference in its entirety). Of these cytokines, TNFα is well known to stimulate expression of E-selectin. Previous data show that a high serum TNFα level is an adverse prognostic factor for overall survival and event-free survival in patients with untreated AML or high-risk MDS. In contrast, low TNFα levels (<10 pg/ml) were associated with higher rates of complete remission (P=0.003), survival (P=0.0003), and event-free survival (EFS) (P=0.0009). High expression of TNFα is associated with poor survival and also poor event free survival (See Tsimberidou A. M. et al., Cancer, 113: 1605-1613 (2008), FIG. 49).

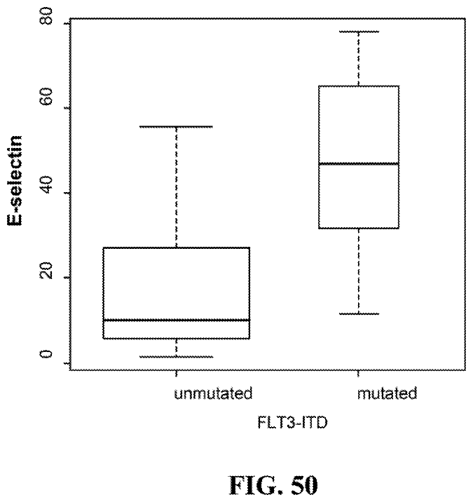

The hallmark of the AML cells containing mutations in the FLT3 gene is the constitutive kinase activation of these cancer cells. These highly activated cells are expected to produce higher levels of cytokines. A previous group examined relationships among cytokines, adhesion molecules and AML status. They showed that the FLT3-ITD mutation in AML patients was significantly associated with the expression of E-selectin. (See Kupsa T. et al., Biomed Pap Med Fac Univ Palacky Olomouc Czech Repub., 160: 94-99 (2016), which is incorporated by reference in its entirety). The correlation of higher E-selectin expression in patients containing the FLT3-ITD mutation in their AML cells is strongly significant (P=0.0010) (See id., FIG. 50). The authors conclude that “both TNF-α and the activity of FLT3-ITD positive AML cells are key factors involved in endothelial cell activation.” (See id.). Endothelial activation results in expression of E-selectin.

### Example 257

Analysis of a public database of AML patients, which is known as TCGA (The Cancer Genome Atlas) from NCI containing 151 paired data with Overall Survival was performed and expression of the gene (FUT7) which codes for the fucosyltransferase that adds the terminal fucose required for binding activity of the E-selectin ligand, sialyl Le^{x} was correlated. This synthetic pathway is shown in FIG. 41.

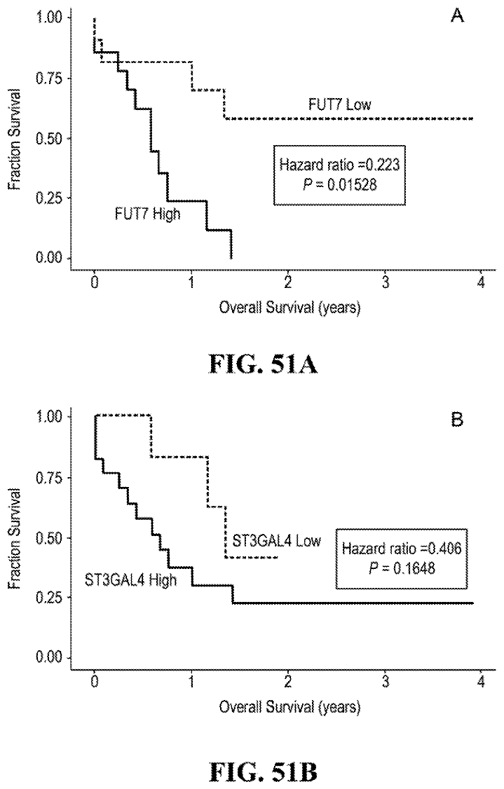

As discussed supra, FUT7 gene expression correlates to expression of the E-selectin ligand (sialyl Le^{x}) on the surface of AML cells in patients. As shown in FIG. 51A, poor survival is only observed in FLT3-ITD AML patients that express the E-selectin ligand as determined by FUT7 expression.

Correlation of poor survival with expression of the E-selectin ligand as determined by FUT7 expression in FLT3-ITD patients is statistically significant (P=0.015). This suggests that the binding of AML cells to E-selectin drives the poor survival observed with AML patients containing the FLT3 mutations.

Collectively, these studies extend the prognostic importance of the E-selectin ligand glycosylation genes, ST3GAL4 and FUT7, to adult AML, where these genes may be useful as predictive biomarkers. In addition, these studies suggest potential additional tumor types beyond AML where treatment protocols with E-selectin inhibitors may have therapeutic benefits.

### Example 258

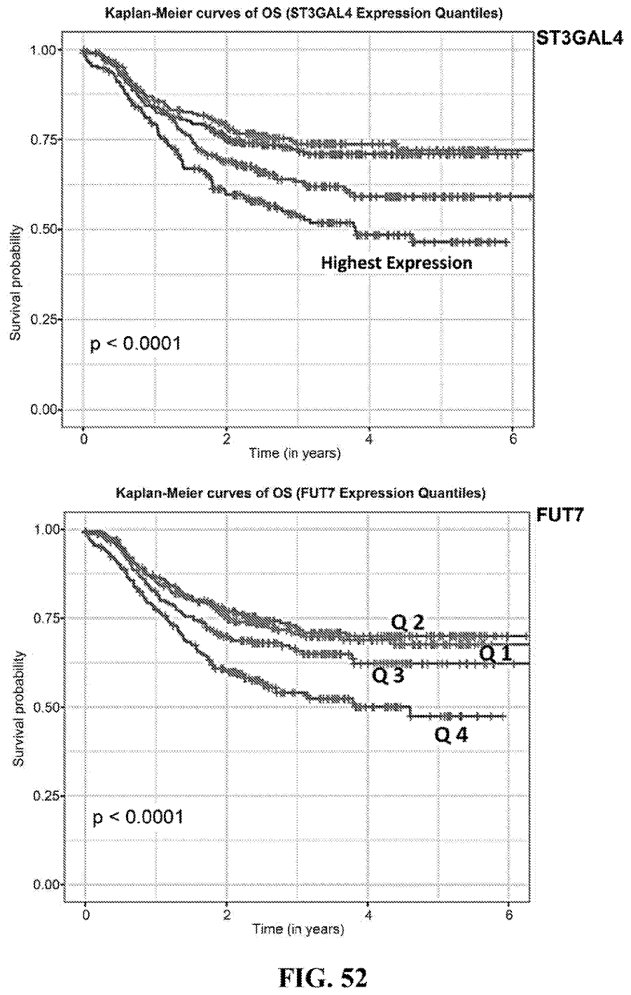

As shown in FIG. 52, a high coverage single strand mRNA sequencing was performed on samples from 1111 pediatric AML patients from the COG AAML1031 trial. The data from this analysis was screened for expression of the 24 different genes listed in FIG. 38. Expression was then correlated with the clinical outcome of overall survival (OS). Out of all the 24 genes evaluated, the expression of ST3GAL4 and FUT7 showed the strongest correlation with poor OS. The observed correlation was highly statistically significant (P<0.0001). FIG. 52 shows the correlation of gene expression with OS in each quartile for ST3GAL4 and FUT7.

### Example 259

The gene products of the ST3GAL4 and FUT7 genes, sialyltransferase ST3GAL4 (see Mondal N. et al., Blood 125: 687-696 (2015)) and fucosyltransferase FUT7 (see Maly P. et al., Cell 86: 643-653 (1996)), respectively, are known to add terminal sugars to synthesize the E-selectin ligand, sialyl Le^{x}, as shown in FIG. 41.

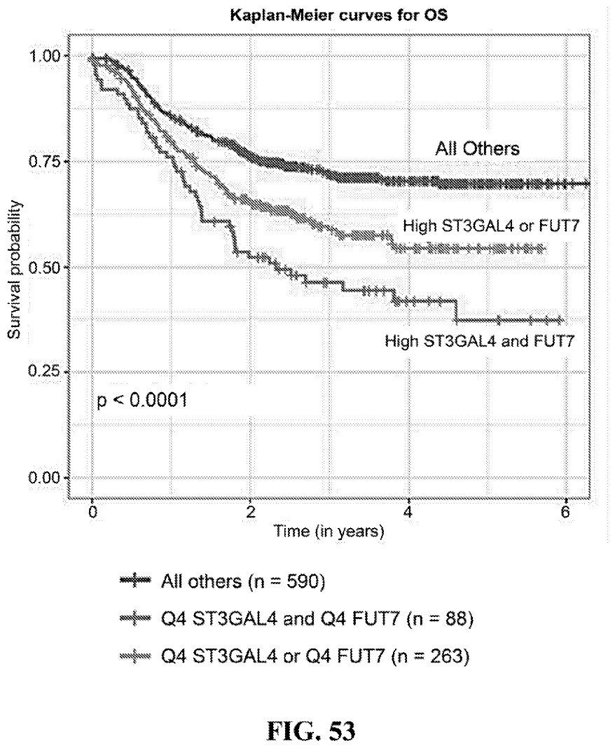

The database of gene expression from AML patients was therefore screened for the expression of both ST3GAL4 and FUT7 and correlated with OS. As can be seen in FIG. 53, expression of both genes is more strongly correlated with poor OS than expression of either one of these genes alone. The data clearly demonstrate that expression of the genes that synthesize the E-selectin ligand sialyl Le^{x} correlates strongly with poor survival. This supports the role of E-selectin in chemoresistance of AML blasts.

### Example 260

These data support the observation that cancer patients that express high levels of the E-selectin ligand (sialyl Le^{a/x}) on their tumors have a poorer outcome, as was reviewed in a meta-analysis of over ten years of publications on the role of sialyl Le^{x} in cancer. In this review, the authors conclude that “our meta-analysis showed that a high level of sialyl Le^{x} expression was significantly associated with lymphatic invasion, venous invasion, deep invasion, lymph node metastasis, distant metastasis, tumor stage, tumor recurrence, and OS in cancer.” Liang J. et al., Oncotargets and Therapy 9: 3113-3125 (2016).

Interestingly, those relapsed/refractory AML patients expressing high levels of sialyl Le^{x} on their blasts show the greatest therapeutic response when treated with the compound of Formula I. This clinical observation supports the action of the compound of Formula I in inhibiting the binding of tumor sialyl Le^{x} to E-selectin and preventing or breaking the E-selectin mediated chemoresistance as well.

### Example 261

Expression of E-selectin ligand on AML blasts: AML blasts from relapsed patients were isolated. The expression of E-selectin ligand was measured by immunofluorescence. As shown in FIG. 37, blasts from relapsed patients express significantly higher levels of E-selectin ligand compared to blasts from newly diagnosed patients, as measured by the mean fluorescence intensity (p value=0.0040).

### Example 262

Phase I/II trial of Formula I in combination with chemotherapy for AML: A specific glycomimetic antagonist of E-selectin (Formula I) was rationally designed based on the bioactive conformation of sialyl Le^{a/x} in the binding site of E-selectin. Treatment of AML relapsed/refractory patients with the compound of Formula I and chemotherapy were evaluated for response. In a Phase I trial, 19 patients were treated with the compound of Formula I twice a day at 5 mg/kg (n=6), 10 mg/kg (n=7), and 20 mg/kg doses (n=6), in combination with induction MEC (mitoxantrone, epotoside, and cytarabine) chemotherapy. In a Phase II trial, the treatment regimen comprised one 10 mg/kg dose of the compound of Formula I 24 hours prior to chemotherapy, then twice daily 10 mg/kg doses of the compound of Formula I throughout either MEC (mitoxantrone, etoposide, and cytarabine) or 7+3 (cytarabine for 7 days followed by 3 days of daunorubicin, idarubicin or mitoxantrone) chemotherapy up till 48 hours post-chemotherapy. AML blasts from the patients' bone marrow and peripheral blood were isolated, and the expression of E-selectin ligand on the blasts was measured using immunofluorescence. Patient response to treatment was also assessed.

For patients with relapsed or refractory AML, the response rate (CR/CRi) was 41% and this was higher than expected given the high-risk cytogenetic and other disease features. After a single course of induction treatment with the compound of Formula I, a higher CR/CRi rate (47%) was seen compared to historical controls of similar populations treated with MEC. The durability of response was sufficient to allow patients to proceed to stem cell transplant (n=9).

Interestingly, those patients with higher percentages of AML blasts expressing E-selectin ligands either in the BM (FIG. 45) or in the peripheral blood (FIG. 46) were more likely to have a complete response compared with those patients with lower percentages of blasts expressing E-selectin ligand. HECA-452 is a monoclonal antibody (mAb) that recognizes sialylated-Le^{x}. As shown in FIG. 45, patients with higher percentages of bone marrow AML blasts reactive to the anti-sialyl Le^{a/x} antibody HECA-452 were more likely (p=0.004) to experience complete response (CR) to treatment. Patients with lower percentages of HECA-452 reactive blasts were more likely to have progressive disease (PD), partial response (PR), morphologic leukemia free state (MLFS), or complete response with incomplete hematologic recovery (CRi).

Similarly, FIG. 46 shows that patients with higher expression of E-selectin ligand on blasts in the peripheral blood were more likely to have a complete response (CR) or complete response with incomplete hematologic recovery (CRi), while patients with lower expression of E-selectin ligand were more likely to have progressive disease (PD). Measurements were done at 12 hours and 48 hours after treatment with a compound of Formula I.

The association was also observed to contribute to better overall survival (OS). As shown in FIG. 47, treatment with the compound of Formula I has a much greater effect on extending overall survival to those patients whose AML blasts express higher levels of the E-selectin ligand as determined by binding to the anti-sialyl Le^{a/x} antibody, HECA-452. Patients with less than 10% blasts expressing the E-selectin ligand (“low expressers”) have an OS of 5.2 months. Patients with greater than 10% of blasts expressing the E-selectin ligand (“high expressers”) have an OS of 12.7 months. The clear benefit (highly significant, p=0.0056) of treatment with the compound of Formula I is observed in patients expressing the E-selectin ligand, as their E-selectin-mediated chemoresistance is broken by treatment with the E-selectin antagonist, the compound of Formula I. Without being bound by theory, treatment with the E-selectin antagonist, Formula I, may disrupt E-selectin-mediated chemoresistance in patients expressing higher levels of E-selectin ligand (≥10%). Conversely, those patients with lower percentages (<10%) of blasts expressing E-selectin may be chemoresistant (relapsed/refractory) by a different mechanism not involving E-selectin and therefore, Formula I showed less efficacy with a significantly lower OS (5.2 months).

### Example 263

Biomarkers for clinical outcome and overall survival: High coverage single strand mRNA sequencing was performed on clinical samples from 1111 pediatric AML patients (0 to 30 years old) from the COG AAML1031 trial. The data from this analysis was screened for expression of the 24 different genes listed in FIG. 38. Expression was then correlated with the clinical outcome of overall survival (OS). Out of the 24 genes evaluated, the expression of ST3GAL4 and FUT7 showed the strongest correlation with poor OS that was highly statistically significant (P<0.0001). FIG. 52 shows the correlation of gene expression with OS binned in each quartile for ST3GAL4 or FUT7 expression. As shown, the overall survival probability is decreased when the expression of ST3GAL4 or FUT7 is increased. For example, the 25% of patients with the highest expression of ST3GAL4 (Q4) have survival probability of less than 0.5 after 5 years.

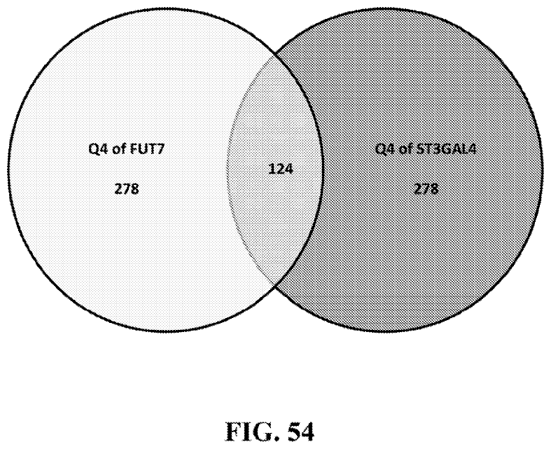

The difference in survival probability is starker when the highest-expressing quartiles of ST3GAL4 and FUT7 patients are compared to all other patients. As shown in FIG. 53, patients with the highest-expressing quartiles of ST3GAL4 and FUT7 have lower survival probability than patients with the highest-expressing quartiles of ST3GAL4 or FUT7, who in turn have lower survival probability when compared to all other patients. The number of patients shared between the highest-expressing quartile of ST3GAL4 and FUT7 are shown in FIG. 54.

### Example 264

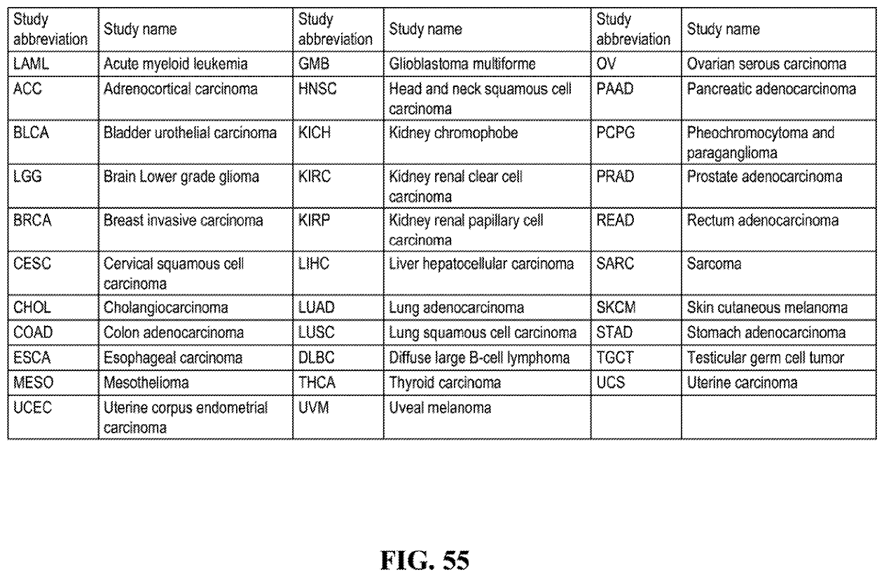

Clinical and RNAseq expression data for 10,258 samples covering 33 cancer types from the PanCanAtlas of The Cancer Genome Atlas (TCGA) were accessed via the NIH Genomics Data Commons (GDC) (FIG. 55).

The number of samples from each tumor type varied, ranging from 45 samples which were available for cholangiocarcinoma (CHOL) to 1,188 for breast invasive carcinoma (BRCA), with a median of 198 samples/tumor type.

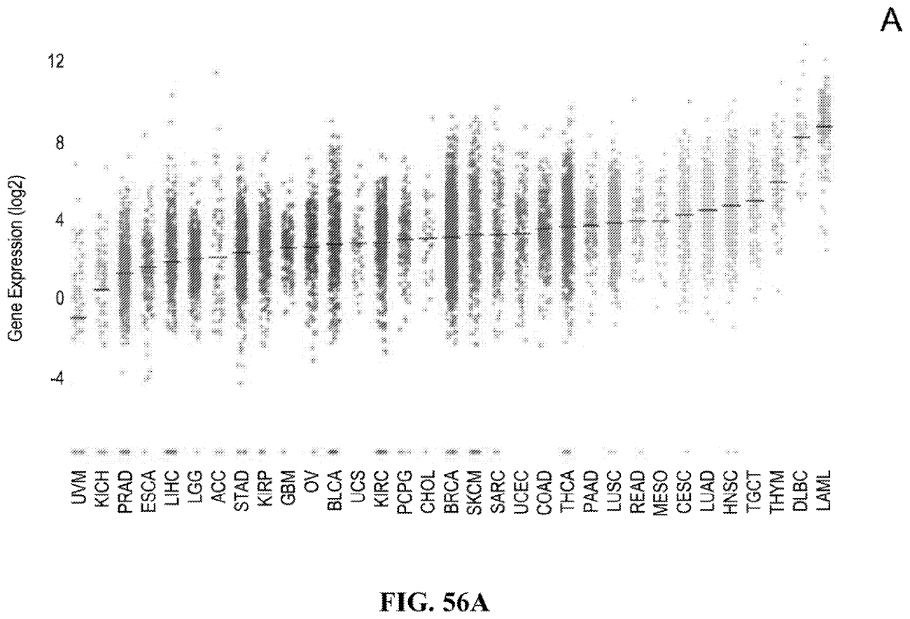

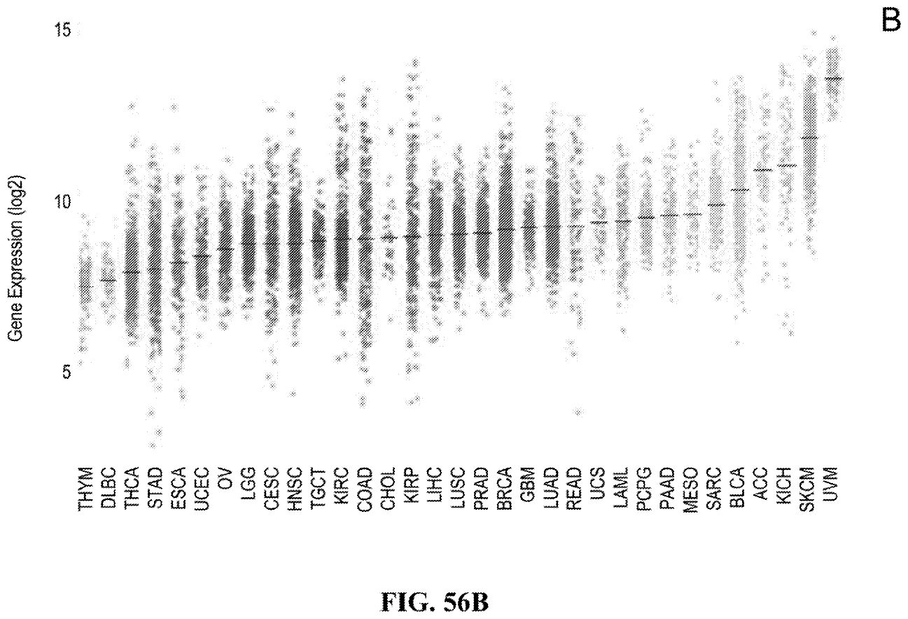

Expression data was log 2 transformed. (FIGS. 56A and 56B). Where no sequencing reads for a gene were detected in a sample, a low (non-zero) value was assigned to that sample by the PanCanAtlas project. In many genes this can be seen as a line at the bottom of the plot for particular cancer types. The black bar in each tumor type represents mean expression.

The E-selectin ligand glycosylation genes, FUT7 and ST3GAL4 are consistently expressed in the majority of cancer subtypes. The top five cancer types, based in mean expression:
FUT7: Acute Myeloid Leukemia (LAML), Lymphoid Neoplasm Diffuse Large B cell Lymphoma (DBLC), Thymoma (THYM), Testicular Germ Cell Tumors (TGCT), and Head and Neck Squamous Cell Carcinoma (HNSC).
ST3GAL4: Uveal Melanoma (UVM), Skin Cutaneous Melanoma (SKCM), Kidney Chromophobe (KICH), Adrenocortical Carcinoma (ACC), and Bladder Urothelial Carcinoma.

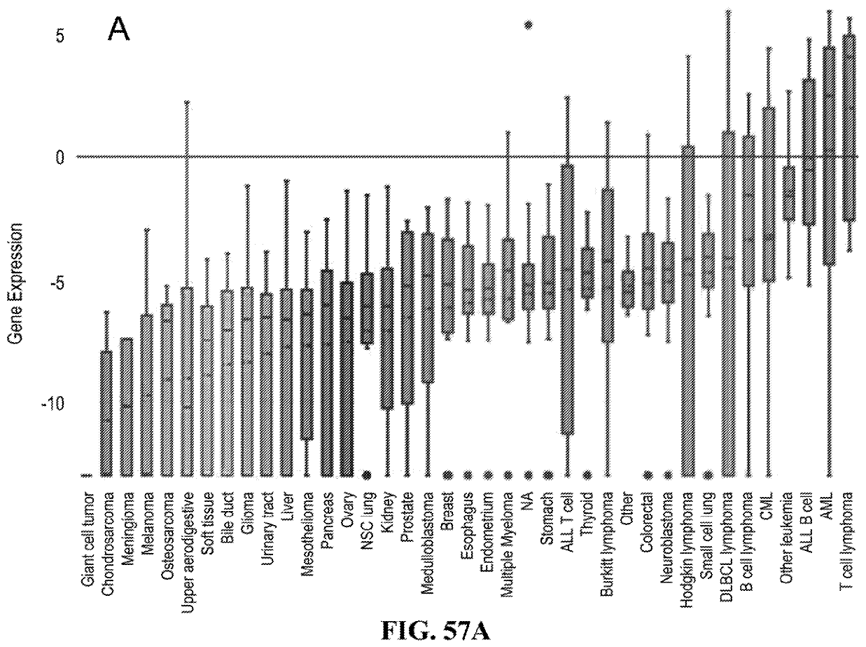

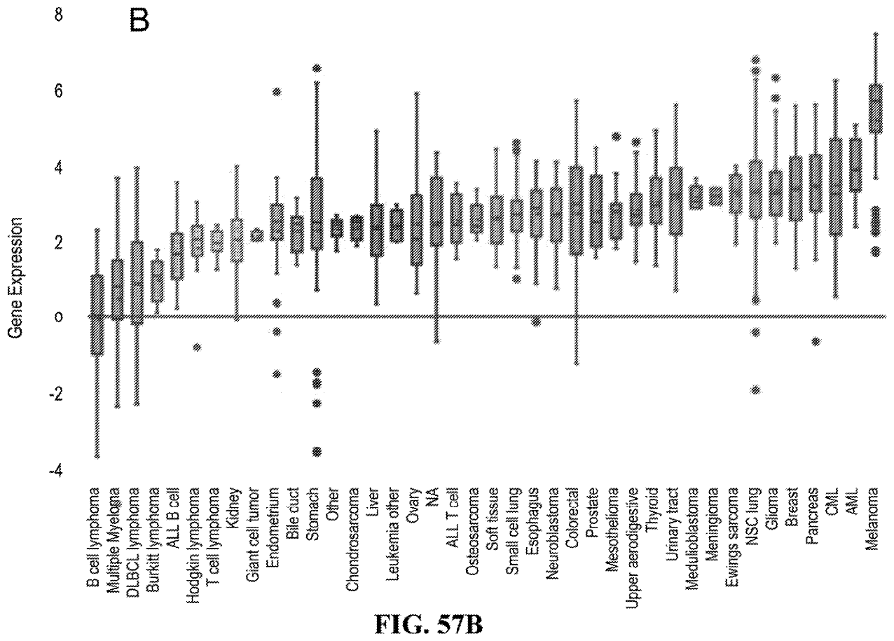

The E-selectin ligan glycosylation genes, FUT7 and ST3GAL4 are also consistently expressed in tumor cell lines comprising the Cancer Cell Line Encyclopedia database (FIGS. 57A and 57B). The top five cancer types, based on mean expression:
FUT7: T-cell Lymphoma, AML, B-cell Acute Lymphoblastic Leukemia, Other Leukemias and Chronic Myelogenous Leukemia (CML).
ST3GAL4: Melanoma, AML, CML, Pancreas, and Breast.

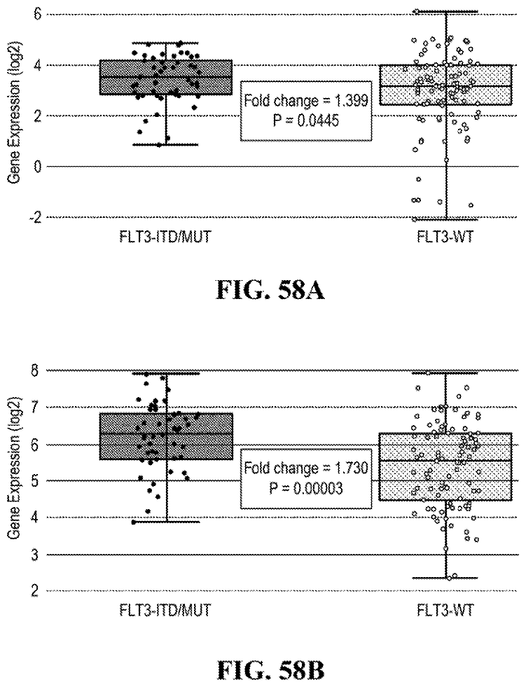

The TCGA-LAML RNAseq dataset was characterized for expression of FUT7 and ST3GAL4. (FIGS. 58A and 58B). The dataset included 142 RNAseq profiles of bone marrow samples from patients with AML with corresponding survival time data, and within this dataset the status of the FMS-like tyrosine kinase 3 (FLT3) proto-oncogene was considered. Samples characterized by FLT3 internal tandem duplication (ITD) were obtained from Rustagi et al BMC Bioinformatics, 2016 and FLT mutation (MUT) status (SNP, INS, or DEL) from the TCGA research network paper supplementary material, also available on the TCGA website. The remaining samples were classified as FLT3 wild-type (WT). The total number of samples in the FLT3-WT and the FLT3-ITD/MUT groups were 96 and 46, respectively.

Survival analysis was performed with the Cox proportional model, associating the expression levels of FUT7 and ST3GAL4 with overall survival (OS) (FIGS. 51A and 51B) using the FLT3-ITD sample set. Expression levels for each gene were dichotomized (high or low) using the median expression value over the full set of samples.

These studies extend the prognostic importance of the E-selectin ligand glycosylation genes FUT7 and ST3GAL4 to adult AML. AML patients harboring the FLT3 ITD mutation with high expressions of FUT7 and ST3GAL4 experience poor survival compared to patients with low expression of FUT7 and ST3GAL4. These studies suggest additional tumor types beyond AML where treatment protocols with an E-selectin antagonist of Formula I may have therapeutic benefits.

1. A method of screening a cancer patient for treatment, the method comprising:
(a) obtaining or having obtained a biological sample comprising blast cells from the cancer patient;
(b) performing or having performed an assay on the biological sample to determine the gene expression level of one or more E-selectin ligand-forming genes in the sample; and
(c) if the blast cells in the sample have an increased expression level of the one or more E-selectin ligand-forming genes relative to a control sample from a non-cancer subject, a newly-diagnosed cancer subject, or a subject having the same cancer as the patient, or
if at least 10% of the blast cells in the sample express the one or more E-selectin ligand-forming genes,
then selecting the patient for treatment comprising one or more E-selectin inhibitors.

2. The method according to claim 1, wherein the cancer patient is a relapsed cancer patient.

3. The method according to claim 1, wherein the cancer patient has a cancer chosen from solid tumors and liquid tumors.

4. The method according to claim 1, wherein the cancer patient has one or more cancers chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

5. The method according to claim 1, wherein the cancer patient has one or more cancers chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

6. The method according to claim 5, wherein the leukemia is chosen from acute myeloid leukemia, acute lymphocytic leukemia, chronic lymphocytic leukemia, and chronic myelogenous leukemia.

7. The method according to claim 5, wherein the lymphoma is chosen from non-Hodgkins lymphoma and Hodgkins lymphoma.

8. The method according to claim 5, wherein the myeloma is multiple myeloma.

9. The method according to claim 5, wherein the melanoma is chosen from uvual melanoma and skin melanoma.

10. The method according to claim 1, wherein the one or more E-selectin ligand-forming genes are glycosylation genes.

11. The method according to claim 1, wherein the one or more E-selectin-ligand forming genes are chosen from ST3GAL3, ST3GAL4, FUCA2, FUT5, and FUT7.

12. The method according to claim 1, wherein the one or more E-selectin-ligand forming genes are chosen from ST3GAL4, FUT5, and FUT7.

13. The method according to claim 1, wherein the one or more E-selectin-ligand forming genes are chosen from ST3GAL4 and FUT7.

14. The method according to claim 1, wherein at least one of the one or more E-selectin-ligand forming genes is ST3GAL4.

15. The method according to claim 1, wherein at least one of the one or more E-selectin-ligand forming genes is FUT7.

16. The method according to claim 1, wherein the method further includes determining the presence of one or more mutational alterations of FLT3.

17. The method according to claim 16, wherein the mutational alterations are chosen from internal tandem duplications and missense mutations within the tyrosine kinase domain activation loop of FLT3.

18. The method according to claim 1, wherein the sample is a bone marrow sample.

19. The method according to claim 1, wherein the sample is a peripheral blood sample.

20. The method according to claim 1, wherein performing or having performed an assay on the biological sample to determine the gene expression level of one or more E-selectin ligand-forming genes in the sample further comprises measuring the number of mRNA transcripts or the amount of protein expressed.

21. The method according to claim 20, wherein the assay is chosen from Sanger sequencing, high throughput sequencing, quantitative polymerase chain reaction, reverse transcriptase qPCR, RNA sequencing, microarray analysis, Northern blots, RNA-seq, high coverage mRNA sequencing, flow analysis, flow cytometry, immunohistology, immunostaining, immunohistochemistry, affinity purification, mass spectrometry, Western blotting, enzyme-linked immunoadsorbent assay, and multidimensional flow cytometry.

22. The method according to claim 21, wherein the assay uses reagents chosen from a HECA-452-FITC monoclonal antibody, an E-selectin/hIg chimera, and chimera/PE.

23. The method according to claim 1, wherein the one or more E-selectin inhibitors are chosen from compounds of Formula I: 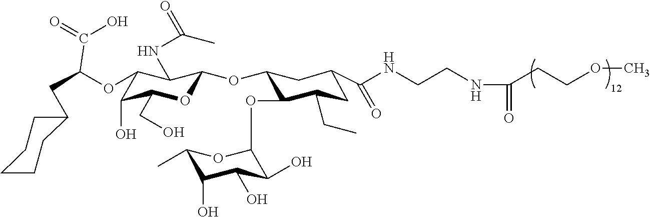
and pharmaceutically acceptable salts thereof.

24. The method according to claim 23, wherein the patient selected for treatment comprising one or more E-selectin inhibitors is being treated with chemotherapy and/or radiotherapy.

25. The method according to claim 23, wherein the patient selected for treatment comprising one or more E-selectin inhibitors is being treated with one or more anti-cancer agents.

26. The method according to claim 25, wherein the one or more anti-cancer agents are chosen from mitoxantrone, etoposide, cytarabine, daunomycin, idarubicin, cyclophosphamide, methotrexate, 6-mercaptopurine, 6-thioguanine, aminopterin, arsenic trioxide, asparaginase, cladribine, clofarabine, cyclophosphamide, cytosine arabinoside, dasatinib, decitabine, dexamethasone, fludarabine, gemtuzumab ozogamicin, imatinib mesylate, interferon-α, interleukin-2, melphalan, nelarabine, nilotinib, oblimersen pegaspargase, pentostatin, ponatinib, prednisone, rituximab, tretinoin, and vincristine.

27. A method of treating a cancer patient, the method comprising:
(a) obtaining or having obtained a biological sample comprising blast cells from the cancer patient;
(b) performing or having performed an assay on the biological sample to determine the gene expression level of one or more E-selectin ligand-forming genes in the sample; and
(c) if the blast cells in the sample have an increased gene expression level of the one or more E-selectin ligand-forming genes relative to a control sample from a non-cancer subject, a newly-diagnosed cancer subject, or a subject having the same cancer as the patient, or
if at least 10% of the blast cells in the sample express the one or more E-selectin ligand-forming genes, then
administering a therapeutically effective amount of a composition comprising one or more E-selectin inhibitors.

28. The method according to claim 27, wherein the one or more E-selectin inhibitors are chosen from compounds of Formula I: 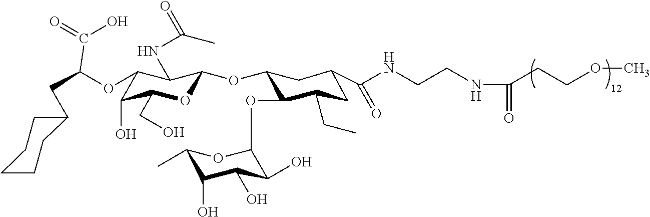
and pharmaceutically acceptable salts thereof.

29. The method according to 28, wherein the patient to whom the one or more E-selectin inhibitors are administered is being further treated with chemotherapy and/or radiotherapy.

30. The method according to claim 28, wherein the patient to whom the one or more E-selectin inhibitors are administered is also being administered one or more anti-cancer agents.

31. The method according to claim 30, wherein the one or more anti-cancer agents are chosen from mitoxantrone, etoposide, cytarabine, daunomycin, idarubicin, cyclophosphamide, methotrexate, 6-mercaptopurine, 6-thioguanine, aminopterin, arsenic trioxide, asparaginase, cladribine, clofarabine, cyclophosphamide, cytosine arabinoside, dasatinib, decitabine, dexamethasone, fludarabine, gemtuzumab ozogamicin, imatinib mesylate, interferon-α, interleukin-2, melphalan, nelarabine, nilotinib, oblimersen pegaspargase, pentostatin, ponatinib, prednisone, rituximab, tretinoin, and vincristine.

32. The method according to claim 28, wherein the cancer patient is a relapsed cancer patient.

33. The method according to claim 28, wherein the cancer patient has a cancer chosen from solid tumors and liquid tumors.

34. The method according to claim 28, wherein the cancer patient has one or more cancers chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

35. The method according to claim 28, wherein the cancer patient has one or more cancers chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

36. The method according to claim 35, wherein the leukemia is chosen from acute myeloid leukemia, acute lymphocytic leukemia, chronic lymphocytic leukemia, and chronic myelogenous leukemia.

37. The method according to claim 35, wherein the lymphoma is chosen from non-Hodgkins lymphoma and Hodgkins lymphoma.

38. The method according to claim 35, wherein the myeloma is multiple myeloma.

39. The method according to claim 35, wherein the melanoma is chosen from uvual melanoma and skin melanoma.

40. The method according to claim 28, wherein the one or more E-selectin ligand-forming genes are glycosylation genes.

41. The method according to claim 28, wherein the one or more E-selectin-ligand forming genes are chosen from ST3GAL3, ST3GAL4, FUCA2, FUT5, and FUT7.

42. The method according to claim 28, wherein the one or more E-selectin-ligand forming genes are chosen from ST3GAL4, FUT5, and FUT7.

43. The method according to claim 28, wherein the one or more E-selectin-ligand forming genes are chosen from ST3GAL4 and FUT7.

44. The method according to claim 28, wherein at least one of the one or more E-selectin-ligand forming genes is ST3GAL4.

45. The method according to claim 28, wherein at least one of the one or more E-selectin-ligand forming genes is FUT7.

46. The method according to claim 28, wherein the method further includes determining the presence of one or more mutational alterations of FLT3.

47. The method according to claim 46, wherein the mutational alterations are chosen from internal tandem duplications and missense mutations within the tyrosine kinase domain activation loop of FLT3.

48. The method according to claim 28, wherein the sample is a bone marrow sample.

49. The method according to claim 28, wherein the sample is a peripheral blood sample.

50. The method according to claim 28, wherein performing or having performed an assay on the biological sample to determine the gene expression level of one or more E-selectin ligand-forming genes in the sample further comprises measuring the number of mRNA transcripts or the amount of protein expressed.

51. The method according to claim 28, wherein the assay is chosen from Sanger sequencing, high throughput sequencing, quantitative polymerase chain reaction, reverse transcriptase qPCR, RNA sequencing, microarray analysis, Northern blots, RNA-seq, high coverage mRNA sequencing, flow analysis, flow cytometry, immunohistology, immunostaining, immunohistochemistry, affinity purification, mass spectrometry, Western blotting, enzyme-linked immunoadsorbent assay, and multidimensional flow cytometry.

52. The method according to claim 28, wherein the assay uses reagents chosen from a HECA-452-FITC monoclonal antibody, an E-selectin/hIg chimera, and chimera/PE.
